(19) United States
(12) Patent Application Publication
(10) Pub. No.: US 2026/0049089 A1
(43) Pub. Date: Feb. 19, 2026

(54) KRAS INHIBITOR COMPOUND

(71) Applicant: SHANGHAI APEIRON THERAPEUTICS COMPANY LIMITED, Shanghai (CN)

(72) Inventors: Yisong XIAO, Shanghai (CN); Kunmin LAI, Shanghai (CN); Xiaohui GU, Shanghai (CN)

(73) Assignee: SHANGHAI APEIRON THERAPEUTICS COMPANY LIMITED, Shanghai (CN)

(21) Appl. No.: 18/996,024

(22) PCT Filed: Jul. 28, 2023
(86) PCT No.: PCT/CN2023/109750

§ 371 Date: Jan. 17, 2025

(30) Foreign Application Priority Data
Jul. 28, 2022 (CN) 202210900237.X
Sep. 28, 2022 (CN) 202211193916.4
Jan. 3, 2023 (CN) 202310004924.8
Apr. 14, 2023 (CN) 202310403398.2
May 23, 2023 (CN) 202310590062.1

(51) Int. Cl. C07D 487/08; A61K 31/517; A61K 31/5377; C07B 59/00; C07D 519/00

(52) U.S. Cl. CPC C07D 487/08; A61K 31/517; A61K 31/5377; C07B 59/002; C07D 519/00; C07B 2200/05

(57) ABSTRACT

A KRAS inhibitor compound and use thereof are provided. Specifically, a heterocyclic compound represented by Formula I-1 or Formula I-2, a pharmaceutically acceptable salt thereof, a stereoisomer thereof, a deuterate thereof, or a solvate thereof are provided. The KRAS inhibitor compound exhibits a novel structure with superior activity and selectivity.

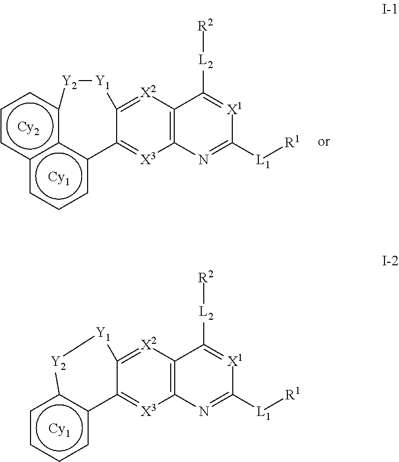

### CROSS REFERENCE TO THE RELATED APPLICATIONS

This application is the national phase entry of International Application No. PCT/CN2023/109750, filed on Jul. 28, 2023, which is based upon and claims priority to Chinese Patent Application No. 202210900237.X, filed on Jul. 28, 2022, Chinese Patent Application No. 202211193916.4, filed on Sep. 28, 2022, Chinese Patent Application No. 202310004924.8, filed on Jan. 3, 2023, Chinese Patent Application No. 202310403398.2, filed on Apr. 14, 2023, and Chinese Patent Application No. 202310590062.1, filed on May 23, 2023, the entire contents of which are incorporated herein by reference.

### TECHNICAL FIELD

The present invention relates to the field of medicine, specifically to a KRAS inhibitor compound and use thereof.

### BACKGROUND

Mutations in the RAS oncogene are the most common activating mutations in human cancers, occurring in 30% of human tumors. The RAS gene family consists of three subtypes: KRAS, HRAS, and NRAS. Among these, 85% of RAS-driven cancers are caused by mutations in the KRAS subtype. KRAS mutations are frequently observed in solid tumors such as lung adenocarcinoma, pancreatic ductal adenocarcinoma, and colorectal cancer. In KRAS-mutant tumors, 80% of oncogenic mutations occur at codon 12, with the most common mutations being p.G12D (41%), p.G12V (28%), and p.G12C (14%).

The full name of the KRAS gene is Kirsten rat sarcoma viral oncogene homolog. KRAS plays a pivotal role in regulating signaling pathways that control cell growth. Upstream receptors, such as EGFR (ErbB1), HER2 (ErbB2), ErbB3, and ErbB4, transmit extracellular signals to downstream pathways through RAS proteins. In its inactive state, the KRAS protein is tightly bound to GDP (guanosine diphosphate). Upon activation by guanine nucleotide exchange factors such as SOS1, KRAS binds to GTP (guanosine triphosphate), switching to its active kinase state. Mutations in the KRAS gene enable it to transmit growth and proliferation signals to downstream pathways independently of upstream growth factor receptor signaling. This leads to uncontrolled cell growth and tumor progression. KRAS mutation status is also a significant prognostic marker in cancers. Statistical analyses indicate that among KRAS subtypes, KRAS G12D is a common submutation, accounting for 12% of colorectal cancers, 36% of pancreatic cancers, and 4% of non-small cell lung cancers. Therefore, the development of novel KRAS inhibitors is of great importance. These inhibitors hold substantial potential as new therapeutic options in the field of oncology. There is a pressing need to develop more effective, safer, and pharmacokinetically favorable KRAS inhibitors to meet clinical demands.

### SUMMARY

The present invention provides a KRAS inhibitor compound and use thereof. Specifically, disclosed is a heterocyclic compound represented by Formula I-1 or Formula I-2, a pharmaceutically acceptable salt thereof, a stereoisomer thereof, a deuterate thereof, or a solvate thereof. The compound of the present invention exhibits a novel structure with superior activity and selectivity.

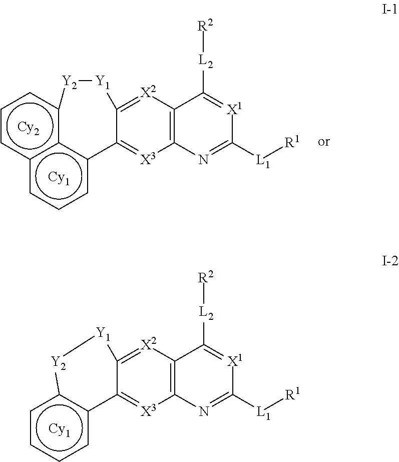

wherein, in Formula I-1 and Formula I-2, Cy₁ represents a 6-membered aryl group or a 6-membered heteroaryl group; further, Cy₁ is optionally substituted with 0-3 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein, in Formula I-1, Cy₂ represents a 6-membered aryl group or a 6-membered heteroaryl group; further, Cy₂ is optionally substituted with 0-3 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein X¹ is N or CR^{X1}; X² is N or CR^{X2}; X³ is N or CR^{X3};
wherein R^{X1}, R^{X2}, and R^{X3} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein L₁ and L₂ each independently represent absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀-C₆ alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR—(C₁-C₆ alkylene)-, —(C₁-C₆ alkylene)-NR^{a}—, —(C₁-C₆ alkylene)-C(O)—, —NR^{a}C(O)(C₁-C₆ alkylene)-, —(C₁-C₆ alkylene)-C(O)NR^{a}—, —O—(C₀-C₆ alkylene)-(CR^{T}R^{T′})—(C₀-C₆ alkylene)-, wherein R^{T} and R^{T′} together with the carbon atom to which they are attached form a 3-6 membered saturated or unsaturated ring optionally containing 0, 1, or 2 heteroatoms selected from O, N, and S;
wherein R¹ and R² each independently represent a 5-16 membered saturated or unsaturated cycloalkyl or a 5-16 membered saturated or unsaturated heterocycloalkyl, optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein Y¹ and Y² together form a C₁-C₁₀ alkylene or C₂-C₁₀ alkenylene, wherein CR^{a}R^{b} may be optionally substituted with O, NH, —C(O)—, —OC(O)—, —C(O)O—, —CONR^{a}—, —NR^{a}CO—, —N(S(O)₂CH₃)—, —N(C(O)CH₃)—, —S(O)—, —S(O)₂—, —P(O)R^{a}—;
or, Y¹ and Y² together form a 5-10 membered saturated or unsaturated ring, a 6-10 membered aromatic ring, or a 5-10 membered heteroaromatic ring;
wherein R^{a} and R^{b} each independently represent hydrogen, C₁-C₆ alkyl, halogenated (C₁-C₆ alkyl), or C₃-C₆-cycloalkyl; or R^{a} and R^{b} together with the atom to which they are attached form a 3-6 membered ring optionally containing 0, 1, or 2 heteroatoms selected from O, N, and S.

In addition, the present invention provides a heterocyclic compound represented by Formula I-1′ or Formula I-2′, a pharmaceutically acceptable salt thereof, a stereoisomer thereof, a deuterate thereof, or a solvate thereof:

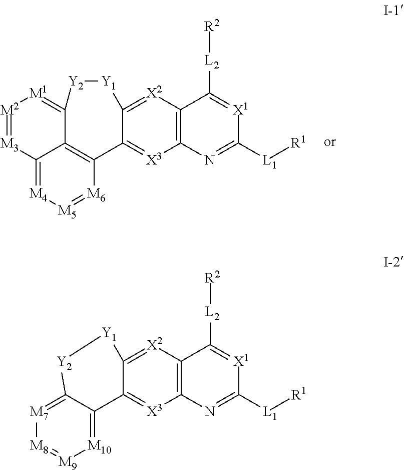

wherein, in Formula I-1′ and Formula I-2′,
M¹ represents CR^{M1} or N; M² represents CR^{M2} or N; M³ represents CR^{M3} or N; M⁴ represents CR^{M4} or N; M⁵ represents CR^{M5} or N; M⁶ represents CR^{M6} or N; M⁷ represents CR^{M7} or N; M⁸ represents CR^{M8} or N; M⁹ represents CR^{M9} or N; M¹⁰ represents CR^{M10} or N;
wherein each of R^{M1}, R^{M2}, R^{M3}, R^{M4}, R^{M5}, R^{M6}, R^{M7}, R^{M8}, R^{M9}, and R^{M10} independently represents hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}, optionally substituted with substituents;
wherein X¹ is N or CR^{X1}; X² is N or CR^{X2}; X³ is N or CR^{X3};
wherein R^{X1}, R^{X2}, and R^{X3} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein L₁ and L₂ each independently represent absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀-C₆ alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR^{a}—(C₁-C₆ alkylene)-, —(C₁-C₆ alkylene)-NR^{a}—, —(C₁-C₆ alkylene)-C(O)—, —NR^{a}C(O)(C₁-C₆ alkylene)-, —(C₁-C₆ alkylene)-C(O)NR^{a}—, —O—(C₀-C₆ alkylene)-(CR^{T}R^{T′})—(C₀-C₆ alkylene)-, wherein R^{T} and R^{T′} together with the carbon atom to which they are attached form a 3-6 membered saturated or unsaturated ring optionally containing 0, 1, or 2 heteroatoms selected from O, N, and S;
wherein R¹ and R² each independently represent a 5-16 membered saturated or unsaturated cycloalkyl or a 5-16 membered saturated or unsaturated heterocycloalkyl, optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein Y¹ and Y² together form a C₁-C₁₀ alkylene or C₂-C₁₀ alkenylene, and wherein CR^{a}R^{b} may be optionally substituted with O, NH, NR^{a}, —C(O)—, —OC(O)—, —C(O)O—, —CONR^{a}—, —NR^{a}CO—, —N(S(O)₂CH₃)—, —N(C(O)CH₃)—, —S(O)—, —S(O)₂—, or —P(O)R^{a}—,
or, Y¹ and Y² together form a 5-10 membered saturated or unsaturated ring, a 6-10 membered aromatic ring, or a 5-10 membered heteroaromatic ring;
wherein R^{a} and R^{b} each independently represent hydrogen, C₁-C₆ alkyl, halogenated (C₁-C₆ alkyl), or C₃-C₆-cycloalkyl; or R^{a} and R^{b} together with the atom to which they are attached form a 3-6 membered ring optionally containing 0, 1, or 2 heteroatoms selected from O, N, and S.

In a preferred embodiment of the present invention, wherein X² represents CH or N.

In a preferred embodiment of the present invention, wherein X³ represents CR^{X3} or N, and R^{X3} represents hydrogen, halogen, —OR^{a}, CN, —NO₂, —SF₅, —POMe₂, —NH₂, C₁-C₆ alkyl, halogenated (C₁-C₆ alkyl), halogenated (C₁-C₆ alkoxy), or hydroxy (C₁-C₆ alkyl).

In a preferred embodiment of the present invention, wherein L₁ represents absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀-C₆ alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR^{a}—(C₁-C₆ alkylene)-, —(C₁-C₆ alkylene)-NR^{a}—, or —O—(C₀-C₆ alkylene)-(CR^{T}R^{T′})—(C₀-C₆ alkylene)-, wherein R^{T} and R^{T′} together with the carbon atom to which they are attached form a 3-6 membered saturated or unsaturated ring optionally containing 0, 1, or 2 heteroatoms selected from O, N, and S.

In a preferred embodiment of the present invention, wherein L₁ represents —O—C₁-C₆ alkylene-.

In a preferred embodiment of the present invention, wherein L₁ represents —O—(C₀-C₆ alkylene)-(CR^{T}R^{T′})—(C₀-C₆ alkylene)-, wherein R^{T} and R^{T′} together with the carbon atom to which they are attached form a 3-6 membered saturated or unsaturated ring.

In a preferred embodiment of the present invention, wherein R¹ represents a 5-16 membered saturated or unsaturated heterocycloalkyl, optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

In a preferred embodiment of the present invention, wherein R¹ has any one of the following structures:

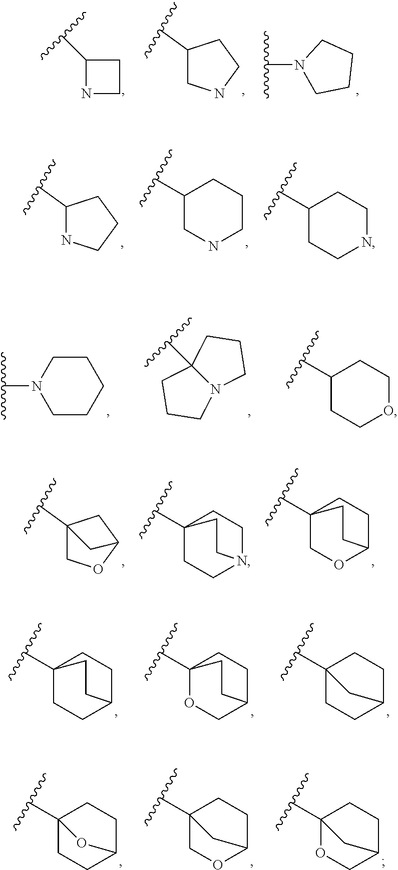

Furthermore, R¹ is optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

In a preferred embodiment of the present invention, wherein R¹ is defined as follows:

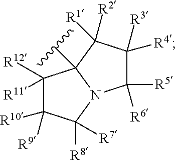

wherein R^{1′}, R^{2′}, R^{3′}, R^{4′}, R^{5′}, R^{6′}, R^{7′}, R^{8′}, R^{9′}, R^{10′}, R^{11′}, R^{12′} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
or R^{1′} and R^{2′} together with the carbon atom to which they are attached form a double bond (C═) or a C═O group, or R^{1′} and R^{2′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or R^{3′} and R^{4′} together with the carbon atom to which they are attached form a double bond (C═) or a C═O group, or R^{3′} and R^{4′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or R^{5′} and R^{6′} together with the carbon atom to which they are attached form a double bond (C═) or a C═O group, or R^{5′} and R^{6′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or R^{7′} and R^{8′} together with the carbon atom to which they are attached form a double bond (C═) or a C═O group, or R^{7′} and R^{8′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or R^{9′} and R^{10′} together with the carbon atom to which they are attached form a double bond (C═) or a C═O group, or R^{9′} and R^{10′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or R^{11′} and R^{12′} together with the carbon atom to which they are attached form a double bond (C═) or a C═O group, or R^{11′} and R^{12′} together with the atoms to which they are attached form a substituted or unsubstituted 3-10 membered saturated or unsaturated ring optionally containing 0, 1, or 2 heteroatoms selected from O, N, and S; the ring is further optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; any CH₂ in the ring may be substituted with a C═O group;
or, R^{2′} and R^{3′} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{4′} and R^{5′} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{8′} and R^{9′} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{10′} and R^{11′} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group.

In a preferred embodiment of the present invention, wherein R^{1′}, R^{2′}, R^{3′}, R^{4′}, R^{5′}, R^{6′}, R^{7′}, R^{8′}, R^{9′}, R^{10′}, R^{11′}, and R^{12′} each independently represent hydrogen, halogen, hydroxy, carbonyl, C₁-C₆ alkyl, halogenated (C₁-C₆ alkyl), halogenated (C₁-C₆ alkoxy), hydroxy (C₁-C₆ alkyl), C₂-C₆ alkenyl, or C₂-C₆ alkynyl.

In a preferred embodiment of the present invention, wherein R¹ has the following structure:

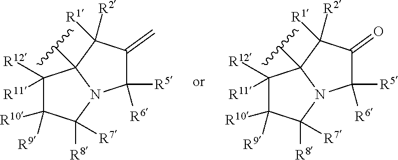

wherein R^{1′}, R^{2′}, R^{5′}, R^{6′}, R^{7′}, R^{8′}, R^{9′}, R^{10′}, R^{11′}, and R^{12′} are defined as in any one of claims.

In a preferred embodiment of the present invention, wherein R¹ has the following structure:

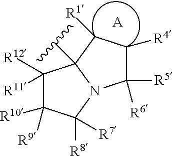

wherein ring A is a 3-6 membered ring, optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a};
wherein R^{1′}, R^{4′}, R^{5′}, R^{6′}, R^{7′}, R^{8′}, R^{9′}, R^{10′}, R^{11′}, and R^{12′} are defined as in any one of claims.

In a preferred embodiment of the present invention, wherein R¹ has the following structure:

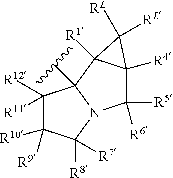

wherein R^{1′}, R^{2′}, R^{5′}, R^{6′}, R^{7′}, R^{8′}, R^{9′}, R^{10′}, R^{11′}, and R^{12′} are defined as in any one of claims, and R^{L} and R^{L′} each independently represent hydrogen, C₁-C₆ alkyl, or halogen.

In a preferred embodiment of the present invention, wherein R^{1′}, R^{2′}, R^{5′}, R^{6′}, R^{7′}, R^{8′}, R^{9′}, R^{10′}, R^{11′}, and R^{12′} each independently represent hydrogen, halogen, hydroxy, C₁-C₆ alkyl, halogenated (C₁-C₆ alkyl), halogenated (C₁-C₆ alkoxy), or hydroxy (C₁-C₆ alkyl); R^{3′} and R^{4′} together with the atoms to which they are respectively attached form a 3-6 membered ring, optionally containing 0, 1, or 2 heteroatoms selected from O, N, and S; further, the ring is optionally substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}:

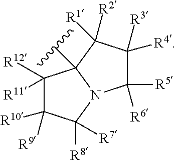

In a preferred embodiment of the present invention, wherein L₂ represents absent, —O—C₁-C₆ alkylene-, —C₁-C₆ alkylene-, —C₁-C₆ alkylene-O—, —NR^{a}—C₁-C₆ alkylene-, or —C₁-C₆ alkylene-NR^{a}—.

In a preferred embodiment of the present invention, wherein L₂ represents absent.

In a preferred embodiment of the present invention, wherein R² is any cyclic structure of Formula (i), Formula (ii), or Formula (iii):

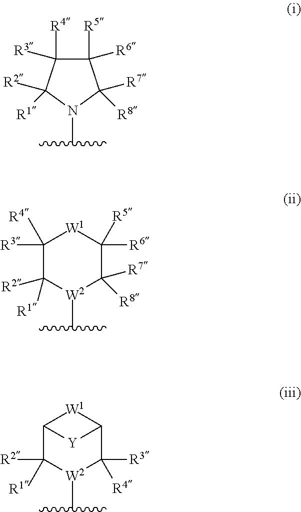

wherein W¹ represents —(CR^{W1}R^{W2})ₚ—, —(CR^{W1}R^{W2})ₚ—O—, —O—(CR^{W1}R^{W2})ₚ—, —NR^{a}—(CR^{W1}R^{W2})ₚ—, —(CR^{W1}R^{W2})ₚ—NR^{a}—, —CH═CH—, or —NR^{W1};
wherein R^{W1} and R^{W2}, each independently, represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}; or R^{W1} and R^{W2} together, with the carbon atom to which they are attached, form a 3-8 membered saturated or unsaturated ring optionally containing 0, 1, or 2 heteroatoms selected from O, N, and S. Furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
wherein R^{1″}, R^{2″}, R^{3′}, R^{4″}, R^{5″}, R^{6″}, R^{7″} and R^{8″} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
or, R^{1″} and R^{2″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR³, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{3″} and R^{4″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{5″} and R^{6″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{7″} and R^{8″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{2″} and R^{3″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{4″} and R^{5″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{6″} and R^{7″} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R³, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;
or, R^{5″} and R^{W1} together with the atoms to which they are respectively attached, form a substituted or unsubstituted 3-10 membered saturated or unsaturated ring, optionally containing 0, 1, or 2 heteroatoms selected from O, N, and S; furthermore, the ring is optionally substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; and any CH₂ in the ring may be substituted with a C═O group;

In a preferred embodiment of the present invention, wherein R² is any one of the following cyclic structures:

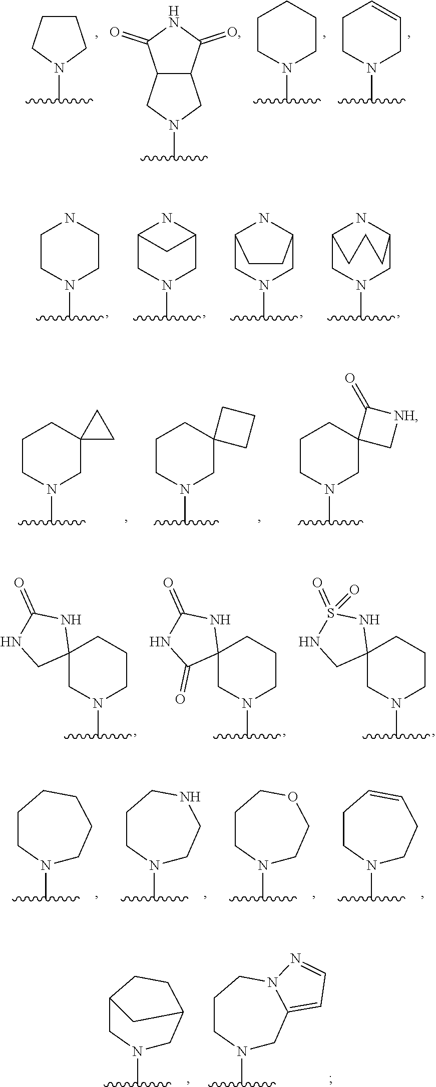

Furthermore, R² is optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

In a preferred embodiment of the present invention, wherein R² is any one of the following cyclic structures:

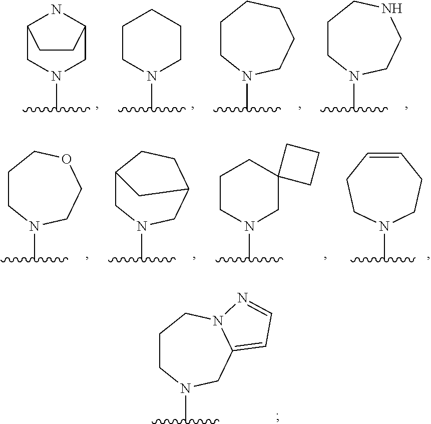

Furthermore, R² is optionally substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

In a preferred embodiment of the present invention, wherein R² is any one of the following cyclic structures:

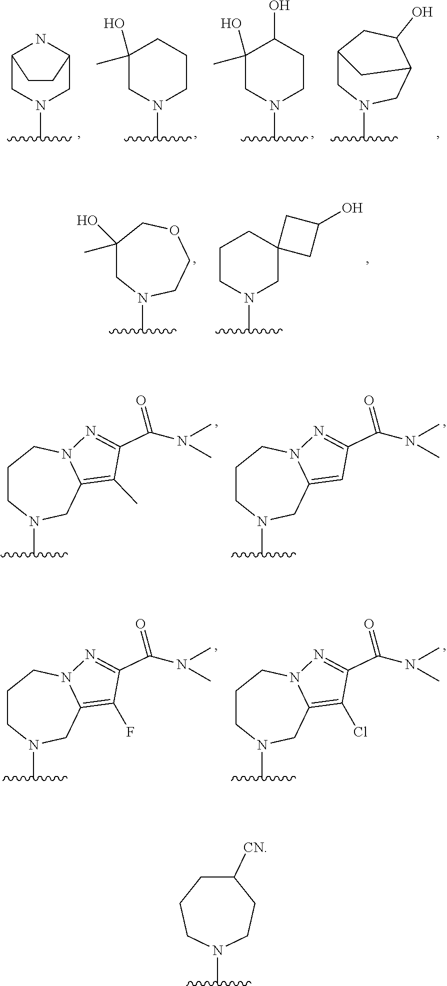

In a preferred embodiment of the present invention, wherein M¹ to M¹⁰ each represent CH or N.

In a preferred embodiment of the present invention, wherein M¹ to M¹⁰ each represent CH.

In a preferred embodiment of the present invention, wherein,
Y¹ and Y² together form a C₁-C₁₀ alkylene or a C₂-C₁₀ alkenylene, and CR^{a}R^{b} is optionally substituted with O, NH, —C(O)—, —OC(O)—, —C(O)O—, —S(O)—, —S(O)₂—, or —P(O)R^{a}—.

In a preferred embodiment of the present invention, wherein Y¹ and Y² together form a —(C₁-C₁₀ alkylene)-NR^{a}—, —O—(C₁-C₁₀ alkylene)-NR^{a}—, —NR^{a}(C₁-C₁₀ alkylene)-O—, —(C₁-C₁₀ alkylene)-O—, —(C₁-C₁₀ alkylene)-C(O)NR^{a}—, —(C₁-C₁₀ alkylene)-NR^{a}C(O)—, —(C₁-C₁₀ alkylene)-O—(C₁-C₁₀ alkylene)-O—, —(C₁-C₁₀ alkylene)-O—(C₁-C₁₀ alkylene)-, —(C₁-C₁₀ alkylene)-NR^{a}—(C₁-C₁₀ alkylene)-, —(C₁-C₁₀ alkylene)-NR^{a}—(C₁-C₁₀ alkylene)-O—, —(C₁-C₁₀ alkylene)-O—(C₁-C₁₀ alkylene)-NR^{a}—, —NR^{a}—(C₁-C₁₀ alkylene)-, —O—(C₁-C₁₀ alkylene)-, —C(O)NR^{a}—(C₁-C₁₀ alkylene)-, —NR^{a}C(O)—(C₁-C₁₀ alkylene)-, —(C₁-C₁₀ alkylene)-O—(C₁-C₁₀ alkylene)-, —O—(C₁-C₁₀ alkylene)-O—(C₁-C₁₀ alkylene)-, —(C₁-C₁₀ alkylene)-O—(C₁-C₁₀ alkylene)-C(O)—, —(C₁-C₁₀ alkylene)-C(O)—, —O—(C₁-C₁₀ alkylene)-C(O)—, —C(O)—(C₁-C₁₀ alkylene)-, —C(O)—(C₁-C₁₀ alkylene)-O—, —O—(C₁-C₁₀ alkylene)-C(O)NR^{a}—, —C(O)NR^{a}—(C₁-C₁₀ alkylene)-O—, —(C₁-C₁₀ alkylene)-S—, —S—(C₁-C₁₀ alkylene)-.

In a preferred embodiment of the present invention, wherein Y¹ and Y² together form a 5-10 membered saturated or unsaturated ring, a 6-10 membered aromatic ring, or a 5-10 membered heteroaromatic ring.

In a preferred embodiment of the present invention, wherein Y¹ and Y² together form any one of the following structures:

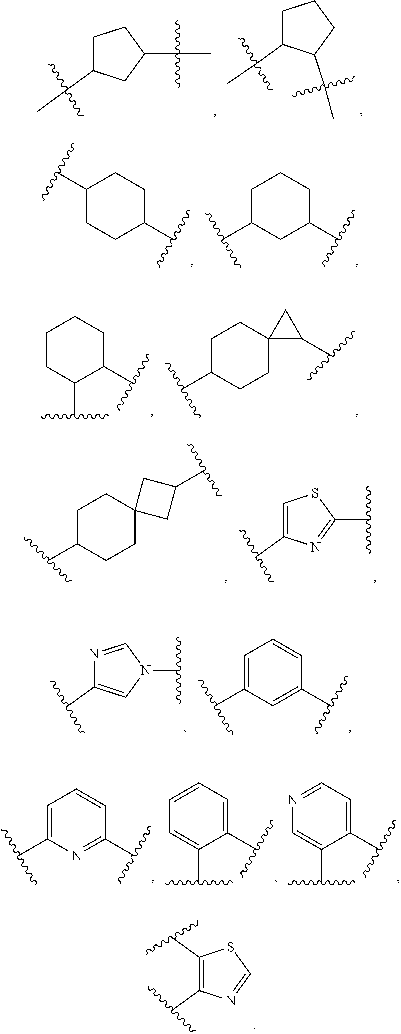

In a preferred embodiment of the present invention, having the following structure:

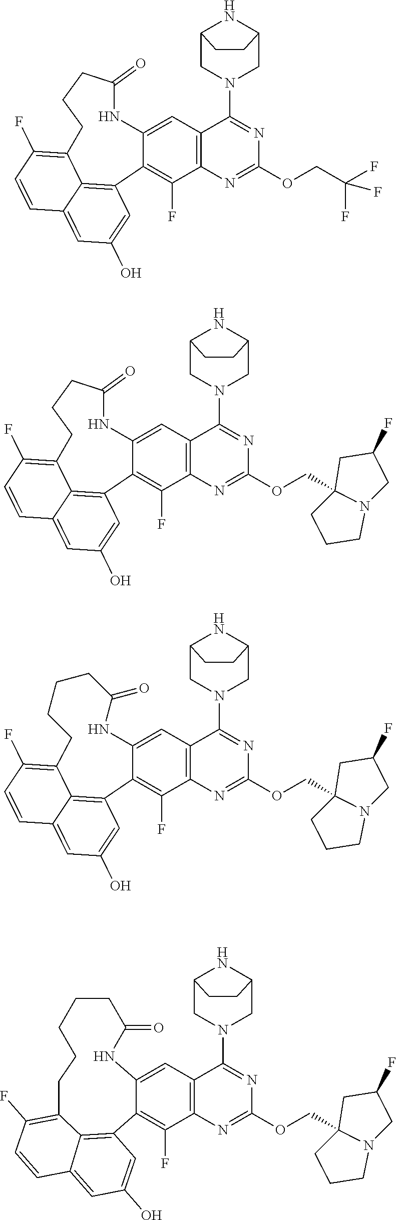 [SMILES: O=C1CCCc2c(F)ccc3cc(O)cc(c23)-c2c(cc3c(N4CC5CCC(C4)N5)nc(OCC(F)(F)F)nc3c2F)N1 ; O=C1CCCc2c(F)ccc3cc(O)cc(c23)-c2c(cc3c(N4CC5CCC(C4)N5)nc(OC[C@@]45CCCN4C[C@H](F)C5)nc3c2F)N1 ; O=C1CCCCc2c(F)ccc3cc(O)cc(c23)-c2c(cc3c(N4CC5CCC(C4)N5)nc(OC[C@@]45CCCN4C[C@H](F)C5)nc3c2F)N1 ; O=C1CCCCCc2c(F)ccc3cc(O)cc(c23)-c2c(cc3c(N4CC5CCC(C4)N5)nc(OC[C@@]45CCCN4C[C@H](F)C5)nc3c2F)N1]

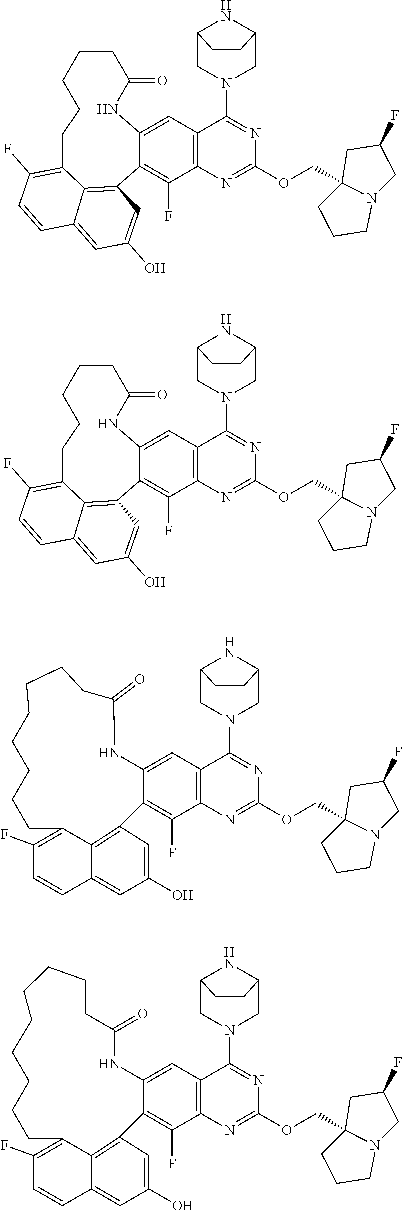

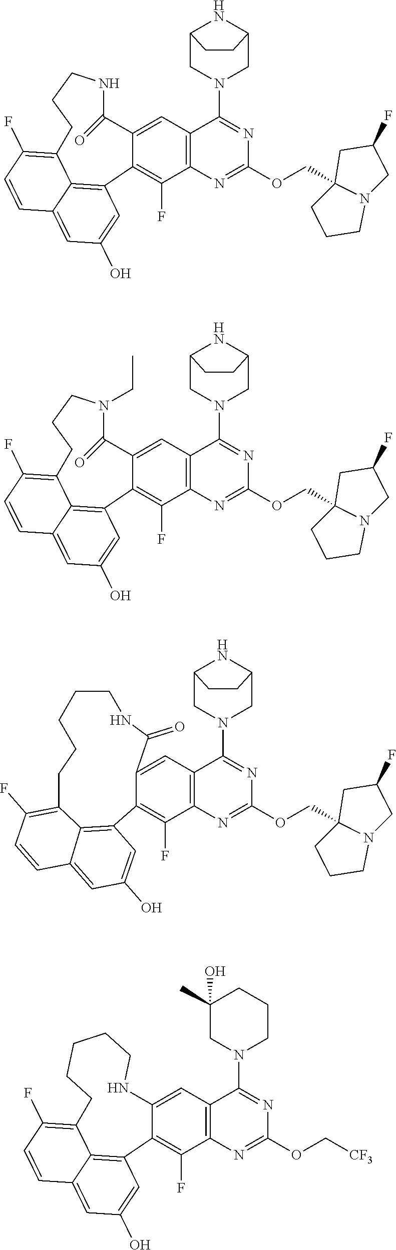 [SMILES: O=C1NCCCc2c(F)ccc3cc(O)cc(c23)-c2c1cc1c(N3CC4CCC(C3)N4)nc(OC[C@@]34CCCN3C[C@H](F)C4)nc1c2F ; CCN1CCCc2c(F)ccc3cc(O)cc(c23)-c2c(cc3c(N4CC5CCC(C4)N5)nc(OC[C@@]45CCCN4C[C@H](F)C5)nc3c2F)C1=O ; O=C1NCCCCCc2c(F)ccc3cc(O)cc(c23)-c2c1cc1c(N3CC4CCC(C3)N4)nc(OC[C@@]34CCCN3C[C@H](F)C4)nc1c2F ; C[C@@]1(O)CCCN(c2nc(OCC(F)(F)F)nc3c(F)c4c(cc23)NCCCCCc2c(F)ccc3cc(O)cc-4c23)C1]

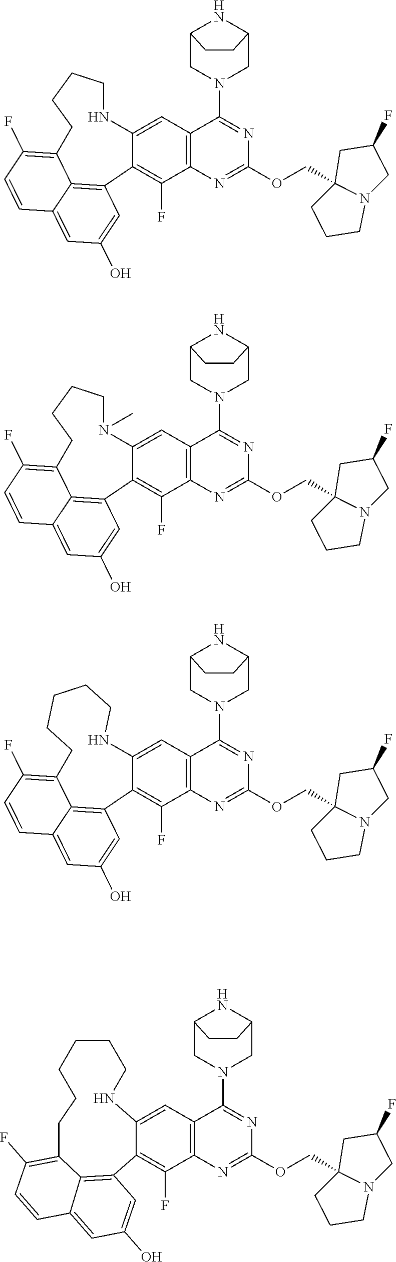 [SMILES: Oc1cc2c3c(c(F)ccc3c1)CCCCNc1cc3c(N4CC5CCC(C4)N5)nc(OC[C@@]45CCCN4C[C@H](F)C5)nc3c(F)c1-2 ; CN1CCCCc2c(F)ccc3cc(O)cc(c23)-c2c1cc1c(N3CC4CCC(C3)N4)nc(OC[C@@]34CCCN3C[C@H](F)C4)nc1c2F ; Oc1cc2c3c(c(F)ccc3c1)CCCCCNc1cc3c(N4CC5CCC(C4)N5)nc(OC[C@@]45CCCN4C[C@H](F)C5)nc3c(F)c1-2 ; Oc1cc2c3c(c(F)ccc3c1)CCCCCCNc1cc3c(N4CC5CCC(C4)N5)nc(OC[C@@]45CCCN4C[C@H](F)C5)nc3c(F)c1-2]

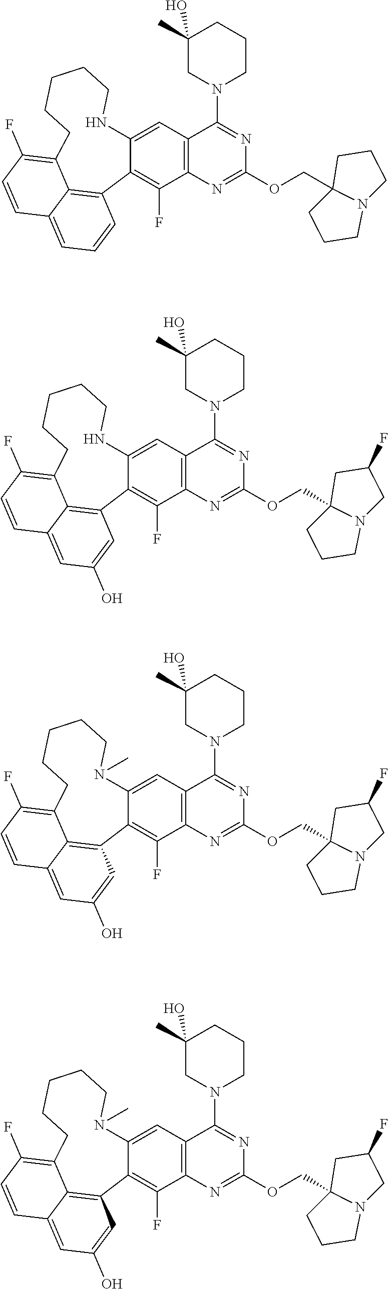

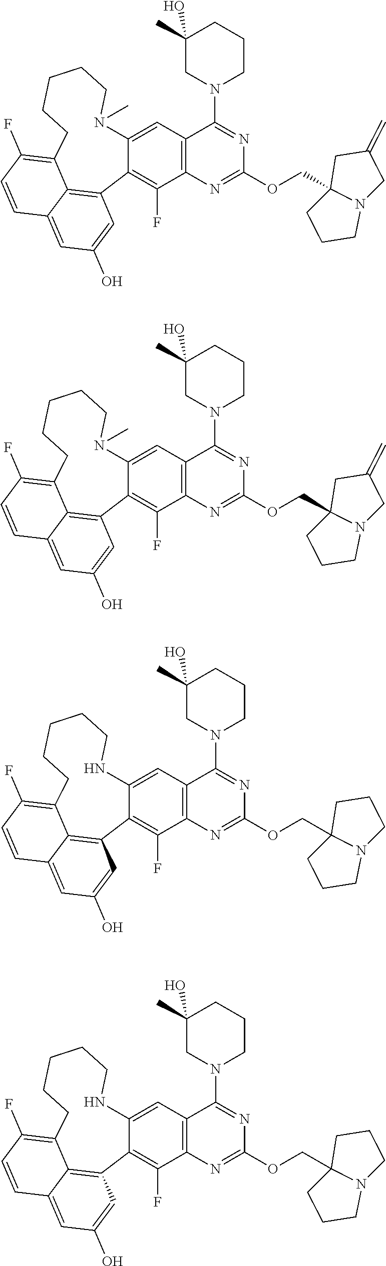

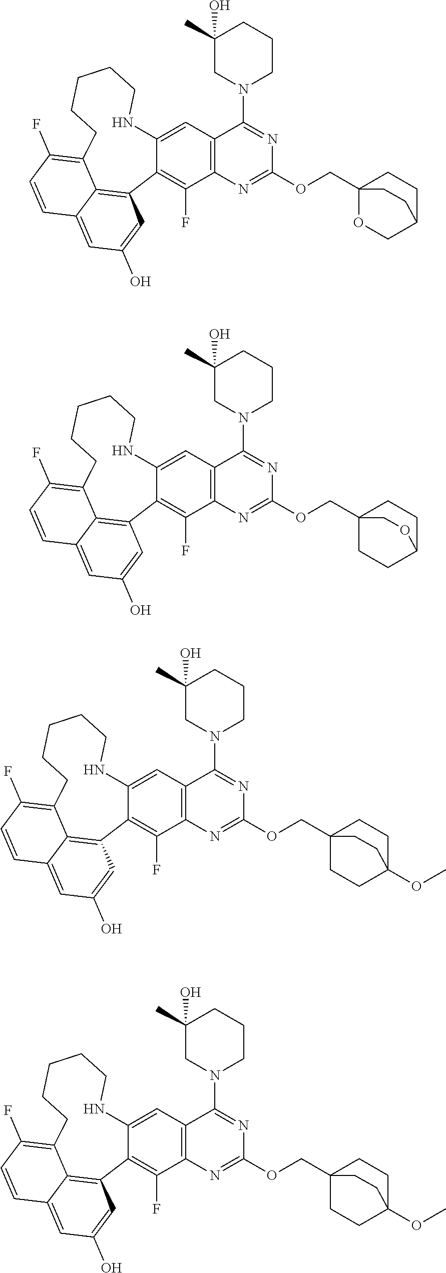

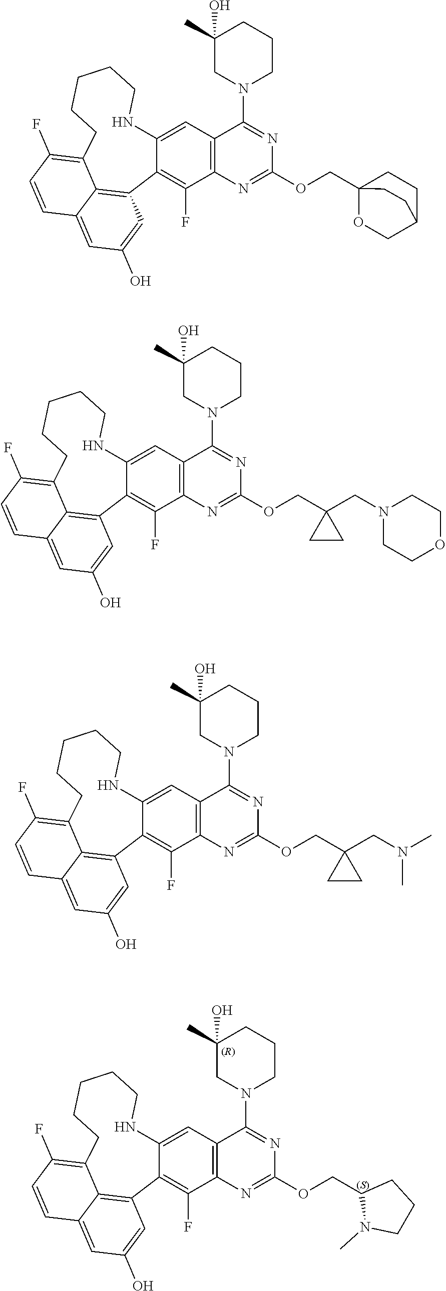

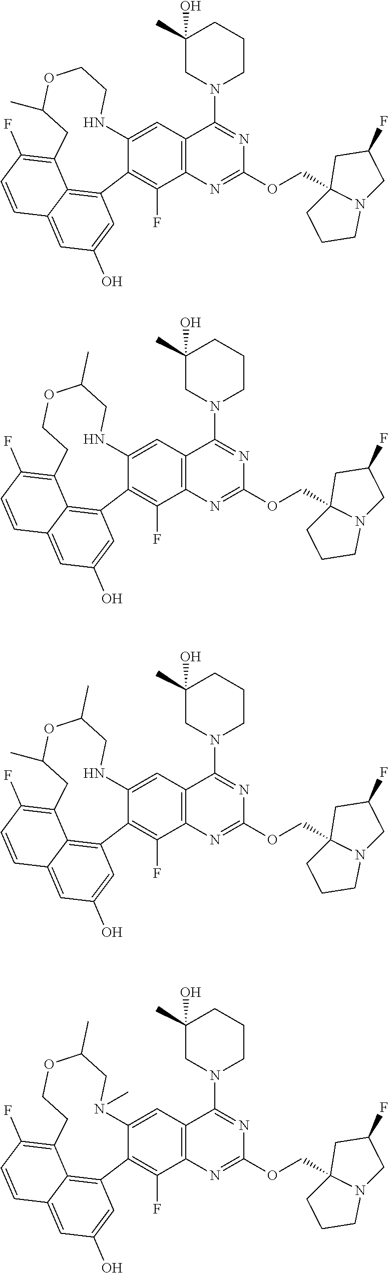 [SMILES: CC1Cc2c(F)ccc3cc(O)cc(c23)-c2c(cc3c(N4CCC[C@@](C)(O)C4)nc(OC[C@@]45CCCN4C[C@H](F)C5)nc3c2F)NCCO1 ; CC1CNc2cc3c(N4CCC[C@@](C)(O)C4)nc(OC[C@@]45CCCN4C[C@H](F)C5)nc3c(F)c2-c2cc(O)cc3ccc(F)c(c23)CCO1 ; CC1CNc2cc3c(N4CCC[C@@](C)(O)C4)nc(OC[C@@]45CCCN4C[C@H](F)C5)nc3c(F)c2-c2cc(O)cc3ccc(F)c(c23)CC(C)O1 ; CC1CN(C)c2cc3c(N4CCC[C@@](C)(O)C4)nc(OC[C@@]45CCCN4C[C@H](F)C5)nc3c(F)c2-c2cc(O)cc3ccc(F)c(c23)CCO1]

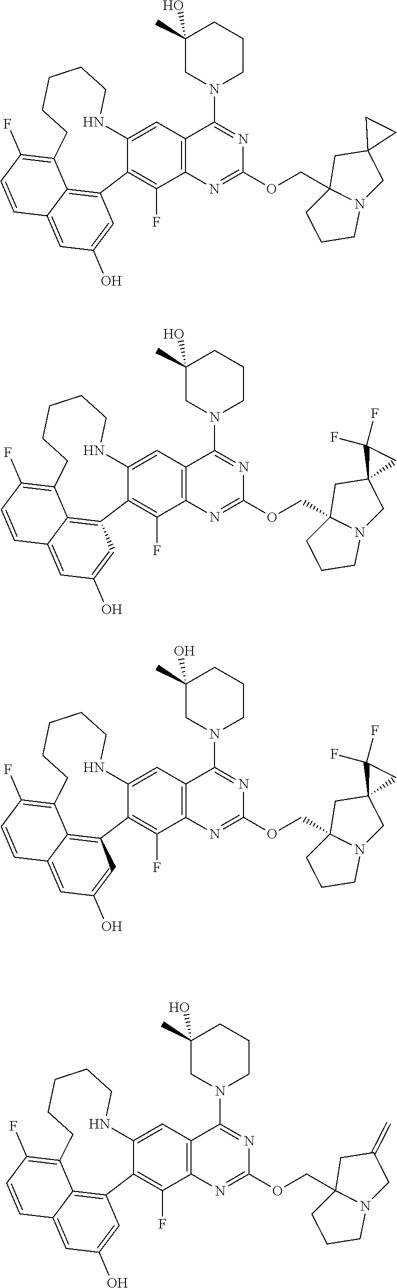

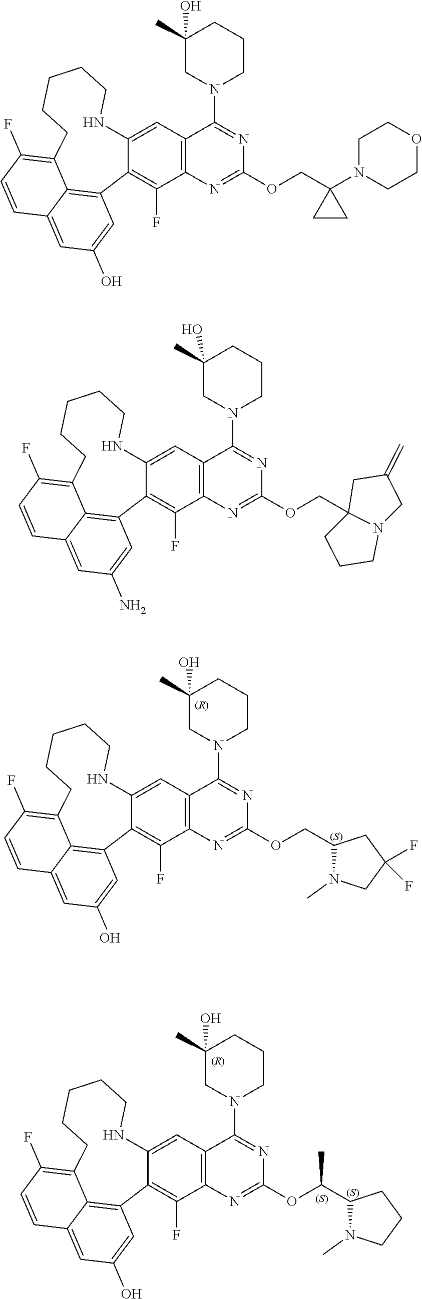

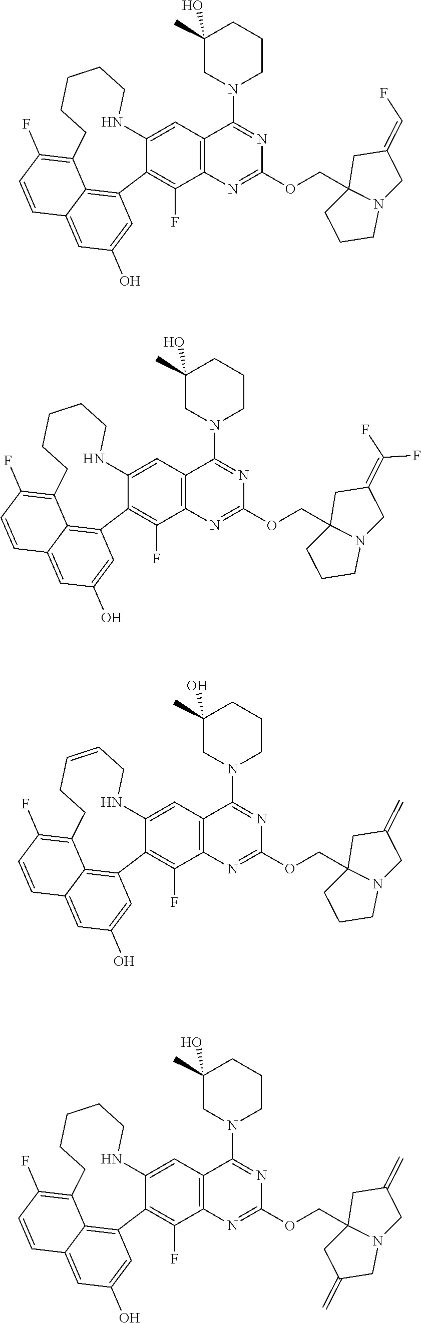 [SMILES: C[C@@]1(O)CCCN(c2nc(OCC34CCCN3C/C(=C/F)C4)nc3c(F)c4c(cc23)NCCCCCc2c(F)ccc3cc(O)cc-4c23)C1 ; C[C@@]1(O)CCCN(c2nc(OCC34CCCN3CC(=C(F)F)C4)nc3c(F)c4c(cc23)NCCCCCc2c(F)ccc3cc(O)cc-4c23)C1 ; C=C1CN2CCCC2(COc2nc(N3CCC[C@@](C)(O)C3)c3cc4c(c(F)c3n2)-c2cc(O)cc3ccc(F)c(c23)CC/C=C\CN4)C1 ; C=C1CN2CC(=C)CC2(COc2nc(N3CCC[C@@](C)(O)C3)c3cc4c(c(F)c3n2)-c2cc(O)cc3ccc(F)c(c23)CCCCCN4)C1]

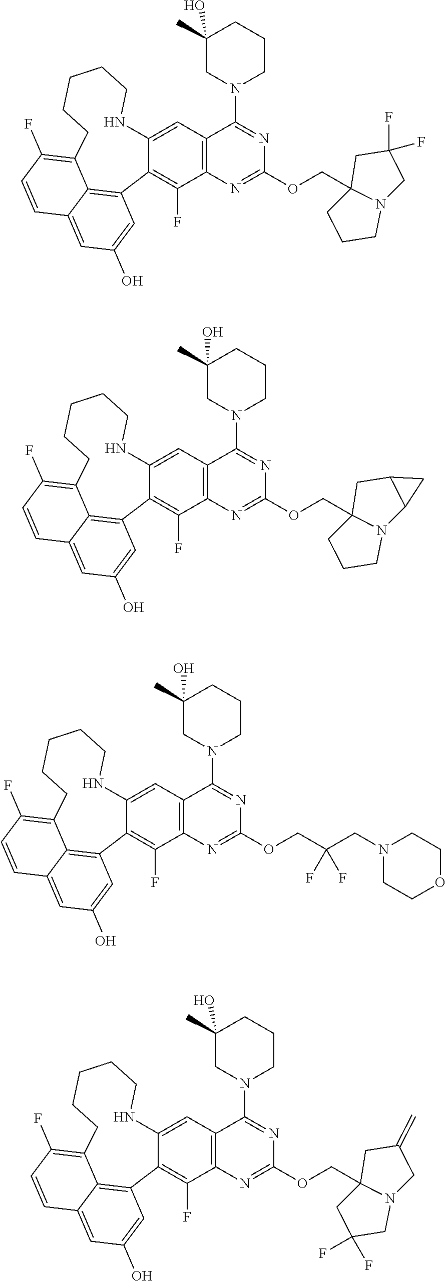 [SMILES: C[C@@]1(O)CCCN(c2nc(OCC34CCCN3CC(F)(F)C4)nc3c(F)c4c(cc23)NCCCCCc2c(F)ccc3cc(O)cc-4c23)C1 ; C[C@@]1(O)CCCN(c2nc(OCC34CCCN3C3CC3C4)nc3c(F)c4c(cc23)NCCCCCc2c(F)ccc3cc(O)cc-4c23)C1 ; C[C@@]1(O)CCCN(c2nc(OCC(F)(F)CN3CCOCC3)nc3c(F)c4c(cc23)NCCCCCc2c(F)ccc3cc(O)cc-4c23)C1 ; C=C1CN2CC(F)(F)CC2(COc2nc(N3CCC[C@@](C)(O)C3)c3cc4c(c(F)c3n2)-c2cc(O)cc3ccc(F)c(c23)CCCCCN4)C1]

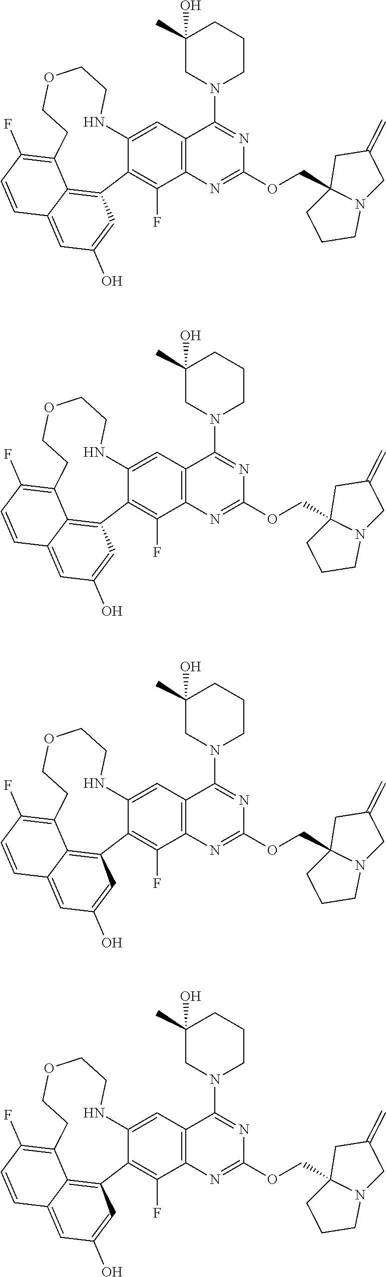

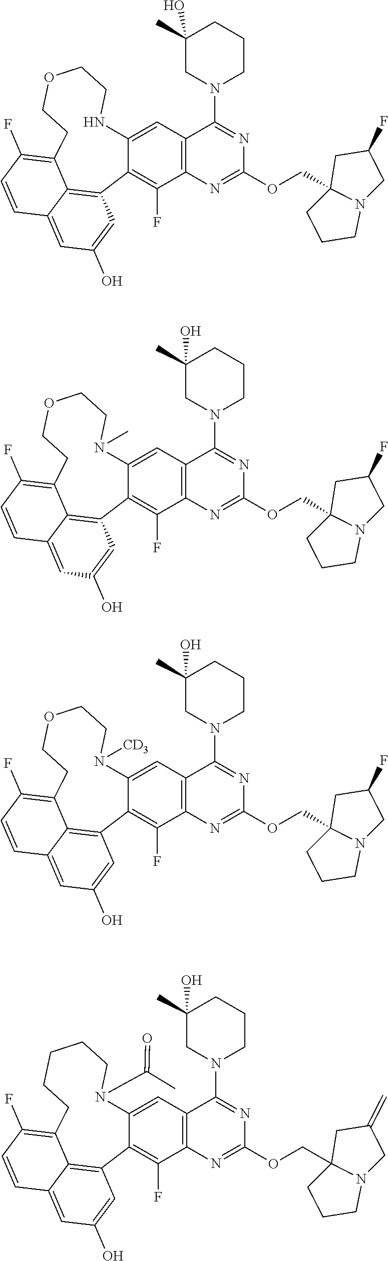

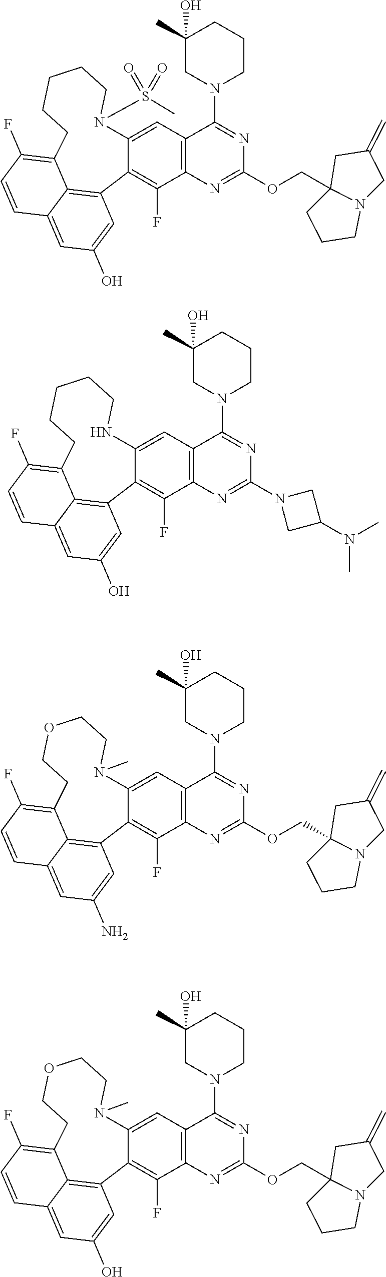 [SMILES: C=C1CN2CCCC2(COc2nc(N3CCC[C@@](C)(O)C3)c3cc4c(c(F)c3n2)-c2cc(O)cc3ccc(F)c(c23)CCCCCN4S(C)(=O)=O)C1 ; CN(C)C1CN(c2nc(N3CCC[C@@](C)(O)C3)c3cc4c(c(F)c3n2)-c2cc(O)cc3ccc(F)c(c23)CCCCCN4)C1 ; C=C1CN2CCC[C@@]2(COc2nc(N3CCC[C@@](C)(O)C3)c3cc4c(c(F)c3n2)-c2cc(N)cc3ccc(F)c(c23)CCOCCN4C)C1 ; C=C1CN2CCCC2(COc2nc(N3CCC[C@@](C)(O)C3)c3cc4c(c(F)c3n2)-c2cc(O)cc3ccc(F)c(c23)CCOCCN4C)C1]

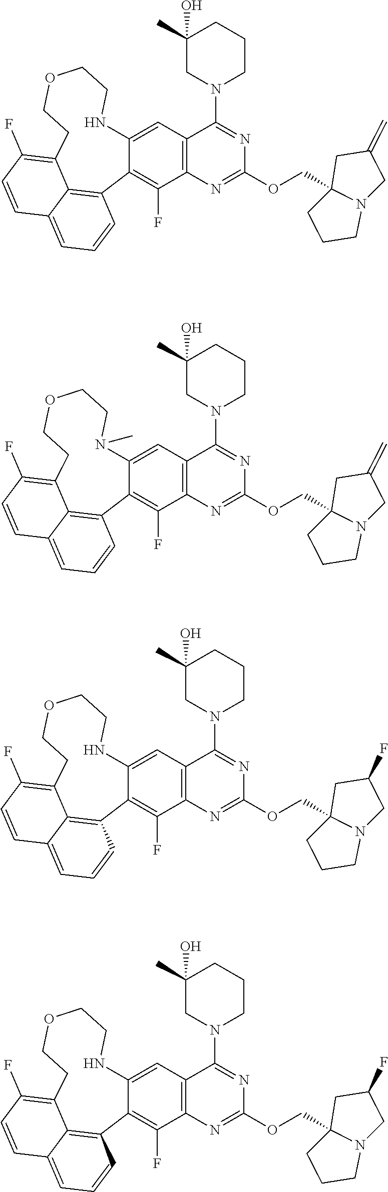

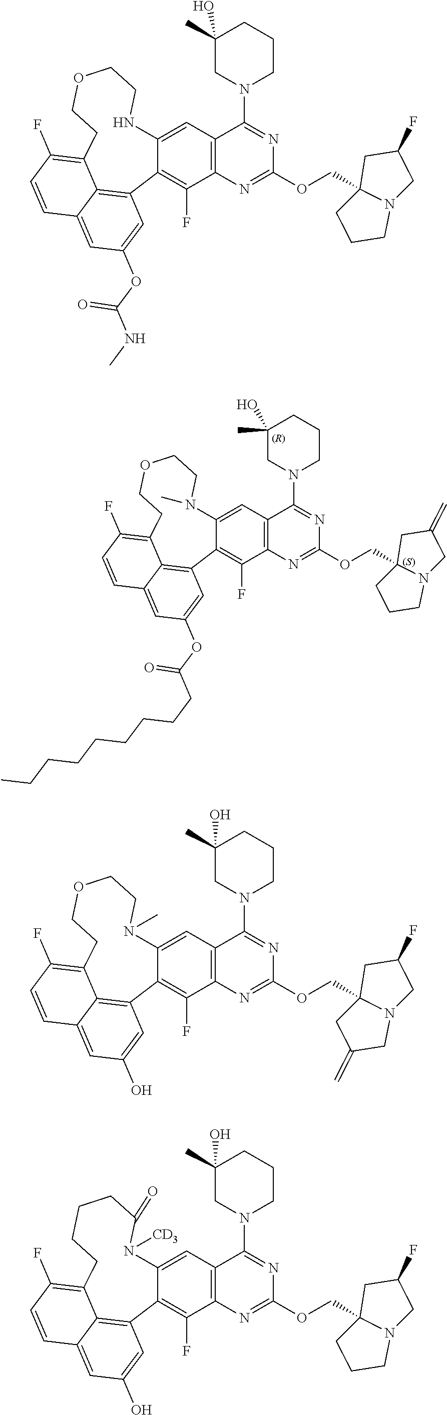

### DEFINITION OF TERMS

It is particularly noted that, herein, references to a “compound” with a specific structural formula generally also encompass its stereoisomers, diastereomers, enantiomers, racemic mixtures, and isotopologues, as well as pharmaceutically acceptable salts, solvates, and hydrates, which are alternative forms of the compound. It is well understood by those skilled in the art that salts, solvates, and hydrates of a compound are alternative forms that can convert into the compound under certain conditions. Therefore, it is expressly noted that any mention of a compound herein generally includes pharmaceutically acceptable salts thereof, and further includes solvates and hydrates thereof.

Similarly, when a compound is mentioned herein, it generally also includes its prodrugs, metabolites, and nitrogen oxides.

The pharmaceutically acceptable salts or medicinal salts described in the present invention can be formed using inorganic or organic acids. The term “pharmaceutically acceptable salt” refers to salts that, within the bounds of reasonable medical judgment, are suitable for contact with the tissues of humans and lower animals without causing excessive toxicity, irritation, allergic response, or other adverse effects, and provide a reasonable benefit/risk ratio. Such salts may be prepared in situ during the final isolation and purification of the compounds of the present invention or separately by reacting a free base or free acid with a suitable reagent, as outlined below. For example, a free base can react with an appropriate acid. Additionally, when the compounds of the present invention contain an acidic moiety, suitable pharmaceutically acceptable salts may include metal salts such as alkali metal salts (e.g., sodium or potassium salts) and alkaline earth metal salts (e.g., calcium or magnesium salts). Examples of pharmaceutically acceptable, non-toxic acid addition salts include those formed between amines and inorganic acids (e.g., hydrochloric acid, hydrobromic acid, phosphoric acid, sulfuric acid, and perchloric acid) or organic acids (e.g., acetic acid, oxalic acid, maleic acid, tartaric acid, citric acid, succinic acid, or malonic acid), or those formed by other methods such as ion exchange, as known in the art. Other pharmaceutically acceptable salts include adipates, alginates, ascorbates, aspartates, benzenesulfonates, benzoates, bisulfates, borates, butyrates, camphorates, camphorsulfonates, citrates, cyclopentanepropionates, digluconates, dodecyl sulfates, ethanesulfonates, formates, fumarates, gluceptates, glycerophosphates, gluconates, hemisulfates, heptanoates, hexanoates, hydroiodides, 2-hydroxyethanesulfonates, lactobionates, lactates, laurates, lauryl sulfates, malates, maleates, malonates, methanesulfonates, 2-naphthalenesulfonates, nicotinates, nitrates, oleates, oxalates, palmitates, pectates, persulfates, 3-phenylpropionates, phosphates, picrates, pivalates, propionates, stearates, succinates, sulfates, tartrates, thiocyanates, tosylates, undecanoates, valerates, and the like. Representative alkali metal or alkaline earth metal salts include sodium salts, lithium salts, potassium salts, calcium salts, magnesium salts, and others. Additional pharmaceutically acceptable salts include (where applicable) non-toxic ammonium salts, quaternary ammonium salts, and ammonium cations formed with counterions such as halides, hydroxides, carboxylates, sulfates, phosphates, nitrates, lower alkyl sulfonates, and aryl sulfonates.

Pharmaceutically acceptable salts described in the present invention can be prepared using conventional methods. For example, the compounds of the present invention can be dissolved in an organic solvent miscible with water (e.g., acetone, methanol, ethanol, or acetonitrile), followed by the addition of an aqueous solution of an organic or inorganic acid in excess, thereby precipitating the salt from the resulting mixture. The solvent and residual free acid can then be removed, and the precipitated salt separated.

The prodrugs or metabolites described in the present invention refer to those known in the art, provided that the prodrugs or metabolites are metabolized in vivo to produce the compound. For example, a “prodrug” refers to a precursor compound of the compounds described in the present invention, which, within the bounds of reasonable medical judgment, is suitable for contact with the tissues of humans and lower animals without undue toxicity, irritation, allergic response, or the like, and provides a reasonable benefit/risk ratio while being effective for its intended use. The term “prodrug” specifically refers to compounds that are rapidly converted in vivo to the parent compounds of the aforementioned structure, such as through metabolism or N-demethylation of the compounds described in the present invention.

The term “solvate,” as used in the present invention, refers to a physical association between the compounds described herein and one or more solvent molecules (whether organic or inorganic). Such physical association may include hydrogen bond. In certain cases, for example, when one or more solvent molecules are incorporated into the crystalline lattice of a solid, the solvate may be isolatable. Solvent molecules within a solvate may be present in an ordered or disordered arrangement. Solvates may include stoichiometric or non-stoichiometric amounts of solvent molecules. The term “solvate” encompasses both solution-phase solvates and isolatable solvates. Exemplary solvates include, but are not limited to, hydrates, ethanolates, methanolates, and isopropanolates. Methods for obtaining solvates are well known in the art.

The term “stereoisomer” as used in the present invention encompasses conformational isomers and configurational isomers. Configurational isomers can be further classified into cis-trans isomers and optical isomers (i.e., enantiomers). Conformational isomerism refers to a type of stereoisomerism where organic molecules with specific configurations exhibit different spatial arrangements of atoms or groups due to the rotation or twisting of carbon-carbon single bonds. Typical examples include the structures of alkanes and cycloalkanes, such as the chair and boat conformations of cyclohexane. “Stereoisomers” refer to compounds of the present invention that may contain one or more asymmetric centers, which may exist as racemates and racemic mixtures, single enantiomers, diastereomeric mixtures, or single diastereomers. The compounds of the present invention may contain asymmetric centers, with each center giving rise to two optical isomers. The scope of the present invention includes all possible optical isomers and diastereomeric mixtures, as well as pure or partially pure compounds. The compounds described in the present invention may exist in tautomeric forms, wherein one or more hydrogen atoms shift positions through the rearrangement of double bonds. For example, ketones and enol forms thereof are keto-enol tautomers. All tautomeric forms and mixtures thereof are included in the compounds of the present invention. The present invention encompasses all stereoisomers of compounds of Formula (I), including enantiomers, diastereomers, racemates, meso compounds, cis-trans isomers, tautomers, geometric isomers, atropisomers, and mixtures thereof. The term “isotopically labeled derivatives” in the present invention refers to molecules of the claimed compounds that are labeled with isotopes. Commonly used isotopes for labeling include: Hydrogen isotopes: ²H and ³H; Carbon isotopes: ¹¹C, ¹³C, and ¹⁴C; Chlorine isotopes: ³⁵Cl and ³⁷Cl; Fluorine isotope: ¹⁸F; Iodine isotopes: ¹²³I and ¹²⁵I; Nitrogen isotopes: ¹³N and ¹⁵N; Oxygen isotopes: ¹⁵O, ¹⁷O, and ¹⁸O; Sulfur isotope: ³⁵S. These isotopically labeled compounds are typically used to study the distribution of pharmaceutical molecules in tissues. In particular, deuterium (²H) and carbon-13 (¹³C) are widely utilized due to their ease of labeling and convenient detection. Certain heavy isotopes, such as deuterium (²H), can enhance metabolic stability, extend half-life, and thereby reduce dosage requirements while providing therapeutic advantages. The synthesis of isotopically labeled compounds typically begins with isotopically labeled starting materials and proceeds using known synthetic techniques, in the same manner as the synthesis of non-labeled compounds.

The compounds or pharmaceutical compositions of the present invention can be formulated into dosage forms for oral or parenteral administration (including intramuscular, intravenous, subcutaneous routes, and intratumoral injection) by any conventional method. These dosage forms may include tablets, granules, powders, capsules, syrups, emulsions, microemulsions, solutions, or suspensions.

Pharmaceutical compositions of the present invention for oral administration can be prepared by mixing the active ingredient with carriers such as cellulose, calcium silicate, corn starch, lactose, sucrose, dextrose, calcium phosphate, stearic acid, magnesium stearate, calcium stearate, gelatin, talc, surfactants, suspending agents, emulsifiers, and diluents. Examples of carriers used in the injectable compositions of the present invention include water, saline solution, glucose solution, glucose-like solution, alcohols, glycols, ethers (e.g., polyethylene glycol 400), oils, fatty acids, fatty acid esters, glycerides, surfactants, suspending agents, and emulsifiers.

Other features of the present invention will become apparent during the description of exemplary embodiments provided herein. These embodiments are intended to illustrate the present invention and are not to be construed as limiting. The following examples describe the preparation, isolation, and characterization of the present invention using the disclosed methods.

Unless otherwise specified, the definitions of terms used in the present invention (including the specification and claims) are as follows. It should be noted that, in the specification and appended claims, unless explicitly stated otherwise, the singular form “a” or “an” includes plural references. Unless specified otherwise, conventional methods of mass spectrometry, nuclear magnetic resonance (NMR), high-performance liquid chromatography (HPLC), protein chemistry, biochemistry, recombinant DNA technology, and pharmacology are used. In the present application, unless otherwise indicated, the terms “or” and “and” refer to “and/or.”.

In the specification and claims, the given chemical formula or name is intended to encompass all stereoisomers and optical isomers, as well as racemates containing such isomers. Unless otherwise indicated, all chiral forms (enantiomers and diastereomers) and racemic forms are within the scope of the present invention. The compounds described herein may also exist in various geometric isomers, including those arising from C═C double bonds, C═N double bonds, and ring systems, all of which are included within the present invention. The invention encompasses the cis- and trans- (or E- and Z-) geometric isomers of the described compounds, which can exist as mixtures of isomers or as isolated forms of individual isomers. The compounds of the present invention may be separated into optically active or racemic forms. All methods for preparing the compounds of the present invention and intermediates formed during their synthesis are considered part of the invention. When preparing products containing enantiomers or diastereomers, the isomers can be separated using conventional techniques (such as chromatography or fractional crystallization). Depending on the conditions of the method, the final products of the present invention can be obtained in their free (neutral) form or as salts. Both the free forms and salts of these final products fall within the scope of the invention. If necessary, one form of a compound can be converted into another. Free bases or acids can be converted to salts, salts can be converted to free compounds or other salts, and mixtures of isomeric compounds can be separated into individual isomers. The compounds of the present invention, in their free forms and salts, may exist in various tautomeric forms, where hydrogen atoms migrate within the molecule, leading to a rearrangement of chemical bonds between the atoms of the molecule. It is understood that all tautomeric forms that may exist are included within the scope of the present invention.

Unless otherwise defined, the substituents in the present invention are defined as being independently assigned rather than interdependently related. For example, for a substituent R^{a} (or R^{b}), its definition in different substituents is independently assigned. Specifically, when R^{a} (or R^{b}) is selected as one definition in one substituent, this does not imply that R^{a} (or R^{b}) in other substituents must share the same definition. More specifically, when R^{a} (or R^{b}) is defined as hydrogen in NR^{a}R^{b}, for example (non-exhaustive), it does not imply that R^{a} (or R^{b}) in —C(O)—NR^{a}R^{b} must be hydrogen.

Unless otherwise defined, when a substituent is described as “optionally substituted,” the substituent is selected from, for example, the following groups: alkyl, cycloalkyl, aromatic ring, heterocyclyl, halogen, hydroxyl, alkoxy, oxo, alkanoyl, aryloxy, alkanoyloxy, amino, alkylamino, arylamino, arylalkylamino, disubstituted amino groups (where the two amino substituents are independently selected from alkyl, aryl, or arylalkyl), alkanoylamino, arylcarbonylamino, arylalkanoylamino, substituted alkanoylamino, substituted arylamino, substituted arylalkanoylamino, thio, alkylthio, arylthio, arylalkylthio, arylthiocarbonyl, arylalkylthiocarbonyl, alkylsulfonyl, arylsulfonyl, arylalkylsulfonyl, sulfonamido groups such as —SO₂NH₂, substituted sulfonamido, nitro, cyano, carboxyl, carbamoyl groups such as —CONH₂, substituted carbamoyl groups such as —CONH(alkyl), —CONH(aromatic ring), —CONH(arylalkyl), or cases where the nitrogen has two substituents independently selected from alkyl, aromatic ring, or arylalkyl, alkoxycarbonyl, aromatic ring, substituted aromatic ring, guanidino, heterocyclyl groups such as indolyl, imidazolyl, furyl, thienyl, thiazolyl, pyrrolyl, pyridinyl, pyrimidinyl, pyrrolidinyl, piperidinyl, morpholinyl, piperazinyl, homopiperazinyl, and substituted heterocyclyl groups.

The term “alkyl” or “alkylene” as used herein is intended to include both branched and straight-chain saturated aliphatic hydrocarbon groups having the specified number of carbon atoms. For example, “C₁-C₆ alkyl” refers to an alkyl group containing 1 to 6 carbon atoms. Examples of alkyl groups include, but are not limited to, methyl (Me), ethyl (Et), n-propyl, isopropyl, n-butyl, isobutyl, tert-butyl, n-pentyl, isopentyl, and neopentyl.

The term “alkenyl” refers to hydrocarbon groups containing one or more double bonds, which are typically straight-chain or branched and range in length from 2 to 20 carbon atoms. For instance, “C₂-C₆ alkenyl” refers to alkenyl groups containing 2 to 6 carbon atoms. Examples of alkenyl groups include, but are not limited to, vinyl, allyl, butenyl, and 1-methyl-2-buten-1-yl.

The term “alkynyl” refers to a linear or branched hydrocarbyl containing one or more triple bonds and typically comprising 2-20 carbon atoms. For example, “C₂-C₆ alkynyl” refers to alkynyl groups containing 2 to 6 carbon atoms. Representative alkynyl groups include, but are not limited to, ethynyl, 1-propynyl, and 1-butynyl.

The term “alkoxy” or “alkyl oxy” refers to groups of the formula —O-alkyl. “C₁-C₆ alkoxy” (or alkyl oxy) is intended to include alkoxy groups containing C1, C2, C3, C4, C5 and C6. Examples of alkoxy groups include, but are not limited to, methoxy, ethoxy, propoxy (e.g., n-propoxy and isopropoxy), and tert-butoxy. Similarly, the term “alkylthio” or “thioalkoxy” refers to groups containing the specified number of carbon atoms, as defined above for alkyl, connected through a sulfur atom, such as methylthio and ethylthio.

The term “carbonyl” refers to an organic functional group consisting of a carbon atom doubly bonded to an oxygen atom (C═O).

The term “aromatic ring,” whether used alone or as part of a larger group such as “aralkyl,” “aryloxyalkyl,” or “aryloxy,” refers to a cyclic system containing a total of 5 to 12 ring members in mono-, bi-, or tri-cyclic structures, where at least one ring in the system is aromatic and each ring contains 3 to 7 ring members. In certain embodiments of the present invention, “aryl” refers to aromatic ring systems, including but not limited to phenyl, biphenyl, indanyl, 1-naphthyl, 2-naphthyl, and tetrahydronaphthyl. The term “aralkyl” or “arylalkyl” refers to alkyl residues attached to an aryl ring. Non-limiting examples include benzyl and phenylethyl. Fused aryl groups may be connected to another group at any suitable position on a cycloalkyl ring or aromatic ring. Dotted lines extending from a cyclic system indicate that the bond may connect to any appropriate ring atom.

The term “cycloalkyl” refers to mono- or bi-cyclic cyclic alkyl groups. Mono-cyclic cycloalkyl groups refer to C₃-C₈ cycloalkyls, including but not limited to cyclopropyl, cyclobutyl, cyclopentyl, cyclohexyl, and norbornyl. Branched cycloalkyls, such as 1-methylcyclopropyl and 2-methylcyclopropyl, are also included in the definition of “cycloalkyl.”. Bi-cyclic cycloalkyl groups include bicyclic structures such as bridged, spiro, or fused cycloalkyls.

The term “heterocycloalkyl” refers to the cycloalkyl structures described above, where at least 1 carbon atom in the ring is replaced by a heteroatom selected from O, N, S, or Se.

The term “cycloalkenyl” refers to mono- or bi-cyclic alkenyl. Mono-cyclic cycloalkenyl groups refer to C₃-C₈ cycloalkenyls, including but not limited to cyclopropenyl, cyclobutenyl, cyclopentenyl, cyclohexenyl, and norbornenyl. Branched cycloalkenyl, such as 1-methylcyclopropyl and 2-methylcyclopropyl, are also included in the definition of “cycloalkenyl”. Bi-cyclic cycloalkenyl groups include bicyclic structures such as bridged, spiro, or fused cycloalkenyls.

The term “halogenated” or “halogen” includes fluorine, chlorine, bromine, and iodine. “Haloalkyl” refers to saturated aliphatic hydrocarbon groups with the specified number of carbon atoms, either branched or straight-chain, substituted with 1 or more halogens. Examples of haloalkyl groups include but are not limited to fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” which is intended to include branched and linear saturated aliphatic hydrocarbyl having a specified number of carbon atoms and substituted with 1 or more fluorine atoms.

The terms “haloalkoxy” or “haloalkyloxy” refer to a haloalkyl group, as defined above, connected via an oxygen bridge, with the specified number of carbon atoms. For example, “C₁-C₆ haloalkoxy” is intended to include C₁, C₂, C₃, C₄, C₅, and C₆ haloalkoxy groups. Examples of haloalkoxy groups include but are not limited to trifluoromethoxy, 2,2,2-trifluoroethoxy, and pentafluoroethoxy. Similarly, the terms “haloalkylthio” or “thiohaloalkyloxy” refer to a haloalkyl group, as defined above, connected via a sulfur bridge, with the specified number of carbon atoms; for example, trifluoromethyl-S— and pentafluoroethyl-S—.

In the disclosure, when certain substituent groups are described using the notation Cₓ₁-Cₓ₂, this indicates that the number of carbon atoms in the substituent group can range from x1 to x2. For example, C₀-C₈ indicates that the group may contain 0, 1, 2, 3, 4, 5, 6, 7, or 8 carbon atoms; C₁-C₈ indicates that the group may contain 1, 2, 3, 4, 5, 6, 7, or 8 carbon atoms; C₂-C₈ indicates that the group may contain 2, 3, 4, 5, 6, 7, or 8 carbon atoms; C₃-C₈ indicates that the group may contain 3, 4, 5, 6, 7, or 8 carbon atoms; C₄-C₈ indicates that the group may contain 4, 5, 6, 7, or 8 carbon atoms; C₀-C₆ indicates that the group may contain 0, 1, 2, 3, 4, 5, or 6 carbon atoms; C₁-C₆ indicates that the group may contain 1, 2, 3, 4, 5, or 6 carbon atoms; C₂-C₆ indicates that the group may contain 2, 3, 4, 5, or 6 carbon atoms; and C₃-C₆ indicates that the group may contain 3, 4, 5, or 6 carbon atoms.

In the disclosure, when cyclic groups (e.g., aryl, heteroaryl, cycloalkyl, and heterocycloalkyl) are mentioned with the expression “x1-x2-membered ring,” this indicates that the cyclic group may contain x1 to x2 ring atoms. For example, a 3-12-membered ring indicates that the cyclic group may be 3-, 4-, 5-, 6-, 7-, 8-, 9-, 10-, 11-, or 12-ring, with 3, 4, 5, 6, 7, 8, 9, 10, 11, or 12 ring atoms; a 3-6-membered ring indicates that the cyclic group may be a 3-, 4-, 5-, or 6-membered ring, with 3, 4, 5, or 6 ring atoms; a 3-8-membered ring indicates that the cyclic group may be a 3-, 4-, 5-, 6-, 7-, or 8-membered ring, with 3, 4, 5, 6, 7, or 8 ring atoms; a 3-9-membered ring indicates that the cyclic group may be a 3-, 4-, 5-, 6-, 7-, 8-, or 9-membered ring, with 3, 4, 5, 6, 7, 8, or 9 ring atoms; a 4-7-membered ring indicates that the cyclic group may be a 4-, 5-, 6-, or 7-membered ring, with 4, 5, 6, or 7 ring atoms; a 5-8-membered ring indicates that the cyclic group may be a 5-, 6-, 7-, or 8-membered ring, with 5, 6, 7, or 8 ring atoms; a 5-12-membered ring indicates that the cyclic group may be a 5-, 6-, 7-, 8-, 9-, 10-, 11-, or 12-membered ring, with 5, 6, 7, 8, 9, 10, 11, or 12 ring atoms; and a 6-12-membered ring indicates that the cyclic group may be a 6-, 7-, 8-, 9-, 10-, 11-, or 12-membered ring, with 6, 7, 8, 9, 10, 11, or 12 ring atoms. The ring atoms may include carbon atoms or heteroatoms, such as heteroatoms selected from N, O, and S. When the ring is a heterocycle, it may contain 1, 2, 3, 4, 5, 6, 7, 8, 9, 10, or more heteroatoms, for example, heteroatoms selected from N, O, and S.

In the context of the present invention, one or more halogens may independently be selected from fluorine, chlorine, bromine, and iodine.

The term “heteroaryl” refers to stable 3-membered, 4-membered, 5-membered, 6-membered, or 7-membered aromatic monocyclic rings, aromatic bicyclic rings, or 7-membered, 8-membered, 9-membered, 10-membered, 11-membered, 12-membered aromatic polycyclic heterocycles. These heterocycles are fully unsaturated, partially unsaturated, and contain carbon atoms along with 1, 2, 3, or 4 heteroatoms independently selected from N, O, and S. The term also includes any polycyclic groups in which any of the above-defined heterocycles are fused with a benzene ring. The nitrogen and sulfur heteroatoms may optionally be oxidized. The nitrogen atom may be substituted or unsubstituted (i.e., N or NR, where R is H or another substituent if defined). Heterocycles can be attached to their side groups at any heteroatom or carbon atom that results in a stable structure. If the resulting compound is stable, the heterocyclic group described herein may be substituted at a carbon or nitrogen atom. The nitrogen atom in the heterocycle may optionally be quaternized. Preferably, when the total number of sulfur and oxygen atoms in the heterocycle exceeds 1, these heteroatoms are not adjacent to one another. More preferably, the total number of sulfur and oxygen atoms in the heterocycle does not exceed 1.

The reagents and starting materials used in the present invention are commercially available.

The advantageous effects of the present invention are as follows: the compounds described in the present invention exhibit strong cell proliferation inhibitory activity against KRAS G12D-mutated gastric cancer AGS cell lines and metastatic pancreatic adenocarcinoma AsPC-1 cell lines. They demonstrate excellent stability in liver microsomes, hepatocytes, plasma, and whole blood, along with favorable pharmacokinetic (PK) properties. Furthermore, the compounds display remarkable tumor suppression activity.

### DETAILED DESCRIPTION OF THE EMBODIMENTS

The present invention is further illustrated through the following examples, which are provided for explanatory purposes only and should not be construed as limiting the scope of the present invention. Experimental methods with no specific conditions indicated in the following Examples should be selected according to conventional methods and conditions, or commercial specifications.

NMR spectroscopy was conducted using a Bruker AVANCE-400 NMR spectrometer. The solvent used in the measurements is specified in the spectrum interpretation.

MS analysis was performed using Agilent 1200-G1956A/1200-6110A/1200-6140A/1260-6125B/Prime-6125B/1260-6120 Liquid Chromatography-Mass Spectrometry (LC-MS) systems, SHIMADZU 20A-2010/20A-2020 LC-MS systems, and Waters ACQ-QDA LC-MS systems.

HPLC analysis was conducted using a SHIMADZU 20A HPLC system.

SFC analysis was performed using Waters UPCC with PDA Detector and QDa Detector systems, Waters UPC² with PDA Detector systems, Agilent 1260 with DAD Detector systems, Shimadzu LC-20AB with PDA Detector systems, and Shimadzu LC-20AD with PDA Detector systems.

Preparative HPLC separation was performed using Shimadzu LC-20AP Pump, Shimadzu LH-40 Liquid Handler, Shimadzu SPD-20A Detector, Gilson GX-281 Liquid Handler, Gilson 322 Pump, and Gilson 156 UV Detector preparative chromatography systems.

SFC separations were conducted using The Berger MG II, MG III, Sepiatec's Prep SFC 100 System, Waters Prep 80Q SFC SYSTEM, Prep 150 AP SFC SYSTEM, Prep 200 SFC SYSTEM, and Prep 350 SFC SYSTEM.

Flash column chromatography was performed using a Biotage IsoleraOne preparative chromatography system.

TLC plates used were GF254 acrylic adhesive silica gel plates provided by Anhui Liangchen Silica Source Materials Co., Ltd. The TLC silica gel plates used for analysis were 0.25 mm in thickness, while those used for product purification were 0.5 mm in thickness.

Pressurized hydrogenation reactions utilized hydrogenation bottles and hydrogen gas cylinders.

Microwave reactions were performed using a Biotage Initiator+ microwave synthesizer.

A custom glove box from DELLIX was used.

### Intermediate 1: tert-Butyl (1R,5S)-3-(6-amino-7-(8-ethynyl-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate

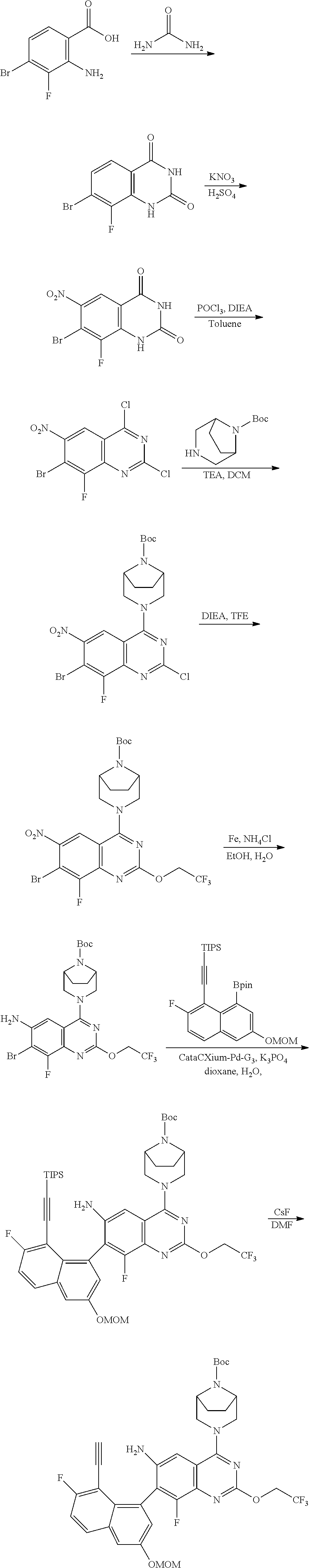

Step I: A mixture of 2-amino-4-bromo-3-fluorobenzoic acid (3.50 g, 14.96 mmol) and urea (8.98 g, 149.56 mmol) was stirred at 200° C. for 2 hours. The reaction mixture was cooled to 100° C., and water (30 mL) was added, followed by stirring for 1 hour. The resulting solid was filtered, washed with water (25 mL), and stirred at 50° C. for 1 hour. After filtration, the solid was washed with water (25 mL) again and stirred at 25° C. for 12 hours. The final solid was filtered, vacuum dried, and gave crude 7-bromo-8-fluoroquinazoline-2,4(1H,3H)-dione (4.40 g) as a gray solid. LCMS (ESI): [M+H]⁺=258.8.

Step II: At 0° C., to a concentrated sulfuric acid solution (35 mL) containing 7-bromo-8-fluoroquinazoline-2,4(1H,3H)-dione (4.40 g, 14.96 mmol) was added potassium nitrate (3.43 g, 33.97 mmol). The mixture was stirred at 0° C. for 1 hour. And then poured into water (35 mL). The resulting solid was filtered, vacuum dried, and gave gray solid 7-bromo-8-fluoro-6-nitroquinazoline-2,4(1H,3H)-dione (3.50 g, 11.51 mmol, yield: 77%). LCMS (ESI): [M+H]⁺=303.9. ¹H NMR (400 MHz, DMSO-de) S ppm 11.98 (br s, 1H), 11.85 (s, 1H), 8.34 (s, 1H).

Step III: At 0° C., a solution of 7-bromo-8-fluoro-6-nitroquinazoline-2,4(1H,3H)-dione (1.50 g, 4.93 mmol) and phosphorus oxychloride (3.67 mL, 39.47 mmol) in toluene (22 mL) was added diisopropylethylamine (2.45 mL, 14.80 mmol). The mixture was stirred under nitrogen at 100° C. for 3 hours. After the reaction, the mixture was concentrated under reduced pressure to dryness. The residue was dissolved in ethyl acetate (30 mL) and then treated with ice and ice water (30 mL). The mixture was extracted with ethyl acetate (20 mL×3), and the combined organic layers were dried over anhydrous sodium sulfate, filtered, and concentrated under reduced pressure to dryness. This yielded crude 7-bromo-2,4-dichloro-8-fluoro-6-nitroquinazoline (1.55 g) as a gray solid. LCMS (ESI): [M+H]⁺=341.8.

Step IV: To a solution of 7-bromo-2,4-dichloro-8-fluoro-6-nitroquinazoline (1.55 g, 4.55 mmol) and tert-butyl 3,8-diazabicyclo[3.2.1]octane-8-carboxylate (965 mg, 4.55 mmol) in dichloromethane (15 mL) was added triethylamine (1.84 mL, 13.20 mmol). The mixture was stirred at 25° C. for 2 hours. The reaction mixture was diluted with water (10 mL) and extracted with dichloromethane (15 mL×3). The combined organic phases were dried over anhydrous sodium sulfate, filtered, and concentrated under reduced pressure. The residue was purified by flash column chromatography on silica gel (0-30% gradient of ethyl acetate/petroleum ether) to give the gray solid (1R,5S)-3-(7-bromo-2-chloro-8-fluoro-6-nitroquinazolin-4-yl)-3,8-diazabicyclo[3.2.1]octane-8-carboxylate (590 mg, 1.14 mmol, yield: 25%). LCMS (ESI): [M+H]⁺=517.7.

Step V: To a solution of (1R,5S)-3-(7-bromo-2-chloro-8-fluoro-6-nitroquinazolin-4-yl)-3,8-diazabicyclo[3.2.1]octane-8-carboxylate (590 mg, 1.14 mmol) in trifluoroethanol (4.4 mL) was added diisopropylethylamine (944 μL, 5.71 mmol). The mixture was stirred at 70° C. for 12 hours and then concentrated under reduced pressure. The residue was purified by flash chromatography on silica gel (0-30% gradient of ethyl acetate/petroleum ether) to give the yellow solid compound tert-butyl (1R,5S)-3-(7-bromo-8-fluoro-6-nitro-2-(2,2,2-trifluoroethoxy)quinazolin-4-yl)-3,8-diazabicyclo[3.2.1]octane-8-carboxylate (500 mg, 0.86 mmol, yield: 75%). LCMS (ESI): [M+H]⁺=581.7.

Step VI: To a solution of tert-butyl (1R,5S)-3-(7-bromo-8-fluoro-6-nitro-2-(2,2,2-trifluoroethoxy)quinazolin-4-yl)-3,8-diazabicyclo[3.2.1]octane-8-carboxylate (2.00 g, 3.45 mmol) in ethanol (40 mL) and water (20 mL) were added ammonium chloride (1.84 g, 34.46 mmol) and iron powder (0.96 g, 17.23 mmol). The mixture was stirred under nitrogen at 80° C. for 2 hours and then filtered. The solid residue was washed with dichloromethane (60 mL). The filtrate was poured into water (100 mL) and extracted with dichloromethane (100 mL×3). The combined organic phases were dried over anhydrous sodium sulfate, filtered, and the filtrate was concentrated to dryness under reduced pressure to give the crude compound tert-butyl (1R,5S)-3-(6-amino-7-bromo-8-fluoro-2-(2,2,2-trifluoroethoxy)quinazolin-4-yl)-3,8-diazabicyclo[3.2.1]octane-8-carboxylate (1.95 g), which was a brown solid. LCMS (ESI): [M+H]⁺=551.9.

Step VII: Under a nitrogen atmosphere, to a stirred solution of tert-butyl (1R,5S)-3-(6-amino-7-bromo-8-fluoro-2-(2,2,2-trifluoroethoxy)quinazolin-4-yl)-3,8-diazabicyclo[3.2.1]octane-8-carboxylate (1.93 g, 3.51 mmol) and ((2-fluoro-6-(methoxymethoxy)-8-(4,4,5,5-tetramethyl-1,3,2-dioxaborolane)naphthalen-1-yl)ethynyl)triisopropylsilane (2.34 g, 4.56 mmol) in 1,4-dioxane (40 mL) were added potassium phosphate (1.5 M, 7.0 mL, 10.52 mmol) and methanesulfonate (diadamantyl-n-butylphosphine)-2-amino-1,1′-biphenyl-2-yl palladium(II) (255.4 mg, 0.35 mmol). The mixture was stirred under nitrogen at 100° C. for 2 hours. Water (20 mL) was added to dilute the mixture, which was then extracted with ethyl acetate (20 mL×3). The combined organic layers were dried over anhydrous sodium sulfate, filtered, and concentrated under reduced pressure. The residue was purified by flash chromatography on silica gel (0-30% gradient of ethyl acetate/petroleum ether) to give the brown solid compound tert-butyl (1R,5S)-3-(6-amino-8-fluoro-7-(7-fluoro-3-(methoxymethoxy)-8-((triisopropylsilyl)ethynyl)naphthalen-1-yl)-2-(2,2,2-trifluoroethoxy)quinazolin-4-yl)-3,8-diazabicyclo[3.2.1]octane-8-carboxylate (1.40 g, 1.64 mmol, yield: 47%). LCMS (ESI): [M+H]⁺=856.5.

Step VIII: To a solution of tert-butyl (1R,5S)-3-(6-amino-8-fluoro-7-(7-fluoro-3-(methoxymethoxy)-8-((triisopropylsilyl)ethynyl)naphthalen-1-yl)-2-(2,2,2-trifluoroethoxy)quinazolin-4-yl)-3,8-diazabicyclo[3.2.1]octane-8-carboxylate (1370.0 mg, 1.60 mmol) in N,N-dimethylformamide (27 mL) was added cesium fluoride (2431.1 mg, 16.00 mmol). The mixture was stirred at 25° C. for 1 hour under a nitrogen atmosphere. The reaction solution was diluted with water (90 mL) and extracted with ethyl acetate (90 mL×3). The organic layers were combined, dried over anhydrous sodium sulfate, filtered, and concentrated under reduced pressure. The residue was purified by flash chromatography on silica gel (0-30% gradient of ethyl acetate/petroleum ether) to give the brown solid compound tert-butyl (1R,5S)-3-(6-amino-7-(8-ethynyl-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (720.0 mg, 1.03 mmol, yield: 64%). LCMS (ESI): [M+H]⁺=700.2.

### Intermediate 2: methyl 4-((1R,5S)-8-(tert-butoxycarbonyl)-3,8-diazabicyclo[3.2.1]octane-3-yl)-7-(8-ethynyl-7-fluoro-3-(methoxymethoxy)naphthalen-1-yl)-8-fluoro-2-(2,2,2-trifluoroethoxy)quinazoline-6-carboxylate

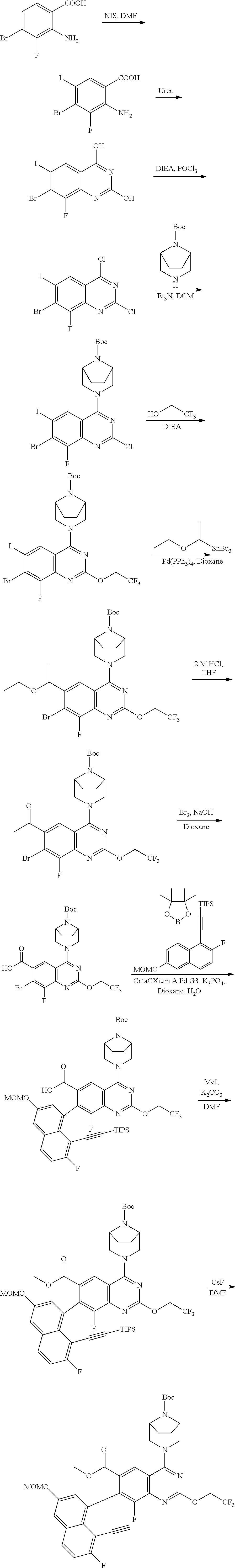

Step I: To a solution of 2-amino-4-bromo-3-fluorobenzoic acid (25.0 g, 106.83 mmol) in N,N-dimethylformamide (100 mL) was added N-iodosuccinimide (36.0 g, 160.24 mmol). The mixture was stirred at 80° C. for 2 hours. The reaction mixture was diluted with water (3 L) and stirred at room temperature for 1 hour. The mixture was filtered, and the resulting solid was washed with water (300 mL×3). The residue was dried under reduced pressure to give the yellow compound 2-amino-4-bromo-3-fluoro-5-iodobenzoic acid (34.0 g, 94.47 mmol, yield: 88%). LCMS (ESI): [M+H]⁺=359.9.

Step II: 2-Amino-4-bromo-3-fluoro-5-iodobenzoic acid (30.0 g, 83.35 mmol) and urea (50.0 g, 833.52 mmol) were placed in a reaction flask. The mixture was heated to 200° C. and reacted for 4 hours. After cooling to room temperature, water (300 mL) was added, and the mixture was stirred at 80° C. for 30 minutes. The resulting solid was filtered while hot. The process of slurrying and filtration was repeated twice, and the solid was dried under reduced pressure to give the yellow compound 7-bromo-8-fluoro-6-iodoquinazoline-2,4-diol (26.7 g, 69.36 mmol, yield: 83%). LCMS (ESI): [M+H]⁺=387.0.

Step III: 7-Bromo-8-fluoro-6-iodoquinazoline-2,4-diol (24.7 g, 64.17 mmol) was added to phosphorus oxychloride (300 mL). At 0° C., diisopropylethylamine (40.0 mL, 224.59 mmol) was added, and the mixture was stirred at 130° C. for 4 hours. After cooling to room temperature, the reaction mixture was concentrated under reduced pressure, and the residue was poured into ice water (500 mL). The mixture was extracted with dichloromethane (500 mL×3), and the combined organic layers were dried over anhydrous magnesium sulfate, filtered, and concentrated under reduced pressure. The residue was purified by flash chromatography on silica gel (0-4% gradient of methanol/dichloromethane) to give the compound 7-bromo-2,4-dichloro-8-fluoro-6-iodoquinazoline (26.7 g, 62.88 mmol, yield: 98%). LCMS (ESI): [M+H]⁺=420.8.

Step IV: To a solution of 7-bromo-2,4-dichloro-8-fluoro-6-iodoquinazoline (10.0 g, 23.71 mmol) in dichloromethane (100 mL) were added (1R,5S)-3,8-diazabicyclo[3.2.1]octane-8-tert-butyl ester (5.54 g, 26.08 mmol) and triethylamine (10.0 mL, 71.12 mmol). The reaction mixture was stirred at 20° C. for 2 hours. The mixture was concentrated under reduced pressure, and the residue was purified by flash chromatography on silica gel (0-36% gradient of ethyl acetate/petroleum ether) to give the compound tert-butyl (1R,5S)-3-(7-bromo-2-chloro-8-fluoro-6-iodoquinazolin-4-yl)-3,8-diazabicyclo[3.2.1]octane-8-carboxylate (3.60 g, 5.93 mmol, yield: 25%). LCMS (ESI): [M+H]⁺=597.0.

Step V: To a solution of tert-butyl (1R,5S)-3-(7-bromo-2-chloro-8-fluoro-6-iodoquinazolin-4-yl)-3,8-diazabicyclo[3.2.1]octane-8-carboxylate (25.4 g, 42.50 mmol) in 2,2,2-trifluoroethanol (193 mL) was added diisopropylethylamine (37.7 mL, 212.51 mmol). The reaction mixture was stirred at 70° C. for 16 hours. After cooling to room temperature, the mixture was concentrated under reduced pressure, and the residue was purified by flash chromatography on silica gel (0-40% gradient of tetrahydrofuran/petroleum ether) to give the compound tert-butyl (1S,5R)-3-(7-bromo-8-fluoro-6-iodo-2-(2,2,2-trifluoroethoxy)quinazolin-4-yl)-3,8-diazabicyclo[3.2.1]octane-8-carboxylate (20.1 g, 30.6 mmol, yield: 72%). LCMS (ESI): [M+H]⁺=661.3.

Step VI: At 25° C., To a stirred solution of tert-butyl (1S,5R)-3-(7-bromo-8-fluoro-6-iodo-2-(2,2,2-trifluoroethoxy)quinazolin-4-yl)-3,8-diazabicyclo[3.2.1]octane-8-carboxylate (1.00 g, 1.51 mmol) in 1,4-dioxane (20.0 mL) were added tributyltin (1-ethoxyvinyl) (510 mg, 1.41 mmol) and tetrakis(triphenylphosphine)palladium (170 mg, 0.15 mmol) under a nitrogen atmosphere. The mixture was stirred at 100° C. for 16 hours. After cooling to room temperature, the mixture was concentrated under reduced pressure, and the residue was purified by flash chromatography on silica gel (0-30% gradient of ethyl acetate/petroleum ether) to give a white solid compound tert-butyl (1R,5S)-3-(7-bromo-6-(1-ethoxyvinyl)-8-fluoro-2-(2,2,2-trifluoroethoxy)quinazolin-4-yl)-3,8-diazabicyclo[3.2.1]octane-8-carboxylate (680 mg, 1.12 mmol, yield: 74%). LCMS (ESI): [M+H]⁺=605.0.

Step VII: At 25° C., a solution of tert-butyl (1R,5S)-3-(7-bromo-6-(1-ethoxyvinyl)-8-fluoro-2-(2,2,2-trifluoroethoxy)quinazolin-4-yl)-3,8-diazabicyclo[3.2.1]octane-8-carboxylate (400 mg, 0.66 mmol) and hydrochloric acid (2 M aqueous solution, 1.65 mL, 3.30 mmol) in tetrahydrofuran (8.00 mL) was prepared. The reaction mixture was stirred at 20° C. for 2 hours. The pH was adjusted to 7 by using saturated sodium bicarbonate, and the mixture was extracted with ethyl acetate (4 mL×3). The organic phase was concentrated under reduced pressure, and the residue was purified by flash column chromatography (silica gel, 0-15% gradient of ethyl acetate/petroleum ether) to give a white solid compound: tert-butyl (1R,5S)-3-(6-acetyl-7-bromo-8-fluoro-2-(2,2,2-trifluoroethoxy)quinazolin-4-yl)-3,8-diaza-bicyclo[3.2.1]octane-8-carboxylate (220 mg, 0.38 mmol, yield: 58%). LCMS (ESI): [M+H]⁺=577.2.

Step VIII: To a stirred solution of tert-butyl (1R,5S)-3-(6-acetyl-7-bromo-8-fluoro-2-(2,2,2-trifluoroethoxy)quinazolin-4-yl)-3,8-diazabicyclo[3.2.1]octane-8-carboxylate (2.00 g, 3.46 mmol) in dioxane (20 mL) and sodium hydroxide (1.5 M aqueous solution, 18.5 mL, 27.7 mmol) was added bromine (0.53 mL, 10.4 mmol) at 0° C. The reaction mixture was stirred at 0° C. for 3 hours and then at 20° C. for 16 hours. To the reaction mixture, water (50 mL) was added, and the pH was adjusted to 4 with 2 M diluted hydrochloric acid. The mixture was extracted with ethyl acetate (50 mL×3). The organic layers were washed with saturated brine (50 mL), dried over sodium sulfate, filtered, and concentrated under reduced pressure. The residue was purified by flash column chromatography (silica gel, 0-40% gradient of ethyl acetate/petroleum ether) to afford 7-bromo-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (1.00 g, 1.73 mmol, yield: 50%). LCMS (ESI): [M+H]⁺=581.3.

Step IX: To a stirred solution of 7-bromo-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (350 mg, 0.60 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (402 mg, 0.78 mmol), and potassium phosphate (1.5 M aqueous solution, 1.20 mL, 1.81 mmol) in dioxane (3.50 mL) was added (methanesulfonyl)(diadamantyl-tert-butylphosphino)-2-amino-1,1′-biphenyl-2-yl)palladium(II) (44 mg, 0.06 mmol) under a nitrogen atmosphere. The reaction mixture was stirred at 100° C. for 16 hours. Four identical batches of this reaction were combined, diluted with water (20 mL), and extracted with ethyl acetate (20 mL×2). The organic layers were combined, dried, filtered, and concentrated under reduced pressure. The residue was purified by flash chromatography on silica gel (0-50% gradient of ethyl acetate/petroleum ether) to give 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylic acid (650 mg, 0.72 mmol, yield: 30%). LCMS (ESI): [M+H]⁺=885.3.

Step X: At 0° C., To a suspension of 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylic acid (650 mg, 0.72 mmol) and potassium carbonate (329 mg, 2.38 mmol) in N,N-dimethylformamide (7 mL) was added methyl iodide (224 mg, 1.58 mmol) and the reaction mixture was stirred at 20° C. for 16 hours. After completion, the reaction mixture was diluted with water (20 mL) and extracted with ethyl acetate (20 mL×2). The organic phases were combined, dried over magnesium sulfate, filtered, and concentrated under reduced pressure to give a crude product. The crude compound, methyl 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylate (600 mg), was obtained as a yellow oily liquid. LCMS (ESI): [M+H]⁺=899.3.

Step XI: To a solution of methyl 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylate (600 mg, 0.66 mmol) in N,N-dimethylformamide (6 mL) was added cesium fluoride (1.00 g, 6.60 mmol) and the reaction mixture was stirred at 20° C. for 3 hours. After completion, the reaction mixture was diluted with water (10 mL) and extracted with ethyl acetate (10 mL×3). The organic layers were combined and concentrated under reduced pressure. The residue was purified by flash chromatography on silica gel (0-50% gradient of ethyl acetate/petroleum ether) to give a compound methyl 4-((1R,5S)-8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoro-2-(2,2,2-trifluoroethoxy)quinazolin-6-carboxylate (400 mg, 0.53 mmol, yield: 80%). LCMS (ESI): [M+H]⁺=743.6.

### Intermediate 3: (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7,8,9-hexahydronaphtho[1′,8′:4,5,6][1]azacycloundecino[2,3-g]quinazoline

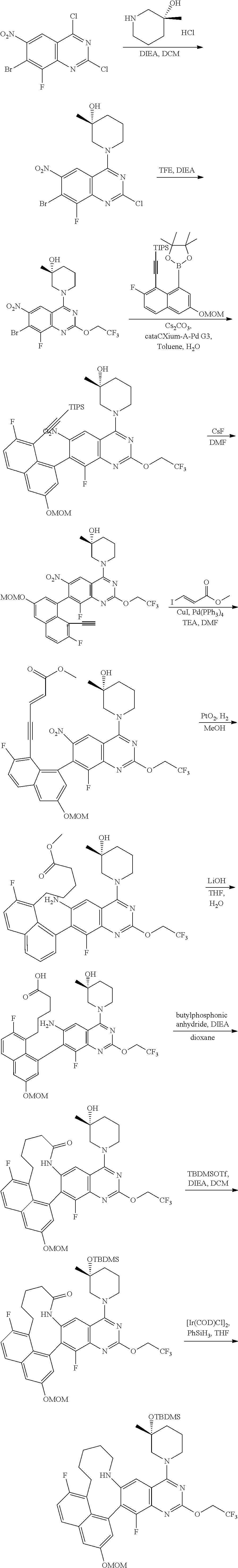

Step I: At −40° C., To a solution of 7-bromo-2,4-dichloro-8-fluoro-6-nitroquinazoline (8.00 g, 23.47 mmol) in dichloromethane (50 mL) were added (R)-3-methylpiperidin-3-ol hydrochloride (2.49 g, 16.43 mmol) and diisopropylethylamine (25.0 mL, 140.8 mmol). The mixture was stirred at −40° C. for 4 hours. The reaction mixture was diluted with water (50 mL), extracted with dichloromethane (50 mL×3). The combined organic phases were dried over anhydrous sodium sulfate, filtered, and concentrated under reduced pressure. The residue was purified by flash chromatography on silica gel (0-35% gradient of ethyl acetate/petroleum ether) to give a yellow solid (R)-1-(7-bromo-2-chloro-8-fluoro-6-nitroquinazolin-4-yl)-3-methylpiperidin-3-ol (7.50 g, 17.87 mmol, yield: 76%). LCMS (ESI): [M+H]⁺=420.9.

Step II: At 20° C., To a solution of (R)-1-(7-bromo-2-chloro-8-fluoro-6-nitroquinazolin-4-yl)-3-methylpiperidin-3-ol (7.50 g, 17.87 mmol) in trifluoroethanol (80 mL) was added diisopropylethylamine (12.5 mL, 71.49 mmol). The mixture was stirred at 70° C. for 3 hours. The reaction mixture was filtered, and the filtrate was concentrated under reduced pressure. The residue was purified by flash chromatography on silica gel (0-35% gradient of ethyl acetate/petroleum ether) to give a yellow solid compound (R)-1-(7-bromo-2-chloro-8-fluoro-6-nitroquinazolin-4-yl)-3-methylpiperidin-3-ol (7.50 g, 17.87 mmol, yield: 76%). LCMS (ESI): [M+H]⁺=483.0.

Step III: To a solution of (R)-1-(7-bromo-8-fluoro-6-nitro-2-(2,2,2-trifluoroethoxy)quinazolin-4-yl)-3-methylpiperidin-3-ol (61 g, 126.24 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (67.94 g, 132.55 mmol), and cesium carbonate (123.39 g, 378.71 mmol) in toluene (1000 mL) and water (250 mL) was added methylsulfonate-[2-(dicyclohexylphosphanyl)-2′,6′-dimethoxybiphenyl]palladium(II) (18.39 g, 25.25 mmol) under a nitrogen atmosphere at 25° C. The solution was stirred at 100° C. for 16 hours. The reaction mixture was then concentrated and purified by flash column chromatography (silica gel, 0-20% gradient of tetrahydrofuran/petroleum ether), to give a brown solid compound (3R)-1-(8-fluoro-7-(7-fluoro-3-(methoxymethoxy)-8-((triisopropylsilyl)ethynyl)naphthalen-1-yl)-6-nitro-2-(2,2,2-trifluoroethoxy)quinazolin-4-yl)-3-methylpiperidin-3-ol (90.00 g, 107.24 mmol, yield: 85%). LCMS (ESI): [M+H]⁺=789.5.

Step IV: At 25° C., to a solution of (3R)-1-(7-(8-ethynyl-7-fluoro-3-(methoxymethoxy)naphthalen-1-yl)-8-fluoro-6-nitro-2-(2,2,2-trifluoroethoxy)quinazolin-4-yl)-3-methylpiperidin-3-ol (64.00 g, 81.13 mmol) in dimethylformamide (500 mL) was added cesium fluoride (61.62 g, 405.64 mmol), and the reaction mixture was stirred at 25° C. for 2 hours. The reaction mixture was diluted with water (500 mL) and extracted with ethyl acetate (500 mL×3). The combined organic phases were dried over anhydrous sodium sulfate, filtered, and concentrated under reduced pressure. The crude product was purified by flash chromatography on silica gel (0-30% gradient of tetrahydrofuran/petroleum ether) to give a yellow solid compound (3R)-1-(7-(8-ethynyl-7-fluoro-3-(methoxymethoxy)naphthalen-1-yl)-8-fluoro-6-nitro-2-(2,2,2-trifluoroethoxy)quinazolin-4-yl)-3-methylpiperidin-3-ol (35.00 g, 55.33 mmol, yield: 68%). LCMS (ESI): [M+H]⁴=633.1.

Step V: To a solution of (3R)-1-(7-(8-ethynyl-7-fluoro-3-(methoxymethoxy)naphthalen-1-yl)-8-fluoro-6-nitro-2-(2,2,2-trifluoroethoxy)quinazolin-4-yl)-3-methylpiperidin-3-ol (35.00 g, 55.33 mmol) and (2E)-3-iodoacrylate methyl ester (17.60 g, 83.00 mmol) in triethylamine (175 mL) and dimethylformamide (260 mL) were added copper(I) iodide (1.05 g, 5.53 mmol) and tetrakis(triphenylphosphine)palladium (12.79 g, 11.07 mmol) at 25° C. under a nitrogen atmosphere. The mixture was stirred at 50° C. for 2 hours. The reaction mixture was filtered, and the filtrate was concentrated under reduced pressure. The crude product was purified by flash chromatography on silica gel (0-35% gradient of tetrahydrofuran/petroleum ether) to give a brown solid compound (E)-5-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)pent-2-en-4-ynoate methyl ester (40.00 g, 47.45 mmol, yield: 86%). LCMS (ESI): [M+H]⁺=717.0.

Step VI: At 25° C., To a solution of (E)-5-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)pent-2-en-4-ynoate methyl ester (27.00 g, 37.68 mmol) in methanol (1500 mL) was added platinum dioxide (2.70 g, 11.89 mmol). The reaction mixture was stirred under hydrogen atmosphere (15 psi) and 25° C. for 16 hours. After completion, the reaction mixture was filtered, and the filtrate was concentrated. The crude product was purified by flash chromatography on silica gel (0-15% gradient of tetrahydrofuran/dichloromethane) to give a red solid compound 5-(8-(6-amino-8-fluoro-4-((R)-3-hydroxy-3-methylpiperidin-1-yl)-2-(2,2,2-trifluoroethoxy)quinazolin-7-yl)-2-fluoro-6-(methoxymethoxy)naphthalen-1-yl)pentanoate methyl ester (16.00 g, 23.01 mmol, yield: 61%). LCMS (ESI): [M+H]⁺=693.2.

Step VII: At 25° C., To a solution of 5-(8-(6-amino-8-fluoro-4-((R)-3-hydroxy-3-methylpiperidin-1-yl)-2-(2,2,2-trifluoroethoxy)quinazolin-7-yl)-2-fluoro-6-(methoxymethoxy)naphthalen-1-yl)pentanoate methyl ester (17.00 g, 24.54 mmol) in tetrahydrofuran (170 mL) and water (34 mL) was added lithium hydroxide (3.09 g, 73.63 mmol). The mixture was stirred at 50° C. for 16 hours. The reaction mixture was diluted with water (200 mL), and the pH was adjusted to 4 by using concentrated hydrochloric acid. The reaction mixture was extracted with ethyl acetate (200 mL×3), and the combined organic layers were dried over anhydrous sodium sulfate, filtered, and concentrated under reduced pressure to give a brown solid compound 5-(8-(6-amino-8-fluoro-4-((S)-3-hydroxy-3-methylpiperidin-1-yl)-2-(2,2,2-trifluoroethoxy)quinazolin-7-yl)-2-fluoro-6-(methoxymethoxy)naphthalen-1-yl)pentanoic acid (17.00 g, 22.54 mmol, yield: 92%). LCMS (ESI): [M+H]⁺=679.3.

Step VIII: At 25° C., To a solution of 5-(8-(6-amino-8-fluoro-4-((S)-3-hydroxy-3-methylpiperidin-1-yl)-2-(2,2,2-trifluoroethoxy)quinazolin-7-yl)-2-fluoro-6-(methoxymethoxy)naphthalen-1-yl)pentanoic acid (17.00 g, 25.05 mmol) and diisopropylethylamine (175 mL, 1002.01 mmol) in dioxane (660 mL) was added tributylphosphine anhydride (40.91 g, 75.15 mmol), and the reaction mixture was stirred at 60° C. for 16 hours. The reaction was quenched with water (600 mL) and extracted with ethyl acetate (600 mL×3). The combined organic layers were dried over anhydrous sodium sulfate, filtered, and concentrated under reduced pressure. The crude product was purified by flash chromatography on silica gel (0-50% gradient of tetrahydrofuran/petroleum ether) to give a brown solid compound (R)-3,15-difluoro-11-(3-hydroxy-3-methylpiperidin-1-yl)-17-(methoxymethoxy)-13-(2,2,2-trifluoroethoxy)-4,5,6,7-tetrahydronaphtho[1′,8′:4,5,6][1]azacycloundecine[2,3-g]quinazolin-8(9H)-one (13.00 g, 19.68 mmol, yield: 79%). LCMS (ESI): [M+H]⁺=661.3.

Step IX: At 0° C., To a solution of (R)-3,15-difluoro-11-(3-hydroxy-3-methylpiperidin-1-yl)-17-(methoxymethoxy)-13-(2,2,2-trifluoroethoxy)-4,5,6,7-tetrahydronaphtho[1′,8′:4,5,6][1]azacycloundecino[2,3-g]quinazolin-8(9H)-one (2.0 g, 3.03 mmol) in dichloromethane (20 mL) were added tert-butyldimethylsilyl trifluoromethanesulfonate (2.28 mL, 18.16 mmol) and diisopropylethylamine (2.0 mL, 12.11 mmol). The mixture was stirred at 20° C. for 3 hour under nitrogen atmosphere. and the reaction mixture was diluted with water (20 mL), extracted with dichloromethane (20 mL×3). The combined organic phases were dried over anhydrous sodium sulfate, filtered, and concentrated under reduced pressure. The residue was purified by flash column chromatography (silica gel, gradient elution with 0-20% tetrahydrofuran/(petroleum ether/dichloromethane, v/v 3:1)), to give a yellow solid compound (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7-tetrahydronaphtho[1′,8′:4,5,6][1]azacycloundecino[2,3-g]quinazolin-8(9H)-one (1.92 g, 2.48 mmol, yield: 82%). LCMS (ESI): [M+H]⁺=775.3.

Step X: At 25° C., a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7-tetrahydronaphtho[1′,8′:4,5,6][1]azacycloundecino[2,3-g]quinazolin-8(9H)-one (600.0 mg, 0.77 mmol) and (1,5-cyclooctadiene)chloroiridium(I) dimer (52.0 mg, 0.08 mmol) in tetrahydrofuran (48 mL) was treated with phenylsilane (1.91 mL, 15.49 mmol). The reaction mixture was stirred under a nitrogen atmosphere at 25° C. for 12 hours. The residue was purified by flash column chromatography (silica gel, gradient elution with 0-20% tetrahydrofuran/(petroleum ether/dichloromethane, v/v 3:1)), to give a yellow solid compound (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7,8,9-hexahydronaphtho[1′,8′:4,5,6][1]azacycloundecino[2,3-g]quinazolin (430 mg, 0.57 mmol, yield: 73%). LCMS (ESI): [M+H]⁺=761.3.

### Intermediate 4: (R)-11-(3-((tert-butyldimethylsilyl)oxy)-3-methylpiperidin-1-yl)-3,15-difluoro-17-(methoxymethoxy)-9-methyl-13-(2,2,2-trifluoroethoxy)-4,5,8,9-tetrahydro-7H-naphtho[1′,8′:7,8,9][1]oxa[4]azacycloundeca[5,6-g]quinazoline

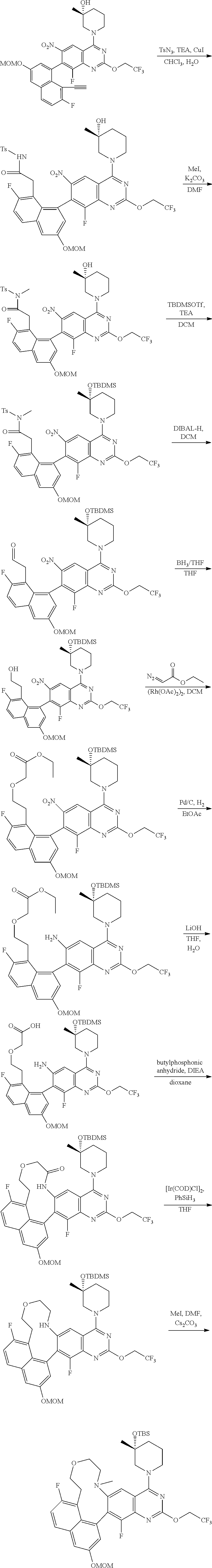

Step I: To a solution of (3R)-1-(7-(8-ethynyl-7-fluoro-3-(methoxymethoxy)naphthalen-1-yl)-8-fluoro-6-nitro-2-(2,2,2-trifluoroethoxy)quinazolin-4-yl)-3-methylpiperidin-3-ol (3.9 g, 6.17 mmol), 4-methylbenzenesulfonyl azide (1.95 g, 7.40 mmol), and copper(I) iodide (0.12 g, 0.62 mmol) in chloroform (50 mL) and water (0.28 mL, 15.41 mmol) was added triethylamine (1.03 mL, 7.40 mmol) at 20° C. under a nitrogen atmosphere. The reaction mixture was stirred at 20° C. for 12 hours, then diluted with water (50 mL) and extracted with ethyl acetate (20 mL×3). The organic layers were combined, dried over anhydrous sodium sulfate, filtered, and concentrated under reduced pressure to give a crude compound 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-(p-toluenesulfonyl)acetamide (5.0 g) as a brown solid. LCMS (ESI): [M+H]⁺=820.0.

Step II: To a solution of 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-(p-toluenesulfonyl)acetamide (5.4 g, 6.59 mmol) in N,N-dimethylformamide (50 mL) were added iodomethane (1.23 mL, 19.76 mmol) and potassium carbonate (2.73 g, 19.76 mmol) at 20° C. The reaction mixture was stirred at 20° C. for 12 hours, then diluted with water (100 mL) and extracted with ethyl acetate (50 mL×3). The organic layers were combined, dried over anhydrous sodium sulfate, filtered, and concentrated under reduced pressure. The residue was purified by flash column chromatography (silica gel, 0-30% gradient of tetrahydrofuran/petroleum ether) to give a brown solid compound 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-methyl-N-(p-toluenesulfonyl)acetamide (2.38 g, 2.85 mmol, yield: 43%). LCMS (ESI): [M+H]⁺=834.2.

Step III: To a solution of 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-methyl-N-(p-toluenesulfonyl)acetamide (11.5 g, 13.79 mmol) and triethylamine (9.59 mL, 68.96 mmol) in dichloromethane (140 mL) was added dropwise tert-butyldimethylsilyl trifluoromethanesulfonate (5.20 mL, 41.38 mmol) at 0° C. The reaction mixture was stirred at 20° C. for 16 hours. and the reaction mixture was diluted with water (40 mL), extracted with dichloromethane (40 mL×3). The combined organic phases were dried over anhydrous sodium sulfate, filtered, and concentrated under reduced pressure. The residue was purified by flash column chromatography (silica gel, 0-5% gradient of tetrahydrofuran in a mixture of petroleum ether and dichloromethane at a 3:1 ratio), to give a brown solid compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)-N-methyl-N-(p-toluenesulfonyl)acetamide (12.7 g, 13.41 mmol, yield: 97%). LCMS (ESI): [M+H]⁺=948.4.

Step IV: To a solution of 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)-N-methyl-N-(p-toluenesulfonyl)acetamide (12.50 g, 13.19 mmol) in dichloromethane (155 mL) was added 1 M diisobutylaluminum hydride (42.19 mL, 42.19 mmol) at −70° C. The reaction mixture was stirred at −70° C. for 1 hour, then quenched with acetic acid (7.25 mL, 126.58 mmol). The reaction mixture was diluted with water (100 mL) and stirred for 16 hours. The reaction mixture was extracted with dichloromethane (100 mL×3). The combined organic phases were dried over anhydrous sodium sulfate, filtered, and concentrated under reduced pressure. The residue was purified by flash column chromatography (silica gel, 0-20% gradient of tetrahydrofuran/(petroleum ether:dichloromethane=3:1)) to give a compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)acetaldehyde (7.70 g, 10.08 mmol, yield: 76%) as a brown solid. LCMS (ESI): [M+H]⁺=765.3.

Step V: To a solution of 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)acetaldehyde (18.50 g, 24.19 mmol) in tetrahydrofuran (190 mL) was added borane/tetrahydrofuran (1 M, 41 mL, 40.71 mmol) at 0° C. The reaction mixture was stirred at 10° C. for 16 hours. The reaction was quenched with methanol at 0° C. The mixture was concentrated under reduced pressure to give a brown solid compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethanol (18.50 g, 21.71 mmol, yield: 90%). LCMS (ESI): [M+H]⁺=767.8.

Step VI: To a solution of 2-(8-(4-((R)-3-((tert-butyldimethylsilyl)oxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethanol (1.0 g, 1.30 mmol) and rhodium acetate dimer (57.6 mg, 0.13 mmol) in dichloromethane (10.0 mL) was added ethyl diazoacetate (0.62 mL, 5.87 mmol) dropwise under a nitrogen atmosphere. The mixture was stirred at 25° C. for 1 hour under a nitrogen atmosphere. The reaction was quenched with methanol (5 mL), and the solvent was removed under reduced pressure. The residue was purified by flash column chromatography on silica gel (gradient elution: 0-20% tetrahydrofuran in a mixture of petroleum ether and dichloromethane in a 10:1 ratio) to give a brown oily crude compound ethyl 2-(2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethoxy)acetate (1.20 g). LCMS (ESI): [M+H]⁺=853.3.

Step VII: To a solution of ethyl 2-(2-(8-(4-((R)-3-((tert-butyldimethylsilyl)oxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethoxy)acetate (1.0 g, 0.28 mmol) in ethyl acetate (40 mL) was added palladium on carbon (wet) (200 mg). The mixture was stirred under hydrogen atmosphere (45 psi) at 20° C. for 12 hours, then filtered. The filtrate was concentrated under reduced pressure to give a brown oily crude compound ethyl 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)acetate (1.0 g). LCMS (ESI): [M+H]⁺=823.3.

Step VIII: To a solution of ethyl 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)acetate (1.0 g, 1.22 mmol) in tetrahydrofuran (8.0 mL) and water (2.0 mL) was added lithium hydroxide (153.0 mg, 3.65 mmol). The mixture was stirred at 50° C. for 3 hours and at 25° C. for 12 hours, then the pH was adjusted to 5-6 with 1 M hydrochloric acid. The mixture was extracted with ethyl acetate (5 mL×3), and the combined organic layers were dried over anhydrous sodium sulfate, filtered, and concentrated under reduced pressure to give a yellow solid compound 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)acetic acid (0.8 g, 1.01 mmol, yield: 83%). LCMS (ESI): [M+H]⁺=795.3.

Step IX: To a solution of 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)acetic acid (3.30 g, 4.15 mmol) and diisopropylethylamine (29 mL, 166.06 mmol) in dioxane (135 mL) was added butylphosphonic anhydride (50% ethyl acetate solution, 6.78 g, 12.45 mmol) at 25° C. under a nitrogen atmosphere. The reaction mixture was stirred at 60° C. for 2 hours. The mixture was diluted with water (160 mL), extracted with ethyl acetate (160 mL×3). The combined organic phases were dried over anhydrous sodium sulfate, filtered, and concentrated to give a brown solid compound (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5-dihydro-7H-naphtho[1′,8′:7,8,9][1]oxa[4]azacycloundeca[5,6-g]quinazoline-8(9H)-one (3.04 g, 3.33 mmol, yield: 80%). LCMS (ESI): [M+H]⁺=777.5.

Step X: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5-dihydro-7H-naphtho[1′,8′:7,8,9][1]oxa[4]azacycloundeca[5,6-g]quinazoline-8(9H)-one (900 mg, 1.2 mmol) and phenylsilane (2.86 mL, 23.2 mmol) in tetrahydrofuran (70 mL) was added bis(1,5-cyclooctadiene)chloroiridium(I) dimer (78 mg, 0.1 mmol) at 0° C. under a nitrogen atmosphere. The mixture was stirred at 20° C. for 16 hours, then concentrated under reduced pressure. The residue was purified by flash chromatography on silica gel (0-20% gradient of tetrahydrofuran/(3:1 petroleum ether/dichloromethane)) to afford a yellow solid, (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundeca[5,6-g]quinazoline (500 mg, 0.7 mmol, yield: 56%). LCMS (ESI): [M+H]⁺=763.3.

Step XI: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundeca[5,6-g]quinazoline (500 mg, 0.66 mmol) in N,N-dimethylformamide (10 mL) were added iodomethane (81.0 LL, 1.32 mmol) and cesium carbonate (235 mg, 0.72 mmol). The reaction mixture was stirred at 25° C. for 16 hours. then diluted with water (3 mL) and extracted with ethyl acetate (5 mL×5). The combined organic layers were dried over anhydrous sodium sulfate, filtered, and concentrated under reduced pressure. The residue was purified by flash column chromatography on silica gel (gradient elution: 0-15% tetrahydrofuran in a mixture of petroleum ether and dichloromethane in a 3:1 ratio) to give a yellow solid compound (R)-11-(3-((tert-butyldimethylsilyloxy)-3-methylpiperidin-1-yl)-3,15-difluoro-17-(methoxymethoxy)-9-methyl-13-(2,2,2-trifluoroethoxy)-4,5,8,9-tetrahydro-7H-naphtho[1′,8′:7,8,9][1]oxa[4]azacycloundeca[5,6-g]quinazoline (460 mg, 0.59 mmol, yield: 90%). LCMS (ESI): [M+H]⁺=777.3.

### Intermediate 5: tert-Butyl (1R,5S)-3-(3,14-difluoro-16-(methoxymethoxy)-7-oxo-12-(2,2,2-trifluoroethoxy)-5,6,7,8-tetrahydro-4H-naphtho[1′,8′:4,5,6]acridino[2,3-g]quinazolin-10-yl)-3,8-diaza-bicyclo[3.2.1]octane-8-carboxylate

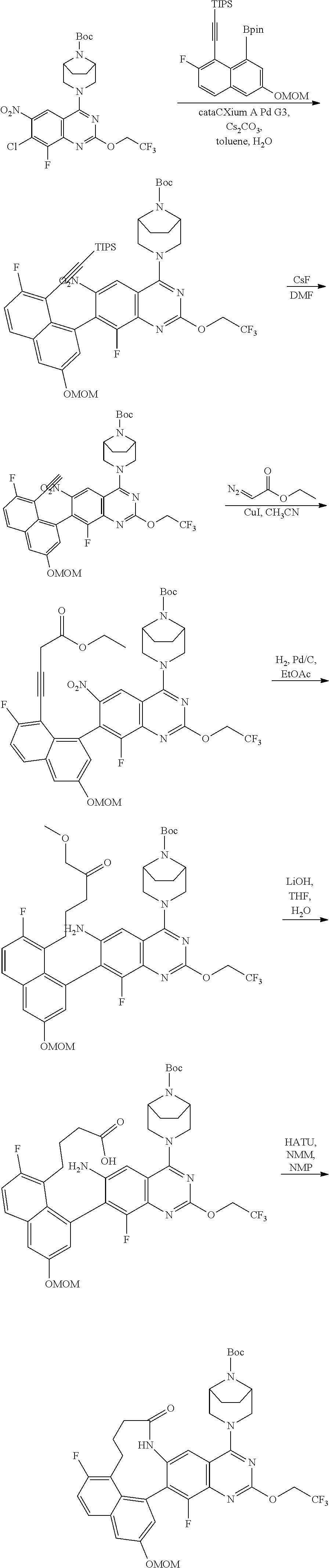

Step I: A mixed solution of tert-butyl (1R,5S)-3-(7-bromo-8-fluoro-6-nitro-2-(2,2,2-trifluoroethoxy)quinazolin-4-yl)-3,8-diaza-bicyclo[3.2.1]octane-8-carboxylate (3.10 g, 5.34 mmol) and (((2-fluoro-6-(methoxymethoxy)-8-(4,4,5,5-tetramethyl-1,3,2-dioxaborolane-2-yl)naphthalen-1-yl)ethynyl)tris(isopropyl)silane (3.56 g, 6.94 mmol) in toluene (124 mL) and water (31.0 mL) were added cesium carbonate (5.22 g, 16.03 mmol) and (mesylate-(bis(adamantyl)-n-butylphosphine)-2-amino-1,1′-biphenyl) palladium(II) catalyst (0.78 g, 1.07 mmol) under a nitrogen atmosphere. The mixture was stirred at 100° C. for 16 h. The residue was diluted with water (150 mL) and extracted with ethyl acetate (150 mL×3). The combined organic layers were washed with saturated brine (150 mL), dried over anhydrous magnesium sulfate, and filtered. The filtrate was concentrated under reduced pressure. The residue was purified by flash column chromatography (silica gel, 0-10% gradient of tetrahydrofuran/petroleum ether) to give a brown solid compound tert-butyl (1R,5S)-3-(8-fluoro-7-(7-fluoro-3-(methoxymethoxy)-8-((tris(isopropyl)silyl)ethynyl)naphthalen-1-yl)-6-nitro-2-(2,2,2-trifluoroethoxy)quinazolin-4-yl)-3,8-diaza-bicyclo[3.2.1]octane-8-carboxylate (3.27 g, 3.69 mmol, yield: 69%). LCMS (ESI): [M+H]⁺=886.1.

Step II: To a solution of tert-butyl (1R,5S)-3-(8-fluoro-7-(7-fluoro-3-(methoxymethoxy)-8-(triisopropylsilyl)ethynyl)naphthalen-1-yl)-6-nitro-2-(2,2,2-trifluoroethoxy)quinazolin-4-yl)-3,8-diaza-bicyclo[3.2.1]octane-8-carboxylate (5.00 g, 5.64 mmol) in N,N-dimethylformamide (50.0 mL) was added cesium fluoride (4.29 g, 28.22 mmol). The reaction was stirred at 20° C. for 16 hours. The reaction mixture was diluted with water (30 mL), extracted with ethyl acetate (15 mL×3). The combined organic layers were dried over anhydrous magnesium sulfate, filtered, and concentrated. The residue was purified by flash chromatography (silica gel, 0-20% gradient of tetrahydrofuran/petroleum ether) to give a white solid compound tert-butyl (1R,5S)-3-(7-(8-ethynyl-7-fluoro-3-(methoxymethoxy)naphthalen-1-yl)-8-fluoro-6-nitro-2-(2,2,2-trifluoroethoxy)quinazolin-4-yl)-3,8-diaza-bicyclo[3.2.1]octane-8-carboxylate (1.50 g, 2.03 mmol, yield: 36%). LCMS (ESI): [M+H]⁺=730.0.

Step III: To a solution of tert-butyl (1R,5S)-3-(7-(8-ethynyl-7-fluoro-3-(methoxymethoxy)naphthalen-1-yl)-8-fluoro-6-nitro-2-(2,2,2-trifluoroethoxy)quinazolin-4-yl)-3,8-diaza-bicyclo[3.2.1]octane-8-carboxylate (300 mg, 0.41 mmol) and ethyl diazoacetate (56.3 mg, 0.49 mmol) in acetonitrile (6.00 mL) was added copper(I) iodide (7.83 mg, 0.04 mmol). The reaction mixture was stirred at 20° C. for 16 hours, then concentrated under reduced pressure. The residue was purified by flash chromatography (silica gel, 0-30% gradient of tetrahydrofuran/petroleum ether) to give a brown oily compound tert-butyl (1R,5S)-3-(7-(8-(4-ethoxy-4-oxobut-1-yn-1-yl)-7-fluoro-3-(methoxymethoxy)naphthalen-1-yl)-8-fluoro-6-nitro-2-(2,2,2-trifluoroethoxy)quinazolin-4-yl)-3,8-diaza-bicyclo[3.2.1]octane-8-carboxylate (65% purity, 310 mg, 0.24 mmol, yield: 60%). LCMS (ESI): [M+H]⁺=816.3.

Step IV: To a stirred solution of tert-butyl (1R,5S)-3-(7-(8-(4-ethoxy-4-oxobut-1-yn-1-yl)-7-fluoro-3-(methoxymethoxy)naphthalen-1-yl)-8-fluoro-6-nitro-2-(2,2,2-trifluoroethoxy)quinazolin-4-yl)-3,8-diaza-bicyclo[3.2.1]octane-8-carboxylate (65% purity, 280 mg, 0.22 mmol) in ethyl acetate (10.0 mL) was added dry palladium on carbon (200 mg) at 25° C., and the reaction mixture was stirred at 20° C. for 16 hours under hydrogen atmosphere (15 psi). The reaction mixture was filtered, and the filtrate was concentrated under reduced pressure to give a brown oily compound tert-butyl (1R,5S)-3-(6-amino-7-(8-(4-ethoxy-4-oxobutyl)-7-fluoro-3-(methoxymethoxy)naphthalen-1-yl)-8-fluoro-2-(2,2,2-trifluoroethoxy)quinazolin-4-yl)-3,8-diaza-bicyclo[3.2.1]octane-8-carboxylate (65% purity, 241 mg, 0.20 mmol, yield: 89%). LCMS (ESI): [M+H]⁺=790.5.

Step V: To a stirred solution of tert-butyl (1R,5S)-3-(6-amino-7-(8-(4-ethoxy-4-oxobutyl)-7-fluoro-3-(methoxymethoxy)naphthalen-1-yl)-8-fluoro-2-(2,2,2-trifluoroethoxy)quinazolin-4-yl)-3,8-diaza-bicyclo[3.2.1]octane-8-carboxylate (65% purity, 230 mg, 0.20 mmol) in the solvent of tetrahydrofuran (4.60 mL) and water (920 μL) was added lithium hydroxide (73.3 mg, 1.74 mmol) at 25° C. The reaction mixture was stirred at 60° C. for 16 hours. The reaction mixture was cooled to room temperature, and the pH was adjusted to 1 by using 1 N hydrochloric acid. The mixture was extracted with ethyl acetate (5 mL×3), and the organic phase was concentrated under reduced pressure to give a brown solid compound 4-(8-(6-amino-4-((1R,5S)-8-(tert-butoxycarbonyl)-3,8-diaza-bicyclo[3.2.1]octane-3-yl)-8-fluoro-2-(2,2,2-trifluoroethoxy)quinazolin-7-yl)-2-fluoro-6-(methoxymethoxy)naphthalen-1-yl)butanoic acid (130 mg, 0.17 mmol, yield: 85%). LCMS (ESI): [M+H]⁺=762.3

Step VI: To a solution of 4-(8-(6-amino-4-((1R,5S)-8-(tert-butoxycarbonyl)-3,8-diaza-bicyclo[3.2.1]octan-3-yl)-8-fluoro-2-(2,2,2-trifluoroethoxy)quinazolin-7-yl)-2-fluoro-6-(methoxymethoxy)naphthalen-1-yl)butanoic acid (130 mg, 0.17 mmol) and N-methylmorpholine (66.3 mg, 0.66 mmol) in N-methyl-2-pyrrolidone (2.00 mL) was added 0-(7-azabenzotriazol-1-yl)-N,N,N,N-tetramethyluronium hexafluorophosphate (150 mg, 0.39 mmol) at 25° C. The reaction mixture was stirred at 20° C. for 2 hours and then heated to 80° C. for 16 hours. After cooling to room temperature, the reaction mixture was diluted with water (3 mL) and extracted with ethyl acetate (3 mL×3). The combined organic layers were concentrated under reduced pressure, and the residue was purified by flash chromatography (silica gel, 0-40% gradient of tetrahydrofuran/petroleum ether) to give a brown solid compound tert-butyl (1R,5S)-3-(3,14-difluoro-16-(methoxymethoxy)-7-oxo-12-(2,2,2-trifluoroethoxy)-5,6,7,8-tetrahydro-4H-naphtho[1′,8′:4,5,6]acridine[2,3-g]quinazolin-10-yl)-3,8-diaza-bicyclo[3.2.1]octane-8-carboxylate (80% purity, 160 mg, 0.17 mmol, yield: 99%). LCMS (ESI): [M+H]⁺=744.1

### Intermediate 6: (R)-1-(3,15-Difluoro-13-(2,2,2-trifluoroethoxy)-4,5,8,9-tetrahydro-7H-naphtho[1′,8′:7,8,9111]oxa[4]azacycloundeca[5,6-g]quinazoline-11-yl)-3-methylpiperidin-3-ol

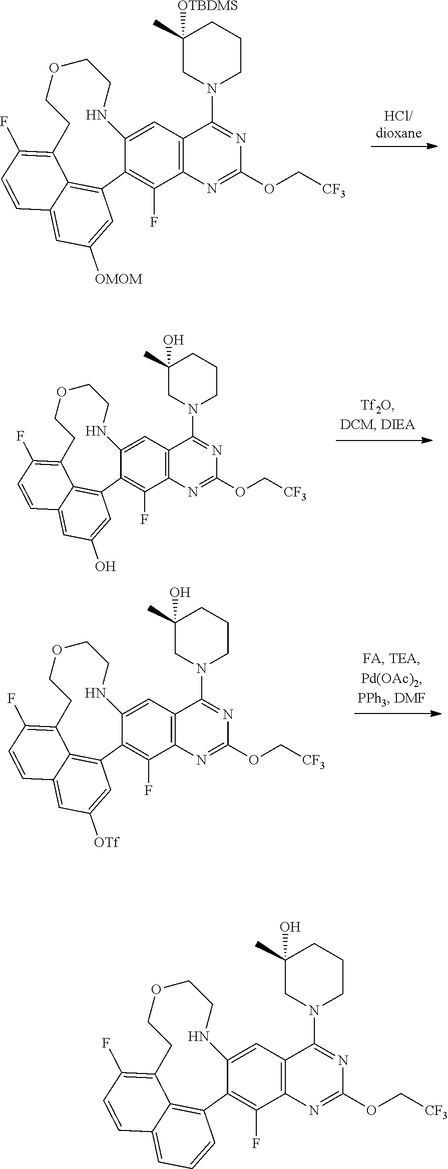

Step I: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundeca[5,6-g]quinazoline (200 mg, 0.26 mmol) in acetonitrile (2.00 mL) was added 4 M hydrochloric acid in dioxane (7.86 mL, 31.46 mmol). The mixture was stirred at 25° C. for 48 hours. After concentration under reduced pressure, the residue was purified by flash chromatography (silica gel, 0-15% gradient methanol/dichloromethane) to give a brown solid compound (R)-3,15-difluoro-11-(3-hydroxy-3-methylpiperidin-1-yl)-13-(2,2,2-trifluoroethoxy)-4,5,8,9-tetrahydro-7H-naphtho[1′,8′:7,8,9][1]oxa[4]azacycloundeca[5,6-g]quinazoline-17-ol (157 mg, 0.26 mmol, yield: 99%/). LCMS (ESI): [M+H]⁺=605.1. ¹H NMR (400 MHz, CD₃OD) δ ppm 8.18-7.94 (m, 1H), 7.77 (dd, J=6.1, 9.0 Hz, 1H), 7.48-7.25 (m, 2H), 7.07-6.93 (m, 1H), 5.18 (br d, J=7.8 Hz, 2H), 3.86 (br s, 1H), 3.70-3.53 (m, 4H), 3.53-3.37 (m, 3H), 3.29-3.10 (m, 2H), 2.79-2.68 (m, 1H), 2.62-2.47 (m, 1H), 2.07-1.96 (m, 4H), 1.46-1.34 (m, 3H).

Step II: To a solution of (R)-3,15-difluoro-11-(3-hydroxy-3-methylpiperidin-1-yl)-13-(2,2,2-trifluoroethoxy)-4,5,8,9-tetrahydro-7H-naphtho[1′,8′:7,8,9][1]oxa[4]azacycloundeca[5,6-g]quinazoline-17-ol (140 mg, 0.23 mmol) in dichloromethane (1.4 mL) was added diisopropylethylamine (123 μL, 0.69 mmol). The mixture was cooled to −78° C., and trifluoromethanesulfonic anhydride (39.0 μL, 0.23 mmol) was slowly added. The reaction was stirred at −70° C. for 4 hours and quenched with water (1 mL). The mixture was extracted with dichloromethane (1 mL×3), and the combined organic layers were dried over anhydrous sodium sulfate, filtered, and concentrated. The residue was purified by flash chromatography (silica gel, 0-30% gradient tetrahydrofuran/dichloromethane) to give a yellow solid compound (R)-3,15-difluoro-11-(3-hydroxy-3-methylpiperidin-1-yl)-13-(2,2,2-trifluoroethoxy)-4,5,8,9-tetrahydro-7H-naphtho[1′,8′:7,8,9][1]oxa[4]azacycloundeca[5,6-g]quinazoline-17-yl trifluoromethanesulfonate (49.0 mg, 0.07 mmol, yield: 29%). LCMS (ESI): [M+H]⁺=737.2.

Step III: To a solution of (R)-3,15-difluoro-11-(3-hydroxy-3-methylpiperidin-1-yl)-13-(2,2,2-trifluoroethoxy)-4,5,8,9-tetrahydro-7H-naphtho[1′,8′:7,8,9][1]oxa[4]azacycloundeca[5,6-g]quinazoline-17-yl trifluoromethanesulfonate (45.0 mg, 61.09 μmol) in N,N-dimethylformamide (2.25 mL) were added palladium acetate (4.11 mg, 18.33 μmol) and triphenylphosphine (9.64 mg, 36.65 μmol), followed by triethylamine (25.5 μL, 0.18 mmol) and formic acid (6.91 μL, 0.18 mmol) under a nitrogen atmosphere. The reaction mixture was stirred at 80° C. for 30 minutes. After the reaction was complete, the reaction mixture was cooled to room temperature, diluted with water (1 mL), and extracted with ethyl acetate (1 mL×3). The combined organic layers were dried over anhydrous sodium sulfate, filtered, and concentrated. The residue was purified using flash column chromatography (silica gel, 0-30% gradient of tetrahydrofuran/dichloromethane), to give a brown solid compound (R)-1-(3,15-difluoro-13-(2,2,2-trifluoroethoxy)-4,5,8,9-tetrahydro-7H-naphtho[1′,8′:7,8,9][1]oxa[4]azacycloundeca[5,6-g]quinazoline-11-yl)-3-methylpiperidin-3-ol (27.0 mg, 45.87 μmol, yield: 75%). LCMS (ESI): [M+H]⁺=589.2. ¹H NMR (400 MHz, CDCl3) δ ppm 7.99 (d, J=8.3 Hz, 1H), 7.89 (dd, J=6.0, 8.9 Hz, 1H), 7.52 (t, J=7.2 Hz, 1H), 7.39-7.27 (m, 3H), 4.95-4.75 (m, 2H), 4.36-4.07 (m, 3H), 4.01-3.86 (m, 1H), 3.62-3.30 (m, 4H), 3.19-3.05 (m, 2H), 2.79-2.57 (m, 2H), 2.12-1.67 (m, 4H), 1.38-1.31 (m, 3H).

### Intermediate 7: (R)-1-(3,15-Difluoro-13-(2,2,2-trifluoroethoxy)-4,5,8,9-tetrahydro-7H-naphtho[1′,8′:7,8,9][1]oxa[4]azacycloundeca[5,6-g]quinazoline-11-yl)-3-methylpiperidin-3-ol

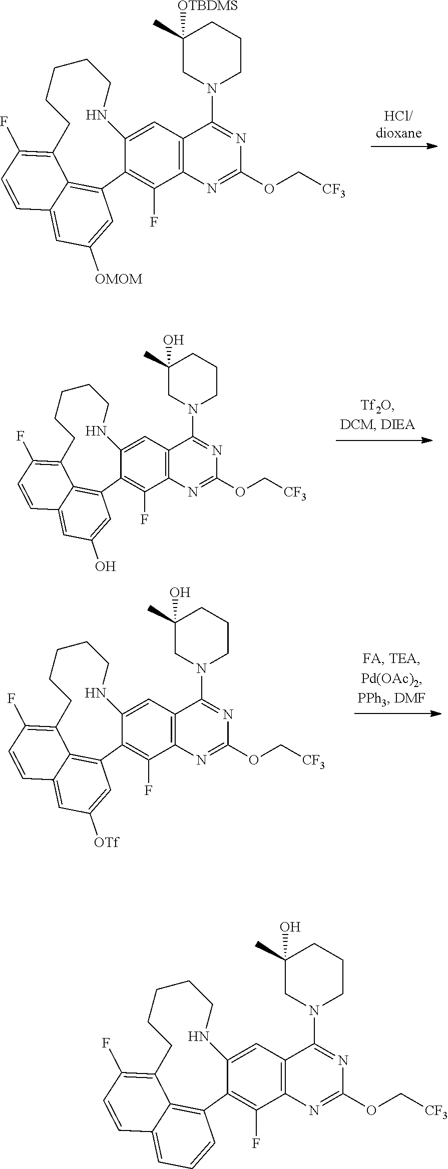

Step I: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7,8,9-hexahydronaphtho[1′,8′:4,5,6][1]azacycloundecino[2,3-g]quinazolin (200 mg, 0.26 mmol) in acetonitrile (2.00 mL) was added 4 M hydrochloric acid in dioxane (7.86 mL, 31.46 mmol). The mixture was stirred at 25° C. for 48 hours. After concentration under reduced pressure, the residue was purified by flash chromatography (silica gel, 0-15% gradient methanol/dichloromethane) to give a brown solid compound (R)-3,15-difluoro-1-(3-hydroxy-3-methylpiperidin-1-yl)-13-(2,2,2-trifluoroethoxy)-4,5,6,7,8,9-hexahydronaphtho[1′,8′:4,5,6][1]azacycloundecino[2,3-g]quinazolin-17-ol (157 mg, 0.26 mmol, yield: 99%). LCMS (ESI): [M+H]⁺=603.2

Step II: To a solution of (R)-3,15-difluoro-11-(3-hydroxy-3-methylpiperidin-1-yl)-13-(2,2,2-trifluoroethoxy)-4,5,6,7,8,9-hexahydronaphtho[1′,8′:4,5,6][1]azacycloundecane[2,3-g]quinazolin-17-ol (140 mg, 0.23 mmol) in dichloromethane (1.4 mL) was added diisopropylethylamine (123 μL, 0.69 mmol). The mixture was cooled to −78° C., and trifluoromethanesulfonic anhydride (39.0 μL, 0.23 mmol) was slowly added. The reaction was stirred at −70° C. for 4 hours and quenched with water (1 mL). The mixture was extracted with dichloromethane (1 mL×3), and the combined organic layers were dried over anhydrous sodium sulfate, filtered, and concentrated. The residue was purified by flash chromatography (silica gel, 0-30% gradient tetrahydrofuran/dichloromethane) to give a yellow solid compound (R)-3,15-difluoro-11-(3-hydroxy-3-methylpiperidin-1-yl)-13-(2,2,2-trifluoroethoxy)-4,5,6,7,8,9-hexahydronaphtho[1′,8′:4,5,6][1]azacycloundecane [2,3-g]quinazolin-17-yl trifluoromethanesulfonate (49.0 mg, 0.07 mmol, yield: 29%). LCMS (ESI): [M+H]⁺=735.2.

Step III: To a solution of (R)-3,15-difluoro-11-(3-hydroxy-3-methylpiperidin-1-yl)-13-(2,2,2-trifluoroethoxy)-4,5,6,7,8,9-hexahydronaphtho[1′,8′:4,5,6][1]azacycloundecane [2,3-g]quinazolin-17-yl trifluoromethanesulfonate (45.0 mg, 61.09 μmol) in N,N-dimethylformamide (2.25 mL) were added palladium acetate (4.11 mg, 18.33 μmol) and triphenylphosphine (9.64 mg, 36.65 μmol) under a nitrogen atmosphere, followed by triethylamine (25.5 μL, 0.18 mmol) and formic acid (6.91 μL, 0.18 mmol). The reaction mixture was stirred at 80° C. for 30 minutes. After the reaction was complete, the reaction mixture was cooled to room temperature, diluted with water (1 mL), and extracted with ethyl acetate (1 mL×3). The combined organic layers were dried over anhydrous sodium sulfate, filtered, and concentrated. The residue was purified using flash column chromatography (silica gel, 0-30% gradient of tetrahydrofuran/dichloromethane), to give a brown solid compound (R)-1-(3,15-difluoro-13-(2,2,2-trifluoroethoxy)-4,5,6,7,8,9-hexahydronaphthalene [1′,8′:4,5,6][1]azacycloundecane [2,3-g]quinazolin-11-yl)-3-methylpiperidin-3-ol (27.0 mg, 45.87 mol, yield: 75%). LCMS (ESI): [M+H]⁺=587.3.

### Intermediate 8: 11-((1R,5S)-3,8-Diazabicyclo[3.2.1]octane-3-yl)-3,15-difluoro-13-(((2R,7aS)-2-fluorotetrahydro-1H-pyrrolin-7a(5H)-yl)methoxy)-4,5,6,7,8,9-hexahydro-naphtho[1′,8′:4,5,6][1]azacycloundecino[2,3-g]quinazolin-17-ol

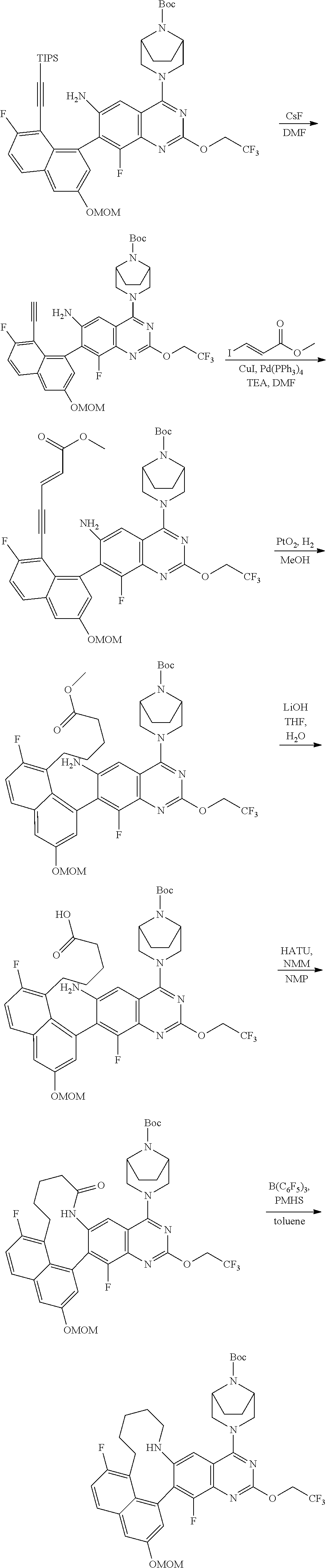

Step I: To a solution of tert-butyl (1R,5S)-3-(6-amino-8-fluoro-7-(7-fluoro-3-(methoxymethoxy)-8-((triisopropylsilyl)ethynyl)naphthalen-1-yl)-2-(2,2,2-trifluoroethoxy)quinazolin-4-yl)-3,8-diazabicyclo[3.2.1]octane-8-carboxylate (1370.0 mg, 1.60 mmol) in N,N-dimethylformamide (27 mL) was added cesium fluoride (2431.1 mg, 16.00 mmol). The mixture was stirred at 25° C. for 1 hour under a nitrogen atmosphere. then diluted with water (90 mL) and extracted with ethyl acetate (90 mL×3). The organic layers were combined, dried over anhydrous sodium sulfate, filtered, and concentrated under reduced pressure. The residue was purified by flash chromatography on silica gel (0-30% gradient of ethyl acetate/petroleum ether) to give the brown solid compound tert-butyl (1R,5S)-3-(6-amino-7-(8-ethynyl-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (720.0 mg, 1.03 mmol, yield: 64%). LCMS (ESI): [M+H]⁺=700.2.

Step II: To a solution of tert-butyl (1R,5S)-3-(6-amino-7-(8-ethynyl-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (500.0 mg, 0.71 mmol) and methyl 3-iodopropenoate (227.2 mg, 1.07 mmol) in N,N-dimethylformamide (3.75 mL) and triethylamine (2.5 mL) were added tetrakis(triphenylphosphine)palladium(0) (165.2 mg, 0.14 mmol) and copper(I) iodide (13.6 mg, 0.07 mmol) in a glove box. The reaction mixture was stirred at 50° C. for 2 hours under a nitrogen atmosphere. The residue was purified by flash column chromatography (silica gel, 0-30% gradient of ethyl acetate/petroleum ether) to give a brown solid compound tert-butyl (1R,5S)-3-(6-amino-8-fluoro-7-(7-fluoro-8-((E)-5-methoxy-5-oxopent-3-en-1-yn-1-yl)-3-(methoxymethoxy)naphthalen-1-yl)-2-(2,2,2-trifluoroethoxy)quinazolin-4-yl)-3,8-diazabicyclo[3.2.1]octane-8-carboxylate (530.0 mg, 0.68 mmol, yield: 95%). LCMS (ESI): [M+H]⁺=784.2.

Step III: To a solution of tert-butyl (1R,5S)-3-(6-amino-8-fluoro-7-[7-fluoro-8-((E)-5-methoxy-5-oxopent-3-en-1-yn-1-yl)-3-(methoxymethoxy)naphthalen-1-yl]-2-(2,2,2-trifluoroethoxy)quinazolin-4-yl)-3,8-diazabicyclo[3.2.1]octane-8-carboxylate (500.0 mg, 0.64 mmol) in methanol (50 mL) was added platinum dioxide (400.0 mg, 1.76 mmol). The mixture was stirred at 25° C. for 2 days under hydrogen atmosphere (15 psi). The reaction mixture was filtered, and the solid was washed with methanol (50 mL). The filtrate was concentrated under reduced pressure. The residue was purified by flash column chromatography (silica gel, gradient elution with 0-30% ethyl acetate/petroleum ether) to give a yellow solid compound tert-butyl (1R,5S)-3-(6-amino-8-fluoro-7-[7-fluoro-8-(5-methoxy-5-oxopentyl)-3-(methoxymethoxy)naphthalen-1-yl]-2-(2,2,2-trifluoroethoxy)quinazolin-4-yl)-3,8-diazabicyclo[3.2.1]octane-8-carboxylate (260.0 mg, 0.33 mmol, yield: 48%). LCMS (ESI): [M+H]⁺=790.4.

Step IV: To a solution of tert-butyl (1R,5S)-3-(6-amino-8-fluoro-7-[7-fluoro-8-(5-methoxy-5-oxopentyl)-3-(methoxymethoxy)naphthalen-1-yl]-2-(2,2,2-trifluoroethoxy)quinazolin-4-yl)-3,8-diazabicyclo[3.2.1]octane-8-carboxylate (240 mg, 0.30 mmol) in tetrahydrofuran (2.4 mL) were added lithium hydroxide (38.3 mg, 0.91 mmol) and water (0.48 mL). The reaction mixture was stirred at 50° C. for 24 hours, After completion, water (5 mL) was added to dilute the reaction mixture, and the pH was adjusted to 5-6 with 1 M hydrochloric acid. The aqueous phase was extracted with ethyl acetate (5 mL×3), and the combined organic layers was dried over anhydrous sodium sulfate, filtered, and concentrated under reduced pressure to give the crude compound 5-(8-(6-amino-4-[(1R,5S)-8-(tert-butoxycarbonyl)-3,8-diazabicyclo[3.2.1]octan-3-yl]-8-fluoro-2-(2,2,2-trifluoroethoxy)quinazolin-7-yl)-2-fluoro-6-(methoxymethoxy)naphthalen-1-yl)pentanoic acid (240 mg) as a yellow solid. LCMS (ESI): [M+H]⁺=776.3.

Step V: To a solution of 5-(8-(6-amino-4-[(1R,5S)-8-(tert-butoxycarbonyl)-3,8-diazabicyclo[3.2.1]octan-3-yl]-8-fluoro-2-(2,2,2-trifluoroethoxy)quinazolin-7-yl)-2-fluoro-6-(methoxymethoxy)naphthalen-1-yl)pentanoic acid (210 mg, 0.27 mmol) and N-methylmorpholine (273.4 mg, 2.71 mmol) in N-methylpyrrolidone (20 mL) was added 0-(7-azabenzotriazol-1-yl)-N,N,N′,N′-tetramethyluronium hexafluorophosphate (308.8 mg, 0.81 mmol). The mixture was stirred at 25° C. for 2 hours and then stirred at 80° C. for 16 hours under a nitrogen atmosphere. Water (30 mL) and saturated sodium bicarbonate solution (30 mL) were added, and the mixture was extracted with ethyl acetate (60 mL×3). The combined organic layers were dried over anhydrous sodium sulfate, filtered, and concentrated under reduced pressure. The residue was purified by flash column chromatography (silica gel, gradient elution with 0-30% ethyl acetate/petroleum ether) to give a crude brown oil (300 mg). A portion of the crude product (200 mg) was further purified by flash column chromatography (C18 column, 0-70% gradient of acetonitrile/water). to give a white solid compound tert-butyl (1R,5S)-3-(3,15-difluoro-17-(methoxymethoxy)-8-oxo-13-(2,2,2-trifluoroethoxy)-4,5,6,7,8,9-hexahydro-naphtho[1′,8′:4,5,6][1]azacycloundecin-11-yl)-3,8-diazabicyclo[3.2.]octane-8-carboxylate (72 mg, 0.10 mmol, yield: 53%). LCMS (ESI): [M+H]⁺=758.2.

Step VI: To a solution of tert-butyl (1R,5S)-3-(3,15-difluoro-17-(methoxymethoxy)-8-oxo-13-(2,2,2-trifluoroethoxy)-4,5,6,7,8,9-hexahydro-naphtho[1′,8′:4,5,6][1]azacycloundecino[2,3-g]quinazolin-11-yl)-3,8-diazabicyclo[3.2.1]octane-8-carboxylate (50 mg, 0.07 mmol) and tris(pentafluorophenyl)borane (33.8 mg, 0.07 mmol) in toluene (2 mL) was added polymethylsiloxane (500 μL). The reaction mixture was stirred at 110° C. for 2 days under a nitrogen atmosphere. The mixture was concentrated under reduced pressure. The residue was purified by flash column chromatography (silica gel, gradient elution with 0-30% ethyl acetate/petroleum ether) to give a yellow solid compound tert-butyl (1R,5S)-3-(3,15-difluoro-17-(methoxymethoxy)-13-(2,2,2-trifluoroethoxy)-4,5,6,7,8,9-hexahydronaphtho[1′,8′:4,5,6][1]azacycloondecano[2,3-g]quinazolin-1-yl)-3,8-diazabicyclo[3.2.1]octane-8-carboxylate (30 mg. 0.04 mmol, yield: 61%). LCMS (ESI): [M+H]⁺=744.3.

### Intermediate 9: (2,2-Difluoro-6-methylene-tetrahydro-1H-pyrazin-7a(5H)-yl)methanol

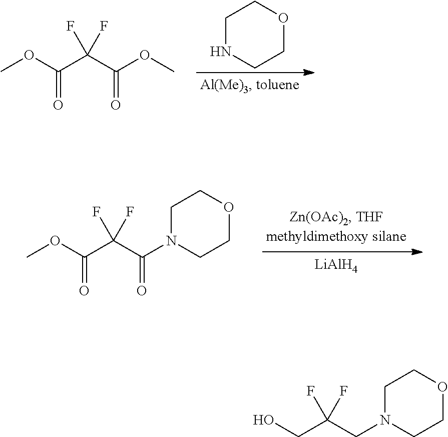

Step I: To a solution of 1-(tert-butyl)-2-methyl-4,4-difluoropyrrolidine-1,2-dicarboxylate (9.00 g, 33.9 mmol) in tetrahydrofuran (90 mL) was added lithium bis(trimethylsilyl)amide (1 M in tetrahydrofuran, 68.0 mL, 68.0 mmol) at −78° C. under a nitrogen atmosphere and the reaction mixture was stirred at −78° C. for 1 hour. 3-Chloro-2-(chloromethyl)prop-1-ene (12.7 g, 102 mmol) was then added, and the reaction mixture was stirred at 20° C. for 16 hours. The reaction was quenched with water (100 mL), and the mixture was extracted with ethyl acetate (100 mL×3). The combined organic layers were washed with saturated brine (100 mL), dried over sodium sulfate, filtered, and concentrated under reduced pressure. The residue was purified by flash column chromatography (silica gel, 0-5% gradient of tetrahydrofuran/hexane) to give a colorless oily compound 1-(tert-butyl)-2-methyl-2-(2-(chloromethyl)allyl)-4,4-difluoropyrrolidine-1,2-dicarboxylate (8.90 g, 25.2 mmol, yield: 74%). LCMS (ESI): [M−56+H]⁺=298.0; ¹H NMR (400 MHz, CDCl3) δ ppm 5.52-5.38 (m, 11H), 5.11 (br s, 1H), 4.13-3.87 (m, 3H), 3.84-3.63 (m, 4H), 3.46-3.21 (m, 1H), 2.86-2.45 (m, 3H), 1.52-1.43 (m, 9H).

Step II: To a solution of 1-(tert-butyl)-2-methyl-2-(2-(chloromethyl)allyl)-4,4-difluoropyrrolidine-1,2-dicarboxylate (2.00 g, 5.65 mmol) in dichloromethane (20 mL) was added trifluoroacetic acid (6.30 mL, 84.8 mmol). The reaction mixture was stirred at 20° C. for 16 hours. The reaction mixture was concentrated under reduced pressure, diluted with dichloromethane (100 mL), and neutralized with triethylamine. After concentration under reduced pressure, the residue was purified by flash column chromatography (silica gel, 0-10% gradient of methanol/dichloromethane) to give a yellow oily compound methyl 2,2-difluoro-6-methylenetetrahydro-1H-pyrazin-7a(5H)-carboxylate (0.50 g, 2.26 mmol, yield: 40%). LCMS (ESI): [M+H]⁺=218.1; ¹H NMR (400 MHz, CDCl3) δ ppm 5.01-4.95 (m, 2H), 3.91 (br d, J=14.6 Hz, 1H), 3.70 (s, 3H), 3.60-3.49 (m, 1H), 3.47-3.34 (m, 2H), 3.00-2.82 (m, 2H), 2.65 (br d, J=16.4 Hz, 1H), 2.44-2.29 (m, 1H). To a solution of lithium aluminum hydride (2.5 M in tetrahydrofuran, 4.00 mL, 10.0 mmol) in tetrahydrofuran (4 mL) was added methyl 2,2-difluoro-6-methylenetetrahydro-1H-pyrazin-7a(5H)-carboxylate (500 mg, 2.26 mmol) at 0° C. The reaction mixture was stirred at 60° C. for 2 hours. The reaction was quenched by sequential addition of water (370 μL), sodium hydroxide (15% aqueous solution, 370 μL), and water (1.00 mL). The mixture was filtered, and the filtrate was concentrated under reduced pressure to give a compound (2,2-difluoro-6-methylenetetrahydro-1H-pyrazin-7a(5H)-yl)methanol (220 mg, 1.13 mmol, yield: 50%). LCMS (ESI): [M+H]⁺=182.1.

### Intermediate 10: (Hexahydrocycloprop[b]pyrazin-5a(3H)-yl)methanol

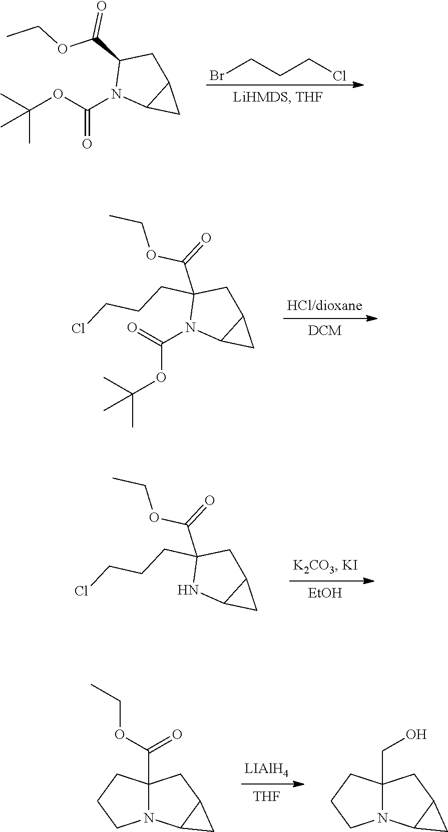

Step I: A solution of (3R)-tert-butyl 3-ethyl-2-azabicyclo[3.1.0]hexane-2,3-dicarboxylate (950 mg, 3.72 mmol) and 1-bromo-3-chloropropane (1.76 g, 11.16 mmol) in tetrahydrofuran (10 mL) was cooled to −65° C. under a nitrogen atmosphere. Lithium bis(trimethylsilyl)amide (1 M solution in tetrahydrofuran, 7.44 mL, 7.44 mmol) was added dropwise. The mixture was stirred at 25° C. for 16 hours. Saturated ammonium chloride solution (10 mL) was added to quench the reaction, and the mixture was extracted with ethyl acetate (10 mL×3). The combined organic layers were dried over anhydrous sodium sulfate, filtered, and concentrated under reduced pressure. The residue was purified by flash column chromatography (silica gel, 0-10% gradient of tetrahydrofuran/dichloromethane) to give a colorless oily compound tert-butyl 3-ethyl-3-(3-chloropropyl)-2-azabicyclo[3.1.0]hexane-2,3-dicarboxylate (630 mg, 1.90 mmol, yield: 51%). LCMS (ESI): [M+Na]⁺=354.3.

Step II: To a solution of tert-butyl 3-ethyl-3-(3-chloropropyl)-2-azabicyclo[3.1.0]hexane-2,3-dicarboxylate (610 mg, 1.84 mmol) in dichloromethane (6 mL) was added hydrogen chloride (4 M solution in dioxane, 1.38 mL, 5.51 mmol). The mixture was stirred at 25° C. for 16 hours. The mixture was concentrated under reduced pressure to give a colorless oily crude compound ethyl 3-(3-chloropropyl)-2-azabicyclo[3.1.0]hexane-3-carboxylate (60% purity, 690 mg). LCMS (ESI): [M+H]⁺=232.2.

Step III: To a solution of ethyl 3-(3-chloropropyl)-2-azabicyclo[3.1.0]hexane-3-carboxylate (670 mg, 60% purity, 1.73 mmol) in ethanol (26 mL) were added potassium iodide (29 mg, 0.17 mmol) and potassium carbonate (719 mg, 5.20 mmol). The mixture was stirred at 25° C. for 16 hours. The reaction mixture was filtered and concentrated. The residue was purified by flash column chromatography (silica gel, 0-10% gradient of ethanol/dichloromethane) to give a yellow oily compound ethyl hexahydrocyclopropa[b]pyrrolizine-5a(3H)-carboxylate (95% purity, 320 mg, 1.56 mmol, yield: 90%). LCMS (ESI): [M+H]⁺=196.1.

Step IV: To a solution of ethyl hexahydrocyclopropa[b]pyrrolizine-5a(3H)-carboxylate (95% purity, 310 mg, 1.50 mmol) in tetrahydrofuran (3 mL) at 0° C. was added lithium aluminum hydride (72 mg, 1.91 mmol). The reaction mixture was stirred at 0° C. for 1 hour, The reaction was quenched by the sequential addition of water (72 IL), 15% aqueous sodium hydroxide (72 μL), and water (216 μL). Anhydrous sodium sulfate was added to dry the mixture. The resulting mixture was filtered and the filter cake was washed with tetrahydrofuran (30 mL). The filtrate was concentrated under reduced pressure to give a crude product hexahydrocyclopropa[b]pyrrolizine-5a(3H)-methanol (230 mg), as a yellow oily liquid. LCMS (ESI): [M+H]⁺=154.1.

### Intermediate 11: (1-Morpholinocyclopropyl)methanol

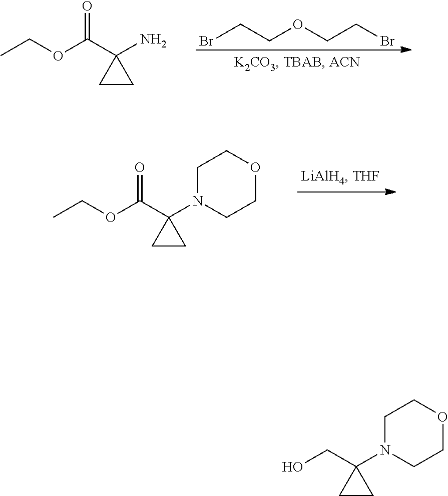

Step I: A solution of ethyl 1-aminocyclopropanecarboxylate (200 mg, 1.55 mmol) in acetonitrile (2.00 mL) was prepared, followed by the addition of potassium carbonate (899 mg, 6.50 mmol) and tetrabutylammonium bromide (74.9 mg, 0.23 mmol). A solution of 1-bromo-2-(2-bromoethoxy)ethane (431 mg, 1.86 mmol) in acetonitrile was added dropwise to the reaction mixture. The reaction mixture was stirred at 80° C. under nitrogen protection for 16 hours. The reaction mixture was filtered, and the filtrate was concentrated under reduced pressure. The residue was purified by flash column chromatography (silica gel, 0-10% gradient of tetrahydrofuran/hexane) to give a colorless oily compound ethyl 1-morpholinocyclopropanecarboxylate (160 mg, 0.81 mmol, yield: 52%). ¹H NMR (400 MHz, CDCl3) δ ppm 4.15 (q, J=7.1 Hz, 2H), 3.59 (br s, 4H), 2.96 (br s, 4H), 1.36-1.18 (m, 5H), 0.94 (q, J=3.7 Hz, 2H).

Step II: To a solution of ethyl 1-morpholinocyclopropanecarboxylate (160 mg, 0.80 mmol) in tetrahydrofuran (1.50 mL) at 0° C. was added lithium aluminum hydride (0.64 mL, 1.61 mmol). The reaction mixture was stirred at 0° C. for 1 hour, The reaction was quenched by the sequential addition of water (0.64 mL), 15% aqueous sodium hydroxide (0.64 mL), and water (1.92 mL). Anhydrous sodium sulfate was added to dry the mixture, filtered, and the filter cake was washed with tetrahydrofuran (30 mL). The filtrate was concentrated under reduced pressure to give a crude compound (1-morpholinocyclopropyl)methanol (100 mg), as a colorless oily liquid. ¹H NMR (400 MHz, CDCl3) δ ppm 3.68-3.62 (m, 4H), 3.59 (s, 2H), 2.80-2.64 (m, 4H), 0.76-0.70 (m, 2H), 0.56-0.50 (m, 2H).

### Intermediate 12: (2-(Difluoromethylene)-tetrahydro-1H-pyrazin-7a(5H)-yl)methanol and (2-(Fluoromethylene)-tetrahydro-1H-pyrazin-7a(5H)-yl)methanol

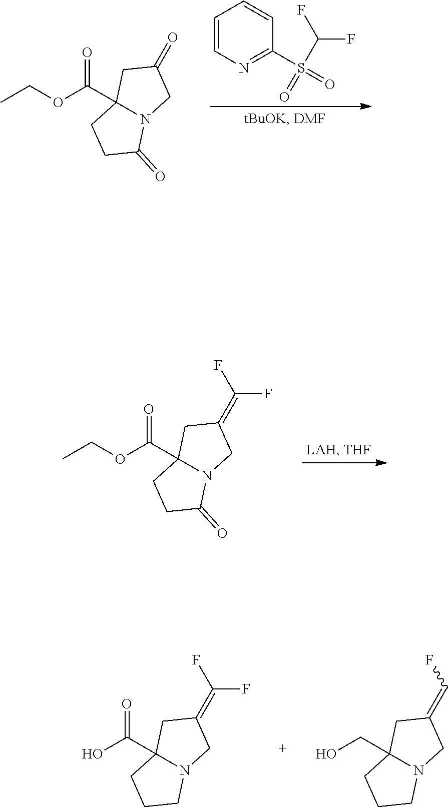

Step I: To a solution of potassium tert-butoxide (0.95 g, 8.52 mmol) in N,N-dimethylformamide (11 mL) at −50° C., a solution of ethyl 2,5-dioxotetrahydro-1H-pyrazin-7a(5H)-carboxylate (1.0 g, 4.73 mmol) and 2-((difluoromethyl)sulfonyl)pyridine (0.82 g, 4.26 mmol) in N,N-dimethylformamide (5 mL) was added. The mixture was slowly warmed to 25° C. under a nitrogen atmosphere and stirred at 25° C. for 0.5 hours. The reaction mixture was cooled in an ice bath, and saturated ammonium chloride solution (5 mL) and 2 M hydrochloric acid (9 mL) were added, followed by water (30 mL). The mixture was extracted with ethyl acetate (30 mL×3). The combined organic layers were dried over anhydrous sodium sulfate, filtered, and concentrated under reduced pressure. The residue was purified by flash column chromatography (silica gel, 0-50% gradient of tetrahydrofuran/hexane) to give a yellow oily compound 2-(difluoromethylene)-5-oxo-tetrahydro-1H-pyrazin-7a(5H)-carboxylic acid ethyl ester (250 mg, 1.02 mmol, yield: 22%). LCMS (ESI): [M+H]⁴=246.0; ¹H NMR (400 MHz, CDCl₃) δ ppm 4.36 (br d, J=14.7 Hz, 1H), 4.23 (q, J=7.1 Hz, 2H), 3.75 (br d, J=14.7 Hz, 1H), 3.13 (td, J=1.3, 15.4 Hz, 1H), 2.90-2.73 (m, 1H), 2.63 (ddd, J=2.0, 9.2, 13.2 Hz, 1H), 2.55-2.34 (m, 2H), 2.15 (td, J=10.1, 13.2 Hz, 1H), 1.29 (t, J=7.2 Hz, 3H).

Step III: At 0° C., to a solution of lithium aluminum hydride (15.5 mg, 0.41 mmol) in tetrahydrofuran (1 mL) was added a solution of 2-(difluoromethylene)-5-oxo-tetrahydro-1H-pyrazin-7a(5H)-carboxylic acid ethyl ester (100.0 mg, 0.41 mmol) in tetrahydrofuran (1 mL). The mixture was stirred at 65° C. for 5 hours, followed by the addition of lithium aluminum hydride (7.7 mg, 0.20 mmol), and stirring was continued at 65° C. for an additional 3 hours. After cooling to 0° C., water (23 μL), 15% aqueous sodium hydroxide solution (23 μL), and water (69 μL) were sequentially added. The mixture was dried over anhydrous sodium sulfate, filtered, and the filtrate was concentrated under reduced pressure to give a yellow oily crude compound comprising a mixture of (2-(difluoromethylene)tetrahydro-1H-pyrazin-7a(5H)-yl)methanol and (2-(fluoromethylene)tetrahydro-1H-pyrazin-7a(5H)-yl)methanol (74.0 mg). LCMS (ESI): [M+H]⁺=190.1; 172.1.

### Example 1: 10-((1R,5S)-3,8-Diazabicyclo[3.2.1]octan-3-yl)-3,14-difluoro-12-(((2R,7aS)-2-fluorotetrahydro-1H-pyrazin-7a(5H)-yl)methoxy)-16-hydroxy-5,6-dihydro-4H-naphtho[1′,8′:4,5,6]acridin[2,3-g]quinazolin-7(8H)-one

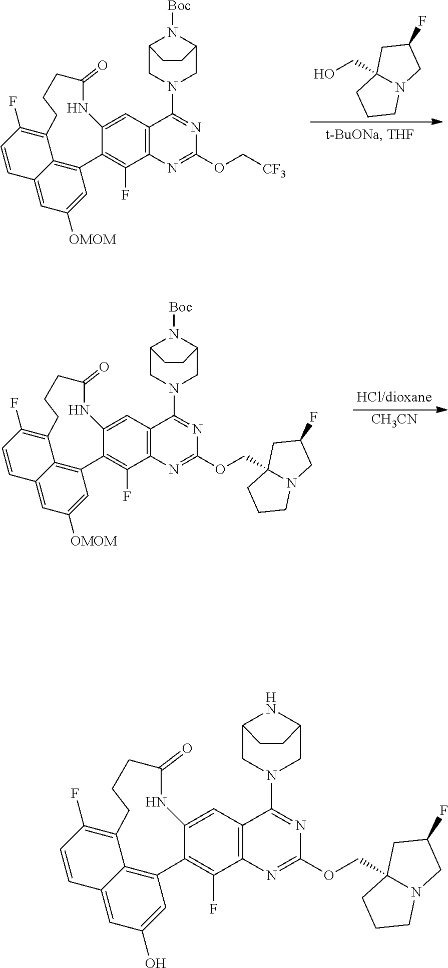

Step I: At 25° C., to a solution of tert-butyl (1R,5S)-3-(3,14-difluoro-16-(methoxymethoxy)-7-oxo-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (80% purity, 150 mg, 0.16 mmol) and ((2R,7aS)-2-fluorotetrahydro-1H-pyrazin-7a(5H)-yl)methanol (77.1 mg, 0.48 mmol) in tetrahydrofuran (2.00 mL) was added sodium tert-butoxide (79.0 mg, 0.80 mmol). The reaction mixture was stirred at 50° C. for 16 hours. After cooling to room temperature, water (3 mL) was added to quench the reaction, and the mixture was extracted with ethyl acetate (3 mL×3). The combined organic layers were concentrated under reduced pressure, and the residue was purified by flash column chromatography (silica gel, 0-8% gradient methanol/dichloromethane) to give a yellow solid compound tert-butyl (1R,5S)-3-(3,14-difluoro-12-(((2R,7aS)-2-fluorotetrahydro-1H-pyrazin-7a(5H)-yl)methoxy)-16-(methoxymethoxy)-7-oxo-5,6,7,8-tetrahydro-4H-naphtho[1′,8′:4,5,6]acridin[2,3-g]quinazolin-10-yl)-3,8-diazabicyclo[3.2.1]octane-8-carboxylate (40.0 mg, 0.05 mmol, yield: 30%). LCMS (ESI): [M+H]⁺=802.9

Step II: At 25° C., to a solution of tert-butyl (1R,5S)-3-(3,14-difluoro-12-(((2R,7aS)-2-fluorotetrahydro-1H-pyrazin-7a(5H)-yl)methoxy)-16-(methoxymethoxy)-7-oxo-5,6,7,8-tetrahydro-4H-naphtho[1′,8′:4,5,6]acridin[2,3-g]quinazolin-10-yl)-3,8-diazabicyclo[3.2.1]octane-8-carboxylate (40.0 mg, 0.05 mmol) in acetonitrile (800 μL) was treated with hydrogen chloride (4 M solution in dioxane, 200 μL, 0.80 mmol). The reaction mixture was stirred at 20° C. for 2 hours. After concentration under reduced pressure, the residue was dissolved in acetonitrile (4 mL) and the pH was adjusted to 8 using triethylamine. The solution was concentrated under reduced pressure, and the residue was purified by preparative HPLC to give a white solid compound 10-((1R,5S)-3,8-diazabicyclo[3.2.1]octan-3-yl)-3,14-difluoro-12-(((2R,7aS)-2-fluorotetrahydro-1H-pyrazin-7a(5H)-yl)methoxy)-16-hydroxy-5,6-dihydro-4H-naphtho[1′,8′:4,5,6]acridin[2,3-g]quinazolin-7(8H)-one (7.68 mg, 11.6 μmol, yield: 26%). LCMS (ESI): [M+H]⁺=659.3. ¹H NMR (400 MHz, CD₃OD) δ ppm 7.80 (s, 1H), 7.70 (dd, J=6.1, 9.0 Hz, 1H), 7.32-7.20 (m, 2H), 6.91 (s, 1H), 5.46-5.24 (m, 1H), 4.65 (br d, J=12.3 Hz, 1H), 4.44-4.19 (m, 3H), 3.77 (br d, J=12.7 Hz, 1H), 3.71-3.57 (m, 3H), 3.31-3.19 (m, 3H), 3.05 (dt, J=6.1, 9.3 Hz, 1H), 2.97-2.87 (m, 1H), 2.76 (br t, J=12.2 Hz, 1H), 2.46-2.23 (m, 3H), 2.22-2.15 (m, 1H), 2.14-2.08 (m, 1H), 2.08-1.98 (m, 4H), 1.95-1.83 (m, 4H), 1.82-1.74 (m, 1H).

### Example 2: 10-((1R,5S)-3,8-Diazabicyclo[3.2.1]octan-3-yl)-3,14-difluoro-12-(((2R,7aS)-2-fluorotetrahydro-1H-pyrazin-7a(5H)-yl)methoxy)-5,6,7,8-tetrahydro-4H-naphtho[1′,8′:4,5,6]acridin[2,3-g]quinazolin-16-ol

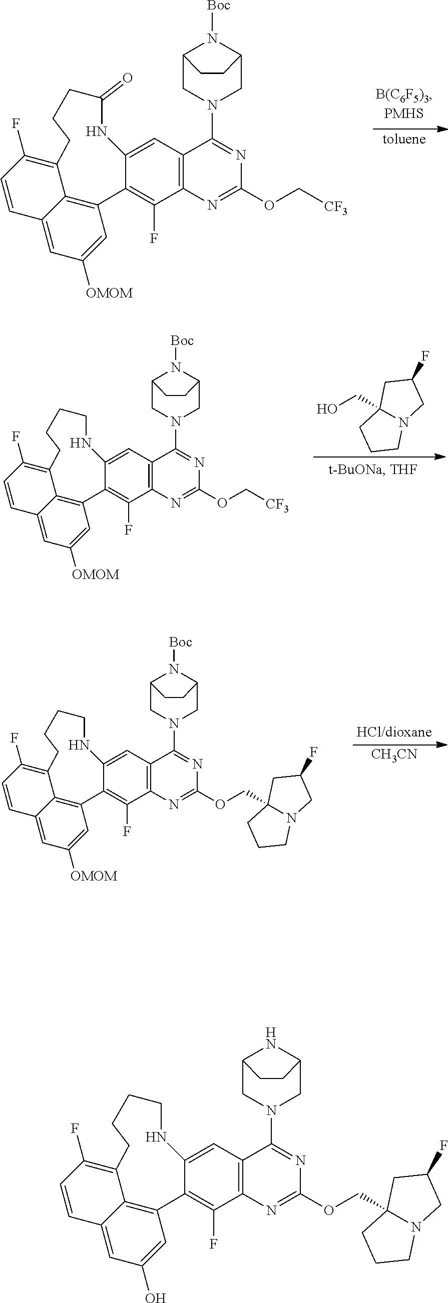

Step I: To a solution of tert-butyl (1R,5S)-3-(3,14-difluoro-16-(methoxymethoxy)-7-oxo-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (150 mg, 0.20 mmol) in toluene (6.00 mL) were added tris(pentafluorophenyl)borane (206 mg, 0.40 mmol) and polymethylsiloxane (2.50 mL, 1.00 mmol). The reaction mixture was stirred at 110° C. for 12 hours. The reaction mixture was cooled to room temperature, concentrated under reduced pressure, and the crude product was purified by flash column chromatography (silica gel, 0-30% gradient of tetrahydrofuran/hexane) to give a brown solid compound tert-butyl (1R,5S)-3-(3,14-difluoro-16-(methoxymethoxy)-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (140 mg, 0.19 mmol, yield: 95%). LCMS (ESI): [M+H]⁺=730.2.

Step II: To a solution of tert-butyl (1R,5S)-3-(3,14-difluoro-16-(methoxymethoxy)-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (140 mg, 0.19 mmol) and ((2R,7aS)-2-fluorotetrahydro-1H-pyrazin-7a(5H)-yl)methanol (91.6 mg, 0.58 mmol) in tetrahydrofuran (2.00 mL) was added sodium tert-butoxide (55.3 mg, 0.58 mmol). The reaction mixture was stirred at 60° C. for 24 hours. The reaction mixture was cooled to room temperature, diluted with ethyl acetate (1 mL) and water (1 mL), and the organic layer was separated. The aqueous layer was extracted with ethyl acetate (1 mL×2). The combined organic layers were dried over anhydrous magnesium sulfate, filtered, and concentrated under reduced pressure. The crude product was purified by flash chromatography (silica gel, 0-10% methanol/dichloromethane gradient) to give a yellow solid compound tert-butyl (1R,5S)-3-(3,14-difluoro-12-(((2R,7aS)-2-fluorotetrahydro-1H-pyrazin-7a(5H)-yl)methoxy)-16-(methoxymethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (48.0 mg, 0.06 mmol, yield: 32%). LCMS (ESI): [M+H]⁺=789.5.

Step III: To a solution of tert-butyl (1R,5S)-3-(3,14-difluoro-12-(((2R,7aS)-2-fluorotetrahydro-1H-pyrazin-7a(5H)-yl)methoxy)-16-(methoxymethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (46.0 mg, 0.06 mmol) in acetonitrile (2.00 mL) was added hydrochloric acid (4 M solution in dioxane, 292 μL, 1.17 mmol). The reaction mixture was stirred at 25° C. for 2 hours. The reaction mixture was concentrated under reduced pressure, and the residue was dissolved in acetonitrile (1 mL). The solution was neutralized with triethylamine to pH>7, concentrated under reduced pressure, and the residue was purified by preparative HPLC to give a yellow solid compound 10-((1R,5S)-3,8-diazabicyclo[3.2.1]octan-3-yl)-3,14-difluoro-12-(((2R,7aS)-2-fluorotetrahydro-1H-pyrazin-7a(5H)-yl)methoxy)-5,6,7,8-tetrahydro-4H-naphtho[1′,8′:4,5,6]acridin[2,3-g]quinazolin-16-ol, was obtained (18.4 mg, 0.03 mmol, yield: 49%). LCMS (ESI): [M+H]⁺=645.4. ¹H NMR (400 MHz, CD₃OD) δ ppm 7.78-7.70 (m, 1H), 7.36-7.24 (m, 2H), 7.08 (s, 1H), 7.04-6.99 (m, 1H), 5.41-5.26 (m, 1H), 4.45-4.34 (m, 2H), 4.32-4.26 (m, 1H), 4.24-4.18 (m, 1H), 3.71-3.52 (m, 4H), 3.47-3.38 (m, 1H), 3.28-3.22 (m, 3H), 3.10-2.98 (m, 1H), 2.87-2.75 (m, 1H), 2.61-2.49 (m, 1H), 2.44-2.13 (m, 3H), 2.07-1.98 (m, 2H), 1.97-1.85 (m, 6H), 1.70-1.53 (m, 2H), 1.41-1.03 (m, 2H).

### Example 3: 10-((1R,5S)-3,8-Diazabicyclo[3.2.1]octan-3-yl)-3,14-difluoro-12-(((2R,7aS)-2-fluorotetrahydro-1H-pyrazin-7a(5H)-yl)methoxy)-8-methyl-5,6,7,8-tetrahydro-4H-naphtho[1′,8′:4,5,6]acridin[2,3-g]quinazolin-16-ol

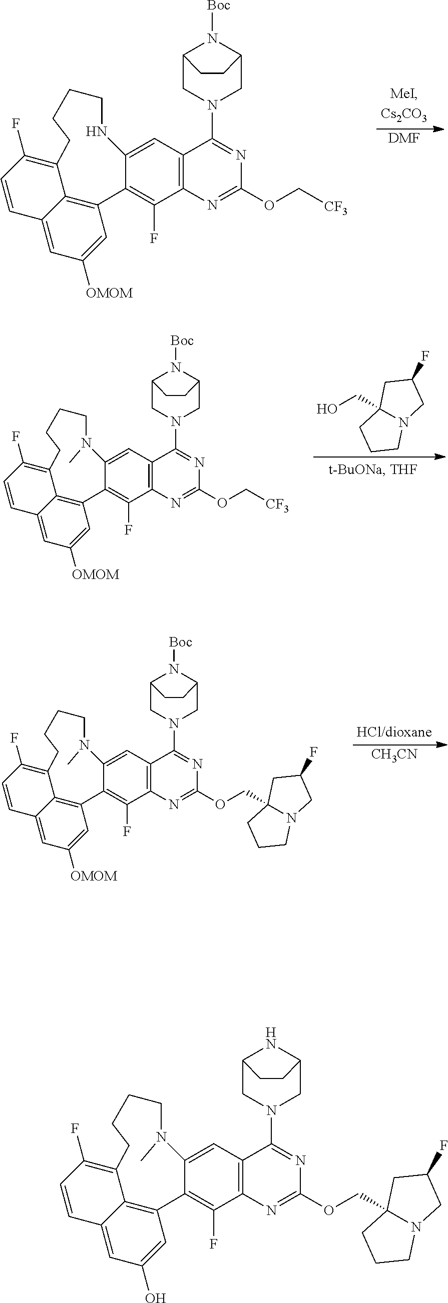

Step I: To a solution of tert-butyl (1R,5S)-3-(3,14-difluoro-16-(methoxymethoxy)-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (27.0 mg, 37.0 μmol) in dimethylformamide (200 μL) were added cesium carbonate (13.0 mg, 40.70 μmol) and iodomethane (4.59 μL, 74.33 μmol). The reaction mixture was stirred at 25° C. for 16 hours. The reaction solution was slowly poured into ice water (100 μL), followed by the addition of ethyl acetate (100 μL). The organic layer was separated, and the aqueous layer was extracted with ethyl acetate (100 μL×2). The organic phases were combined, dried over anhydrous magnesium sulfate, and filtered, and the filtrate was concentrated. The crude product was purified by flash column chromatography (silica gel, 0-40% gradient of tetrahydrofuran/petroleum ether) to give a yellow solid compound tert-butyl (1R,5S)-3-(3,14-difluoro-16-(methoxymethoxy)-8-methyl-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (22.0 mg, 29.58 μmol, yield: 80%). LCMS (ESI): [M+H]⁺=744.3.

Step II: To a solution of tert-butyl (1R,5S)-3-(3,14-difluoro-16-(methoxymethoxy)-8-methyl-12-(2,2,2-trifluoroethoxy)-5,6,7,8-tetrahydro-4H-naphtho[1′,8′:4,5,6]acridin[2,3-g]quinazolin-10-yl)-3,8-diazabicyclo[3.2.1]octane-8-carboxylate (20.0 mg, 26.89 μmol) and ((2R,7aS)-2-fluorotetrahydro-1H-pyrazin-7a(5H)-yl)methanol (12.8 mg, 80.67 μmol) in tetrahydrofuran (400 μL) was added sodium tert-butoxide (7.75 mg, 80.67 μmol). The reaction mixture was stirred at 60° C. for 6 hours. The reaction mixture was cooled to room temperature and diluted with ethyl acetate (200 μL) and water (200 μL). The organic layer was separated, and the aqueous layer was extracted with ethyl acetate (100 μL×2). The combined organic layers were dried over anhydrous magnesium sulfate, filtered, and concentrated under reduced pressure to give a brown oily compound tert-butyl (1R,5S)-3-(3,14-difluoro-12-(((2R,7aS)-2-fluorotetrahydro-1H-pyrazin-7a(5H)-yl)methoxy)-16-(methoxymethoxy)-8-methyl-5,6,7,8-tetrahydro-4H-naphtho[1′,8′:4,5,6]acridin[2,3-g]quinazolin-10-yl)-3,8-diazabicyclo[3.2.1]octane-8-carboxylate (20.0 mg, 24.90 μmol, yield: 93%). LCMS (ESI): [M+H]⁺=803.5.

Step III: To a solution of tert-butyl (1R,5S)-3-(3,14-difluoro-12-(((2R,7aS)-2-fluorotetrahydro-1H-pyrazin-7a(5H)-yl)methoxy)-16-(methoxymethoxy)-8-methyl-5,6,7,8-tetrahydro-4H-naphtho[1′,8′:4,5,6]acridin[2,3-g]quinazolin-10-yl)-3,8-diazabicyclo[3.2.1]octane-8-carboxylate (20.0 mg, 24.90 μmol) in acetonitrile (400 μL) was added hydrochloric acid (4 M solution in dioxane, 62.3 μL, 0.25 mmol). The reaction mixture was stirred at 25° C. for 2 hours. The reaction mixture was concentrated under reduced pressure, and the residue was dissolved in acetonitrile (500 μL). The solution was neutralized with triethylamine to pH>7, concentrated under reduced pressure to remove the solvent, and the residue was purified by preparative HPLC to give a yellow solid compound 10-((1R,5S)-3,8-diazabicyclo[3.2.1]octan-3-yl)-3,14-difluoro-12-(((2R,7aS)-2-fluorotetrahydro-1H-pyrazin-7a(5H)-yl)methoxy)-8-methyl-5,6,7,8-tetrahydro-4H-naphtho[1′,8′:4,5,6]acridin[2,3-g]quinazolin-16-ol, was obtained (9.40 mg, 14.19 μmol, yield: 57%). LCMS (ESI): [M+H]⁺=659.3. ¹H NMR (400 MHz, CD₃OD) δ ppm 7.65 (dd, J=6.0, 9.0 Hz, 1H), 7.41 (s, 1H), 7.26-7.18 (m, 2H), 6.94 (d, J=2.4 Hz, 1H), 5.41-5.23 (m, 1H), 4.48 (br dd, J=6.3, 12.8 Hz, 1H), 4.37 (br d, J=15.5 Hz, 1H), 4.32-4.26 (m, 1H), 4.24-4.18 (m, 1H), 3.71-3.54 (m, 4H), 3.32-3.15 (m, 3H), 3.09-2.96 (m, 1H), 2.95-2.73 (m, 2H), 2.40 (s, 3H), 2.37-2.12 (m, 4H), 2.06-1.83 (m, 8H), 1.52-1.29 (m, 4H).

### Example 4: 11-((1R,5S)-3,8-diazabicyclo[3.2.1]octan-3-yl)-3,15-difluoro-13-(((2R,7aS)-2-fluorotetrahydro-1H-pyrazin-7a(5H)-yl)methoxy)-17-hydroxy-4,5,6,7-tetrahydronaphtho[1′,8′:4,5,6][1]azacycloundecin[2,3-g]quinazolin-8(9H)-one

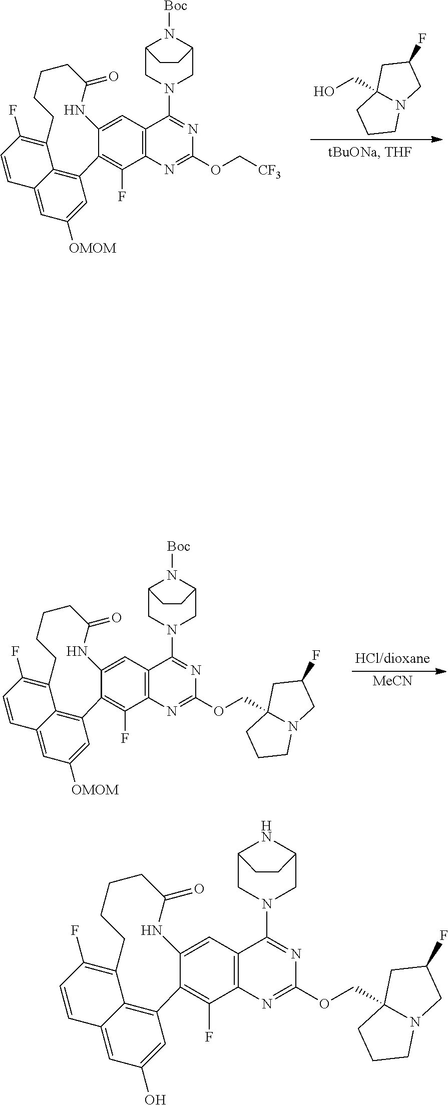

Step I: To a solution of tert-butyl (1R,5S)-3-(3,15-difluoro-17-(methoxymethoxy)-8-oxo-13-(2,2,2-trifluoroethoxy)-4,5,6,7,8,9-hexahydronaphtho[1′,8′:4,5,6][1]azacycloundecin[2,3-g]quinazolin-11-yl)-3,8-diazabicyclo[3.2.1]octane-8-carboxylate (100 mg, 0.13 mmol) and ((2R,7aS)-2-fluorotetrahydro-1H-pyrazin-7a(5H)-yl)methanol (63.0 mg, 0.40 mmol) in tetrahydrofuran (2 mL) was added sodium tert-butoxide (25.4 mg, 0.26 mmol) under a nitrogen atmosphere. The mixture was stirred at 50° C. for 24 hours under a nitrogen atmosphere. The reaction mixture was cooled to 0° C., diluted with water (5 mL), and extracted with ethyl acetate (10 mL×3). The combined organic layers were dried over anhydrous sodium sulfate, filtered, and concentrated under reduced pressure to give a crude compound tert-butyl (1R,5S)-3-(3,15-difluoro-13-(((2R,7aS)-2-fluorotetrahydro-1H-pyrazin-7a(5H)-yl)methoxy)-17-(methoxymethoxy)-8-oxo-4,5,6,7,8,9-hexahydronaphtho[1′,8′:4,5,6][1]azacycloundecin[2,3-g]quinazolin-11-yl)-3,8-diazabicyclo[3.2.1]octane-8-carboxylate (140 mg), was obtained as a brown oily liquid. LCMS (ESI): [M+H]⁺=817.6.

Step II: To a solution of tert-butyl (1R,5S)-3-(3,15-difluoro-13-(((2R,7aS)-2-fluorotetrahydro-1H-pyrazin-7a(5H)-yl)methoxy)-17-(methoxymethoxy)-8-oxo-4,5,6,7,8,9-hexahydronaphtho[1′,8′:4,5,6][1]azacycloundecin[2,3-g]quinazolin-11-yl)-3,8-diazabicyclo[3.2.1]octane-8-carboxylate (120 mg, 0.15 mmol) in acetonitrile (2400 μL) was added hydrogen chloride (4 M in dioxane, 550 μL, 2.20 mmol). The mixture was stirred at 25° C. for 1 hour. The solution was evaporated under a nitrogen stream, and the residue was purified by preparative HPLC to give a white solid compound 11-((1R,5S)-3,8-diazabicyclo[3.2.1]octan-3-yl)-3,15-difluoro-13-(((2R,7aS)-2-fluorotetrahydro-1H-pyrazin-7a(5H)-yl)methoxy)-17-hydroxy-4,5,6,7-tetrahydronaphtho[1′,8′:4,5,6][1]azacycloundecin[2,3-g]quinazolin-8(9H)-one (6.76 mg, 0.01 mmol, yield: 7%). LCMS (ESI): [M+H]⁺=673.2. ¹H NMR (400 MHz, CD₃OD) δ ppm 7.82 (s, 1H), 7.67 (dd, J=5.9, 9.0 Hz, 1H), 7.34-7.19 (m, 2H), 6.94 (d, J=2.3 Hz, 1H), 5.40-5.23 (m, 1H), 4.49-4.42 (m, 2H), 4.33-4.18 (m, 2H), 3.83-3.59 (m, 4H), 3.29-3.19 (m, 3H), 3.08-2.98 (m, 1H), 2.75 (br s, 1H), 2.59-2.40 (m, 2H), 2.36-2.20 (m, 2H), 2.13 (d, J=9.2 Hz, 2H), 2.06-1.77 (m, 8H), 1.58-1.41 (m, 3H).

### Example 5: 11-((1R,5S)-3,8-Diazabicyclo[3.2.1]octane-3-yl)-3,15-difluoro-13-(((2R,7aS)-2-fluorotetrahydro-1H-pyrrolin-7a(5H)-yl)methoxy)-4,5,6,7,8,9-hexahydro-naphtho[1′,8′:4,5,6][1]azacycloundecino[2,3-g]quinazolin-17-ol

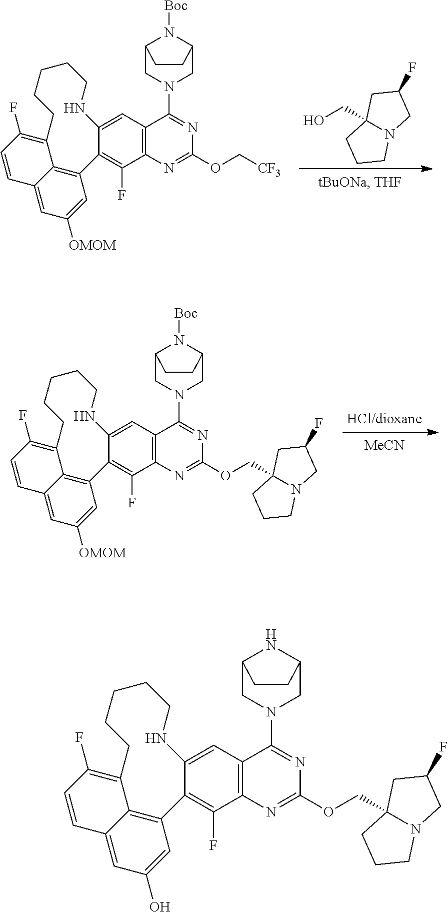

Step I: To a solution of tert-butyl (1R,5S)-3-(3,15-difluoro-17-(methoxymethoxy)-13-(2,2,2-trifluoroethoxy)-4,5,6,7,8,9-hexahydronaphtho[1′,8′:4,5,6][1]azacycloundecin[2,3-g]quinazolin-11-yl)-3,8-diazabicyclo[3.2.1]octane-8-carboxylate (15 mg, 0.02 mmol) and ((2R,7aS)-2-fluorotetrahydro-1H-pyrazin-7a(5H)-yl)methanol (9.6 mg, 0.06 mmol) in tetrahydrofuran (0.3 mL) was added sodium tert-butoxide (3.8 mg, 0.04 mmol) under a nitrogen atmosphere. The mixture was stirred at 50° C. for 24 hours under a nitrogen atmosphere. The reaction was diluted with water (2 mL) at 0° C., and extracted with ethyl acetate (5 mL×3). The combined organic layers were dried over anhydrous sodium sulfate, filtered, and concentrated under reduced pressure to give a crude compound tert-butyl (1R,5S)-3-(3,15-difluoro-13-(((2R,7aS)-2-fluorotetrahydro-1H-pyrrolo[1,2-c]-pyrrolidine-7a(5H)-yl)methoxy)-17-(methoxymethoxy)-4,5,6,7,8,9-hexahydronaphtho[1′,8′:4,5,6][1]azacycloundecin[2,3-g]quinazolin-11-yl)-3,8-diazabicyclo[3.2.1]octane-8-carboxylate (20 mg), was obtained as a brown oily liquid. LCMS (ESI): [M+H]⁺=803.4.

Step II: To a solution of tert-butyl (1R,5S)-3-(3,15-difluoro-13-(((2R,7aS)-2-fluorotetrahydro-1H-pyrrolo[1,2-c]-pyrrolidin-7a(5H)-yl)methoxy)-17-(methoxymethoxy)-4,5,6,7,8,9-hexahydronaphtho[1′,8′:4,5,6][1]azacycloundecin[2,3-g]quinazolin-1-yl)-3,8-diazabicyclo[3.2.1]octane-8-carboxylate (20 mg, 0.02 mmol) in acetonitrile (400 μL) was added hydrogen chloride (4 M in dioxane, 75 μL, 0.30 mmol). The mixture was stirred at 25° C. for 1 hour. The mixture was evaporated under a nitrogen stream. The residue was purified by preparative HPLC to give a white solid compound 11-((1R,5S)-3,8-diazabicyclo[3.2.1]octan-3-yl)-3,15-difluoro-13-(((2R,7aS)-2-fluorotetrahydro-1H-pyrrolo[1,2-c]pyrimidin-7a(5H)-yl)methoxy)-4,5,6,7,8,9-hexahydronaphtho[1′,8′:4,5,6][1]azacycloundecin[2,3-g]quinazolin-17-ol (2.03 mg, 3.0 μmol, yield: 15%). LCMS (ESI): [M+H]⁺=659.3. ¹H NMR (400 MHz, CD₃OD) δ ppm 8.56 (br s, 1H), 7.69 (dd, J=5.8, 9.0 Hz, 1H), 7.31 (d, J=2.6 Hz, 1H), 7.25 (t, J=9.5 Hz, 1H), 7.06 (s, 1H), 6.94 (d, J=2.6 Hz, 1H), 5.43-5.28 (m, 1H), 4.48 (br d, J=12.0 Hz, 1H), 4.41-4.23 (m, 3H), 3.78 (br s, 2H), 3.72-3.55 (m, 3H), 3.38 (br s, 2H), 3.18-2.93 (m, 2H), 2.64 (br d, J=11.9 Hz, 1H), 2.48-2.26 (m, 3H), 2.19 (br d, J=11.5 Hz, 1H), 2.13-1.86 (m, 8H), 1.78-1.49 (m, 3H), 1.46-1.29 (m, 3H).

### Example 6A: 3,15-Difluoro-13-(((2R,7aS)-2-fluorotetrahydro-1H-pyrazin-7a(5H)-yl)methoxy)-11-((R)-3-hydroxy-3-methylpiperidin-1-yl)-9-methyl-4,5,6,7,8,9-hexahydronaphtho[1′,8′:4,5,6][1]azacycloundecin[2,3-g]quinazolin-17-ol

### Example 6B: 3,15-Difluoro-13-(((2R,7aS)-2-fluorotetrahydro-1H-pyrazin-7a(5H)-yl)methoxy)-11-((R)-3-hydroxy-3-methylpiperidin-1-yl)-9-methyl-4,5,6,7,8,9-hexahydronaphtho[1′,8′:4,5,6][1]azacycloundecin[2,3-g]quinazolin-17-ol

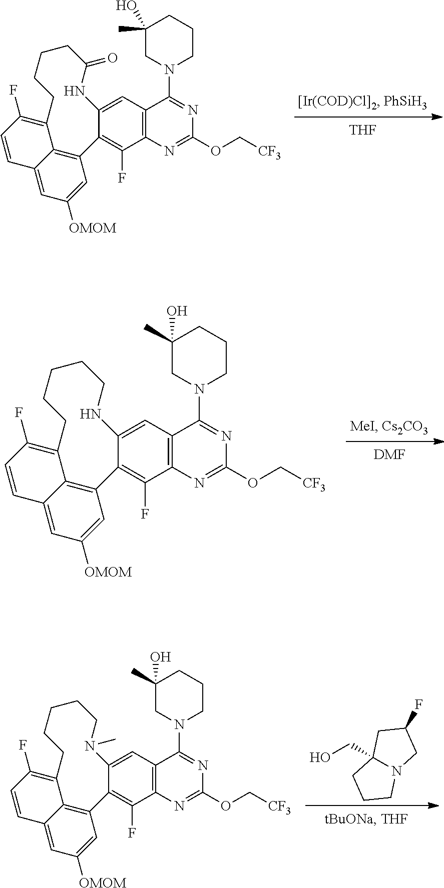

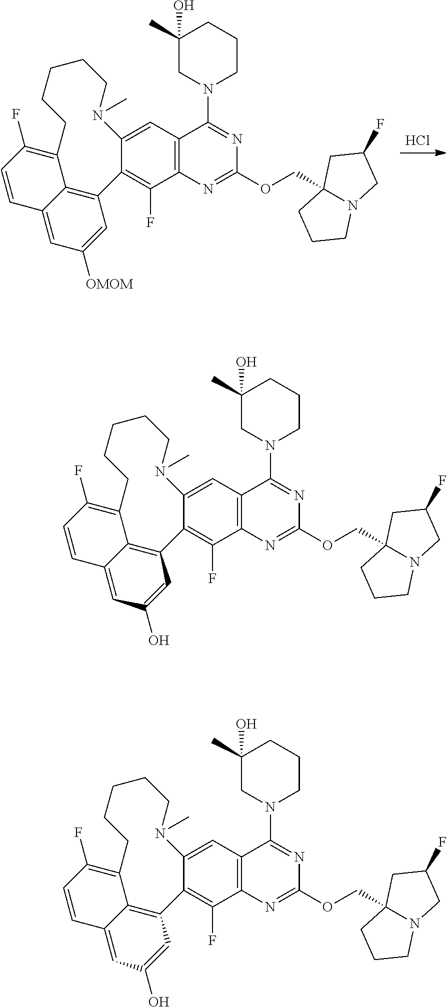

Step I: To a solution of (R)-3,15-difluoro-11-(3-hydroxy-3-methylpiperidin-1-yl)-17-(methoxymethoxy)-13-(2,2,2-trifluoroethoxy)-4,5,6,7-tetrahydronaphtho[1′,8′:4,5,6][1]azacycloundecin[2,3-g]quinazolin-8(9H)-one (80 mg, 0.12 mmol) and 1,5-cyclooctadiene iridium(I) chloride dimer (8.13 mg, 0.01 mmol) in tetrahydrofuran (12 mL) was added phenylsilane (262.1 mg, 2.42 mmol). The reaction mixture was stirred at 55° C. under a nitrogen atmosphere for 10 hours and concentrated under reduced pressure. The residue was purified by flash column chromatography (silica gel, 0-35% gradient of ethyl acetate/petroleum ether) to give a yellow solid compound (R)-1-(3,15-difluoro-17-(methoxymethoxy)-13-(2,2,2-trifluoroethoxy)-4,5,6,7,8,9-hexahydronaphtho[1′,8′:4,5,6][1]azacycloundecin[2,3-g]quinazolin-11-yl)-3-methylpiperidin-3-ol (24 mg, 0.04 mmol, yield: 31%). LCMS (ESI): [M+H]⁺=647.2.

Step II: To a solution of (R)-1-(3,15-difluoro-17-(methoxymethoxy)-13-(2,2,2-trifluoroethoxy)-4,5,6,7,8,9-hexahydronaphtho[1′,8′:4,5,6][1]azacycloundecin[2,3-g]quinazolin-11-yl)-3-methylpiperidin-3-ol (43 mg, 0.07 mmol) in N,N-dimethylformamide (1.075 mL) were added cesium carbonate (23.8 mg, 0.07 mmol) and iodomethane (8.2 μL, 0.13 mmol). The mixture was stirred under a nitrogen atmosphere at 25° C. for 16 hours. The mixture was then diluted with water (3 mL) and extracted with ethyl acetate (5 mL×3). The combined organic layers were dried over anhydrous sodium sulfate, filtered, and concentrated under reduced pressure to give a crude compound (R)-1-(3,15-difluoro-17-(methoxymethoxy)-9-methyl-13-(2,2,2-trifluoroethoxy)-4,5,6,7,8,9-hexahydronaphtho[1′,8′:4,5,6][1]azacycloundecin[2,3-g]quinazolin-11-yl)-3-methylpiperidin-3-ol (62 mg). LCMS (ESI): [M+H]⁺=661.3.

Step III: To a solution of (R)-1-(3,15-difluoro-17-(methoxymethoxy)-9-methyl-13-(2,2,2-trifluoroethoxy)-4,5,6,7,8,9-hexahydronaphtho[1′,8′:4,5,6][1]azacycloundecin[2,3-g]quinazolin-11-yl)-3-methylpiperidin-3-ol (60 mg, 0.09 mmol) and ((2R,7aS)-2-fluorotetrahydro-1H-pyrazin-7a(5H)-yl)methanol (57.8 mg, 0.36 mmol) in tetrahydrofuran (1.5 mL) was added sodium tert-butoxide (26.2 mg, 0.27 mmol). The mixture was stirred at 60° C. for 12 hour under a nitrogen atmosphere. The reaction was diluted with water (3 mL), and the mixture was extracted with ethyl acetate (3 mL×3). The combined organic layers were dried over anhydrous sodium sulfate, filtered, and concentrated under reduced pressure to give a crude compound (R)-1-(3,15-difluoro-13-(((2R,7aS)-2-fluorotetrahydro-1H-pyrazin-7a(5H)-yl)methoxy)-17-(methoxymethoxy)-9-methyl-4,5,6,7,8,9-hexahydronaphtho[1′,8′:4,5,6][1]azacycloundecin[2,3-g]quinazolin-11-yl)-3-methylpiperidin-3-ol (100 mg), a brown oily liquid. LCMS (ESI): [M+H]⁺=720.4.

Step IV: To a solution of (R)-1-(3,15-difluoro-13-(((2R,7aS)-2-fluorotetrahydro-1H-pyrazin-7a(5H)-yl)methoxy)-17-(methoxymethoxy)-9-methyl-4,5,6,7,8,9-hexahydronaphtho[1′,8′:4,5,6][1]azacycloundecin[2,3-g]quinazolin-11-yl)-3-methylpiperidin-3-ol (100 mg, 0.09 mmol) in acetonitrile (2000 μL) was added hydrogen chloride (4 M in dioxane, 520 μL, 2.08 mmol). The mixture was stirred at 25° C. for 1 hour. The mixture was evaporated under a nitrogen stream. The residue was purified by preparative HPLC to give two isomers of the compound 3,15-difluoro-13-(((2R,7aS)-2-fluorotetrahydro-1H-pyrazin-7a(5H)-yl)methoxy)-11-((R)-3-hydroxy-3-methylpiperidin-1-yl)-9-methyl-4,5,6,7,8,9-hexahydronaphtho[1′,8′:4,5,6][1]azacycloundecin[2,3-g]quinazolin-17-ol.

Example 6A: LCMS (ESI): [M+H]⁺=676.4. ¹H NMR (400 MHz, CD₃OD) δ ppm 7.84 (s, 1H), 7.66 (dd, J=5.9, 9.0 Hz, 1H), 7.29-7.17 (m, 2H), 6.90 (d, J=2.6 Hz, 1H), 5.48-5.26 (m, 1H), 4.47-4.26 (m, 3H), 4.15 (br d, J=13.4 Hz, 1H), 3.52-3.35 (m, 4H), 3.21-3.11 (m, 1H), 2.95-2.84 (m, 1H), 2.78 (s, 3H), 2.69-2.44 (m, 3H), 2.40-2.32 (m, 1H), 2.28-2.05 (m, 4H), 2.03-1.65 (m, 6H), 1.58 (br s, 1H), 1.47-1.18 (m, 8H).

Example 6B: LCMS (ESI): [M+H]⁺=676.4. ¹H NMR (400 MHz, CD₃OD) δ ppm 7.80 (s, 1H), 7.66 (dd, J=5.9, 9.0 Hz, 1H), 7.29-7.17 (m, 2H), 6.90 (d, J=2.6 Hz, 1H), 5.49-5.26 (m, 1H), 4.45-4.38 (m, 1H), 4.33 (d, J=11.3 Hz, 2H), 4.18 (d, J=13.3 Hz, 1H), 3.47-3.36 (m, 4H), 3.28 (d, J=3.3 Hz, 1H), 3.19-3.10 (m, 1H), 2.91 (br t, J=10.1 Hz, 1H), 2.80 (s, 3H), 2.59 (br d, J=6.4 Hz, 2H), 2.39-2.31 (m, 1H), 2.27-2.15 (m, 2H), 2.15-2.03 (m, 2H), 2.01-1.64 (m, 6H), 1.54 (br s, 1H), 1.43-1.24 (m, 8H).

### Example 7: (R)-1-(3,15-Difluoro-13-((tetrahydro-1H-pyrazin-7a(5H)-yl)methoxy)-4,5,6,7,8,9-hexahydronaphtho[1′,8′:4,5,6]azecino[2,3-g]quinazolin-11-yl)-3-methylpiperidin-3-ol

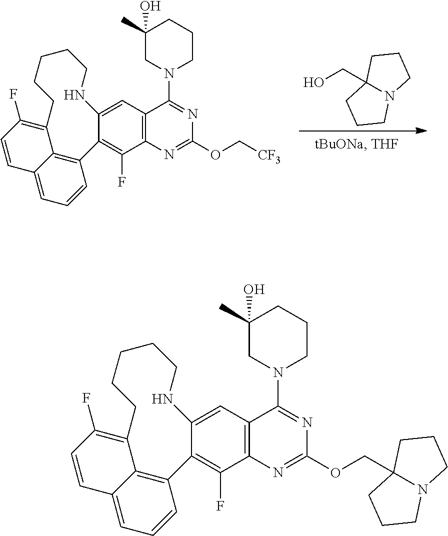 [SMILES: C[C@@]1(O)CCCN(c2nc(OCC(F)(F)F)nc3c(F)c4c(cc23)NCCCCCc2c(F)ccc3cccc-4c23)C1 ; OCC12CCCN1CCC2 ; C[C@@]1(O)CCCN(c2nc(OCC34CCCN3CCC4)nc3c(F)c4c(cc23)NCCCCCc2c(F)ccc3cccc-4c23)C1]

Step I: To a solution of (R)-1-(3,15-difluoro-13-(2,2,2-trifluoroethoxy)-4,5,6,7,8,9-hexahydronaphtho[1′,8′:4,5,6]azecino[2,3-g]quinazolin-11-yl)-3-methylpiperidin-3-ol (32 mg, 0.05 mmol) and (tetrahydro-1H-pyrazin-7a(5H)-yl)methanol (30.8 mg, 0.22 mmol) in tetrahydrofuran (0.8 mL) was added sodium tert-butoxide (15.7 mg, 0.16 mmol). The mixture was stirred under a nitrogen atmosphere at 60° C. for 30 hours. then diluted with water (2 mL) and extracted with ethyl acetate (3 mL×3). The organic layers were combined, dried over anhydrous sodium sulfate, filtered, and concentrated under reduced pressure. The residue was purified by preparative HPLC to give a yellow solid compound (R)-1-(3,15-difluoro-13-((tetrahydro-1H-pyrazin-7a(5H)-yl)methoxy)-4,5,6,7,8,9-hexahydronaphtho[1′,8′:4,5,6]azecino[2,3-g]quinazolin-11-yl)-3-methylpiperidin-3-ol (5.81 mg, 9.2 μmol, yield: 14%). LCMS (ESI): [M+H]⁺=628.2. ¹H NMR (400 MHz, CD₃OD) δ ppm 8.06 (d, J=8.1 Hz, 1H), 7.94 (dd, J=6.0, 9.0 Hz, 1H), 7.57 (t, J=7.6 Hz, 1H), 7.50-7.29 (m, 3H), 4.38 (br s, 2H), 4.24 (br d, J=13.3 Hz, 1H), 4.17-4.06 (m, 1H), 3.83-3.70 (m, 1H), 3.45-3.38 (m, 1H), 3.29 (br s, 1H), 3.06-2.85 (m, 3H), 2.77-2.65 (m, 1H), 2.48 (br d, J=5.5 Hz, 1H), 2.16 (br dd, J=6.4, 12.4 Hz, 3H), 2.09-1.93 (m, 4H), 1.92-1.71 (m, 7H), 1.59 (br d, J=8.1 Hz, 2H), 1.43 (br s, 2H), 1.32 (br d, J=3.1 Hz, 4H), 1.02 (br s, 1H).

### Example 8: 12-((1R,5S)-3,8-diazabicyclo[3.2.1]octan-3-yl)-3,16-difluoro-14-(((2R,7aS)-2-fluorotetrahydro-1H-pyrazin-7a(5H)-yl)methoxy)-5,6,7,8,9,10-hexahydro-4H-naphtho[1′,8′:4,5,6][1]azecino[2,3-g]quinazolin-18-ol

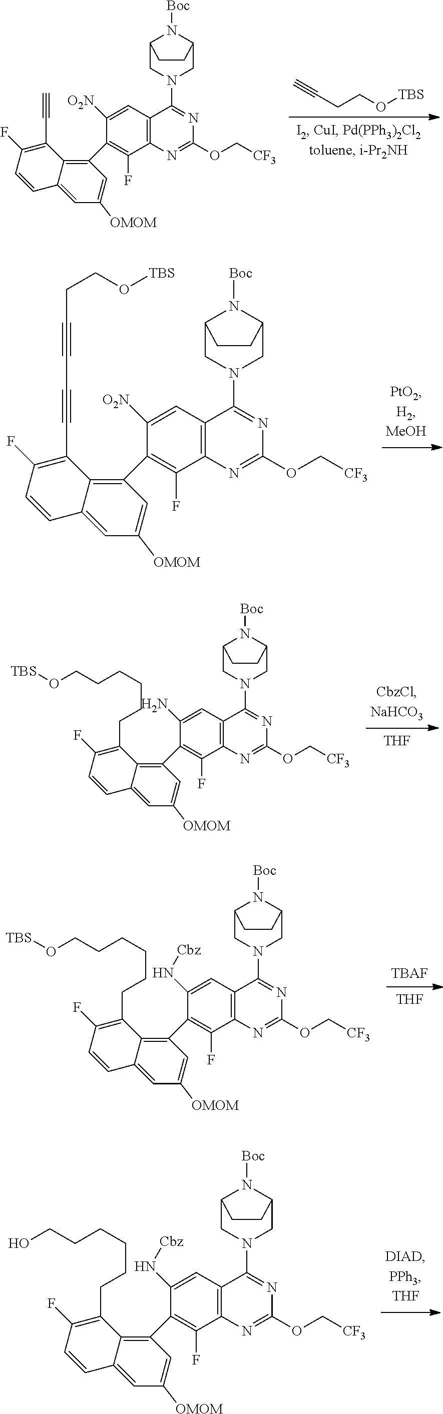

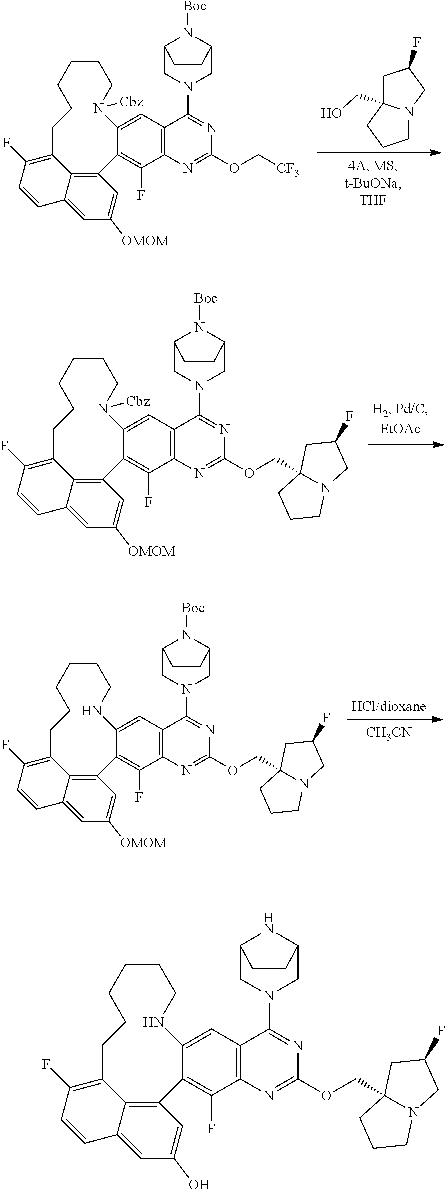

Step I: To a solution of tert-butyl (1R,5S)-3-(7-(8-ethynyl-7-fluoro-3-(methoxymethoxy)naphthalen-1-yl)-8-fluoro-6-nitro-2-(2,2,2-trifluoroethoxy)quinazolin-4-yl)-3,8-diazabicyclo[3.2.]octane-8-carboxylate (300 mg, 0.41 mmol) and 4-(tert-butyldimethylsilyloxy)-1-butyne (424 μL 2.06 mmol) in a 1:1 mixture of toluene/diisopropylamine (4.5 mL) were slowly added copper(I) iodide (7.80 mg, 0.04 mmol), iodine (313 mg, 1.23 mmol), and dichlorobis(triphenylphosphine)palladium(II) (28.8 mg, 0.04 mmol) at 25° C. The mixture was stirred at 25° C. for 16 hours. After completion, saturated aqueous sodium sulfite solution (3 mL) was added to the reaction mixture. The mixture was extracted with ethyl acetate (3 mL×2). The organic phases were combined, washed with water (3 mL), dried over anhydrous sodium sulfate, filtered, and concentrated under reduced pressure. The residue was purified by flash column chromatography (silica gel, 0-40% gradient of tetrahydrofuran/hexane) to give a brown solid compound tert-butyl (1R,5S)-3-(7-(8-(6-((tert-butyldimethylsilyl)oxy)hexa-1,3-diyne-11-yl)-7-fluoro-3-(methoxymethoxy)naphthalen-1-yl)-8-fluoro-6-nitro-2-(2,2,2-trifluoroethoxy)quinazolin-4-yl))-3,8-diazabicyclo[3.2.1]octane-8-carboxylate (364 mg, 0.40 mmol, yield: 97%). LCMS (ESI): [M+H]⁺=912.4.

Step II: Under a nitrogen atmosphere at 25° C., to a solution of tert-butyl (1R,5S)-3-(7-(8-(6-((tert-butyldimethylsilyl)oxy)hexa-1,3-diyne-1-yl)-7-fluoro-3-(methoxymethoxy)naphthalen-11-yl)-8-fluoro-6-nitro-2-(2,2,2-trifluoroethoxy)quinazolin-4-yl)-3,8-diazabicyclo[3.2.1]octane-8-carboxylate (100 mg, 0.11 mmol) in methanol (1 mL) was added platinum dioxide (100 mg). The solution was subjected to hydrogen atmosphere (15 psi) and stirred at 20° C. for 16 hours. The reaction mixture was filtered, and the filtrate was concentrated under reduced pressure to give a crude compound tert-butyl (1R,5S)-3-(6-amino-7-(8-(6-((tert-butyldimethylsilyl)oxy)hexyl)-7-fluoro-3-(methoxymethoxy)naphthalen-11-yl)-8-fluoro-2-(2,2,2-trifluoroethoxy)quinazolin-4-yl)-3,8-diazabicyclo[3.2.1]octane-8-carboxylate, as a brown solid (110 mg). LCMS (ESI): [M+H]⁺=890.4.

Step III: To a solution of tert-butyl (1R,5S)-3-(6-amino-7-(8-(6-((tert-butyldimethylsilyl)oxy)hexyl)-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (300 mg, 0.34 mmol) in tetrahydrofuran (4 mL) at 0° C. was added sodium bicarbonate (56.6 mg, 0.67 mmol). The mixture was stirred at 0° C. for 5 hours. Maintaining the temperature at 0° C., benzyl chloroformate (95.0 μL, 0.67 mmol) was added dropwise. The mixture was stirred at 25° C. for 16 hours. The reaction mixture was quenched with water (5 mL) and extracted with ethyl acetate (3 mL×3). The combined organic layers were dried over anhydrous sodium sulfate, filtered, and concentrated under reduced pressure to give a crude compound tert-butyl (1R,5S)-3-(6-(((benzyloxy)carbonyl)amino)-7-(8-(6-((tert-butyldimethylsilyl)oxy)hexyl)-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate, as a brown solid (400 mg). LCMS (ESI): [M+H]⁺=1024.5.

Step IV: At 20° C., tert-butyl (1R,5S)-3-(6-(((benzyloxy)carbonyl)amino)-7-(8-(6-((tert-butyldimethylsilyl)oxy)hexyl)-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (400.0 mg, 0.39 mmol) was dissolved in tetra-n-butylammonium fluoride (1 M in tetrahydrofuran, 8.0 mL, 8.0 mmol). The mixture was stirred at 20° C. for 1 hour. The reaction mixture was diluted with water (5 mL) and extracted with ethyl acetate (5 mL×3). The combined organic layers were dried over anhydrous sodium sulfate, filtered, and concentrated under reduced pressure. The residue was purified by flash column chromatography (silica gel, 0-25% gradient of tetrahydrofuran/petroleum ether) to give a yellow solid compound tert-butyl (1R,5S)-3-(6-(((benzyloxy)carbonyl)amino)-8-fluoro-7-(7-fluoro-8-(6-hydroxyhexyl)-3-(methoxymethoxy)naphthalen-1-yl)-2-(2,2,2-trifluoroethoxy)quinazolin-4-yl)-3,8-diazabicyclo[3.2.1]octane-8-carboxylate (240 mg, 0.26 mmol, yield: 68%). LCMS (ESI): [M+H]⁺=910.4.

Step V: Under a nitrogen atmosphere at 20° C., to a solution of tert-butyl (1R,5S)-3-(6-(((benzyloxy)carbonyl)amino)-8-fluoro-7-(7-fluoro-8-(6-hydroxyhexyl)-3-(methoxymethoxy)naphthalen-1-yl)-2-(2,2,2-trifluoroethoxy)quinazolin-4-yl)-3,8-diazabicyclo[3.2.1]octane-8-carboxylate (240 mg, 0.26 mmol) in tetrahydrofuran (8 mL) were added slowly triphenylphosphine (553 mg, 2.11 mmol) and diisopropyl azodicarboxylate (780 μL, 3.96 mmol). The mixture was stirred at 65° C. for 1 hour. The reaction mixture was diluted with water (8 mL) and extracted with ethyl acetate (3×6 mL). The combined organic layers were dried over anhydrous sodium sulfate, filtered, and concentrated under reduced pressure. The residue was purified by flash column chromatography (silica gel, 0-20% gradient of tetrahydrofuran/petroleum ether) to give a pinkish oily compound benzyl 12-((1R,5S)-8-(tert-butoxycarbonyl)-3,8-diazabicyclo[3.2.1]octan-3-yl)-3,16-difluoro-18-(methoxymethoxy)-14-(2,2,2-trifluoroethoxy)-4,5,6,7,8,9-hexahydro-10H-naphtho[1′,8′:4,5,6][1]azacyclododecino[2,3-g]quinazoline-10-carboxylate (purity 40%, 200 mg, 0.09 mmol, yield: 34%). LCMS (ESI): [M+H]⁺=892.5.

Step VI: At 0° C., to a solution of benzyl 12-((1R,5S)-8-(tert-butoxycarbonyl)-3,8-diazabicyclo[3.2.1]octan-3-yl)-3,16-difluoro-18-(methoxymethoxy)-14-(2,2,2-trifluoroethoxy)-4,5,6,7,8,9-hexahydro-10H-naphtho[1′,8′:4,5,6][1]azacyclododecino[2,3-g]quinazoline-10-carboxylate (180 mg, 0.08 mmol) in tetrahydrofuran (3 mL) were added 4A molecular sieves (180 mg), ((2R,7aS)-2-fluoro-tetrahydro-1H-pyrazin-7a(5H)-yl)methanol (102 mg, 0.65 mmol), and sodium tert-butoxide (62.0 mg, 0.65 mmol). The reaction mixture was stirred at 25° C. for 2 hours. The reaction mixture was diluted with water (4 mL) and extracted with ethyl acetate (3×3 mL). The combined organic layers were dried over anhydrous magnesium sulfate, filtered, and concentrated under reduced pressure. The residue was purified by flash column chromatography (silica gel, 0-10% gradient of methanol/dichloromethane) to give a yellow oily compound benzyl 12-((1R,5S)-8-(tert-butoxycarbonyl)-3,8-diazabicyclo[3.2.1]octan-3-yl)-3,16-difluoro-14-(((2R,7aS)-2-fluoro-tetrahydro-1H-pyrazin-7a(5H)-yl)methoxy)-18-(methoxymethoxy)-4,5,6,7,8,9-hexahydro-10H-naphtho[1′,8′:4,5,6][1]azacyclododecino[2,3-g]quinazoline-10-carboxylate (60.0 mg, 0.06 mmol, yield: 78%). LCMS (ESI): [M+H]⁺=951.7.

Step VII: Under a nitrogen atmosphere at 25° C., to a solution of benzyl 12-((1R,5S)-8-(tert-butoxycarbonyl)-3,8-diazabicyclo[3.2.1]octan-3-yl)-3,16-difluoro-14-(((2R,7aS)-2-fluoro-tetrahydro-1H-pyrazin-7a(5H)-yl)methoxy)-18-(methoxymethoxy)-4,5,6,7,8,9-hexahydro-10H-naphtho[1′,8′:4,5,6][1]azacyclododecino[2,3-g]quinazoline-10-carboxylate (55.0 mg, 0.06 mmol) in ethyl acetate (2 mL) was added wet palladium on carbon (55.0 mg). The reaction mixture was stirred at 20° C. under a hydrogen atmosphere (15 psi) for 16 hours. The mixture was filtered, and the filtrate was concentrated under reduced pressure to give a yellow solid compound tert-butyl (1R,5S)-3-(3,16-difluoro-14-(((2R,7aS)-2-fluoro-tetrahydro-1H-pyrazin-7a(5H)-yl)methoxy)-18-(methoxymethoxy)-5,6,7,8,9,10-hexahydro-4H-naphtho[1′,8′:4,5,6][1]azacyclododecino[2,3-g]quinazoline-12-yl)-3,8-diazabicyclo[3.2.1]octane-8-carboxylate (40.0 mg, 0.05 mmol, yield: 85%). LCMS (ESI): [M+H]⁺=817.2.

Step VIII: At 25° C., to a solution of tert-butyl (1R,5S)-3-(3,16-difluoro-14-(((2R,7aS)-2-fluorotetrahydro-1H-pyrazin-7a(5H)-yl)methoxy)-18-(methoxymethoxy)-5,6,7,8,9,10-hexahydro-4H-naphtho[1′,8′:4,5,6][1]azacyclododecino[2,3-g]quinazolin-12-yl)-3,8-diaza-bicyclo[3.2.1]octane-8-carboxylate (35.0 mg, 0.04 mmol) in acetonitrile (I mL) was added hydrogen chloride (4 M in dioxane, 214.0 μL, 0.86 mmol). The mixture was stirred at 20° C. for 1 hour. The reaction mixture was quenched with triethylamine (200 μL) and concentrated. The residue was purified by preparative HPLC to give a yellow solid compound 12-((1R,5S)-3,8-diaza-bicyclo[3.2.1]octan-3-yl)-3,16-difluoro-14-(((2R,7aS)-2-fluorotetrahydro-1H-pyrazin-7a(5H)-yl)methoxy)-5,6,7,8,9,10-hexahydro-4H-naphtho[1′,8′:4,5,6][1]azacyclododecino[2,3-g]quinazolin-18-ol (formate salt, 4.59 mg, 6.55 μmol, yield: 15%/). LCMS (ESI): [M+H]⁺=673.2. ¹H NMR (400 MHz, CD₃OD) δ ppm 8.52 (br s, 1H), 7.65 (dd, J=8.9, 5.9 Hz, 1H), 7.33-7.18 (m, 2H), 6.94 (br s, 2H), 5.48-5.29 (in, 1H), 4.50 (br d, J=12.6 Hz, 1H), 4.46-4.32 (m, 3H), 3.95 (br s, 2H), 3.73-3.59 (m, 3H), 3.54 (br s, 3H), 3.22 (br d, J=11.74H-z, 1H), 2.76-2.63 (m, 1H), 2.52-1.92 (m, 12H), 1.77 (br s, 1H), 1.56-1.39 (m, 3H), 1.27-1.16 (m, 3H), 1.07 (br d, J=6.6 Hz, 1H).

### Examples 9A and 9B: 12-((1R,5S)-3,8-diazabicyclo[3.2.1]octan-3-yl)-3,16-difluoro-14-(((2R,7aS)-2-fluorotetrahydro-1H-pyrazin-7a(5H)-yl)methoxy)-18-hydroxy-5,6,7,8-tetrahydro-4H-naphtho[1′,8′:4,5,6][1]azacyclododecino[2,3-g]quinazolin-9(10H)-one

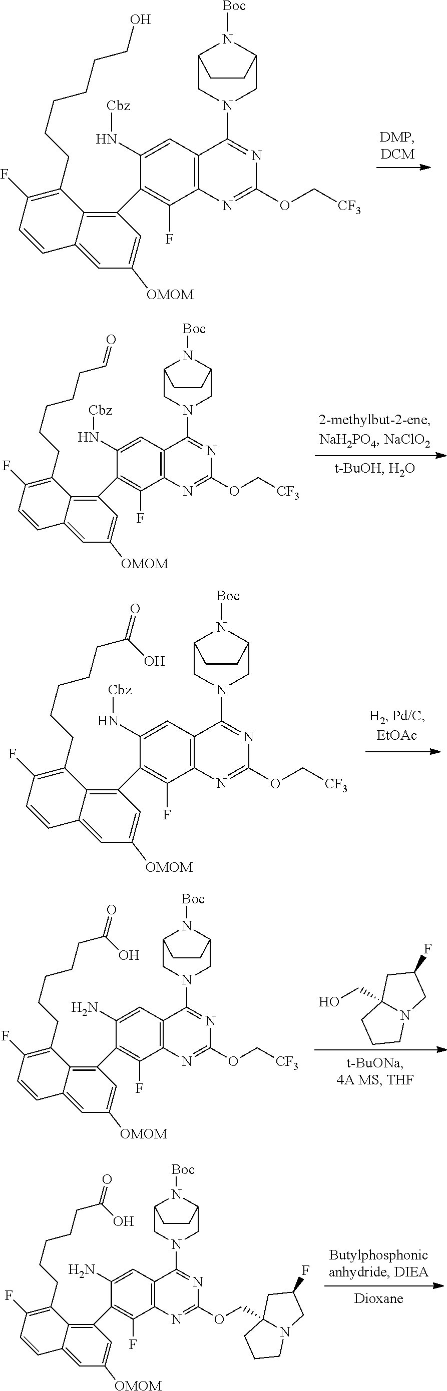

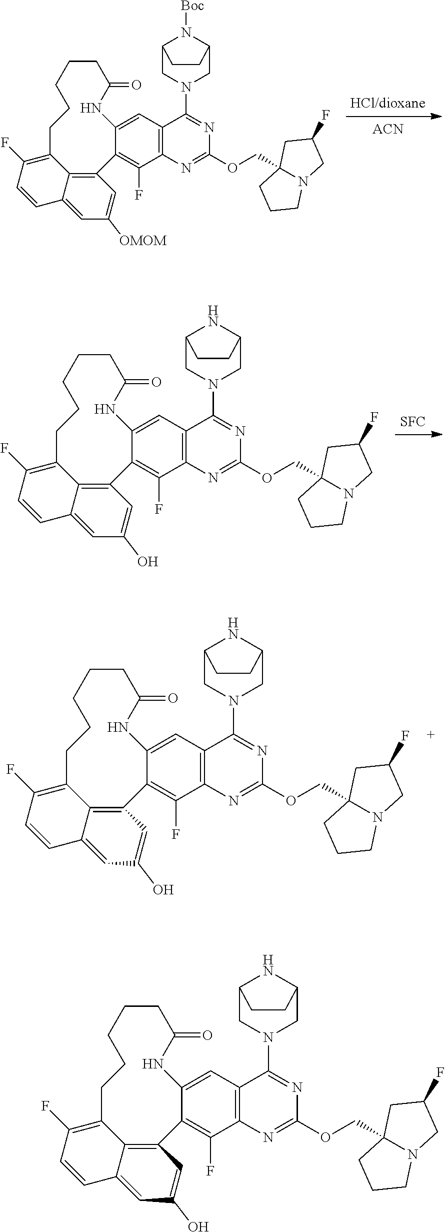

Step I: Under a nitrogen atmosphere at 20° C., to a solution of tert-butyl (1R,5S)-3-(6-(((benzyloxy)carbonyl)amino)-8-fluoro-7-(7-fluoro-8-(6-hydroxyhexyl)-3-(methoxymethoxy)naphthalen-1-yl)-2-(2,2,2-trifluoroethoxy)quinazolin-4-yl)-3,8-diazabicyclo[3.2.1]octane-8-carboxylate (700.0 mg, 0.77 mmol) in dichloromethane (7.00 mL) was slowly added with Dess-Martin periodinane (652.0 mg, 1.53 mmol). The mixture was stirred at 20° C. for 3 hours. The reaction mixture was added with saturated sodium bicarbonate solution (4 mL) and water (6 mL), and extracted with ethyl acetate (3×5 mL). The organic layers were dried over anhydrous sodium sulfate, filtered, and concentrated under reduced pressure to give a crude compound tert-butyl (1R,5S)-3-(6-(((benzyloxy)carbonyl)amino)-8-fluoro-7-(7-fluoro-3-(methoxymethoxy)-8-(6-oxohexyl)naphthalen-1-yl)-2-(2,2,2-trifluoroethoxy)quinazolin-4-yl)-3,8-diazabicyclo[3.2.1]octane-8-carboxylate (1.00 g), which was obtained as a yellow oily liquid. LCMS (ESI): [M+H]⁺=908.3.

Step II: At 0° C., to a solution of tert-butyl (1R,5S)-3-(6-(((benzyloxy)carbonyl)amino)-8-fluoro-7-(7-fluoro-3-(methoxymethoxy)-8-(6-oxohexyl)naphthalen-1-yl)-2-(2,2,2-trifluoroethoxy)quinazolin-4-yl)-3,8-diazabicyclo[3.2.1]octane-8-carboxylate (1.00 g, 1.10 mmol) in tert-butanol (6.00 mL) and water (3.00 mL) were slowly added 2-methyl-2-butene (2.33 mL, 22.03 mmol), disodium hydrogen phosphate (0.66 g, 5.51 mmol), and sodium chlorite (0.50 g, 5.51 mmol). The reaction mixture was diluted with water (10 mL) and extracted with ethyl acetate (3×10 mL). The combined organic layers were washed with saturated sodium bicarbonate solution (8 mL), dried over anhydrous sodium sulfate, filtered, and concentrated under reduced pressure. The residue was purified by flash column chromatography (silica gel, 0-25% gradient of tetrahydrofuran/petroleum ether) to give a yellow solid compound 6-(8-(6-(((benzyloxy)carbonyl)amino)-4-((1R,5S)-8-(tert-butoxycarbonyl)-3,8-diazabicyclo[3.2.1]octan-3-yl)-8-fluoro-2-(2,2,2-trifluoroethoxy)quinazolin-7-yl)-2-fluoro-6-(methoxymethoxy)naphthalen-1-yl)hexanoic acid (620.0 mg, 0.67 mmol, yield: 60%). LCMS (ESI): [M+H]⁺=924.3.

Step III: At 20° C., to a solution of 6-(8-(6-(((benzyloxy)carbonyl)amino)-4-((1R,5S)-8-(tert-butoxycarbonyl)-3,8-diazabicyclo[3.2.1]octan-3-yl)-8-fluoro-2-(2,2,2-trifluoroethoxy)quinazolin-7-yl)-2-fluoro-6-(methoxymethoxy)naphthalen-1-yl)hexanoic acid (620.0 mg, 0.67 mmol) in ethyl acetate (5.00 mL) was added dry palladium on carbon (620.0 mg). The reaction mixture was stirred at 20° C. under a hydrogen atmosphere (15 psi) for 16 hours. The reaction mixture was filtered, and the filtrate was concentrated under reduced pressure. The residue was purified by flash column chromatography (silica gel, 0-20% gradient of tetrahydrofuran/petroleum ether) to give a brown solid compound 6-(8-(6-amino-4-((1R,5S)-8-(tert-butoxycarbonyl)-3,8-diazabicyclo[3.2.1]octan-3-yl)-8-fluoro-2-(2,2,2-trifluoroethoxy)quinazolin-7-yl)-2-fluoro-6-(methoxymethoxy)naphthalen-1-yl)hexanoic acid (500.0 mg, 0.63 mmol, yield: 94%). LCMS (ESI): [M+H]⁺=790.3.

Step IV: At 20° C., to a solution of 6-(8-(6-amino-4-((1R,5S)-8-(tert-butoxycarbonyl)-3,8-diazabicyclo[3.2.1]octan-3-yl)-8-fluoro-2-(2,2,2-trifluoroethoxy)quinazolin-7-yl)-2-fluoro-6-(methoxymethoxy)naphthalen-1-yl)hexanoic acid (500.0 mg, 0.63 mmol) and 4A powdered molecular sieves (500.0 mg) in tetrahydrofuran (7.00 mL) were slowly added ((2R,7aS)-2-fluorotetrahydro-1H-pyrazin-7a(5H)-yl)methanol (806.2 mg, 5.06 mmol) and sodium tert-butoxide (365.0 mg, 3.79 mmol). The mixture was stirred at 80° C. for 16 hours. The reaction mixture was diluted with water (5 mL) and extracted with ethyl acetate (3×5 mL). The combined organic layers were dried over anhydrous sodium sulfate, filtered, and concentrated under reduced pressure to give a crude compound 6-(8-(6-amino-4-((1R,5S)-8-(tert-butoxycarbonyl)-3,8-diazabicyclo[3.2.1]octan-3-yl)-8-fluoro-2-(((2R,7aS)-2-fluorotetrahydro-1H-pyrazin-7a(5H)-yl)methoxy)quinazolin-7-yl)-2-fluoro-6-(methoxymethoxy)naphthalen-1-yl)hexanoic acid (1.00 g), which was obtained as a brown oily liquid. LCMS (ESI): [M+H]⁺=849.4.

Step V: At 20° C., to a solution of 6-(8-(6-amino-4-((1R,5S)-8-(tert-butoxycarbonyl)-3,8-diazabicyclo[3.2.1]octan-3-yl)-8-fluoro-2-(((2R,7aS)-2-fluorotetrahydro-1H-pyrazin-7a(5H)-yl)methoxy)quinazolin-7-yl)-2-fluoro-6-(methoxymethoxy)naphthalen-1-yl)hexanoic acid (1.00 g, 1.17 μmol) in dioxane (40.0 mL) were slowly added diisopropylethylamine (8.39 mL, 47.12 mmol) and tri-n-butylphosphine anhydride (50% ethyl acetate solution, 3.50 mL, 5.88 mmol). The mixture was stirred at 100° C. for 2 hours. The reaction mixture was diluted with water (20 mL) and extracted with ethyl acetate (2×20 mL). The combined organic layers were dried over anhydrous sodium sulfate, filtered, and concentrated under reduced pressure to give a yellow solid compound tert-butyl (1R,5S)-3-(3,16-difluoro-14-(((2R,7aS)-2-fluorotetrahydro-1H-pyrazin-7a(5H)-yl)methoxy)-18-(methoxymethoxy)-9-oxo-5,6,7,8,9,10-hexahydro-4H-naphtho[1′,8′:4,5,6][1]azacyclododecino[2,3-g]quinazolin-12-yl)-3,8-diazabicyclo[3.2.1]octane-8-carboxylate (0.80 g, 0.96 μmol, yield: 82%). LCMS (ESI): [M+H]⁺=831.4.

Step VI: At 20° C., to a solution of tert-butyl (1R,5S)-3-(3,16-difluoro-14-(((2R,7aS)-2-fluorotetrahydro-1H-pyrazin-7a(5H)-yl)methoxy)-18-(methoxymethoxy)-9-oxo-5,6,7,8,9,10-hexahydro-4H-naphtho[1′,8′:4,5,6][1]azacyclododecyl[2,3-g]quinazolin-12-yl)-3,8-diaza-cyclo[3.2.1]octane-8-carboxylate (0.80 g, 0.96 μmol) in acetonitrile (10.0 mL) was added hydrogen chloride (4 M in dioxane, 4.81 mL, 19.2 mmol). The mixture was stirred at 20° C. for 2 hours. the reaction mixture was concentrated under reduced pressure, and the residue was dissolved in dimethyl sulfoxide (3 mL) and neutralized with triethylamine (200 μL). The mixture was purified by preparative HPLC to give a yellow solid compound 12-((1R,5S)-3,8-diazabicyclo[3.2.1]octan-3-yl)-3,16-difluoro-14-(((2R,7aS)-2-fluorotetrahydro-1H-pyrazin-7a(5H)-yl)methoxy)-18-hydroxy-5,6,7,8-tetrahydro-4H-naphtho[1′,8′:4,5,6][1]azacyclododecino[2,3-g]quinazolin-9(10H)-one (Example 10) (43.03 mg, 0.06 μmol, yield: 6%). LCMS (ESI): [M+H]⁺=687.2. ¹H NMR (400 MHz, CH₃OD) δ ppm 8.94-7.75 (m, 1H), 7.68 (dd, J=9.0, 5.8 Hz, 1H), 7.34-7.19 (m, 2H), 6.91 (br s, 1H), 5.38-5.18 (m, 1H), 4.66-4.39 (m, 2H), 4.29-4.14 (m, 2H), 3.66 (br s, 4H), 3.19 (br d, J=19.1 Hz, 3H), 3.06-2.74 (m, 3H), 2.39-2.05 (m, 4H), 2.03-1.81 (m, 9H), 1.52-0.71 (m, 5H).

### Example 9: Two Stereoisomers were Obtained by SFC Separation (Column: DAICEL CHIRALPAK IG, 250 mm×30 mm, 10 μm; Mobile Phase: Phase A, Carbon Dioxide; Phase B, 0.1% Ammonia in Isopropanol; Phase B Maintained at 60%; Flow Rate: 80 mL/min)

Example 9A: LCMS (ESI): [M+H]⁺=687.2; SFC analysis (column: Chiralpak IG-3, 50×4.6 mm I.D., 3 μm; mobile phase: phase A, carbon dioxide; phase B, 0.05% diethylamine in isopropanol; gradient: phase B maintained at 40%; flow rate: 4 mL/min): Retention time on the chiral column was 1.861 min; ¹H NMR (400 MHz, CD₃OD) δ ppm 9.15-7.90 (m, 1H), 7.68 (dd, J=8.9, 5.9 Hz, 1H), 7.35-7.19 (m, 2H), 6.91 (s, 1H), 5.35-5.21 (m, 1H), 4.60-4.39 (m, 2H), 4.31-4.12 (m, 2H), 3.65 (br s, 4H), 3.23-3.12 (m, 3H), 3.05-2.76 (m, 3H), 2.39-2.06 (m, 4H), 2.04-1.75 (m, 9H), 1.52-1.23 (m, 5H).

Example 9B: LCMS (ESI): [M+H]⁺=687.2; SFC analysis (column: Chiralpak IG-3, 50×4.6 mm I.D., 3 μm; mobile phase: phase A, carbon dioxide; phase B, 0.05% diethylamine in isopropanol; gradient: phase B maintained at 40%; flow rate: 4 mL/min): Retention time on the chiral column was 5.520 min; ¹H NMR (400 MHz, CD₃OD) δ ppm 9.10-7.98 (m, 1H), 7.67 (dd, J=9.11, 5.93 Hz, 1H), 7.35-7.19 (m, 2H), 6.90 (s, 1H), 5.35-5.20 (m, 1H), 4.67-4.36 (m, 2H), 4.29-4.11 (m, 2H), 3.75-3.47 (m, 4H), 3.23-3.14 (m, 3H), 3.04-2.75 (m, 3H), 2.40-2.07 (m, 4H), 2.01-1.76 (m, 9H), 1.50-1.28 (m, 5H).

### Example 10: 15-((1R,5S)-3,8-diazabicyclo[3.2.1]octan-3-yl)-3,19-difluoro-17-(((2R,7aS)-2-fluorotetrahydro-1H-pyrazin-7a(5H)-yl)methoxy)-21-hydroxy-4,5,6,7,8,9,10,11-octahydronaphtho[1′,8′:4,5,6][1]azacyclopentadecano[2,3-g]quinazolin-12(13H)-one

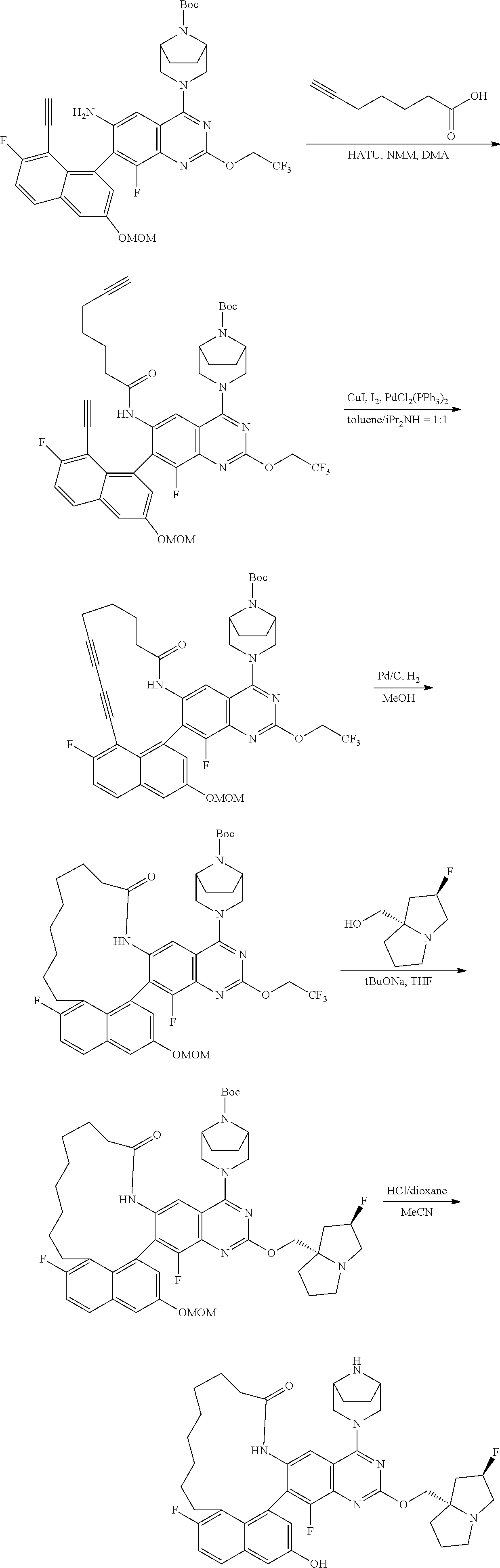

Step I: To a solution of tert-butyl (1R,5S)-3-(6-amino-7-(8-ethynyl-7-fluoro-3-(methoxymethoxy)naphthalen-1-yl)-8-fluoro-2-(2,2,2-trifluoroethoxy)quinazolin-4-yl)-3,8-diazabicyclo[3.2.1]octane-8-carboxylate (200 mg, 0.29 mmol), hept-6-ynoic acid (180.3 mg, 1.43 mmol), and N-methylmorpholine (144.4 mg, 1.43 mmol) in N,N-dimethylacetamide (4 mL) was added 0-(7-azabenzotriazol-1-yl)-N,N,N,N′-tetramethyluronium hexafluorophosphate (326 mg, 0.86 mmol). The mixture is stirred under a nitrogen atmosphere at 25° C. for 1 hour and then stirred at 60° C. for 16 hours. Concentrate under pressure, saturated sodium bicarbonate solution (10 mL) were added, and the mixture was extracted with ethyl acetate (3×10 mL). The combined organic layers were dried over anhydrous sodium sulfate, filtered, and concentrated under reduced pressure. The residue was purified by flash column chromatography (silica gel, 0-30% gradient of ethyl acetate/petroleum ether) to give a yellow solid compound tert-butyl (1R,5S)-3-(7-(8-ethynyl-7-fluoro-3-(methoxymethoxy)naphthalen-1-yl)-8-fluoro-6-(hept-6-ynamido)-2-(2,2,2-trifluoroethoxy)quinazolin-4-yl)-3,8-diazabicyclo[3.2.1]octane-8-carboxylate (150 mg, 0.19 mmol, yield: 65%). LCMS (ESI): [M+H]⁺=808.2.

Step II: To a solution of tert-butyl (1R,5S)-3-(7-(8-ethynyl-7-fluoro-3-(methoxymethoxy)naphthalen-1-yl)-8-fluoro-6-(hept-6-ynamido)-2-(2,2,2-trifluoroethoxy)quinazolin-4-yl)-3,8-diazabicyclo[3.2.1]octane-8-carboxylate (80.0 mg, 0.10 mmol), bis(triphenylphosphine)palladium(II) dichloride (3.5 mg, 5.0 μmol), and copper(I) iodide (1.9 mg, 10.0 μmol) in toluene/diisopropylamine (1:1 v/v, 90 mL) was added iodine (75.4 mg, 0.30 mmol). The mixture was stirred at 80° C. for 2 hours. The reaction was added with saturated sodium bisulfite solution (30 mL) and extracted with ethyl acetate (3×30 mL). The combined organic layers were dried over anhydrous sodium sulfate, filtered, and concentrated under vacuum. The residue was purified by flash column chromatography (silica gel, 0-25% gradient of ethyl acetate/petroleum ether) to give a yellow solid cyclized compound (42.0 mg, 0.05 mmol, yield: 53%). LCMS (ESI): [M+H]⁺=806.3.

Step III: To a solution of the above-obtained cyclized compound (58.0 mg, 0.07 mmol) in methanol (50 mL) was added dry palladium on carbon (100.0 mg). The mixture is stirred under a hydrogen atmosphere at 15 psi and 25° C. for 16 hours. The reaction mixture was filtered, and the solid was washed with methanol (100 mL). The filtrate was concentrated under reduced pressure to give a crude compound tert-butyl (1R,5S)-3-(3,19-difluoro-21-(methoxymethoxy)-12-oxo-17-(2,2,2-trifluoroethoxy)-4,5,6,7,8,9,10,11,12,13-decahydronaphtho[1′,8′:4,5,6][1]azacyclopentadecino[2,3-g]quinazolin-15-yl)-3,8-diazabicyclo[3.2.1]octane-8-carboxylate (56.0 mg), which was obtained as a brown solid. LCMS (ESI): [M+H]⁺=814.4.

Step IV: To a solution of tert-butyl (1R,5S)-3-(3,19-difluoro-21-(methoxymethoxy)-12-oxo-17-(2,2,2-trifluoroethoxy)-4,5,6,7,8,9,10,11,12,13-decahydronaphtho[1′,8′:4,5,6][1]azacyclopentadeca[2,3-g]quinazolin-15-yl)-3,8-diazabicyclo[3.2.1]octane-8-carboxylate (56 mg, 0.07 mmol) and ((2R,7aS)-2-fluorotetrahydro-1H-pyrazin-7a(5H)-yl)methanol (43.8 mg, 0.28 mmol) in tetrahydrofuran (1.1 mL) was added sodium tert-butoxide (19.8 mg, 0.21 mmol) under a nitrogen atmosphere. The mixture was stirred at 50° C. under a nitrogen atmosphere for 16 hours. The reaction was diluted with water (2 mL), and extracted with ethyl acetate (3 mL×3). The combined organic layers were dried over anhydrous sodium sulfate, filtered, and concentrated under reduced pressure to give a crude compound tert-butyl (1R,5S)-3-(3,19-difluoro-17-(((2R,7aS)-2-fluorotetrahydro-1H-pyrazin-7a(5H)-yl)methoxy)-21-(methoxymethoxy)-12-oxo-4,5,6,7,8,9,10,11,12,13-decahydronaphtho[1′,8′:4,5,6][1]azacyclopentadeca [2,3-g]quinazolin-15-yl)-3,8-diazabicyclo[3.2.1]octane-8-carboxylate (120 mg), was obtained as a brown oily liquid. LCMS (ESI): [M+H]⁺=873.7.

Step V: To the solution of tert-butyl (1R,5S)-3-(3,19-difluoro-21-(((2R,7aS)-2-fluorotetrahydro-1H-pyrazin-7a(5H)-yl)methoxy)-17-(methoxymethoxy)-12-oxo-4,5,6,7,8,9,10,11,12,13-decahydronaphtho[1′,8′:4,5,6][1]azacyclopentadeca[2,3-g]quinazolin-15-yl)-3,8-diazabicyclo[3.2.1]octane-8-carboxylate (100 mg, 0.07 mmol) in acetonitrile (2 mL) was added hydrogen chloride (4 M in dioxane, 429 IL, 1.72 mmol). The mixture was stirred at 25° C. for 1 hour. The mixture was evaporated under a nitrogen stream. The residue was purified by preparative HPLC to give a white solid compound 15-((1R,5S)-3,8-diazabicyclo[3.2.1]octan-3-yl)-3,19-difluoro-17-(((2R,7aS)-2-fluorotetrahydro-1H-pyrazin-7a(5H)-yl)methoxy)-21-hydroxy-4,5,6,7,8,9,10,11-octahydronaphtho[1′,8′:4,5,6][1]azacyclopentadecino[2,3-g]quinazolin-12(13H)-one (formate salt, 8.90 mg, 0.01 mmol, yield: 11%). LCMS (ESI): [M+H]⁺=729.2. ¹H NMR (400 MHz, CD₃OD) δ ppm 9.18 (s, 1H), 8.55 (s, 1H), 7.73 (dd, J=5.9, 9.0 Hz, 1H), 7.37 (d, J=2.5 Hz, 1H), 7.29 (t, J=9.4 Hz, 1H), 6.98 (d, J=2.6 Hz, 1H), 5.49-5.21 (m, 1H), 4.65 (br d, J=12.9 Hz, 1H), 4.49 (br d, J=13.1 Hz, 1H), 4.40-4.27 (m, 2H), 3.89 (br s, 2H), 3.74 (br d, J=13.9 Hz, 1H), 3.59 (br d, J=13.5 Hz, 1H), 3.52-3.35 (m, 3H), 3.12 (dt, J=5.4, 9.7 Hz, 1H), 2.90-2.72 (m, 1H), 2.58-2.36 (m, 2H), 2.35-2.28 (m, 2H), 2.20-1.95 (m, 10H), 1.49 (br s, 1H), 1.36-1.12 (m, 10H).

### Example 11: 16-((1R,5S)-3,8-diazabicyclo[3.2.1]octan-3-yl)-3,20-difluoro-18-(((2R,7aS)-2-fluorotetrahydro-1H-pyrazin-7a(5H)-yl)methoxy)-22-hydroxy-5,6,7,8,9,10,11,12-octahydro-4H-naphtho[1′,8′:4,5,61][1]azacyclohexadecano[2,3-g]quinazolin-13(14H)-one

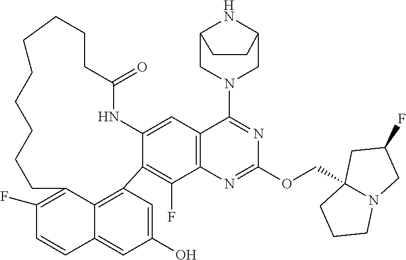 [SMILES: O=C1CCCCCCCCCc2c(F)ccc3cc(O)cc(c23)-c2c(cc3c(N4CC5CCC(C4)N5)nc(OC[C@@]45CCCN4C[C@H](F)C5)nc3c2F)N1]

Example 11: Prepared according to a similar synthetic route as described in Example 10: LCMS (ESI): [M+H]⁺=743.3; ¹H NMR (400 MHz, CD₃OD) δ ppm 9.24 (s, 1H), 7.71 (dd, J=5.8, 9.1 Hz, 1H), 7.35 (d, J=2.6 Hz, 1H), 7.27 (t, J=9.6 Hz, 1H), 7.00 (d, J=2.6 Hz, 1H), 5.36-5.22 (m, 1H), 4.53 (br dd, J=8.3, 12.4 Hz, 2H), 4.26 (d, J=10.6 Hz, 1H), 4.18 (dd, J=2.3, 10.2 Hz, 1H), 3.72-3.53 (m, 4H), 3.30-3.11 (m, 4H), 3.00 (br d, J=5.1 Hz, 1H), 2.78-2.62 (m, 1H), 2.38-2.33 (m, 2H), 2.25-2.18 (m, 1H), 2.15-2.10 (m, 1H), 2.05 (br d, J=4.0 Hz, 2H), 2.02-1.87 (m, 8H), 1.85-1.76 (m, 2H), 1.58 (br s, 1H), 1.45 (br d, J=6.2 Hz, 1H), 1.37-1.27 (m, 2H), 1.23-1.15 (m, 6H).

### Example 12: 10-((1R,5S)-3,8-diazabicyclo[3.2.1]oct-3-yl)-3,14-difluoro-12-(((2R,7aS)-2-fluorotetrahydro-1H-pyrazin-7a(5H)-yl)methoxy)-16-hydroxy-4,5,6,7-tetrahydro-8H-naphtho[1′,8′:5,6,7]acredo [3,4-g]quinazolin-8-one

### Example 13: 10-((1R,5S)-3,8-diazabicyclo[3.2.1]oct-3-yl)-7-ethyl-3,14-difluoro-12-(((2R,7aS)-2-fluorotetrahydro-1H-pyrazin-7a(5H)-yl)methoxy)-16-hydroxy-4,5,6,7-tetrahydro-8H-naphtho[1′,8′:5,6,7]acrylo[3,4-g]quinazolin-8-one

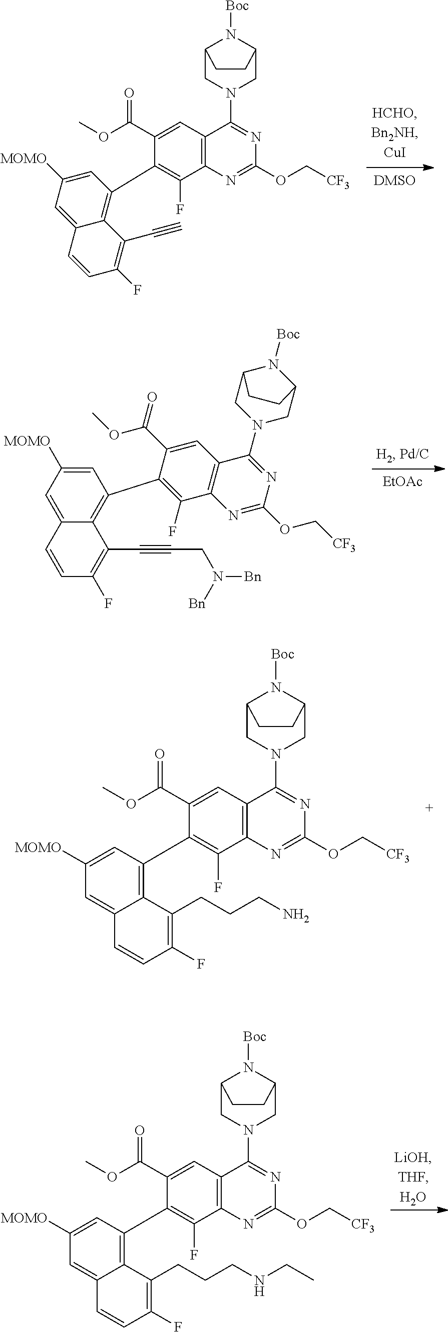

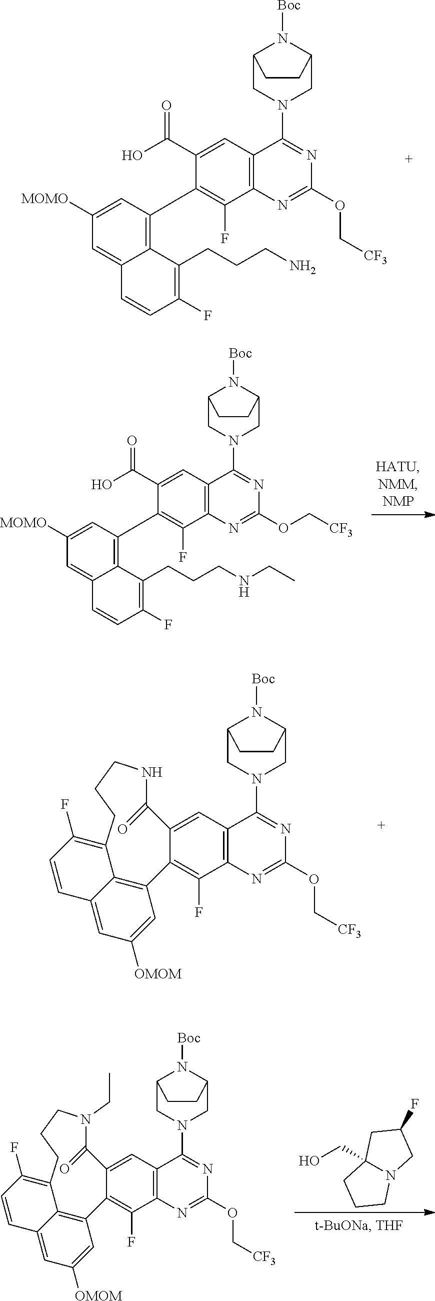

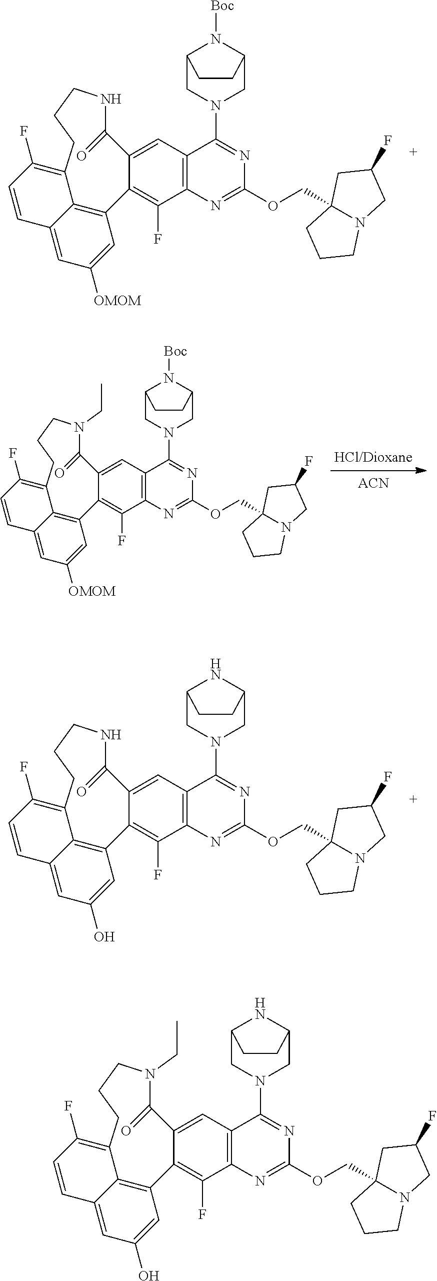

Step I: Methyl 4-((1R,5S)-8-(tert-butoxycarbonyl)-3,8-diazabicyclo[3.2.]octan-3-yl)-7-(8-ethynyl-7-fluoro-3-(methoxymethoxy)naphthalen-1-yl)-8-fluoro-2-(2,2,2-trifluoroethoxy)quinazoline-6-carboxylate (150 mg, 0.2 mmol), dibenzylamine (159 mg, 0.8 mmol), and formaldehyde (37% aqueous solution, 48 μL, 0.6 mmol) were dissolved in dimethyl sulfoxide (0.4 mL). Under a nitrogen atmosphere, copper(I) iodide (8 mg, 0.04 mmol) was added. and the mixture was stirred at 60° C. for 16 hours. The reaction mixture was diluted with water (5 mL) and extracted with ethyl acetate (2×5 mL). The combined organic layers were washed with saturated brine (5 mL), dried over magnesium sulfate, filtered, and concentrated under reduced pressure. The residue was purified by flash column chromatography (silica gel, 0-10% gradient methanol/dichloromethane) to give a yellow solid compound methyl 4-((R,5S)-8-(tert-butoxycarbonyl)-3,8-diazabicyclo[3.2.1]octan-3-yl)-7-(8-(3-(dibenzylamino)prop-1-yn-1-yl)-7-fluoro-3-(methoxymethoxy)naphthalen-11-yl)-8-fluoro-2-(2,2,2-trifluoroethoxy)quinazoline-6-carboxylate (120 mg, 0.12 mmol, yield: 62%). LCMS (ESI): [M+H]⁺=953.1.

Step II: Methyl 4-((1R,5S)-8-(tert-butoxycarbonyl)-3,8-diazabicyclo[3.2.1]octan-3-yl)-7-(8-(3-(dibenzylamino)prop-1-yn-1-yl)-7-fluoro-3-(methoxymethoxy)naphthalen-1-yl)-8-fluoro-2-(2,2,2-trifluoroethoxy)quinazoline-6-carboxylate (120 mg, 0.12 mmol) was dissolved in ethyl acetate (6 mL), and dry palladium on carbon (10% Pd, water content <1%, 100 mg) was added under a nitrogen atmosphere. The reaction vessel was purged with hydrogen gas three times, and the reaction mixture was stirred at 20° C. under a hydrogen atmosphere (15 psi) for 16 hours. The reaction mixture was filtered through diatomaceous earth, and the filtrate was concentrated under reduced pressure to give a crude mixture of methyl 7-(8-(3-aminopropyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate and methyl 4-((1R,5S)-8-(tert-butoxycarbonyl)-3,8-diazabicyclo[3.2.1]octan-3-yl)-7-(8-(3-(ethylamino)propyl)-7-fluoro-3-(methoxymethoxy)naphthalen-1-yl)-8-fluoro-2-(2,2,2-trifluoroethoxy)quinazoline-6-carboxylate (80 mg) as a yellow oily liquid. LCMS (ESI): [M+H]⁺=776.1; 804.3.

Step III: To a mixture of methyl 7-(8-(3-aminopropyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate and methyl 4-((1R,5S)-8-(tert-butoxycarbonyl)-3,8-diazabicyclo[3.2.1]octan-3-yl)-7-(8-(3-(ethylamino)propyl)-7-fluoro-3-(methoxymethoxy)naphthalen-1-yl)-8-fluoro-2-(2,2,2-trifluoroethoxy)quinazoline-6-carboxylate (80 mg, 0.10 mmol) was dissolved in tetrahydrofuran (1 mL) and water (0.2 mL) was added lithium hydroxide (26 mg, 0.60 mmol) at 0° C., and the reaction mixture was stirred at 60° C. for 16 hours. The reaction mixture was adjusted to pH 4 with 1 M dilute hydrochloric acid and lyophilized to give a mixture of 7-(8-(3-aminopropyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid and 4-((1R,5S)-8-(tert-butoxycarbonyl)-3,8-diazabicyclo[3.2.1]octan-3-yl)-7-(8-(3-(ethylamino)propyl)-7-fluoro-3-(methoxymethoxy)naphthalen-1-yl)-8-fluoro-2-(2,2,2-trifluoroethoxy)quinazoline-6-carboxylic acid (60 mg). LCMS (ESI): [M+H]⁺=762.5; 790.3.

Step IV: To a solution of a mixture of 7-(8-(3-aminopropyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid and 4-((1R,5S)-8-(tert-butoxycarbonyl)-3,8-diazabicyclo[3.2.1]octan-3-yl)-7-(8-(3-(ethylamino)propyl)-7-fluoro-3-(methoxymethoxy)naphthalen-1-yl)-8-fluoro-2-(2,2,2-trifluoroethoxy)quinazoline-6-carboxylic acid (60 mg, 0.08 mmol) in N-methylpyrrolidone (2 mL) were added N-methylmorpholine (40 mg, 0.39 mmol) and O-(7-azabenzotriazol-1-yl)-N,N,N′,N-tetramethyluronium hexafluorophosphate (90 mg, 0.24 mmol). The mixture was stirred at 80° C. for 16 hours. The reaction mixture was diluted with water (5 mL), extracted with ethyl acetate (2×5 mL). The combined organic layers were washed with saturated brine (2×5 mL), dried over magnesium sulfate, filtered, and concentrated under reduced pressure to give a crude mixture of tert-butyl (1R,5S)-3-(3,14-difluoro-16-(methoxymethoxy)-8-oxo-12-(2,2,2-trifluoroethoxy)-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate and tert-butyl (1R,5S)-3-(7-ethyl-3,14-difluoro-16-(methoxymethoxy)-8-oxo-12-(2,2,2-trifluoroethoxy)-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate (40 mg), which was obtained as a yellow oily liquid. LCMS (ESI): [M+H]⁺=744.2; 772.2.

Step V: To a solution of a mixture of tert-butyl (1R,5S)-3-(3,14-difluoro-16-(methoxymethoxy)-8-oxo-12-(2,2,2-trifluoroethoxy)-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate and tert-butyl (1R,5S)-3-(7-ethyl-3,14-difluoro-16-(methoxymethoxy)-8-oxo-12-(2,2,2-trifluoroethoxy)-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate (40 mg, 0.05 mmol) in tetrahydrofuran (0.4 mL) were added ((2R,7aS)-2-fluorotetrahydro-1H-pyrazin-7a(5H)-yl)methanol (18 mg, 0.11 mmol) and sodium tert-butoxide (10 mg, 0.10 mmol). The reaction mixture was stirred at 20° C. for 1 hour. The reaction mixture was then diluted with water (5 mL) and extracted with ethyl acetate (2×5 mL). The combined organic layers were washed with saturated brine (5 mL), dried over magnesium sulfate, filtered, and concentrated under reduced pressure to give a crude mixture of tert-butyl (1R,5S)-3-(3,14-difluoro-12-(((2R,7aS)-2-fluorotetrahydro-1H-pyrazin-7a(5H)-yl)methoxy)-16-(methoxymethoxy)-8-oxo-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate and tert-butyl (1R,5S)-3-(7-ethyl-3,14-difluoro-12-(((2R,7aS)-2-fluorotetrahydro-1H-pyrazin-7a(5H)-yl)methoxy)-16-(methoxymethoxy)-8-oxo-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate (30 mg), which was obtained as a yellow oily liquid. LCMS (ESI): [M+H]⁺=804.1; 832.2.

Step VI: To a mixture of tert-butyl (1R,5S)-3-(3,14-difluoro-12-(((2R,7aS)-2-fluorotetrahydro-1H-pyrazin-7a(5H)-yl)methoxy)-16-(methoxymethoxy)-8-oxo-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate and tert-butyl (1R,5S)-3-(7-ethyl-3,14-difluoro-12-(((2R,7aS)-2-fluorotetrahydro-1H-pyrazin-7a(5H)-yl)methoxy)-16-(methoxymethoxy)-8-oxo-5,6,7,8-tetrahydro-4H-naphtho[1′,8′:5,6,7]azacyclododecino[3,4-g]quinazolin-10-yl)-3,8-diazabicyclo[3.2.1]octane-8-carboxylate (30 mg, 37 μmol) in acetonitrile (0.1 mL) was stirred hydrogen chloride (4 M in dioxane, 0.18 mL, 0.74 mmol) was added, and the mixture was stirred at 20° C. for 2 hours. The reaction mixture was concentrated under reduced pressure, and the residue was dissolved in acetonitrile (500 μL). The pH was adjusted to 8 using triethylamine. The residue was purified by preparative HPLC to give two target compounds.

Example 12: 10-((1R,5S)-3,8-diazabicyclo[3.2.1]octan-3-yl)-3,14-difluoro-12-(((2R,7aS)-2-fluorotetrahydro-1H-pyrazin-7a(5H)-yl)methoxy)-16-hydroxy-4,5,6,7-tetrahydro-8H-naphtho[1′,8′:5,6,7]azacyclododecino[3,4-g]quinazolin-8-one (4.66 mg, 6 μmol, yield: 16%) was obtained as a white solid, LCMS (ESI): [M+H]⁺=659.2; ¹H NMR (400 MHz, CD₃OD) S ppm 7.61-7.50 (m, 2H), 7.18-7.06 (m, 2H), 6.83 (d, J=2.5 Hz, 1H), 5.32-5.11 (m, 1H), 4.26-3.90 (m, 3H), 3.74 (br d, J=12.6 Hz, 1H), 3.60-3.46 (m, 2H), 3.39 (br d, J=12.9 Hz, 1H), 3.30-3.23 (m, 1H), 3.19-3.05 (m, 4H), 2.97-2.75 (m, 2H), 2.46-2.35 (m, 1H), 2.31-2.02 (m, 3H), 1.94-1.83 (m, 3H), 1.83-1.64 (m, 5H), 1.61-1.50 (m, 1H), 1.31-1.15 (m, 1H).

Example 13: 10-((1R,5S)-3,8-diazabicyclo[3.2.1]oct-3-yl)-7-ethyl-3,14-difluoro-12-(((2R,7aS)-2-fluorotetrahydro-1H-pyrazin-7a(5H)-yl)methoxy)-16-hydroxy-4,5,6,7-tetrahydro-8H-naphtho[1′,8′:5,6,7]acredeno[3,4-g]quinazolin-8-one (6.41 mg, 8 umol, yield: 23%) as a white solid, LCMS (ESI): [M+H]⁺=687.4; 1H NMR (400 MHz, CD₃OD) δ ppm 7.61-7.49 (m, 2H), 7.19-7.07 (m, 2H), 6.80 (d, J=2.5 Hz, 1H), 5.32-5.10 (m, 1H), 4.25-4.05 (m, 3H), 3.74 (br d, J=12.5 Hz, 1H), 3.67-3.44 (m, 3H), 3.37 (br d, J=12.9 Hz, 1H), 3.17-3.06 (m, 3H), 3.02-2.88 (m, 2H), 2.87-2.72 (m, 2H), 2.50 (br t, J=11.8 Hz, 1H), 2.31-1.66 (m, 12H), 1.61-1.47 (m, 1H), 1.28-1.14 (m, 1H), 0.50 (t, J=7.1 Hz, 3H).

### Example 14: 12-((1R,5S)-3,8-diazabicyclo[3.2.1]oct-3-yl)-3,16-difluoro-14-(((2R,7aS)-2-fluorotetrahydro-1H-pyrazin-7a(5H)-yl)methoxy)-18-hydroxy-4,5,6,7,8,9-hexahydro-10H-naphtho[1′,8′:5,6,7][1]azacyclododecano[3,4-g]quinazolin-10-one

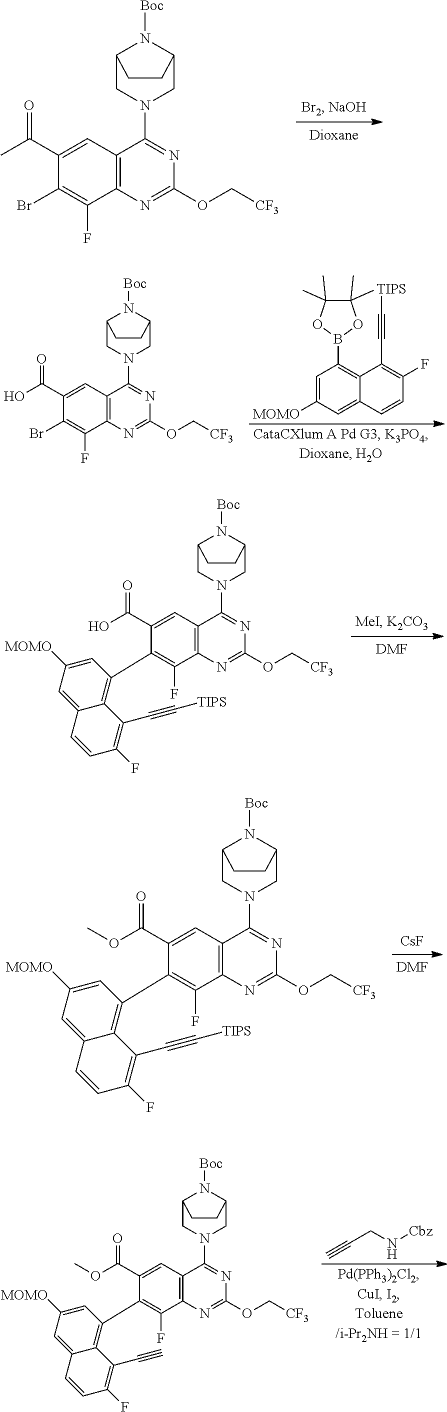

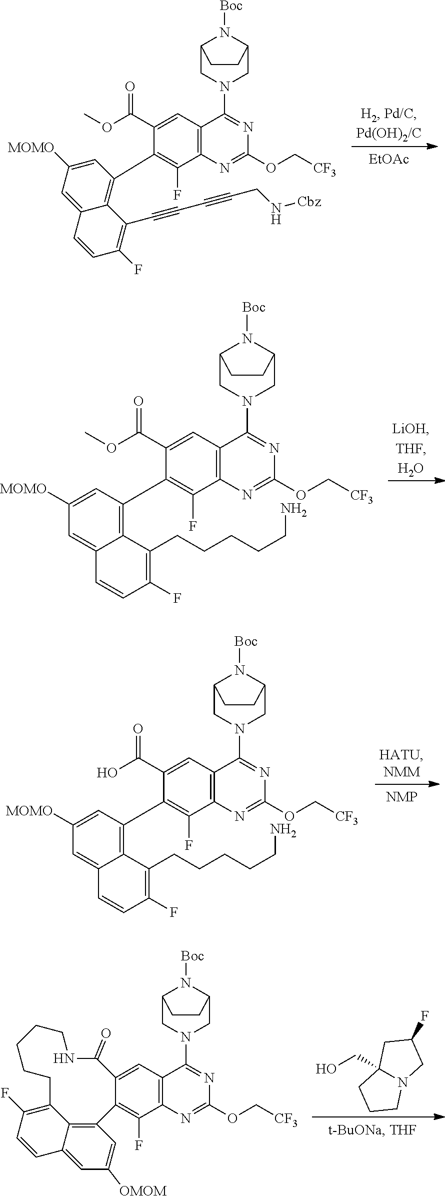

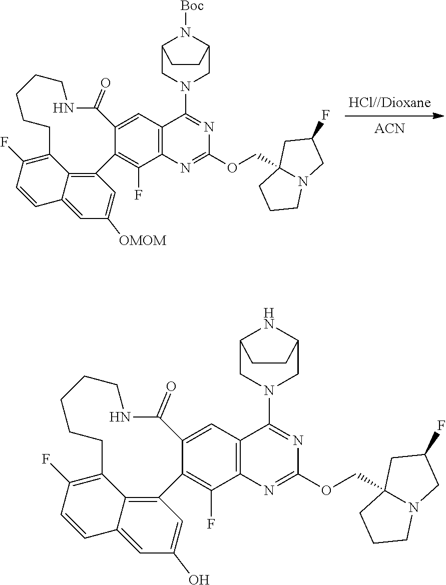

Step I: The solution of tert-butyl (1R,5S)-3-(6-acetyl-7-bromo-8-fluoro-2-(2,2,2-trifluoroethoxy)quinazolin-4-yl)-3,8-diazabicyclo[3.2.1]octane-8-carboxylate (2.00 g, 3.46 mmol) in dioxane (20 mL) and sodium hydroxide (1.5 M aqueous solution, 18.5 mL, 27.7 mmol) was prepared. At 0° C., bromine (0.53 mL, 10.4 mmol) was added dropwise. The reaction mixture was stirred at 0° C. for 3 hours and then at 20° C. for 16 hours. To the reaction mixture, water (50 mL) was added, and the pH was adjusted to 4 with 2 M dilute hydrochloric acid. The mixture was extracted with ethyl acetate (50 mL×3). The organic layers were washed with saturated brine (50 mL), dried over sodium sulfate, filtered, and concentrated under reduced pressure. The residue was purified by flash column chromatography (silica gel, 0-40% gradient of ethyl acetate/petroleum ether) to afford 7-bromo-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (1.00 g, 1.73 mmol, yield: 50%). LCMS (ESI): [M+H]⁺=581.3.

Step II: The solution of 7-bromo-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (350 mg, 0.60 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (402 mg, 0.78 mmol), and potassium phosphate (1.5 M aqueous solution, 1.20 mL, 1.81 mmol) in dioxane (3.50 mL) was prepared under a nitrogen atmosphere. To this solution, (methanesulfonyl)(diadamantyl-tert-butylphosphino)-2-amino-1,1′-biphenyl-2-yl)palladium(II) (44 mg, 0.06 mmol) was added. The reaction mixture was stirred at 100° C. for 16 hours. Four identical batches of this reaction were combined, diluted with water (20 mL), and extracted with ethyl acetate (20 mL×2). The organic layers were combined, dried, filtered, and concentrated under reduced pressure. The residue was purified by flash chromatography on silica gel (0-50% gradient of ethyl acetate/petroleum ether) to give 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylic acid (650 mg, 0.72 mmol, yield: 30%). LCMS (ESI): [M+H]⁺=885.3.

Step III: At 0° C., the suspension of 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylic acid (650 mg, 0.72 mmol) and potassium carbonate (329 mg, 2.38 mmol) in N,N-dimethylformamide (7 mL) was prepared. Methyl iodide (224 mg, 1.58 mmol) was added to the suspension, and the reaction mixture was stirred at 20° C. for 16 hours. After completion, the reaction mixture was diluted with water (20 mL) and extracted with ethyl acetate (20 mL×2). The organic phases were combined, dried over magnesium sulfate, filtered, and concentrated under reduced pressure to give a crude product. The crude compound, methyl 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylate (600 mg), was obtained as a yellow oily liquid. LCMS (ESI): [M+H]⁺=899.3.

Step IV: The solution of methyl 4-((1R,5S)-8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)-2-(2,2,2-trifluoroethoxy)quinazolin-6-carboxylate (600 mg, 0.66 mmol) in N,N-dimethylformamide (6 mL) was prepared. Cesium fluoride (1.00 g, 6.60 mmol) was added, and the reaction mixture was stirred at 20° C. for 3 hours. After completion, the reaction mixture was diluted with water (10 mL) and extracted with ethyl acetate (10 mL×3). The organic layers were combined and concentrated under reduced pressure. The residue was purified by flash chromatography on silica gel (0-50% gradient of ethyl acetate/petroleum ether) to give a compound methyl 4-((1R,5S)-8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoro-2-(2,2,2-trifluoroethoxy)quinazolin-6-carboxylate (400 mg, 0.53 mmol, yield: 80%). LCMS (ESI): [M+H]⁺=743.6.

Step V: Methyl 4-((1R,5S)-8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoro-2-(2,2,2-trifluoroethoxy)quinazoline-6-carboxylate (150 mg, 0.2 mmol), benzyl propargyl carbamate (77 mg, 0.4 mmol), and iodine (154 mg, 0.6 mmol) were dissolved in toluene (1.5 mL) and diisopropylamine (1.5 mL). Under a nitrogen atmosphere, copper(I) iodide (4 mg, 0.02 mmol) and dichlorobis(triphenylphosphine)palladium(II) (14 mg, 0.02 mmol) were added, and the reaction mixture was stirred at 20° C. for 16 hours. The mixture was concentrated under reduced pressure, and the residue was purified by flash column chromatography (silica gel, 0-50% gradient of ethyl acetate/petroleum ether) to give a compound methyl 7-(8-(5-(((benzyloxy)carbonyl)amino)pent-1,3-diyne-1-yl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate (100 mg, 0.10 mmol, yield: 53%). LCMS (ESI): [M+H]⁺=930.6.

Step VI: To a solution of methyl 7-(8-(5-(((benzyloxy)carbonyl)amino)pent-1,3-diyne-1-yl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate (100 mg, 0.1 mmol) in ethyl acetate (6 mL) were added dry palladium on carbon (10% Pd, water content <1%, 50 mg) and dry palladium hydroxide on carbon (20% Pd(OH)₂, water content <1%, 50 mg) under a nitrogen atmosphere. The reaction vessel was purged with hydrogen gas three times, and the mixture was stirred at 20° C. under a hydrogen atmosphere (15 psi) for 16 hours. The reaction mixture was filtered through diatomaceous earth, and the filtrate was concentrated under reduced pressure to give a crude compound methyl 7-(8-(5-aminopentyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate (68 mg) as a yellow oily liquid. LCMS (ESI): [M+H]⁺=804.4.

Step VII: To a solution of methyl 7-(8-(5-aminopentyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylate (68 mg, 0.08 mmol) in tetrahydrofuran (1 mL) and water (0.2 mL) was added lithium hydroxide (22 mg, 0.50 mmol) at 0° C. The reaction mixture was stirred at 60° C. for 16 hours. The pH was adjusted to 4 using 1 M dilute hydrochloric acid, and the solution was lyophilized to give a compound 7-(8-(5-aminopentyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (45 mg, 0.05 mmol). LCMS (ESI): [M+H]⁺=790.4.

Step VIII: To a solution of 7-(8-(5-aminopentyl)-7-fluoro-3-(methoxymethoxy)naphthalen-1-yl)-4-((1R,5S)-8-(tert-butoxycarbonyl)-3,8-diazabicyclo[3.2.1]octan-3-yl)-8-fluoro-2-(2,2,2-trifluoroethoxy)quinazoline-6-carboxylic acid (45 mg, 0.05 mmol) and N-methylmorpholine (28 mg, 0.28 mmol) in N-methylpyrrolidone (2 mL) was added 0-(7-azabenzotriazol-1-yl)-N,N,N,N′-tetramethyluronium hexafluorophosphate (65 mg, 0.17 mmol). The reaction mixture was stirred at 80° C. for 16 hours. The mixture was diluted with water (5 mL), extracted with ethyl acetate (5 mL×2), and the combined organic layers were washed with saturated brine (5 mL×2), dried over magnesium sulfate, filtered, and concentrated under reduced pressure to give a crude compound tert-butyl (1R,5S)-3-(3,16-difluoro-18-(methoxymethoxy)-10-oxo-14-(2,2,2-trifluoroethoxy)-5,6,7,8,9,10-hexahydro-4H-naphtho[1′,8′:5,6,7][1]azacyclododecino[3,4-g]quinazolin-12-yl)-3,8-diazabicyclo[3.2.1]octane-8-carboxylate (40 mg), which was obtained as a yellow oily liquid. LCMS (ESI): [M+H]⁺=772.2.

Step IX: To a solution of tert-butyl (1R,5S)-3-(3,16-difluoro-18-(methoxymethoxy)-10-oxo-14-(2,2,2-trifluoroethoxy)-5,6,7,8,9,10-hexahydro-4H-naphtho[1′,8′:5,6,7][1]azacyclododecino[3,4-g]quinazolin-12-yl)-3,8-diazabicyclo[3.2.1]octane-8-carboxylate (40 mg, 0.05 mmol) and ((2R,7aS)-2-fluorotetrahydro-1H-pyrazin-7a(5H)-yl)methanol (18 mg, 0.11 mmol) in tetrahydrofuran (0.4 mL) was added sodium tert-butoxide (10 mg, 0.10 mmol). The reaction mixture was stirred at 20° C. for 1 hour. Water (5 mL) was added to the reaction mixture, and it was extracted with ethyl acetate (5 mL×2). The combined organic layers were washed with saturated brine (5 mL), dried over sodium sulfate, filtered, and concentrated under reduced pressure to give a crude compound tert-butyl (1R,5S)-3-(3,16-difluoro-14-(((2R,7aS)-2-fluorotetrahydro-1H-pyrazin-7a(5H)-yl)methoxy)-18-(methoxymethoxy)-10-oxo-5,6,7,8,9,10-hexahydro-4H-naphtho[1′,8′:5,6,7][1]azacyclododecino[3,4-g]quinazolin-12-yl)-3,8-diazabicyclo[3.2.1]octane-8-carboxylate (25 mg), which was obtained as a yellow oily liquid. LCMS (ESI): [M+H]⁺=831.3.

Step X: To a solution of tert-butyl (1R,5S)-3-(3,16-difluoro-14-(((2R,7aS)-2-fluorotetrahydro-1H-pyrazin-7a(5H)-yl)methoxy)-18-(methoxymethoxy)-10-oxo-5,6,7,8,9,10-hexahydro-4H-naphtho[1′,8′:5,6,7][1]azacyclododecino[3,4-g]quinazolin-12-yl)-3,8-diazabicyclo[3.2.1]octane-8-carboxylate (25 mg, 30 μmol) in acetonitrile (0.1 mL) was added hydrochloric acid (4 M in dioxane, 0.15 mL, 0.6 mmol). The reaction mixture was stirred at 20° C. for 2 hours and then concentrated under reduced pressure. The residue was dissolved in acetonitrile (500 μL), and triethylamine was added to adjust the pH to 8. The residue was purified by preparative HPLC to give a white solid compound 12-((1R,5S)-3,8-diazabicyclo[3.2.1]octan-3-yl)-3,16-difluoro-14-(((2R,7aS)-2-fluorotetrahydro-1H-pyrazin-7a(5H)-yl)methoxy)-18-hydroxy-4,5,6,7,8,9-hexahydro-10H-naphtho[1′,8′:5,6,7][1]azacyclododecino[3,4-g]quinazolin-10-one (2.36 mg, 3 μmol, yield: 11%). LCMS (ESI): [M+H]⁺=687.5; ¹H NMR (400 MHz, CD₃OD) δ ppm 7.72 (s, 1H), 7.61-7.51 (m, 1H), 7.25-7.01 (m, 3H), 5.28-5.11 (m, 1H), 4.29 (br d, J=6.6 Hz, 1H), 4.22-4.07 (m, 2H), 3.68 (br d, J=7.1 Hz, 1H), 3.59-3.44 (m, 3H), 3.16-2.85 (m, 6H), 2.67 (br d, J=13.1 Hz, 1H), 2.47 (br s, 1H), 2.30-1.99 (m, 4H), 1.96-1.64 (m, 8H), 1.27-1.01 (m, 5H).

### Example 15: 10-((1R,5S)-3,8-diazabicyclo[3.2.1]octan-3-yl)-3,14-difluoro-16-hydroxy-12-(2,2,2-trifluoroethoxy)-5,6-dihydro-4H-naphtho[1′,8′:4,5,6]azacycloundecino[2,3-g]quinazolin-7(8H)-one

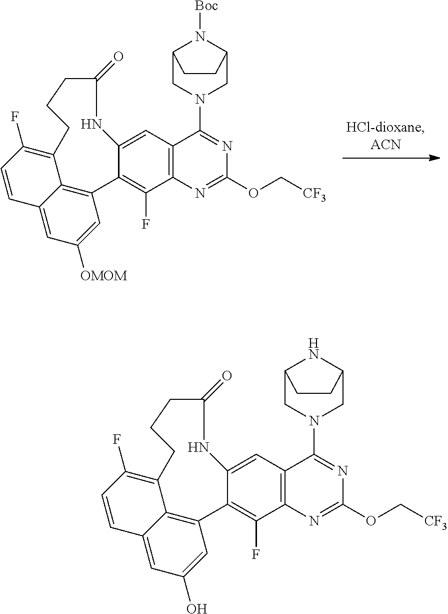

Step I: To a solution of tert-butyl (1R,5S)-3-(3,14-difluoro-16-(methoxymethoxy)-7-oxo-12-(2,2,2-trifluoroethoxy)-5,6,7,8-tetrahydro-4H-naphtho[1′,8′:4,5,6]azacycloundecino[2,3-g]quinazolin-10-yl)-3,8-diazabicyclo[3.2.1]octane-8-carboxylate (50.0 mg, 0.07 mmol) in acetonitrile (1.00 mL) was added hydrochloric acid (4 M in dioxane, 336 μL). The reaction mixture was stirred at 25° C. for 3 hours. The mixture was diluted with acetonitrile (3 mL), and triethylamine was added at 0° C. to adjust the pH to 8. The mixture was concentrated under reduced pressure, and the residue was purified by preparative HPLC to give a yellow solid compound 10-((1R,5S)-3,8-diazabicyclo[3.2.1]octan-3-yl)-3,14-difluoro-16-hydroxy-12-(2,2,2-trifluoroethoxy)-5,6-dihydro-4H-naphtho[1′,8′:4,5,6]azacycloundecino[2,3-g]quinazolin-7(8H)-one (8.72 mg, 14.5 μmol, yield: 21%). LCMS (ESI): [M+H]⁺=600.4; ¹H NMR (400 MHz, CD₃OD) δ ppm 7.86 (s, 1H), 7.71 (dd, J=5.9, 9.0 Hz, 1H), 7.32-7.21 (m, 2H), 6.91 (d, J=2.6 Hz, 1H), 5.03 (q, J=8.8 Hz, 2H), 4.72 (br d, J=13.2 Hz, 1H), 4.48 (br d, J=12.2 Hz, 1H), 3.88 (br s, 1H), 3.84 (br s, 1H), 3.70 (br d, J=13.2 Hz, 1H), 2.99-2.87 (m, 1H), 2.83-2.71 (m, 1H), 2.47-2.34 (m, 1H), 2.21-1.82 (m, 8H).

### Example 16: 3,15-difluoro-13-(((2R,7aS)-2-fluorotetrahydro-1H-pyrrolidin-7a(5H)-yl)methoxy)-11-((R)-3-hydroxy-3-methylpiperidin-1-yl)-4,5,6,7,8,9-hexahydronaphthol[1′,8′:4,5,6][1]azacycloundecane [2,3-g]quinazolin-17-ol

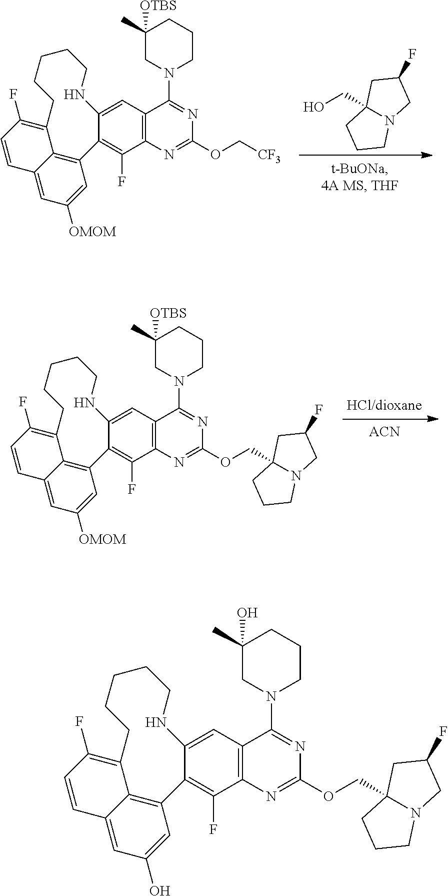

Step I: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7,8,9-hexahydro-naphtho[1′,8′:4,5,6][1]azacycloundecino[2,3-g]quinazoline (60 mg, 0.09 mmol) and ((2R,7aS)-2-fluorotetrahydro-1H-pyrazin-7a(5H)-yl)methanol (59 mg, 0.37 mmol) in tetrahydrofuran (600 μL) were added 4A molecular sieves (60 mg) and sodium tert-butoxide (27 mg, 0.28 mmol). The solution was stirred at 60° C. for 16 hours. The reaction mixture was diluted with water (1 mL) and extracted with ethyl acetate (3×1 mL). The combined organic layers were dried over anhydrous sodium sulfate, filtered, and concentrated under reduced pressure to give a brown solid compound (R)-11-(3-((tert-butyldimethylsilyl)oxy)-3-methylpiperidin-1-yl)-3,15-difluoro-17-(methoxymethoxy)-13-(((2R,7aS)-2-fluorotetrahydro-1H-pyrazin-7a(5H)-yl)methoxy))-4,5,6,7,8,9-hexahydro-naphtho[1′,8′:4,5,6][1]azacycloundecino[2,3-g]quinazoline (100 mg, 85.01 μmol, yield: 92%). LCMS (ESI): [M+H]⁺=706.4

Step II: To a solution of (R)-11-(3-((tert-butyldimethylsilyl)oxy)-3-methylpiperidin-1-yl)-3,15-difluoro-17-(methoxymethoxy)-13-(((2R,7aS)-2-fluorotetrahydro-1H-pyrazin-7a(5H)-yl)methoxy))-4,5,6,7,8,9-hexahydro-naphtho[1′,8′:4,5,6][1]azacycloundecino[2,3-g]quinazoline (100 mg, 0.14 mmol) in acetonitrile (1.0 mL) was added hydrochloric acid (4 M in dioxane, 355 μL, 1.42 mmol). The reaction mixture was stirred at 25° C. for 1 hour. The solution was concentrated under reduced pressure, and dichloromethane (3 mL) was added. The pH was adjusted to 8 using triethylamine. The mixture was concentrated under reduced pressure, and the residue was purified by preparative HPLC to afford the yellow solid compound 3,15-difluoro-13-(((2R,7aS)-2-fluorotetrahydro-1H-pyrazin-7a(5H)-yl)methoxy)-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol (12.54 mg, 18.94 μmol, yield: 13%). LCMS (ESI): [M+H]⁺=662.2; ¹H NMR (400 MHz, CD₃OD) δ=7.69 (dd, J=5.8, 8.9 Hz, 1H), 7.48-7.37 (m, 1H), 7.33-7.22 (m, 2H), 6.94 (dd, J=2.6, 5.4 Hz, 1H), 5.52-5.29 (m, 1H), 4.52-4.35 (m, 2H), 4.26 (br d, J=12.5 Hz, 1H), 4.15 (br dd, J=7.3, 12.9 Hz, 1H), 3.85-3.71 (m, 1H), 3.69-3.41 (m, 4H), 3.24-3.16 (m, 1H), 3.01 (br t, J=13.6 Hz, 1H), 2.66-1.74 (m, 14H), 1.66-1.52 (m, 2H), 1.42 (br d, J=7.0 Hz, 2H), 1.32 (d, J=3.5 Hz, 3H), 1.05 (br s, 1H)

### Example 17A and Example 17B: Two Stereoisomers of (R)-3,15-difluoro-11-(3-hydroxy-3-methylpiperidin-1-yl)-13-(2,2,2-trifluoroethoxy)-4,5,6,7,8,9-hexahydro-naphtho[1′,8′:4,5,6111]azacycloundecino[2,3-g]quinazoline-17-ol

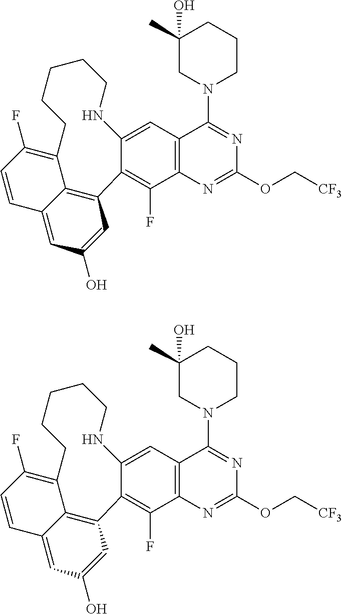

Example 17A: Prepared using a similar synthetic route as described in Example 16. The compound with a shorter retention time in HPLC was obtained as a yellow solid (17.47 mg). LCMS (ESI): [M+H]⁺=603.3; ¹H NMR (400 MHz, CD₃OD) δ ppm 7.66 (dd, J=5.9, 8.8 Hz, 1H), 7.36 (br s, 1H), 7.28 (d, J=2.2 Hz, 1H), 7.22 (t, J=9.5 Hz, 1H), 6.93 (d, J=2.2 Hz, 1H), 4.97-4.89 (m, 2H), 4.24-4.04 (m, 2H), 3.74 (br d, J=15.0 Hz, 1H), 3.44-3.33 (m, 2H), 2.98 (br t, J=13.3 Hz, 1H), 2.69-2.56 (m, 1H), 2.38 (br d, J=7.7 Hz, 1H), 2.18 (br d, J=9.9 Hz, 1H), 1.89-1.72 (m, 4H), 1.68-1.49 (m, 2H), 1.47-1.34 (m, 2H), 1.30 (s, 3H), 1.08-0.94 (m, 1H).

Example 17B: Prepared using a similar synthetic route as described in Example 16. The compound with a longer retention time in HPLC was obtained as a yellow solid (20.64 mg). LCMS (ESI): [M+H]⁺=603.1; ¹H NMR (400 MHz, CD₃OD) δ ppm 7.67 (dd, J=5.8, 8.9 Hz, 1H), 7.45 (s, 1H), 7.29 (d, J=2.4 Hz, 1H), 7.23 (t, J=9.6 Hz, 1H), 6.92 (d, J=2.2 Hz, 1H), 4.98-4.90 (m, 2H), 4.27-4.06 (m, 2H), 3.77 (br d, J=15.4 Hz, 1H), 3.35 (br d, J=13.4 Hz, 2H), 2.98 (br t, J=13.4 Hz, 1H), 2.62 (br s, 1H), 2.37 (br s, 1H), 2.16 (br d, J=10.6 Hz, 1H), 1.91-1.72 (m, 4H), 1.58 (br s, 2H), 1.39 (br s, 2H), 1.29 (s, 3H), 1.01 (br d, J=6.6 Hz, 1H).

### Example 18A and Example 18B: two isomers of (R)-13-(((2-oxabicyclo[2.2.2]octan-1-yl)methoxy)-3,15-difluoro-11-(3-hydroxy-3-methylpiperidin-1-yl)-4,5,6,7,8,9-hexahydro-naphtho[1′,8′:4,5,6][1]azacycloundecino[2,3-g]quinazoline-17-ol

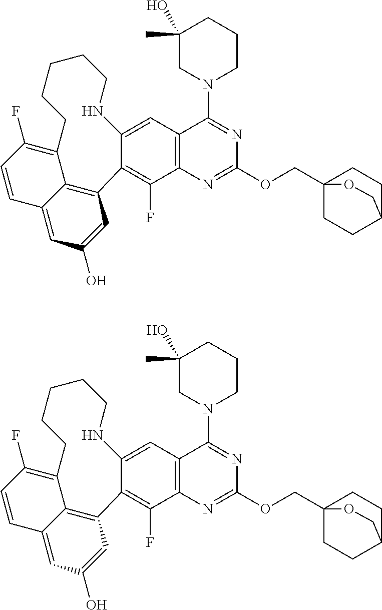

Example 18A: Prepared using a similar synthetic route as described in Example 16: LCMS (ESI): [M+H]⁺=645.3; ¹H NMR (400 MHz, CD₃OD) 5 ppm 7.70-7.63 (m, 1H), 7.35-7.20 (m, 3H), 6.88 (br d, J=4.0 Hz, 1H), 4.24-4.08 (m, 3H), 4.06-3.88 (m, 3H), 3.72 (br d, J=14.8 Hz, 1H), 3.45-3.34 (m, 2H), 2.96 (br t, J=13.0 Hz, 1H), 2.71-2.57 (m, 1H), 2.37 (br s, 1H), 2.16 (br d, J=10.3 Hz, 1H), 2.07-1.95 (m, 2H), 1.91-1.69 (m, 11H), 1.65-1.34 (m, 4H), 1.31 (s, 3H), 1.01 (br d, J=7.9 Hz, 1H).

Example 18B: Prepared using a similar synthetic route as described in Example 16: LCMS (ESI): [M+H]⁺=645.3; ¹H NMR (400 MHz, CD₃OD) 5 ppm 7.73-7.62 (m, 1H), 7.41 (s, 1H), 7.32-7.16 (m, 2H), 6.92 (d, J=2.1 Hz, 1H), 4.24-4.11 (m, 3H), 4.06 (br d, J=13.0 Hz, 1H), 3.97-3.88 (m, 2H), 3.75 (br d, J=14.9 Hz, 1H), 3.35 (br s, 2H), 2.96 (br t, J=13.4 Hz, 1H), 2.64 (br d, J=12.2 Hz, 1H), 2.37 (br s, 1H), 2.14 (br d, J=9.0 Hz, 1H), 2.00 (br s, 2H), 1.91-1.72 (m, 11H), 1.57 (br s, 4H), 1.29 (s, 3H), 0.99 (br s, 1H).

### Example 19: (R)-13-(((2-oxabicyclo[2.2.2]octan-4-yl)methoxy)-3,15-difluoro-11-(3-hydroxy-3-methylpiperidin-1-yl)-4,5,6,7,8,9-hexahydro-naphtho[1′,8′:4,5,6][1]azacycloundecino[2,3-g]quinazoline-17-ol

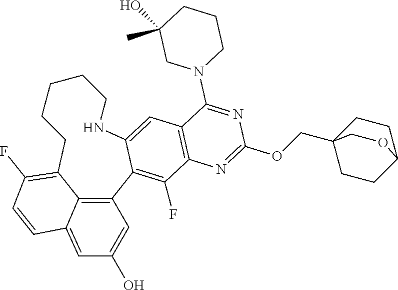 [SMILES: C[C@@]1(O)CCCN(c2nc(OCC34CCC(CC3)OC4)nc3c(F)c4c(cc23)NCCCCCc2c(F)ccc3cc(O)cc-4c23)C1]

Example 19: Prepared using a similar synthetic route as described in Example 16: LCMS (ESI): [M+H]⁺=645.3: ¹H NMR (400 MHz, CD₃OD) δ ppm 7.68 (dd, J=5.9, 8.9 Hz, 1H), 7.47-7.33 (m, 1H), 7.30 (d, J=2.6 Hz, 1H), 7.24 (t, J=9.5 Hz, 1H), 6.99-6.88 (m, 1H), 4.18 (br d, J=12.8 Hz, 1H), 4.13-3.98 (m, 3H), 3.87 (s, 2H), 3.82-3.68 (m, 2H), 3.44-3.37 (m, 1H), 2.98 (br t, J=13.1 Hz, 1H), 2.72-2.57 (m, 1H), 2.38 (br s, 1H), 2.18 (br d, J=9.8 Hz, 1H), 2.10-1.97 (m, 2H), 1.87-1.65 (m, 1H), 1.64-1.52 (m, 2H), 1.41 (br d, J=4.8 Hz, 2H), 1.32 (d, J=5.3 Hz, 3H), 1.01 (br d, J=6.7 Hz, 1H).

### Example 20A and Example 20B: two isomers of (R)-3,15-difluoro-11-(3-hydroxy-3-methylpiperidin-1-yl)-13-(((4-methoxybicyclo[2.2.2]octan-1-yl)methoxy)-4,5,6,7,8,9-hexahydro-naphtho[1′,8′:4,5,6][1]azacycloundecino[2,3-g]quinazoline-17-ol

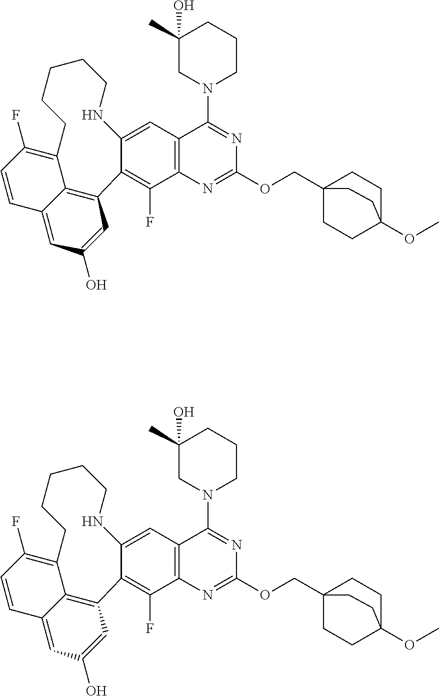

Example 20A: Prepared using a similar synthetic route as described in Example 16: LCMS (ESI): [M+H]⁺=673.5; ¹H NMR (400 MHz, CD₃OD) δ ppm 7.71-7.60 (m, 1H), 7.36-7.31 (m, 1H), 7.31-7.27 (m, 1H), 7.27-7.19 (m, 1H), 6.95-6.86 (m, 1H), 4.19 (br s, 3H), 3.73-3.69 (m, 1H), 3.43-3.36 (m, 1H), 3.17 (d, J=3.7 Hz, 3H), 3.04-2.90 (m, 1H), 2.72-2.56 (m, 1H), 2.45-2.28 (m, 1H), 2.16 (br d, J=12.6 Hz, 2H), 1.82-1.63 (m, 15H), 1.63-1.54 (m, 4H), 1.51-1.35 (m, 2H), 1.31 (s, 3H), 1.12-0.85 (m, 1H).

Example 20B: Prepared using a similar synthetic route as described in Example 16: LCMS (ESI): [M+H]⁺=673.4; ¹H NMR (400 MHz, CD₃OD) δ ppm 7.74-7.64 (m, 1H), 7.47-7.40 (m, 1H), 7.32-7.28 (m, 1H), 7.28-7.21 (m, 1H), 6.94 (d, J=2.6 Hz, 1H), 4.59-4.51 (m, 1H), 4.19-4.06 (m, 3H), 3.85-3.67 (m, 1H), 3.41-3.34 (m, 1H), 3.21-3.17 (m, 3H), 3.08-2.88 (m, 1H), 2.73-2.60 (m, 1H), 2.46-2.32 (m, 1H), 2.21-2.03 (m, 1H), 1.91-1.58 (m, 19H), 1.49-1.34 (m, 2H), 1.34-1.26 (m, 3H), 1.13-0.90 (m, 1H).

### Example 21A and Example 21B: Two Isomers of (R)-3,15-difluoro-11-(3-hydroxy-3-methylpiperidin-1-yl)-13-(((tetrahydro-1H-pyrazin-7a(5H)-yl)methoxy)-4,5,6,7,8,9-hexahydro-naphtho[1′,8′:4,5,6][1]azacycloundecino[2,3-g]quinazoline-17-ol

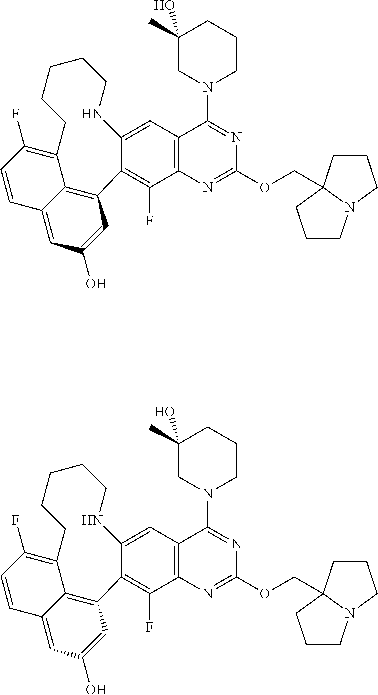

Example 21A: Prepared using a similar synthetic route as described in Example 16: LCMS (ESI): [M+H]⁴=644.3; ¹H NMR (400 MHz, CD₃OD) δ ppm 8.55 (s, 1H), 7.68 (dd, J=5.7, 9.0 Hz, 1H), 7.38 (s, 1H), 7.30 (d, J=2.6 Hz, 1H), 7.24 (t, J=9.5 Hz, 1H), 6.93 (d, J=2.6 Hz, 1H), 4.50 (d, J=4.6 Hz, 2H), 4.27 (td, J=4.7, 9.1 Hz, 1H), 4.15 (br d, J=13.4 Hz, 1H), 3.82-3.70 (m, 1H), 3.53-3.33 (m, 5H), 3.16-2.94 (m, 3H), 2.69-2.55 (m, 1H), 2.45-2.33 (m, 1H), 2.27-2.08 (m, 5H), 2.04-1.93 (m, 4H), 1.88-1.73 (m, 4H), 1.64-1.50 (m, 2H), 1.45-1.35 (m, 2H), 1.31 (s, 3H).

Example 21B: Prepared using a similar synthetic route as described in Example 16: LCMS (ESI): [M+H]⁺=644.2; ¹H NMR (400 MHz, CD₃OD) δ ppm 8.55 (br s, 1H), 7.73-7.65 (m, 1H), 7.46 (s, 1H), 7.30 (d, J=2.6 Hz, 1H), 7.24 (t, J=9.7 Hz, 1H), 6.92 (d, J=2.6 Hz, 1H), 4.50 (s, 2H), 4.34-4.23 (m, 1H), 4.16 (br d, J=13.0 Hz, 1H), 3.77 (br d, J=15.2 Hz, 1H), 3.58-3.33 (m, 5H), 3.11 (br dd, J=5.6, 11.6 Hz, 2H), 3.00 (br t, J=13.0 Hz, 1H), 2.69-2.56 (m, 1H), 2.44-2.31 (m, 1H), 2.30-2.10 (m, 5H), 2.05-1.97 (m, 4H), 1.88-1.74 (m, 4H), 1.64-1.51 (m, 2H), 1.48-1.36 (m, 2H), 1.30 (s, 3H).

### Example 22A and Example 22B: Two Stereoisomers of 3,15-difluoro-11-((R)-3-hydroxy-3-methylpiperidin-1-yl)-9-methyl-13-(((2-methylenetetrahydro-1H-pyrazin-7a(5H)-yl)methoxy)-4,5,6,7,8,9-hexahydro-naphtho[1′,8′:4,5,611]azacycloundecino[2,3-g]quinazoline-17-ol

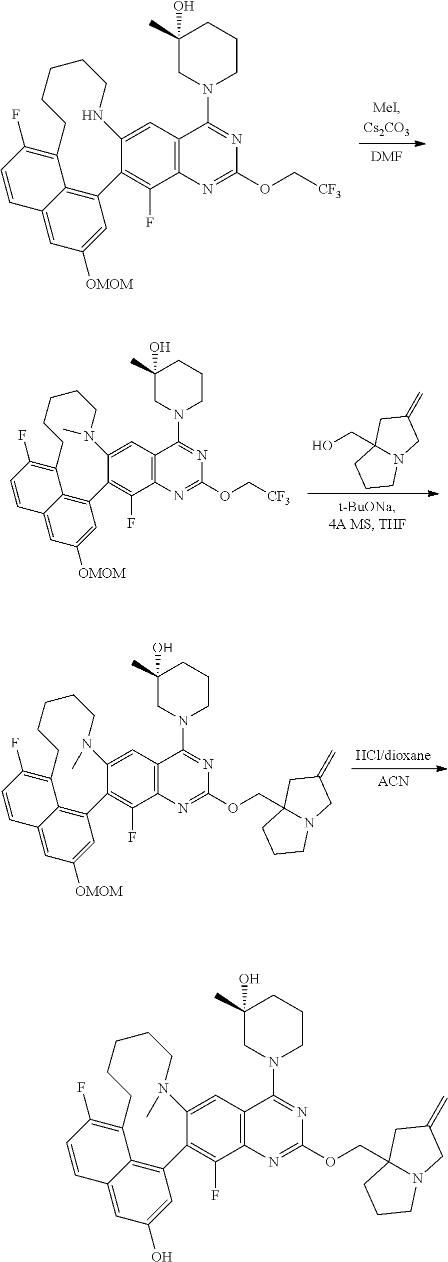

Step I: To the solution of (R)-1-(3,15-difluoro-17-(methoxymethoxy)-13-(2,2,2-trifluoroethoxy)-4,5,6,7,8,9-hexahydronaphtho[1′,8′:4,5,6][1]azacycloundecano[2,3-g]quinazolin-11-yl)-3-methylpiperidin-3-ol (150.00 mg, 0.23 mmol) in N,N-dimethylformamide (1.50 mL) were added cesium carbonate (83.14 mg, 0.26 mmol) and iodomethane (0.03 mL, 0.47 mmol). The mixture was stirred at 25° C. for 16 hours under a nitrogen atmosphere. The mixture was then diluted with water (3 mL) and extracted with ethyl acetate (5 mL×3). The combined organic layers were dried over anhydrous sodium sulfate, filtered, and concentrated under reduced pressure to give a crude compound (R)-1-(3,15-difluoro-17-(methoxymethoxy)-9-methyl-13-(2,2,2-trifluoroethoxy)-4,5,6,7,8,9-hexahydronaphtho[1′,8′:4,5,6][1]azacycloundecano[2,3-g]quinazolin-11-yl)-3-methylpiperidin-3-ol (150 mg). LCMS (ESI): [M+H]⁺=661.2.

Step I: To a solution of (R)-1-(3,15-difluoro-17-(methoxymethoxy)-9-methyl-13-(2,2,2-trifluoroethoxy)-4,5,6,7,8,9-hexahydro-naphtho[1′,8′:4,5,6][1]azacycloundecino[2,3-g]quinazolin-11-yl)-3-methylpiperidin-3-ol (140 mg, 0.21 mmol) and (2-methylenetetrahydro-1H-pyrazin-7a(5H)-yl)methanol (129.87 mg, 0.85 mmol) in tetrahydrofuran (1.50 mL) were added 4A molecular sieves (150 mg) and sodium tert-butoxide (41.6 mg, 0.42 mmol). The solution was stirred at 60° C. for 4 hours. The reaction mixture was diluted with water (2 mL) and extracted with ethyl acetate (1 mL×3). The combined organic layers were dried over anhydrous sodium sulfate, filtered, and concentrated under reduced pressure to give a yellow crude compound (3R)-1-(3,15-difluoro-17-(methoxymethoxy)-9-methyl-13-(((2-methylenetetrahydro-1H-pyrazin-7a(5H)-yl)methoxy)-4,5,6,7,8,9-hexahydro-naphtho[1′,8′:4,5,6][1]azacycloundecino[2,3-g]quinazolin-11-yl)-3-methylpiperidin-3-ol (150 mg). LCMS (ESI): [M+H]⁺=714.4

Step III: At 25° C., hydrogen chloride (4M in dioxane, 1.05 mL, 4.20 mmol) was added to a solution of (3R)-1-(3,15-difluoro-17-(methoxymethoxy)-9-methyl-13-(((2-methylenetetrahydro-1H-pyrazin-7a(5H)-yl)methoxy)-4,5,6,7,8,9-hexahydro-naphtho[1′,8′:4,5,6][1]azacycloundecino[2,3-g]quinazolin-11-yl)-3-methylpiperidin-3-ol (150 mg, 0.21 mmol) in acetonitrile (1.5 mL). The reaction mixture was stirred at 25° C. for 1 hour. The solution was concentrated, acetonitrile (3 mL) was added and the pH was adjusted to 8 with triethylamine. The mixture was concentrated under reduced pressure, and the residue was purified by preparative HPLC to give two stereoisomers of 3,15-difluoro-11-((R)-3-hydroxy-3-methylpiperidin-1-yl)-9-methyl-13-(((2-methylenetetrahydro-1H-pyrazin-7a(5H)-yl)methoxy)-4,5,6,7,8,9-hexahydro-naphtho[1′,8′:4,5,6][1]azacycloundecino[2,3-g]quinazoline-17-ol.

Example 22A: LCMS (ESI): [M+H]⁺=670.3; ¹H NMR (400 MHz, CD₃OD) δ ppm 7.83-7.72 (m, 1H), 7.63 (dd, J=6.0, 9.0 Hz, 1H), 7.28-7.15 (m, 2H), 6.88 (d, J=2.5 Hz, 1H), 5.00 (br s, 2H), 4.31-4.25 (m, 2H), 4.13 (br d, J=13.1 Hz, 1H), 3.79 (br d, J=14.4 Hz, 1H), 3.44-3.33 (m, 3H), 3.29-3.17 (m, 2H), 2.93-2.79 (m, 2H), 2.78-2.75 (m, 3H), 2.64-2.44 (m, 3H), 2.25-2.10 (m, 2H), 2.09-1.58 (m, 8H), 1.55 (br s, 1H), 1.43-1.19 (m, 8H).

Example 22B: LCMS (ESI): [M+H]⁺=670.4; ¹H NMR (400 MHz, CD₃OD) δ ppm 7.80 (s, 1H), 7.63 (dd, J=5.9, 9.0 Hz, 1H), 7.27-7.13 (m, 2H), 6.88 (d, J=2.5 Hz, 1H), 5.00 (br s, 2H), 4.28 (br d, J=7.0 Hz, 2H), 4.09 (br d, J=13.1 Hz, 1H), 3.79 (br d, J=14.3 Hz, 1H), 3.47-3.32 (m, 3H), 3.28-3.10 (m, 2H), 2.89-2.78 (m, 2H), 2.75 (s, 3H), 2.63-2.45 (m, 3H), 2.23-2.09 (m, 2H), 2.07-1.61 (m, 8H), 1.59-1.50 (m, 1H), 1.44-1.21 (m, 8H).

### Example 23: 13-(((dihydro-1′H,3′H-spiro[cyclopropane-1,2′-pyrrolo[2,1-c][1,4]oxazepin]-7a′(5′H)-yl)methoxy)-3,15-difluoro-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol

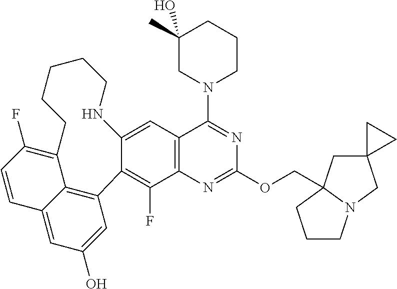 [SMILES: C[C@@]1(O)CCCN(c2nc(OCC34CCCN3CC3(CC3)C4)nc3c(F)c4c(cc23)NCCCCCc2c(F)ccc3cc(O)cc-4c23)C1]

Example 23: Prepared using a similar synthetic route as described in Example 16: LCMS (ESI): [M+H]⁺=670.3; ¹H NMR (400 MHz, CD₃OD) δ ppm 8.56 (br s, 1H), 7.69 (br dd, J=5.8, 8.9 Hz, 1H), 7.51-7.36 (m, 1H), 7.31 (br d, J=2.3 Hz, 1H), 7.26 (br t, J=9.5 Hz, 1H), 6.95 (br s, 1H), 4.61 (br s, 2H), 4.35-4.14 (m, 2H), 3.77 (br d, J=14.5 Hz, 1H), 3.55 (br s, 1H), 3.48-3.25 (m, 3H), 3.28 (br s, 1H), 3.18-2.94 (m, 2H), 2.64 (br s, 1H), 2.47-2.02 (m, 8H), 1.94-1.70 (m, 4H), 1.59 (br d, J=8.2 Hz, 2H), 1.42 (br s, 2H), 1.32 (br d, J=2.7 Hz, 3H), 1.07 (br s, 1H), 0.89-0.54 (m, 4H).

### Example 24A and Example 24B: Two Isomers of 13-(((1s,7a′s)-2,2-difluorodihydro-1′H,3′H-spiro[cyclopropane-1,2′-pyrazin-7a′(5′H)-yl)methoxy)-3,15-difluoro-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol

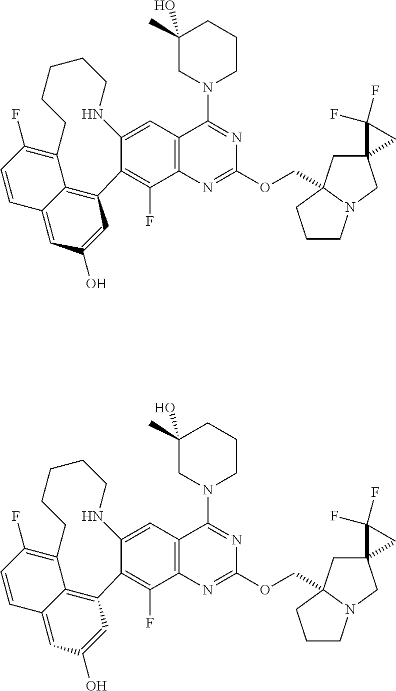

Example 24A: Prepared using a similar synthetic route as described in Example 16: LCMS (ESI): [M+H]⁺=706.5; ¹H NMR (400 MHz, CD₃OD) δ ppm 7.67 (dd, J=5.9, 8.9 Hz, 1H), 7.33 (s, 1H), 7.30-7.27 (m, 1H), 7.23 (t, J=9.6 Hz, 1H), 6.93 (d, J=2.5 Hz, 1H), 4.74-4.50 (m, 1H), 4.41-4.24 (m, 2H), 4.18 (br d, J=12.5 Hz, 1H), 4.10-3.98 (m, 1H), 3.72 (br d, J=14.6 Hz, 1H), 3.49-3.32 (m, 3H), 3.25-3.15 (m, 1H), 3.02-2.83 (m, 2H), 2.78-2.57 (m, 2H), 2.38 (br d, J=6.5 Hz, 1H), 2.29 (br dd, J=6.1, 13.6 Hz, 1H), 2.22-2.11 (m, 2H), 2.03 (s, 1H), 1.97-1.91 (m, 1H), 1.87-1.73 (m, 5H), 1.64-1.50 (m, 2H), 1.49-1.33 (m, 4H), 1.32-1.27 (m, 3H), 1.00 (br d, J=7.3 Hz, 1H).

Example 24B: Prepared using a similar synthetic route as described in Example 16: LCMS (ESI): [M+H]⁺=706.8; ¹H NMR (400 MHz, CD₃OD) δ ppm 7.69 (dd, J=5.8, 9.1 Hz, 1H), 7.43 (s, 1H), 7.31 (d, J=2.6 Hz, 1H), 7.25 (t, J=9.6 Hz, 1H), 6.94 (d, J=2.6 Hz, 1H), 4.63 (br s, 1H), 4.41-4.28 (m, 2H), 4.20 (br d, J=12.5 Hz, 1H), 4.10 (br d, J=13.3 Hz, 1H), 3.77 (br d, J=14.9 Hz, 1H), 3.41-3.34 (m, 2H), 3.30 (br s, 1H), 3.24-3.14 (m, 1H), 2.99 (br t, J=13.2 Hz, 1H), 2.89 (br d, J=12.0 Hz, 1H), 2.78-2.59 (m, 2H), 2.44-2.25 (m, 2H), 2.23-2.10 (m, 2H), 2.04 (br d, J=13.5 Hz, 1H), 1.98-1.92 (m, 1H), 1.89-1.75 (m, 5H), 1.60 (br d, J=8.4 Hz, 2H), 1.52-1.36 (m, 4H), 1.31 (s, 3H), 1.02 (br s, 1H).

### Example 25: 3,15-Difluoro-11-((R)-3-hydroxy-3-methylpiperidin-1-yl)-13-(((2-methylenetetrahydro-1H-pyrazin-7a(5H)-yl)methoxy)-4,5,6,7,8,9-hexahydro-naphtho[1′,8′:4,5,6][1]azacycloundecino[2,3-g]quinazoline-17-ol

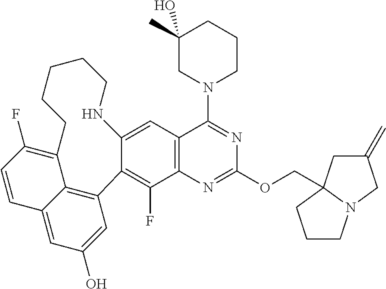 [SMILES: C=C1CN2CCCC2(COc2nc(N3CCC[C@@](C)(O)C3)c3cc4c(c(F)c3n2)-c2cc(O)cc3ccc(F)c(c23)CCCCCN4)C1]

Example 25: Prepared using a similar synthetic route as described in Example 16: LCMS (ESI): [M+H]⁺=656.1; ¹H NMR (400 MHz, CD₃OD) 5 ppm 8.56 (br s, 1H), 7.70 (dd, J=5.9, 8.8 Hz, 1H), 7.49-7.30 (m, 2H), 7.26 (t, J=9.5 Hz, 1H), 6.95 (br s, 1H), 5.19 (br d, J=6.2 Hz, 2H), 4.70-4.47 (m, 2H), 4.29 (br d, J=13.0 Hz, 1H), 4.16 (br d, J=13.4 Hz, 2H), 3.83-3.68 (m, 2H), 3.56 (br s, 1H), 3.46-3.34 (m, 2H), 3.16-2.91 (m, 3H), 2.78-2.59 (m, 2H), 2.48-2.26 (m, 2H), 2.25-1.98 (m, 4H), 1.91-1.71 (m, 4H), 1.67-1.38 (m, 4H), 1.32 (d, J=3.1 Hz, 3H), 1.06 (br s, 1H).

### Example 26: (R)-3,15-Difluoro-11-(3-hydroxy-3-methylpiperidin-1-yl)-13-(((1-(morpholinomethyl)cyclopropyl)methoxy)-4,5,6,7,8,9-hexahydro-naphtho[1′,8′:4,5,6][1]azacycloundecino[2,3-g]quinazoline-17-ol

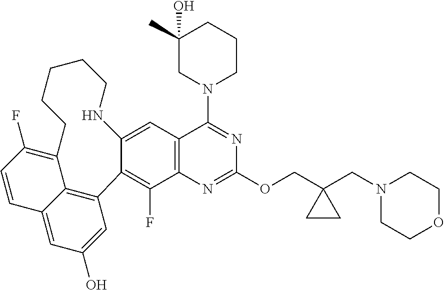 [SMILES: C[C@@]1(O)CCCN(c2nc(OCC3(CN4CCOCC4)CC3)nc3c(F)c4c(cc23)NCCCCCc2c(F)ccc3cc(O)cc-4c23)C1]

Example 26: Prepared using a similar synthetic route as described in Example 16: LCMS (ESI): [M+H]⁺=674.3; ¹H NMR (400 MHz, CDOD) δ ppm 7.69 (dd, J=8.9, 5.9 Hz, 1H), 7.43 (s, 1H), 7.33-7.21 (m, 2H), 6.97-6.92 (m, 1H), 4.44-4.34 (m, 2H), 4.22-4.00 (m, 2H), 3.79-3.64 (m, 5H), 3.46-3.35 (m, 2H), 2.98 (br t, J=13.0 Hz, 1H), 2.80-2.57 (m, 7H), 2.38 (br s, 1H), 2.17 (br s, 1H), 1.91-1.74 (m, 4H), 1.72-1.24 (m, 8H), 1.00 (br s, 1H), 0.82-0.70 (m, 2H), 0.56 (s, 2H).

### Example 27: (R)-13-(((1-((dimethylamino)methyl)cyclopropyl)methoxy)-3,15-difluoro-11-(3-hydroxy-3-methylpiperidin-1-yl)-4,5,6,7,8,9-hexahydro-naphtho[1′,8′:4,5,6][1]azacycloundecino[2,3-g]quinazoline-17-ol

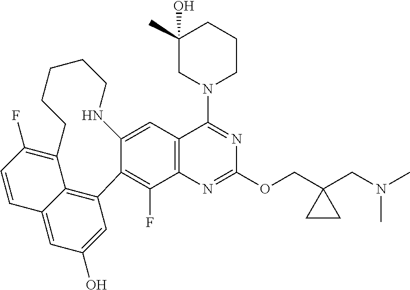 [SMILES: CN(C)CC1(COc2nc(N3CCC[C@@](C)(O)C3)c3cc4c(c(F)c3n2)-c2cc(O)cc3ccc(F)c(c23)CCCCCN4)CC1]

Example 27: Prepared using a similar synthetic route as described in Example 16: LCMS (ESI): [M+H]⁺=632.3; ¹H NMR (400 MHz, CD₃OD) δ ppm 8.56 (br s, 1H), 7.70 (dd, J=5.9, 9.0 Hz, 1H), 7.50-7.38 (m, 1H), 7.32 (d, J=2.6 Hz, 1H), 7.26 (t, J=9.6 Hz, 1H), 6.98-6.88 (m, 1H), 4.64 (br s, 2H), 4.43-4.29 (m, 2H), 4.25 (br d, J=13.2 Hz, 1H), 4.18-4.05 (m, 1H), 3.75 (br d, J=14.7 Hz, 1H), 3.44-3.37 (m, 1H), 3.00 (br s, 2H), 2.72 (br s, 6H), 2.38 (br s, 1H), 2.19 (br s, 1H), 1.92-1.74 (m, 4H), 1.60 (br s, 2H), 1.50-1.34 (m, 3H), 1.32 (d, J=4.2 Hz, 3H), 1.02 (br s, 1H), 0.88 (s, 2H), 0.73 (br s, 2H).

### Examples 28A and 28B: 3,15-Difluoro-11-((R)-3-hydroxy-3-methylpiperidin-1-yl)-13-(((S)-1-methylpyrrolidin-2-yl)methoxy)-4,5,6,7,8,9-hexahydro-naphtho[1′,8′:4,5,6][1]azacycloundecino[2,3-g]quinazoline-17-ol

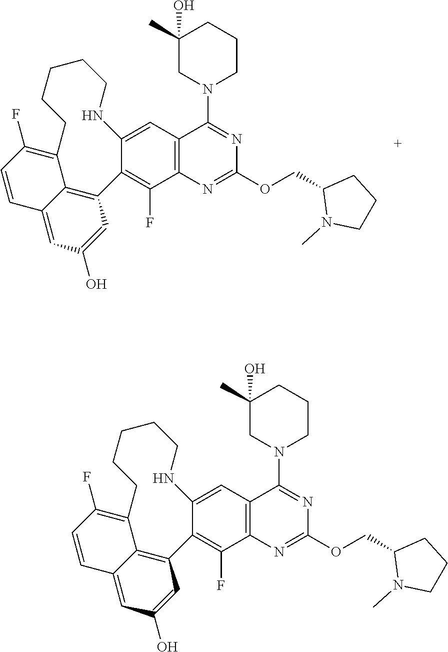

Example 28A: Prepared using a similar synthetic route as described in Example 16. The compound corresponds to the peak with a shorter retention time on HPLC, yielding 0.94 mg as a yellow solid LCMS (ESI): [M+H]⁺=618.2; ¹H NMR (400 MHz, CD₃OD) δ ppm 7.67 (dd, J=8.9, 5.7 Hz, 1H), 7.39-7.17 (m, 3H), 6.93 (d, J=2.4 Hz, 1H), 4.65-4.44 (m, 3H), 4.27-4.01 (m, 2H), 3.73 (br d, J=15.2 Hz, 1H), 3.41 (br d, J=13.0 Hz, 1H), 2.98 (br t, J=13.3 Hz, 1H), 2.74-2.59 (m, 5H), 2.46-2.08 (m, 3H), 2.04-1.70 (m, 8H), 1.65-1.24 (m, 8H), 1.02 (br s, 1H).

Example 28B: Prepared using a similar synthetic route as described in Example 16. The compound corresponds to the peak with a longer retention time on HPLC, yielding 1.04 mg as a yellow solid LCMS (ESI): [M+H]⁺=618.2; ¹H NMR (400 MHz, CD₃OD) δ ppm 7.74-7.61 (m, 1H), 7.48-7.37 (m, 1H), 7.33-7.18 (m, 2H), 6.92 (br s, 1H), 4.65-4.38 (m, 3H), 4.35-4.04 (m, 2H), 3.82-3.65 (m, 1H), 3.42-3.38 (m, 2H), 3.05-2.90 (m, 1H), 2.78 (s, 3H), 2.61 (br s, 2H), 2.43-2.06 (m, 3H), 2.05-1.69 (m, 7H), 1.67-1.16 (m, 8H), 1.02 (br s, 1H).

### Examples 29 and 30: 13-(((2-(difluoromethylene)tetrahydro-1H-pyrazin-7a(5H)-yl)methoxy)-3,15-difluoro-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol and 3,15-difluoro-13-(((2-(fluoromethylene)tetrahydro-1H-pyrazin-7a(5H)-yl)methoxy)-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol

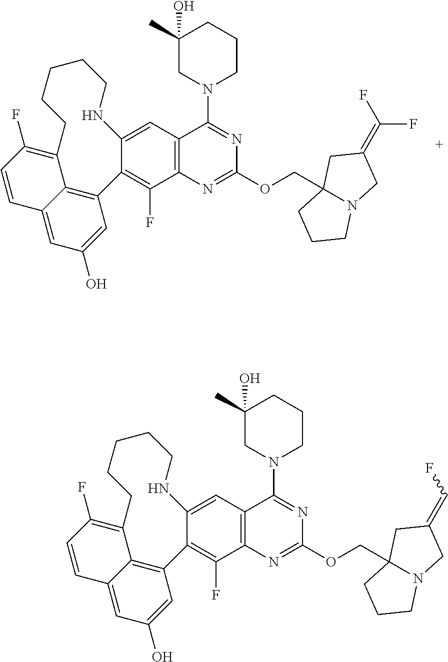 [SMILES: C[C@@]1(O)CCCN(c2nc(OCC34CCCN3CC(=C(F)F)C4)nc3c(F)c4c(cc23)NCCCCCc2c(F)ccc3cc(O)cc-4c23)C1 ; C[C@@]1(O)CCCN(c2nc(OCC34CCCN3CC(=CF)C4)nc3c(F)c4c(cc23)NCCCCCc2c(F)ccc3cc(O)cc-4c23)C1]

Example 29: Prepared using a similar synthetic route as described in Example 16 to obtain (1.5 mg, 2.17 μmol, yield: 1%) as a yellow solid. LCMS (ESI): [M+H]⁺=692.4; ¹H NMR (400 MHz, CD₃OD) δ ppm 7.69 (br dd, J=5.8, 9.0 Hz, 1H), 7.45 (s, 1H), 7.34-7.20 (m, 2H), 6.94 (d, J=2.5 Hz, 1H), 4.50-4.29 (m, 2H), 4.26-4.09 (m, 2H), 4.02-3.59 (m, 4H), 3.38 (br s, 1H), 3.07-2.84 (m, 3H), 2.71-2.56 (m, 2H), 2.39 (br s, 1H), 2.26-1.94 (m, 5H), 1.91-1.73 (m, 5H), 1.60 (br s, 2H), 1.41 (br s, 2H), 1.31 (s, 3H), 1.02 (br s, 1H).

Example 30: Prepared using a similar synthetic route as described in Example 16 to obtain (8.0 mg, 11.89 μmol, yield: 7%) as a yellow solid. LCMS (ESI): [M+H]⁺=674.4; ¹H NMR (400 MHz, CD₃OD) δ ppm 7.74-7.65 (m, 1H), 7.47-7.18 (m, 3H), 6.95 (br s, 1H), 6.93-6.53 (m, 1H), 4.53-4.00 (m, 4H), 3.96-3.53 (m, 2H), 3.48-3.36 (m, 2H), 3.32-3.17 (m, 1H), 3.05-2.52 (m, 5H), 2.45-1.91 (m, 6H), 1.90-1.72 (m, 4H), 1.64-1.36 (m, 5H), 1.32 (br d, 1=3.7 Hz, 3H), 1.03 (br s, 1H).

### Example 31: (R)-13-(((2,6-dimethylene-tetrahydro-1H-pyrazin-7a(5H)-yl)methoxy)-3,15-difluoro-11-(3-hydroxy-3-methylpiperidin-1-yl)-4,5,6,7,8,9-hexahydro-naphtho[1′,8′:4,5,6][1]azacycloundecino[2,3-g]quinazoline-17-ol

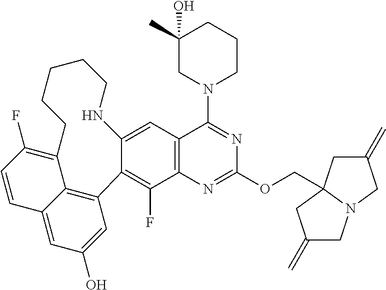 [SMILES: C=C1CN2CC(=C)CC2(COc2nc(N3CCC[C@@](C)(O)C3)c3cc4c(c(F)c3n2)-c2cc(O)cc3ccc(F)c(c23)CCCCCN4)C1]

Example 31: Prepared using a similar synthetic route as described in Example 16: LCMS (ESI): [M+H]⁺=668.3; ¹H NMR (400 MHz, CD₃OD) δ ppm 7.59 (dd, J=5.9, 9.1 Hz, 2H), 7.22 (d, J=2.6 Hz, 1H), 7.18-7.12 (m, 1H), 6.84 (d, J=2.6 Hz, 1H), 5.22 (br s, 4H), 4.61 (br d, J=13.8 Hz, 1H), 4.37 (br d, J=14.8 Hz, 2H), 3.86 (br d, J=15.5 Hz, 2H), 3.69 (br d, J=14.9 Hz, 1H), 3.52 (br d, J=13.5 Hz, 1H), 3.08-2.95 (m, 3H), 2.84-2.73 (m, 2H), 2.61-2.51 (m, 1H), 2.27-2.05 (m, 3H), 1.85-1.64 (m, 4H), 1.54-1.34 (m, 3H), 1.32-1.03 (m, 8H).

### Example 32: (Z)-3,15-difluoro-11-((R)-3-hydroxy-3-methylpiperidin-1-yl)-13-((2-methylenetetrahydro-1H-pyrazin-7a(5H)-yl)methoxy)-4,5,8,9-tetrahydronaphtho[1′,8′:4,5,6][1]azacycloondecano[2,3-g]quinazolin-17-ol

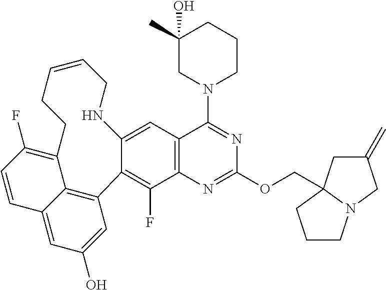 [SMILES: C=C1CN2CCCC2(COc2nc(N3CCC[C@@](C)(O)C3)c3cc4c(c(F)c3n2)-c2cc(O)cc3ccc(F)c(c23)CC/C=C\CN4)C1]

Example 32: Prepared using a similar synthetic route as described in Example 16: LCMS (ESI): [M+H]⁺=654.5; ¹H NMR (400 MHz, CD₃OD) δ ppm 7.81 (dd, J=5.7, 9.0 Hz, 1H), 7.38-7.27 (m, 2H), 7.09-6.98 (m, 1H), 6.88 (br d, J=8.7 Hz, 1H), 5.69-5.59 (m, 1H), 5.14 (br s, 3H), 4.78-4.37 (m, 2H), 4.32-3.95 (m, 3H), 3.69-3.52 (m, 2H), 3.51-3.36 (m, 2H), 3.05-2.53 (m, 4H), 2.40-1.69 (m, 12H), 1.61 (br s, 11H), 1.32 (d, J=3.8 Hz, 31H).

### Examples 33A and 33B: Two isomers of (R)-3,15-difluoro-11-(3-hydroxy-3-methylpiperidin-1-yl)-13-((1-morpholinocyclopropyl)methoxy)-4,5,6,7,8,9-hexahydro-naphtho[1′,8′:4,5,6][1]azacycloundecino[2,3-g]quinazoline-17-ol

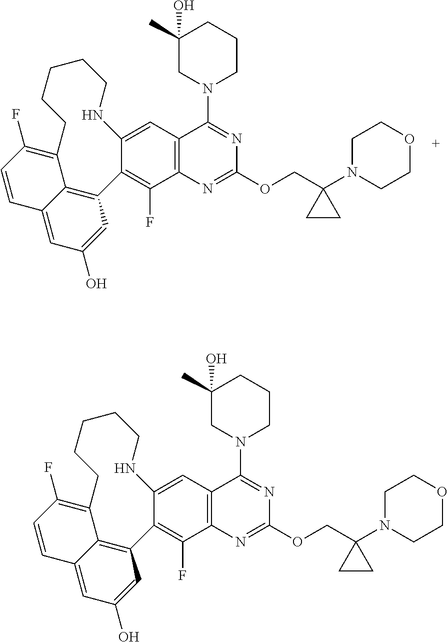

Example 33A: Prepared using a similar synthetic route as described in Example 16: LCMS (ESI): [M+H]⁺=660.3; ¹H NMR (400 MHz, CD₃OD) 5 ppm 7.67 (dd, J=6.2, 8.6 Hz, 1H), 7.41-7.09 (m, 3H), 6.93 (s, 1H), 4.51 (br d, J=3.0 Hz, 3H), 4.17-4.10 (m, 1H), 4.02 (br d, J=12.5 Hz, 1H), 3.72 (br d, J=13.9 Hz, 1H), 3.59 (br d, J=3.9 Hz, 4H), 3.40 (br d, J=13.4 Hz, 2H), 2.88 (br d, J=4.3 Hz, 4H), 2.67-2.62 (m, 1H), 2.43-2.33 (m, 1H), 2.23-2.13 (m, 1H), 1.87-1.68 (m, 4H), 1.64-1.50 (m, 2H), 1.45-1.35 (m, 2H), 1.31 (s, 3H), 1.05-0.95 (m, 1H), 0.75 (br d, J=9.9 Hz, 4H).

Example 33B: Prepared using a similar synthetic route as described in Example 16: LCMS (ESI): [M+H]⁺=660.2; ¹H NMR (400 MHz, CD₃OD) 5 ppm 7.67 (dd, J=5.7, 8.9 Hz, 1H), 7.42 (s, 1H), 7.31-7.19 (m, 2H), 6.92 (d, J=2.1 Hz, 1H), 4.51 (d, J=6.8 Hz, 3H), 4.18-4.04 (m, 2H), 3.75 (br d, J=14.0 Hz, 1H), 3.63-3.55 (m, 4H), 3.36 (br s, 2H), 2.91-2.83 (m, 4H), 2.68-2.62 (m, 1H), 2.42-2.30 (m, 1H), 2.20-2.10 (m, 1H), 1.88-1.71 (m, 4H), 1.64-1.53 (m, 2H), 1.39 (br d, J=6.9 Hz, 2H), 1.30 (s, 3H), 1.04-0.94 (m, 1H), 0.76 (br d, J=10.0 Hz, 4H).

### Example 34: 13-(((S)-4,4-difluoro-1-methylpyrrolidin-2-yl)methoxy)-3,15-difluoro-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol

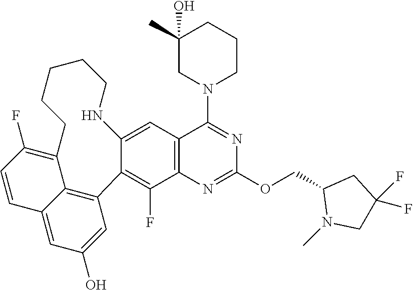 [SMILES: CN1CC(F)(F)C[C@H]1COc1nc(N2CCC[C@@](C)(O)C2)c2cc3c(c(F)c2n1)-c1cc(O)cc2ccc(F)c(c12)CCCCCN3]

Example 34: Prepared using a similar synthetic route as described in Example 16: ¹H NMR (400 MHz, CD₃OD) δ ppm 7.69 (dd, J=6.1, 8.8 Hz, 1H), 7.49-7.17 (m, 3H), 6.94 (br s, 1H), 4.63 (br s, 2H), 4.57-4.44 (m, 2H), 4.23-4.03 (m, 2H), 3.84-3.70 (m, 1H), 3.47-3.36 (m, 2H), 3.12-2.94 (m, 2H), 2.82-2.62 (m, 2H), 2.50 (s, 3H), 2.44-2.15 (m, 3H), 1.94-1.71 (m, 4H), 1.60 (br s, 2H), 1.40 (br s, 2H), 1.32 (d, J=4.4 Hz, 3H), 1.02 (br s, 1H).

### Example 35: 3,15-Difluoro-11-((R)-3-hydroxy-3-methylpiperidin-1-yl)-13-((S)-1-((S)-1-methylpyrrolidin-2-yl)ethoxy)-4,5,6,7,8,9-hexahydro-naphtho[1′,8′:4,5,6][1]azacycloundecino[2,3-g]quinazoline-17-ol

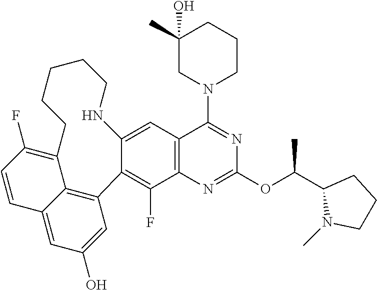 [SMILES: C[C@H](Oc1nc(N2CCC[C@@](C)(O)C2)c2cc3c(c(F)c2n1)-c1cc(O)cc2ccc(F)c(c12)CCCCCN3)[C@@H]1CCCN1C]

Example 35: Prepared using a similar synthetic route as described in Example 16: LCMS (ESI): [M+H]⁺=632.4; ¹H NMR (400 MHz, CD₃OD) δ ppm 8.56 (br s, 1H), 7.69 (dd, J=8.9, 5.9 Hz, 1H), 7.45-7.36 (m, 1H), 7.31 (d, J=2.6 Hz, 1H), 7.25 (t, J=9.5 Hz, 1H), 6.99-6.87 (m, 1H), 5.35-5.27 (m, 1H), 4.29-4.02 (m, 3H), 3.82-3.72 (m, 1H), 3.48-3.37 (m, 3H), 3.07-2.96 (m, 1H), 2.92-2.78 (m, 5H), 2.72-2.60 (m, 1H), 2.44-2.11 (m, 4H), 2.07-1.96 (m, 2H), 1.91-1.74 (m, 6H), 1.59 (br d, J=6.5 Hz, 2H), 1.45 (br d, J=6.1 Hz, 3H), 1.32 (d, J=3.4 Hz, 3H).

### Example 36: 13-(2,2-difluorotetrahydro-1H-pyrazin-7a(5H)-yl)methoxy)-3,15-difluoro-11-((R)-3-hydroxy-3-methylpiperidin-1-yl)-4,5,6,7,8,9-hexahydronaphtho[1′,8′:4,5,6][1]azacycloundecano[2,3-g]quinazolin-17-ol

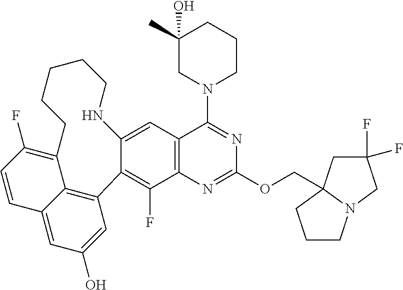 [SMILES: C[C@@]1(O)CCCN(c2nc(OCC34CCCN3CC(F)(F)C4)nc3c(F)c4c(cc23)NCCCCCc2c(F)ccc3cc(O)cc-4c23)C1]

Example 36: Prepared using a similar synthetic route as described in Example 16: LCMS (ESI): [M+H]⁺=680.3; ¹H NMR (400 MHz, CD₃OD) δ ppm 7.69 (dd, J=8.7, 5.9 Hz, 1H), 7.44-7.34 (m, 1H), 7.32-7.21 (m, 2H), 6.95 (br s, 1H), 4.29-4.16 (m, 3H), 4.13-4.03 (m, 1H), 3.81-3.71 (m, 1H), 3.54-3.37 (m, 3H), 3.22-3.09 (m, 2H), 3.04-2.84 (m, 2H), 2.72-2.59 (m, 2H), 2.46-1.70 (m, 12H), 1.59 (br s, 2H), 1.40 (br d, J=5.5 Hz, 2H), 1.32 (br d, J=4.6 Hz, 3H).

### Example 37: 3,15-Difluoro-13-((hexahydrocyclopropano[b]pyrrolizin-5a(3H)-yl)methoxy)-11-((R)-3-hydroxy-3-methylpiperidin-1-yl)-4,5,6,7,8,9-hexahydro-naphtho[1′,8′:4,5,6][1]azacycloundecino[2,3-g]quinazoline-17-ol

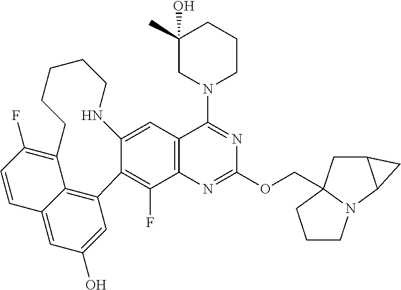 [SMILES: C[C@@]1(O)CCCN(c2nc(OCC34CCCN3C3CC3C4)nc3c(F)c4c(cc23)NCCCCCc2c(F)ccc3cc(O)cc-4c23)C1]

Example 37: Prepared using a similar synthetic route as described in Example 16: LCMS (ESI): [M+H]⁺=656.1; ¹H NMR (400 MHz, CD₃OD) δ ppm 7.67 (dd, J=5.9, 8.9 Hz, 1H), 7.45-7.31 (m, 1H), 7.30-7.18 (m, 2H), 7.02-6.84 (m, 1H), 4.32-3.94 (m, 5H), 3.77-3.69 (m, 1H), 3.44-3.34 (m, 2H), 3.17 (br s, 4H), 2.67-2.58 (m, 1H), 2.42-2.34 (m, 1H), 2.26-2.10 (m, 4H), 1.98 (br d, J=4.4 Hz, 3H), 1.81-1.69 (m, 4H), 1.62-1.51 (m, 2H), 1.44-1.34 (m, 2H), 1.29 (d, J=4.0 Hz, 3H), 1.07-0.92 (m, 1H), 0.88-0.55 (m, 2H).

### Example 38: 13-((2,2-difluoro-6-methylenetetrahydro-1H-pyrazin-7a(5H)-yl)methoxy)-3,15-difluoro-11-((R)-3-hydroxy-3-methylpiperidin-1-yl)-4,5,6,7,8,9-hexahydronaphtho[1′,8′:4,5,6][1]azacycloundecano[2,3-g]quinazolin-17-ol

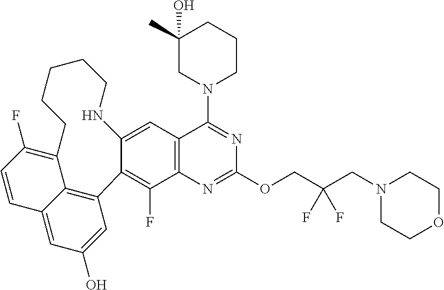 [SMILES: C[C@@]1(O)CCCN(c2nc(OCC(F)(F)CN3CCOCC3)nc3c(F)c4c(cc23)NCCCCCc2c(F)ccc3cc(O)cc-4c23)C1]

Example 38: Prepared using a similar synthetic route as described in Example 16: LCMS (ESI): [M+H]⁺=692.7; ¹H NMR (400 MHz, CD₃OD) δ ppm 7.61-7.53 (m, 1H), 7.28-7.07 (m, 3H), 6.83 (d, J=2.6 Hz, 1H), 4.92 (br d, J=3.8 Hz, 2H), 4.23-4.06 (m, 3H), 3.97 (br d, J=13.1 Hz, 1H), 3.65 (br t, J=14.6 Hz, 2H), 3.41-3.32 (m, 2H), 3.30-3.24 (m, 1H), 3.10-2.98 (m, 1H), 2.93-2.81 (m, 1H), 2.75-2.67 (m, 1H), 2.60-2.46 (m, 3H), 2.31 (q, J=14.4 Hz, 2H), 2.14-2.00 (m, 1H), 1.80-1.60 (m, 4H), 1.55-1.38 (m, 2H), 1.35-1.16 (m, 6H), 0.95-0.84 (m, 1H).

### Example 39: (R)-13-(3-(dimethylamino)azetidinyl)-3,15-difluoro-11-(3-hydroxy-3-methylpiperidin-1-yl)-4,5,6,7,8,9-hexahydro-naphtho[1′,8′:4,5,6][1]azacycloundecino[2,3-g]quinazoline-17-ol

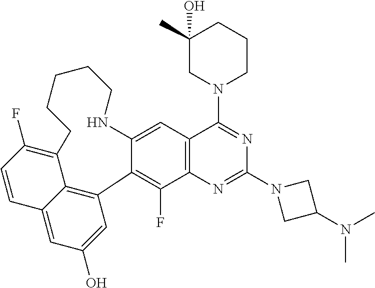 [SMILES: CN(C)C1CN(c2nc(N3CCC[C@@](C)(O)C3)c3cc4c(c(F)c3n2)-c2cc(O)cc3ccc(F)c(c23)CCCCCN4)C1]

Example 39: Prepared using a similar synthetic route as described in Example 16: LCMS (ESI): [M+H]⁺=603.1. ¹H NMR (400 MHz, CD₃OD) δ ppm 7.73-7.63 (m, 1H), 7.41-7.14 (m, 3H), 6.92 (dd, J=2.8, 4.7 Hz, 1H), 4.22 (br s, 2H), 4.13-3.85 (m, 4H), 3.69 (br s, 1H), 3.37 (br s, 2H), 3.29-3.25 (m, 1H), 2.93 (s, 1H), 2.67 (br d, J=4.5 Hz, 1H), 2.44-2.34 (m, 1H), 2.26 (s, 6H), 2.15-2.04 (m, 1H), 1.87-1.68 (m, 4H), 1.56 (br s, 2H), 1.36 (br s, 2H), 1.30 (d, J=5.4 Hz, 3H), 1.01-0.87 (m, 1H).

### Example 40: (3R)-1-(17-amino-3,15-difluoro-13-((2-methylenetetrahydro-1H-pyrazin-7a(5H)-yl)methoxy)-4,5,6,7,8,9-hexahydronaphtho[1′,8′:4,5,6][1]azacycloundecano[2,3-g]quinazolin-11-yl)-3-methylpiperidin-3-ol

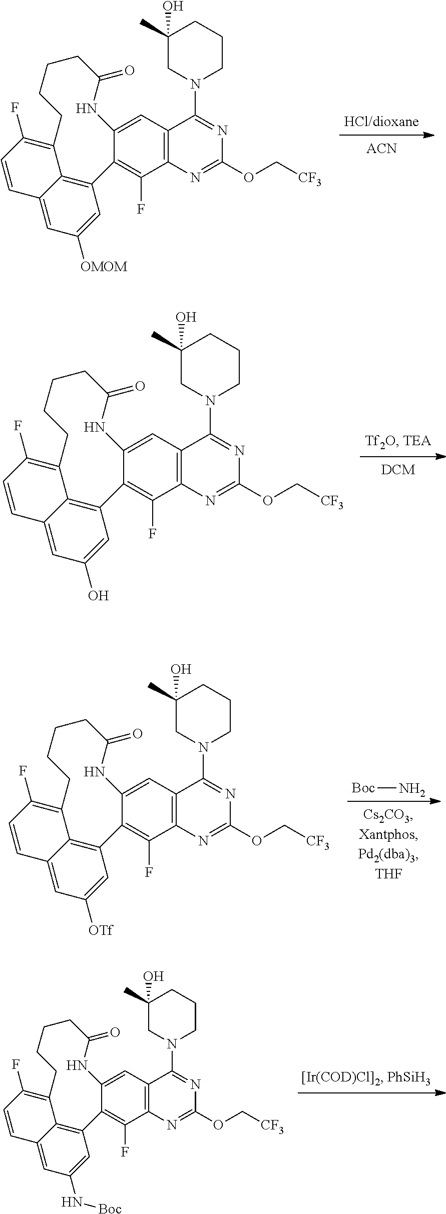

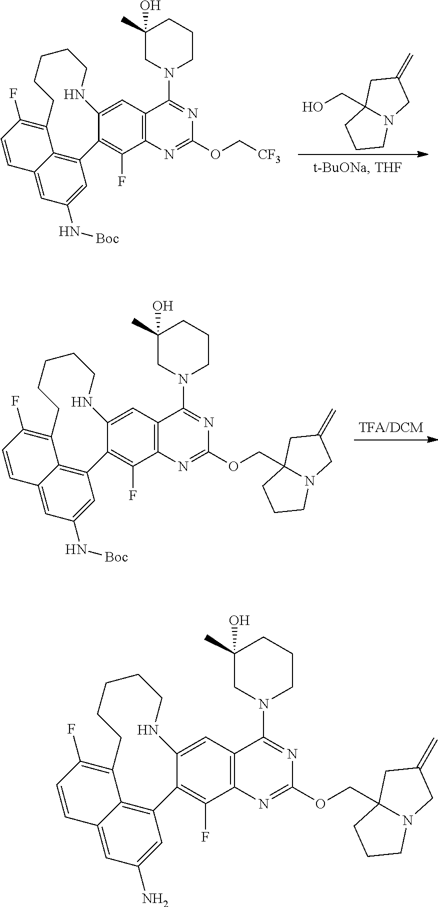 [SMILES: CC(C)(C)OC(=O)Nc1cc2c3c(c(F)ccc3c1)CCCCCNc1cc3c(N4CCC[C@@](C)(O)C4)nc(OCC(F)(F)F)nc3c(F)c1-2 ; C=C1CN2CCCC2(CO)C1 ; C=C1CN2CCCC2(COc2nc(N3CCC[C@@](C)(O)C3)c3cc4c(c(F)c3n2)-c2cc(NC(=O)OC(C)(C)C)cc3ccc(F)c(c23)CCCCCN4)C1 ; "TFA/DCM" ; C=C1CN2CCCC2(COc2nc(N3CCC[C@@](C)(O)C3)c3cc4c(c(F)c3n2)-c2cc(N)cc3ccc(F)c(c23)CCCCCN4)C1]

Step I: At 0° C., to a solution of (R)-3,15-difluoro-11-(3-hydroxy-3-methylpiperidin-1-yl)-17-(methoxymethoxy)-13-(2,2,2-trifluoroethoxy)-4,5,6,7-tetrahydronaphtho[1′,8′:4,5,6][1]azacycloundecino[2,3-g]quinazolin-8(9H)-one (1.00 g, 1.51 mmol) in acetonitrile (20.0 mL) was added hydrogen chloride (4 M in dioxane, 5.68 mL, 22.71 mmol). The solution was stirred at 20° C. for 1 hour. The reaction mixture was concentrated under reduced pressure to give a crude compound (R)-3,15-difluoro-17-hydroxy-11-(3-hydroxy-3-methylpiperidin-1-yl)-13-(2,2,2-trifluoroethoxy)-4,5,6,7-tetrahydronaphtho[1′,8′:4,5,6][1]azacycloundecino[2,3-g]quinazolin-8(9H)-one (1.00 g), which was obtained as a brown solid. LCMS (ESI): [M+H]⁺=617.1.

Step II: at 0° C., to a solution of (R)-3,15-difluoro-17-hydroxy-11-(3-hydroxy-3-methylpiperidin-1-yl)-13-(2,2,2-trifluoroethoxy)-4,5,6,7-tetrahydronaphtho[1′,8′:4,5,6][1]azacycloundecino[2,3-g]quinazolin-8(9H)-one (crude, 920 mg, 1.49 mmol) in dichloromethane (20.0 mL) was added triethylamine (1.04 mL, 7.46 mmol). Trifluoromethanesulfonic anhydride (276 uL, 1.64 mmol) was slowly added dropwise at 0° C. and the solution was stirred at 0° C. for 4 hours. Upon completion, the reaction mixture was quenched with ice water (10 mL) and extracted with dichloromethane (10 mL×3). The combined organic layers were dried over anhydrous sodium sulfate, filtered, and concentrated under reduced pressure. The residue was purified by flash chromatography (silica gel, gradient of 0-30% tetrahydrofuran/petroleum ether) to give a brown solid compound (R)-3,15-difluoro-11-(3-hydroxy-3-methylpiperidin-1-yl)-8-oxo-13-(2,2,2-trifluoroethoxy)-4,5,6,7,8,9-hexahydronaphtho[1′,8′:4,5,6][1]azacycloundecino[2,3-g]quinazolin-17-yl trifluoromethanesulfonate (510 mg, 0.68 mmol, yield: 46%). LCMS (ESI): [M+H]⁺=749.1.

Step III: Under a nitrogen atmosphere, a solution of (R)-3,15-difluoro-11-(3-hydroxy-3-methylpiperidin-1-yl)-8-oxo-13-(2,2,2-trifluoroethoxy)-4,5,6,7,8,9-hexahydronaphtho[1′,8′:4,5,6][1]azacycloundecino[2,3-g]quinazolin-17-yl trifluoromethanesulfonate (500 mg, 0.67 mmol) in tetrahydrofuran (5.00 mL) was treated with tert-butyl carbamate (110 mg, 0.94 mmol), cesium carbonate (653 mg, 2.00 mmol), 4,5-bis(diphenylphosphino)-9,9-dimethylxanthene (38.6 mg, 0.07 mmol), and tris(dibenzylideneacetone)dipalladium(0) (61.2 mg, 0.07 mmol). The mixture was stirred at 70° C. for 2 h. After cooling to room temperature, water (2 mL) was added, and the mixture was extracted with ethyl acetate (2 mL×3). The combined organic layers were dried over anhydrous sodium sulfate, filtered, and concentrated under reduced pressure. The residue was purified by flash chromatography (silica gel, gradient of 0-30% tetrahydrofuran/petroleum ether) to give a brown solid compound (R)3,15-difluoro-11-(3-hydroxy-3-methylpiperidin-1-yl)-8-oxo-13-(2,2,2-trifluoroethoxy)-4,5,6,7,8,9-hexahydronaphtho[1′,8′:4,5,6][1]azacycloundecino[2,3-g]quinazolin-17-yl trifluoromethanesulfonate (110 mg, 0.15 mmol, yield: 23%). LCMS (ESI): [M+H]⁺=716.5.

Step IV: To a solution of (R)-(3,15-difluoro-11-(3-hydroxy-3-methylpiperidin-1-yl)-8-oxo-13-(2,2,2-trifluoroethoxy)-4,5,6,7,8,9-hexahydronaphtho[1′,8′:4,5,6][1]azacycloundecino[2,3-g]quinazolin-17-yl) tert-butylcarbamate (110 mg, 0.15 mmol) in tetrahydrofuran (5.0 mL) and phenylsilane (166 mg, 1.54 mmol) was added bis(1,5-cyclooctadiene)chloroiridium(I) dimer (10.3 mg, 15.37 μmol). The reaction mixture was stirred at 25° C. for 16 hours. The reaction mixture was concentrated under reduced pressure, and the residue was purified by flash chromatography (silica gel, gradient of 0-30% tetrahydrofuran/petroleum ether) to give a yellow solid compound (R)-(3,15-difluoro-11-(3-hydroxy-3-methylpiperidin-1-yl)-13-(2,2,2-trifluoroethoxy)-4,5,6,7,8,9-hexahydronaphtho[1′,8′:4,5,6][1]azacycloundecino[2,3-g]quinazolin-17-yl) tert-butylcarbamate (53.0 mg, 75.51 μmol, yield: 49%). LCMS (ESI): [M+H]⁺=702.1.

Step V: (R)-(3,15-Difluoro-11-(3-hydroxy-3-methylpiperidin-1-yl)-13-(2,2,2-trifluoroethoxy)-4,5,6,7,8,9-hexahydronaphtho[1′,8′:4,5,6][1]azacycloundecino[2,3-g]quinazolin-17-yl) tert-butylcarbamate (50.0 mg, 71.25 μmol), 4 Å molecular sieves (20.0 mg), and sodium tert-butoxide (34.2 mg, 0.36 mmol) were dissolved in tetrahydrofuran (500 μL). To this solution, (2-methylenetetrahydro-1H-pyrazino[2,3-b][1,4]diazepin-7a(5H)-yl)methanol (54.0 mg, 0.36 mmol) was added, and the mixture was stirred at 60° C. for 16 hours. The mixture was diluted with water (300 μL) and extracted with ethyl acetate (300 μL×3). The combined organic layers were dried over anhydrous sodium sulfate, filtered, and concentrated under reduced pressure. The residue was purified by flash column chromatography (silica gel, gradient elution with 0-30% tetrahydrofuran/petroleum ether) to give a brown solid compound tert-butyl (3,15-difluoro-11-((R)-3-hydroxy-3-methylpiperidin-1-yl)-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazolin-17-yl)carbamate (28.0 mg, 37.05 μmol, yield: 52%). LCMS (ESI): [M+H]⁺=755.4.

Step VI: A solution of (3,15-difluoro-11-((R)-3-hydroxy-3-methylpiperidin-1-yl)-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazolin-17-yl)carbamate (30.0 mg, 39.74 μmol) in trifluoroacetic acid/dichloromethane (1:4, v/v, 300 μL) was prepared, the mixture was stirred at 25° C. for 2 hours. The reaction mixture was concentrated under vacuum, and the residue was dissolved in dichloromethane (300 μL) and neutralized with triethylamine (to pH>7). The solvent was removed under reduced pressure, and the residue was purified by preparative HPLC to give a yellow solid compound (3R)-1-(17-amino-3,15-difluoro-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazolin-11-yl)-3-methylpiperidin-3-ol (1.09 mg, 1.59 μmol, yield: 4%). LCMS (ESI): [M+H]⁺=655.3; ¹H NMR (400 MHz, CD₃OD) δ ppm 7.60 (dd, J=6.3, 8.7 Hz, 1H), 7.52-7.41 (m, 1H), 7.37-7.33 (m, 1H), 7.19 (br s, 1H), 6.86 (s, 1H), 5.04 (br s, 2H), 4.41-4.26 (m, 2H), 4.25-4.15 (m, 1H), 4.14-4.04 (m, 1H), 3.89-3.82 (m, 1H), 3.80-3.70 (m, 1H), 3.48-3.38 (m, 2H), 3.07-2.92 (m, 1H), 2.88-2.79 (m, 2H), 2.69-2.48 (m, 2H), 2.42-2.28 (m, 1H), 2.24-2.16 (m, 3H), 2.09-1.96 (m, 3H), 1.92-1.74 (m, 6H), 1.67-1.51 (m, 4H), 1.35 (br s, 3H).

### Example 41: 3,15-Difluoro-13-(((2R,7aS)-2-fluoro-tetrahydro-1H-pyrazin-7a(5H)-yl)methoxy)-17-hydroxy-11-((R)-3-hydroxy-3-methylpiperidin-1-yl)-9-(trideuteromethyl)-4,5,6,7-tetrahydronaphtho[1′,8′:4,5,6][1]azacycloundecino[2,3-g]quinazolin-8(9H)-one

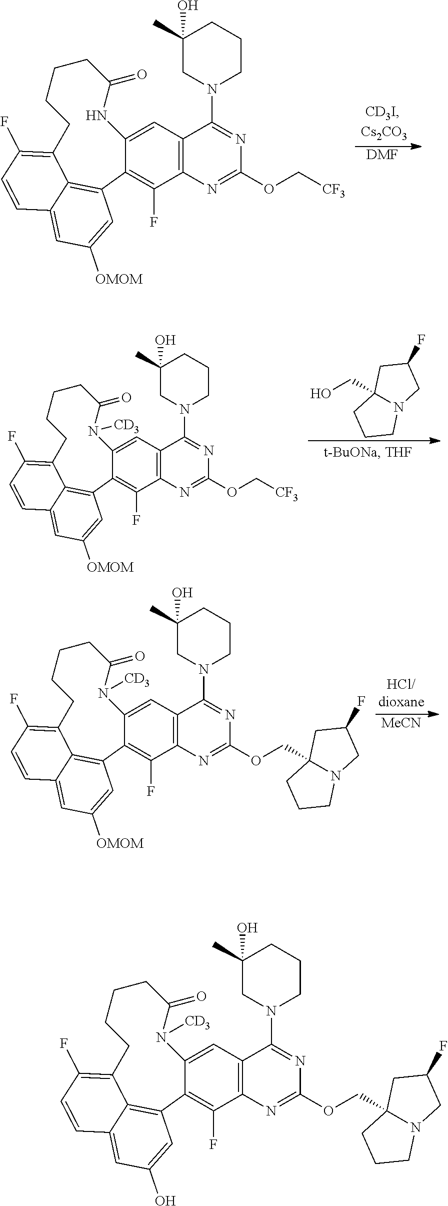

Step I: To a solution of (R)-3,15-difluoro-11-(3-hydroxy-3-methylpiperidin-1-yl)-17-(methoxymethoxy)-13-(2,2,2-trifluoroethoxy)-4,5,6,7-tetrahydronaphtho[1′,8′:4,5,6][1]azacycloundecino[2,3-g]quinazolin-8(9H)-one (100 mg, 0.15 mmol) and cesium carbonate (54.3 mg, 0.17 mmol) in N,N-dimethylformamide (2.0 mL) was added deuterated methyl iodide (19 μL, 0.30 mmol). The solution was stirred at 25° C. for 16 hours, then water (6 mL) was added and extracted with ethyl acetate (6 mL×3). The organic layers were combined, dried over anhydrous sodium sulfate, filtered, and concentrated under reduced pressure. to give a brown oily crude compound (R)-3,15-difluoro-11-(3-hydroxy-3-methylpiperidin-1-yl)-17-(methoxymethoxy)-9-(trideuteromethyl)-13-(2,2,2-trifluoroethoxy)-4,5,6,7-tetrahydronaphtho[1′,8′:4,5,6][1]azacycloundecino[2,3-g]quinazolin-8(9H)-one (120 mg). LCMS (ESI): [M+H]⁺=678.1.

Step II: To a solution of (R)-3,15-difluoro-11-(3-hydroxy-3-methylpiperidin-1-yl)-17-(methoxymethoxy)-9-(trideuteromethyl)-13-(2,2,2-trifluoroethoxy)-4,5,6,7-tetrahydronaphtho[1′,8′:4,5,6][1]azacycloundecino[2,3-g]quinazolin-8(9H)-one (115 mg, 0.17 mmol), 4A molecular sieves (115 mg), and ((2R,7aS)-2-fluorotetrahydro-1H-pyrazin-7a(5H)-yl)methanol (81 mg, 0.51 mmol) in tetrahydrofuran (2.0 mL) was added sodium tert-butoxide (32.6 mg, 0.34 mmol). The mixture was stirred at 60° C. for 16 hours. Water (2 mL) was added to dilute the reaction mixture, followed by extraction with ethyl acetate (3 mL×3). The combined organic layers were dried over anhydrous sodium sulfate, filtered, and concentrated under reduced pressure to give a yellow oily crude compound 3,15-difluoro-13-(((2R,7aS)-2-fluorotetrahydro-1H-pyrazin-7a(5H)-yl)methoxy)-11-((R)-3-hydroxy-3-methylpiperidin-1-yl)-17-(methoxymethoxy)-9-(trideuteromethyl)-4,5,6,7-tetrahydronaphtho[1′,8′:4,5,6][1]azacycloundecino[2,3-g]quinazolin-8(9H)-one (200 mg). LCMS (ESI): [M+H]⁺=737.5.

Step III: To a solution of 3,15-difluoro-13-(((2R,7aS)-2-fluorotetrahydro-1H-pyrazin-7a(5H)-yl)methoxy)-11-((R)-3-hydroxy-3-methylpiperidin-1-yl)-17-(methoxymethoxy)-9-(trideuteromethyl)-4,5,6,7-tetrahydronaphtho[1′,8′:4,5,6][1]azacycloundecino[2,3-g]quinazolin-8(9H)-one (200 mg, 0.27 mmol) in acetonitrile (4.0 mL) was added hydrogen chloride (4 M in dioxane solution, 1018 IL, 4.07 mmol). The mixture was stirred at 25° C. for 1 hour. The reaction solution was evaporated under a nitrogen atmosphere. The residue was purified by preparative HPLC to give a yellow solid compound 3,15-difluoro-13-(((2R,7aS)-2-fluorotetrahydro-1H-pyrazin-7a(5H)-yl)methoxy)-17-hydroxy-11-((R)-3-hydroxy-3-methylpiperidin-1-yl)-9-(trideuteromethyl)-4,5,6,7-tetrahydronaphtho[1′,8′:4,5,6][1]azacycloundecino[2,3-g]quinazolin-8(9H)-one (17.49 mg, 0.03 mmol, yield: 9%). LCMS (ESI): [M+H]⁺=693.3. ¹H NMR (400 MHz, CD₃OD) δ ppm 8.11 (d, J=9.8 Hz, 1H), 7.70 (dd, J=5.9, 8.9 Hz, 1H), 7.37-7.23 (m, 2H), 7.10-6.98 (m, 1H), 5.47-5.30 (m, 1H), 4.49-4.39 (m, 2H), 4.38-4.30 (m, 1H), 4.23-4.13 (m, 1H), 3.60-3.49 (m, 2H), 3.48-3.36 (m, 4H), 3.21-3.10 (m, 1H), 2.76 (br d, J=4.5 Hz, 1H), 2.66-2.55 (m, 1H), 2.48 (br dd, J=6.7, 13.6 Hz, 1H), 2.39-2.31 (m, 1H), 2.24-2.08 (m, 5H), 2.01-1.92 (m, 1H), 1.85 (br s, 1H), 1.82-1.73 (m, 2H), 1.53 (br d, J=6.6 Hz, 2H), 1.41-1.33 (m, 1H), 1.31-1.25 (m, 3H), 1.15 (br s, 1H).

### Example 42: 3,15-difluoro-11-((R)-3-hydroxy-3-methylpiperidin-1-yl)-13-((2-methylenetetrahydro-1H-pyrazin-7a(5H)-yl)methoxy)-4,5,8,9-tetrahydro-7H-naphtho[1′,8′:7,8,9][1]oxa[4]azacycloundecane[5,6-g]quinazolin-17-ol

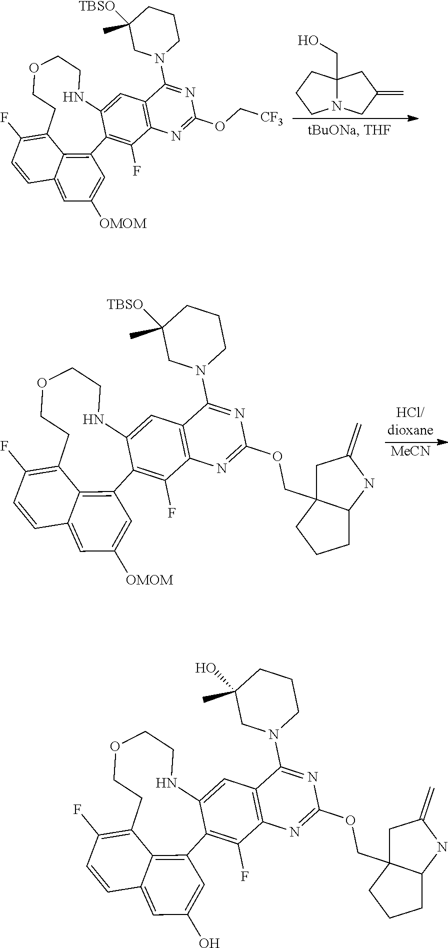

Step I: To a solution of (R)-1-(3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundecino[5,6-g]quinazolin-11-yl)-3-methylpiperidin-3-ol and (2-methylenetetrahydro-1H-pyrazin-7a(5H)-yl)methanol (20.0 mg, 0.03 mmol) in tetrahydrofuran (0.4 mL) was added sodium tert-butoxide (9.0 mg, 0.09 mmol). The mixture was stirred at 60° C. for 16 hours under a nitrogen atmosphere. then diluted with water (2 mL) at 0° C. and extracted with ethyl acetate (2 mL×3). The organic layers were combined, dried over anhydrous sodium sulfate, filtered, and concentrated under reduced pressure. The residue was purified using preparative thin-layer chromatography (silica gel, dichloromethane:methanol=10:1) to give a yellow solid compound (3R)-1-(3,15-difluoro-17-(methoxymethoxy)-13-((2-methylenetetrahydro-1H-pyrazin-7a(5H)-yl)methoxy)-4,5,8,9-tetrahydro-7H-naphtho[1′,8′:7,8,9][1]oxa[4]azacycloundecino[5,6-g]quinazolin-11-yl)-3-methylpiperidin-3-ol (10 mg, 0.01 mmol, yield: 46%). LCMS (ESI): [M+H]⁺=702.4.

Step II: To a solution of (3R)-1-(3,15-difluoro-17-(methoxymethoxy)-13-((2-methylenetetrahydro-1H-pyrazin-7a(5H)-yl)methoxy)-4,5,8,9-tetrahydro-7H-naphtho[1′,8′:7,8,9][1]oxa[4]azacycloundecino[5,6-g]quinazolin-11-yl)-3-methylpiperidin-3-ol (10 mg, 0.01 mmol) in acetonitrile (200 μL) was added hydrochloric acid (4 M in dioxane, 53 μL, 0.21 mmol). The mixture was stirred at 25° C. for 1 hour. The solution was evaporated under a nitrogen stream, and the residue was purified by preparative HPLC to give a yellow solid compound 3,15-difluoro-11-((R)-3-hydroxy-3-methylpiperidin-1-yl)-13-((2-methylenetetrahydro-1H-pyrazin-7a(5H)-yl)methoxy)-4,5,8,9-tetrahydro-7H-naphtho[1′,8′:7,8,9][1]oxa[4]azacycloundecino[5,6-g]quinazolin-17-ol (2.22 mg, 3.38 μmol, yield: 24%). LCMS (ESI): [M+H]⁺=658.4; ¹H NMR (400 MHz, CD₃OD) δ ppm 7.77 (br s, 1H), 7.62-7.50 (m, 1H), 7.40-7.25 (m, 2H), 6.97 (br s, 1H), 5.13 (br s, 2H), 4.53-4.40 (m, 2H), 4.29 (br d, J=12.5 Hz, 1H), 4.22-3.99 (m, 2H), 3.85 (br d, J=8.2 Hz, 1H), 3.69-3.53 (m, 4H), 3.49-3.36 (m, 3H), 3.17-2.92 (m, 2H), 2.72-2.57 (m, 3H), 2.34-1.93 (m, 5H), 1.92-1.71 (m, 3H), 1.40-1.25 (m, 5H).

### Examples 42A to 42D: Four Isomers of 3,15-difluoro-11-((R)-3-hydroxy-3-methylpiperidin-1-yl)-13-(((S)-(2-methylenetetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-17-ol

The compound 3,15-difluoro-11-((R)-3-hydroxy-3-methylpiperidin-1-yl)-13-((2-methylene-tetrahydro-1H-pyrazin-7a(5H)-yl)methoxy)-4,5,8,9-tetrahydro-7H-naphtho[1′,8′:7,8,9][1]oxazino[4]azacycloundecino[5,6-g]quinazolin-17-ol was separated using SFC, yielding four distinct isomers. (Column: DAICEL CHIRALPAK IG (250 mm×30 mm, 10 μm); Mobile Phase: Phase A: carbon dioxide, Phase B: 0.1% ammonium hydroxide/ethanol; Phase B Proportion: 60%; Flow Rate: 80 mL/min)

Example 42A: LCMS (ESI): [M+H]⁺=658.3; SFC Analysis (Column: Chiralpak IG-3, 50×4.6 mm I.D., 3 μm; Mobile Phase: A=Carbon Dioxide, B=0.05% Diethylamine/Ethanol; Gradient: 40% B Phase maintained; Flow Rate: 4 mL/min): The retention time on the chiral column was 0.846 min; ¹H NMR (400 MHz, CD₃OD) δ ppm 7.77-7.73 (dd, J=6.0, 9.0 Hz, 1H), 7.52 (s, 1H), 7.34-7.24 (m, 2H), 6.95 (d, J=2.6 Hz, 1H), 5.14 (br s, 2H), 4.50-4.40 (m, 2H), 4.28 (br d, 0.1=14.1 Hz, 1H), 4.13-4.02 (m, 2H), 3.85-3.82 (m, 1H), 3.65-3.57 (m, 4H), 3.45-3.33 (m, 3H), 3.28-3.22 (m, 1H), 3.19-3.06 (m, 1H), 3.02-2.88 (m, 2H), 2.70-2.58 (m, 3H), 2.29-1.96 (m, 5H), 1.88-1.71 (m, 3H), 1.31-1.26 (m, 3H).

Example 42B: LCMS (ESI): [M+H]⁺=658.3; SFC Analysis (Column: Chiralpak IG-3, 50×4.6 mm I.D., 3 μm; Mobile Phase: A=Carbon Dioxide, B=0.05% Diethylamine/Ethanol; Gradient: 40% B Phase maintained; Flow Rate: 4 mL/min): The retention time on the chiral column was 2.271 min; ¹H NMR (400 MHz, CD₃OD) δ ppm 7.77-7.70 (dd, J=6.0, 9.0 Hz, 1H), 7.49 (s, 1H), 7.34-7.24 (m, 2H), 6.95 (d, J=2.6 Hz, 1H), 5.14 (br s, 2H), 4.59-4.40 (m, 2H), 4.26 (br d, J=14.1 Hz, 1H), 4.17-4.14 (m, 1H), 4.08-4.05 (m, 1H), 3.91-3.78 (m, 1H), 3.66-3.52 (m, 4H), 3.50-3.30 (m, 4H), 3.25-2.88 (m, 3H), 2.65-2.58 (m, 3H), 2.29-1.96 (m, 5H), 1.88-1.71 (m, 3H), 1.31-1.26 (m, 3H).

Example 42C: LCMS (ESI): [M+H]+=658.3; SFC Analysis (Column: Chiralpak IG-3, 50×4.6 mm I.D., 3 μm; Mobile Phase: A=Carbon Dioxide, B=0.05% Diethylamine/Ethanol; Gradient: 40% B Phase maintained; Flow Rate: 4 mL/min): The retention time on the chiral column was 0.893 min; ¹H NMR (400 MHz, CD₃OD) δ ppm 7.76 (dd, J=6.2, 9.0 Hz, 1H), 7.53 (s, 1H), 7.38-7.26 (m, 2H), 6.95 (d, J=2.4 Hz, 1H), 5.12 (br s, 2H), 4.51-4.31 (m, 2H), 4.31-4.19 (m, 1H), 4.18-4.07 (m, 1H), 3.97 (m, 1H), 3.84 (m, 1H), 3.65-3.49 (m, 4H), 3.48-3.30 (m, 4H), 3.18-2.78 (m, 3H), 2.72-2.51 (m, 3H), 2.25-1.69 (m, 8H), 1.32-1.23 (m, 3H).

Example 42D: LCMS (ESI): [M+H]⁺=658.3; SFC Analysis (Column: Chiralpak IG-3, 50×4.6 mm I.D., 3 μm; Mobile Phase: A=Carbon Dioxide, B=0.05% Diethylamine/Ethanol; Gradient: 40% B Phase maintained; Flow Rate: 4 mL/min): The retention time on the chiral column was 3.209 min; ¹H NMR (400 MHz, CD₃OD) δ ppm 7.74 (dd, J=6.2, 9.0 Hz, 1H), 7.57-7.53 (m, 1H), 7.38-7.27 (m, 2H), 6.95 (m, 1H), 5.16 (br s, 2H), 4.55-4.45 (m, 2H), 4.35-4.24 (m, 1H), 4.18-4.06 (m, 2H), 3.92-3.81 (m, 1H), 3.65-3.52 (m, 5H), 3.44-3.30 (m, 3H), 3.16-2.88 (m, 3H), 2.72-2.57 (m, 3H), 2.32-1.75 (m, 8H), 1.32-1.23 (m, 3H).

### Example 43A and Example 43B: 2 Stereoisomers of 3,15-difluoro-13-((2R,7aS)-2-fluorotetrahydro-1H-pyrazin-7a(5H)-yl)methoxy)-11-((R)-3-hydroxy-3-methylpiperidin-1-yl)-4,5,8,9-tetrahydro-7H-naphtho[1′,8′:7,8,9][1]oxa[4]azacycloundeca[5,6-g]quinazoline-17-ol

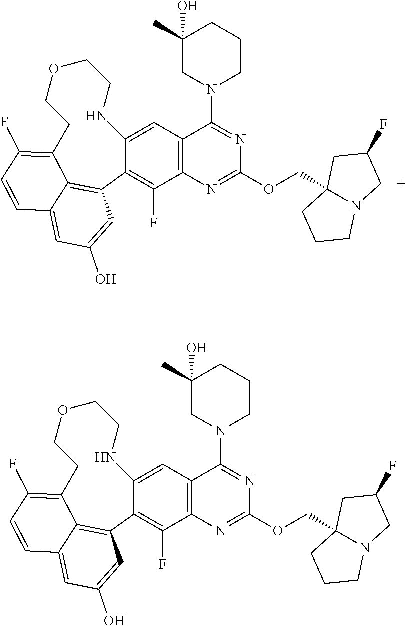

Example 43A: Prepared following a synthetic route similar to that described in Example 42: LCMS (ESI): [M+H]⁺=664.2; ¹H NMR (400 MHz, CD₃OD) δ ppm 7.74 (dd, J=5.9, 9.1 Hz, 1H), 7.51 (s, 1H), 7.35-7.22 (m, 2H), 6.94 (d, J=2.6 Hz, 1H), 5.41-5.18 (m, 1H), 4.31-4.25 (m, 1H), 4.24-4.14 (m, 2H), 4.09 (br d, J=13.1 Hz, 1H), 3.83 (td, J=6.3, 12.5 Hz, 1H), 3.64-3.51 (m, 3H), 3.42 (br d, J=12.8 Hz, 1H), 3.34 (br s, 1H), 3.30-2.93 (m, 6H), 2.63 (br d, J=3.0 Hz, 2H), 2.39-2.09 (m, 4H), 2.04-1.71 (m, 6H), 1.29 (s, 3H).

Example 43B: Prepared following a synthetic route similar to that described in Example 42: [M+H]⁺=664.2; ¹H NMR (400 MHz, CD₃OD) δ ppm 7.74 (dd, J=5.9, 9.1 Hz, 1H), 7.48 (s, 1H), 7.35-7.23 (m, 2H), 6.94 (d, J=2.6 Hz, 1H), 5.41-5.19 (m, 1H), 4.30-4.26 (m, 1H), 4.25-4.16 (m, 2H), 4.06 (br d, 0.1=13.4 Hz, 1H), 3.89-3.80 (m, 1H), 3.66-3.54 (m, 3H), 3.46-3.40 (m, 1H), 3.38-3.33 (m, 1H), 3.29-2.96 (m, 6H), 2.65 (br d, J=3.0 Hz, 2H), 2.39-2.10 (m, 4H), 2.03-1.71 (m, 6H), 1.27 (s, 3H).

### Example 44: 1-(3,15-Difluoro-17-hydroxy-11-((R)-3-hydroxy-3-methylpiperidin-1-yl)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)ethan-1-one

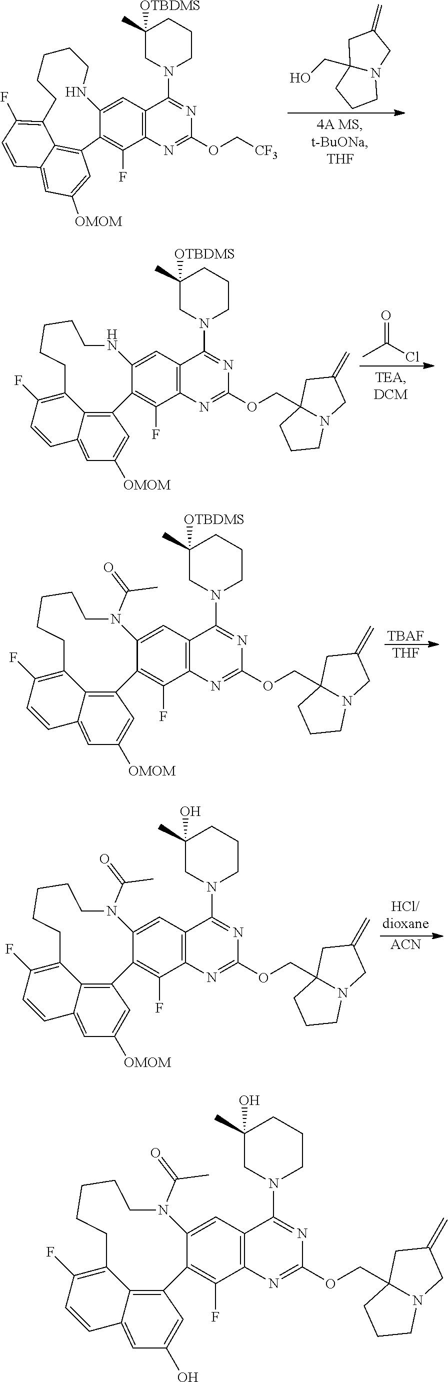

Step I: To the solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,6,7,8,9-hexahydro-naphtho[1′,8′:4,5,6][1]azacycloundecino[2,3-g]quinazoline (60.0 mg, 0.09 mmol) and (2-methylenetetrahydro-1H-pyrazin-7a(5H)-yl)methanol (57.0 mg, 0.37 mmol) in tetrahydrofuran (800.0 μL) were added 4A molecular sieves (60.0 mg) and sodium tert-butoxide (55.0 mg, 0.55 mmol). The solution was stirred at 60° C. for 16 hours. The reaction mixture was diluted with water (1 mL) and extracted with ethyl acetate (3×1 mL). The combined organic layers were dried over anhydrous sodium sulfate, filtered, and concentrated under reduced pressure to afford a yellow solid compound 11-((R)-3-((tert-butyldimethylsilyl)oxy)-3-methylpiperidin-1-yl)-3,15-difluoro-17-(methoxymethoxy)-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazoline (50.0 mg, 0.06 mmol, yield: 66%). LCMS (ESI): [M+H]⁺=814.4.

Step II: To a solution of 11-((R)-3-((tert-butyldimethylsilyl)oxy)-3-methylpiperidin-1-yl)-3,15-difluoro-17-(methoxymethoxy)-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazoline (30.0 mg, 0.04 mmol) in dichloromethane (400.0 μL) were added acetyl chloride (2.60 μL, 0.04 mmol) and triethylamine (15.0 μL, 0.11 mmol) at 0° C. The solution was stirred at 20° C. for 2 hours. The reaction mixture was diluted with water (1 mL) and extracted with dichloromethane (1 mL×2). The combined organic layers were dried over anhydrous sodium sulfate, filtered, and concentrated under reduced pressure. The residue was purified by flash column chromatography (silica gel, 0-80% gradient of ethyl acetate/petroleum ether) to afford a yellow solid compound 1-(11-((R)-3-(tert-butyldimethylsilyl)oxy)-3-methylpiperidin-1-yl)-3,15-difluoro-17-(methoxymethoxy)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)acetone (25.0 mg, 0.03 mmol, yield: 79%). LCMS (ESI): [M+H]⁴=856.4.

Step III: To a solution of 1-(11-((R)-3-(tert-butyldimethylsilyl)oxy)-3-methylpiperidin-1-yl)-3,15-difluoro-17-(methoxymethoxy)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)acetone (20.0 mg, 0.02 mmol) in tetrahydrofuran (400.0 μL) was added tetrabutylammonium fluoride (1 M in tetrahydrofuran, 187.0 μL, 0.18 mmol). The solution was stirred at 20° C. for 2 hours. The reaction mixture was diluted with water (1 mL) and extracted with ethyl acetate (1 mL×3). The combined organic layers were dried over anhydrous sodium sulfate, filtered, and concentrated under reduced pressure. to give a yellow solid crude compound 1-(3,15-difluoro-11-((R)-3-hydroxy-3-methylpiperidin-1-yl)-17-(methoxymethoxy)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)acetone (20.0 mg, 0.03 mmol). LCMS (ESI): [M+H]⁺=742.3.

Step IV: To a solution of 1-(3,15-difluoro-11-((R)-3-hydroxy-3-methylpiperidin-1-yl)-17-(methoxymethoxy)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)ethyl-1-one (14 mg, 0.02 mmol) in acetonitrile (200.0 μL) was added hydrogen chloride (4 M in dioxane, 19 μL, 0.07 mmol). The reaction mixture was stirred at 20° C. for 2 hours. The solution was concentrated under reduced pressure, and dimethylsulfoxide (1 mL) was added. The pH was adjusted to 8 using triethylamine. The residue was purified by flash column chromatography (silica gel, 0-80% gradient of ethyl acetate/petroleum ether) to give a yellow solid compound 1-(11-((R)-3-(tert-butyldimethylsilyl)oxy)-3-methylpiperidin-1-yl)-3,15-difluoro-17-(methoxymethoxy)-13-((2-methylene-tetrahydro-1H-pyrazin-7a(5H)-yl)methoxy)-5,6,7,8-tetrahydronaphtho[1′,8′:4,5,6][1]azacycloundecino[2,3-g]quinazolin-9(4H)-yl)acetone (25.0 mg, 0.03 mmol, yield: 79%) LCMS (ESI): [M+H]⁺=698.2; ¹H NMR (400 MHz, CD₃OD) δ ppm 8.17-7.92 (m, 1H), 7.74-7.62 (m, 1H), 7.32-7.24 (m, 2H), 7.24-6.84 (m, 1H), 5.07 (br d, J=7.09 Hz, 2H), 4.52-4.33 (m, 3H), 4.15 (br d, J=12.96 Hz, 1H), 4.00-3.58 (m, 2H), 3.56-3.44 (m, 2H), 2.99-2.73 (m, 4H), 2.62-2.32 (m, 2H), 2.20 (s, 5H), 2.12-1.51 (m, 8H), 1.42-1.18 (m, 8H), 0.92 (br d, J=17.36 Hz, 1H).

### Example 45: 3,15-difluoro-11-((R)-3-hydroxy-3-methylpiperidin-1-yl)-13-((2-methylenetetrahydro-1H-pyrazin-7a(5H)-yl)methoxy)-9-(methylsulfonyl)-4,5,6,7,8,9-hexahydronaphtho[1′,8′:4,5,6][1]azacycloundecano[2,3-g]quinazolin-17-ol

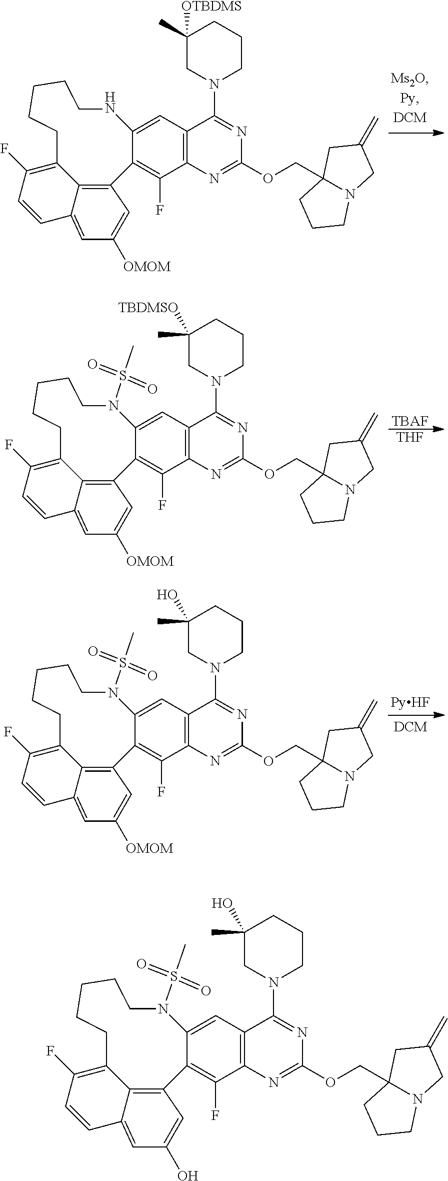

Step I: At 25° C. under a nitrogen atmosphere, to a solution of 11-((R)-3-(tert-butyldimethylsilyloxy)-3-methylpiperidin-1-yl)-3,15-difluoro-17-(methoxymethoxy)-13-((2-methylene-tetrahydro-1H-pyrazin-7a(5H)-yl)methoxy)-4,5,6,7,8,9-hexahydronaphtho[1′,8′:4,5,6][1]azacycloundecino[2,3-g]quinazoline (50 mg, 0.06 mmol) and pyridine (4 μL, 0.03 mmol) in dichloromethane (600 μL) was added methanesulfonic anhydride (43 mg, 0.25 mmol). The mixture was stirred at 20° C. for 2 h. The reaction mixture was diluted with water (1 mL) and extracted with dichloromethane (1 mL×3). The combined organic phases were dried over anhydrous sodium sulfate, filtered, and concentrated under reduced pressure to give a brown solid compound 11-((R)-3-(tert-butyldimethylsilyloxy)-3-methylpiperidin-1-yl)-3,15-difluoro-17-(methoxymethoxy)-13-((2-methylene-tetrahydro-1H-pyrazin-7a(5H)-yl)methoxy)-9-(methanesulfonyl)-4,5,6,7,8,9-hexahydronaphtho[1′,8′:4,5,6][1]azacycloundecino[2,3-g]quinazoline (50 mg, 44.8 μmol, yield: 73%). LCMS (ESI): [M+H]⁺=892.5.

Step II: At 25° C., under a nitrogen atmosphere, to a solution of 11-((R)-3-(tert-butyldimethylsilyloxy)-3-methylpiperidin-1-yl)-3,15-difluoro-17-(methoxymethoxy)-13-((2-methylene-tetrahydro-1H-pyrazin-7a(5H)-yl)methoxy)-9-(methanesulfonyl)-4,5,6,7,8,9-hexahydronaphtho[1′,8′:4,5,6][1]azacycloundecino[2,3-g]quinazoline (48 mg, 0.05 mmol) in tetrahydrofuran (1 mL) was added tetrabutylammonium fluoride (1 M in tetrahydrofuran, 538 μL, 0.538 mmol). The reaction mixture was stirred at 25° C. for 2 hours. Water (1 mL) was added to the reaction mixture, followed by extraction with ethyl acetate (1 mL×3). The combined organic phases were dried over anhydrous sodium sulfate, filtered, and concentrated under reduced pressure to give a crude brown solid compound (3R)-1-(3,15-difluoro-17-(methoxymethoxy)-13-((2-methylene-tetrahydro-1H-pyrazin-7a(5H)-yl)methoxy)-9-(methylsulfonyl)-4,5,6,7,8,9-hexahydronaphtho[1′,8′:4,5,6][1]azacycloundecino[2,3-g]quinazolin-11-yl)-3-methylpiperidin-3-ol (37 mg, 47.6 μmol, yield: 88%). LCMS (ESI): [M+H]⁺=778.4.

Step III: At 25° C., under a nitrogen atmosphere, to a solution of (3R)-1-(3,15-difluoro-17-(methoxymethoxy)-13-((2-methylene-tetrahydro-1H-pyrazin-7a(5H)-yl)methoxy)-9-(methylsulfonyl)-4,5,6,7,8,9-hexahydronaphtho[1′,8′:4,5,6][1]azacycloundecino[2,3-g]quinazolin-11-yl)-3-methylpiperidin-3-ol (36 mg, 0.05 mmol) in dichloromethane (500 μL) was added pyridine hydrofluoride (500 μL, 0.01 mmol). The mixture was stirred at 25° C. for 16 hours. The reaction was quenched with triethylamine to neutrality, followed by filtration. The filtrate was concentrated, and the crude product was purified by preparative HPLC to give a yellow solid compound 3,15-difluoro-11-((R)-3-hydroxy-3-methylpiperidin-1-yl)-13-((2-methylene-tetrahydro-1H-pyrazin-7a(5H)-yl)methoxy)-9-(methylsulfonyl)-4,5,6,7,8,9-hexahydronaphtho[1′,8′:4,5,6][1]azacycloundecino[2,3-g]quinazolin-17-ol (1.68 mg, 2.3 μmol, yield: 5%). LCMS (ESI): [M+H]⁺=734.4; ¹H NMR (400 MHz, CD₃OD) δ ppm 8.41 (s, 1H), 7.69 (dd, J=5.9, 8.9 Hz, 1H), 7.42 (d, J=2.5 Hz, 1H), 7.30-7.21 (m, 2H), 5.16 (br s, 2H), 4.53-4.45 (m, 2H), 4.33-4.05 (m, 3H), 3.76-3.64 (m, 2H), 3.58-3.48 (m, 1H), 3.45-3.36 (m, 2H), 3.03 (br s, 1H), 2.98-2.93 (m, 1H), 2.90 (s, 3H), 2.79 (br d, J=14.1 Hz, 1H), 2.71-2.61 (m, 1H), 2.41-2.25 (m, 2H), 2.19-2.00 (m, 4H), 1.89-1.71 (m, 4H), 1.45 (br s, 1H), 1.38-1.28 (m, 4H), 1.22-1.08 (m, 2H), 1.06-0.90 (m, 2H).

### Example 46: 3,15-difluoro-13-(((2R,7aS)-2-fluorotetrahydro-1H-pyrazin-7a(5H)-yl)methoxy)-11-((R)-3-hydroxy-3-methylpiperidin-1-yl)-9-(trideuteromethyl)-4,5,8,9-tetrahydro-7H-naphtho[1′,8′:7,8,9][1]oxa[4]azacycloundeca[5,6-g]quinazoline-17-ol

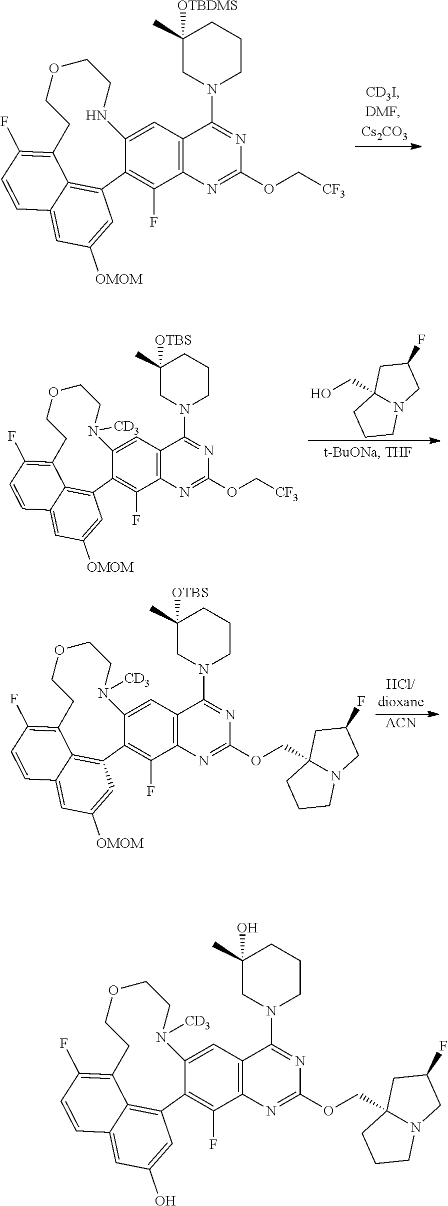

Step I: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundeca[5,6-g]quinazoline (100 mg, 0.13 mmol) in N,N-dimethylformamide (1.00 mL) were added deuterated iodomethane (16.6 μL, 0.26 mmol) and cesium carbonate (47.0 mg, 0.14 mmol). The mixture was stirred at 25° C. for 4 hour, then diluted with water (1 mL) and extracted with ethyl acetate (1 mL×3). The organic layers were combined, dried over anhydrous sodium sulfate, filtered, and concentrated under reduced pressure. The residue was purified by flash column chromatography (silica gel, 0-20% gradient of tetrahydrofuran/petroleum ether) to give a yellow solid compound (R)-11-(3-((tert-butyldimethylsilyl)oxy)-3-methylpiperidin-1-yl)-3,15-difluoro-17-(methoxymethoxy)-9-(trideuteriomethyl)-13-(2,2,2-trifluoroethoxy)-4,5,8,9-tetrahydro-7H-naphtho[1′,8′:7,8,9][1]oxa[4]azacycloundeca[5,6-g]quinazoline (100 mg, 0.13 mmol, yield: 98%). LCMS (ESI): [M+H]⁺=780.3.

Step II: To a solution of (R)-11-(3-((tert-butyldimethylsilyl)oxy)-3-methylpiperidin-1-yl)-3,15-difluoro-17-(methoxymethoxy)-9-(trideuteriomethyl)-13-(2,2,2-trifluoroethoxy)-4,5,8,9-tetrahydro-7H-naphtho[1′,8′:7,8,9][1]oxa[4]azacycloundeca[5,6-g]quinazoline (100 mg, 0.13 mmol) and ((2R,7aS)-2-fluorotetrahydro-1H-pyrazin-7a(5H)-yl)methanol (40.8 mg, 0.27 mmol) in tetrahydrofuran (1.00 mL) were added sodium tert-butoxide (123 mg, 1.28 mmol) and 4 Å molecular sieves (50 mg). The mixture was stirred at 60° C. for 16 hours. The reaction mixture was diluted with water (1 mL) and extracted with ethyl acetate (1 mL×3). The combined organic layers were dried over anhydrous sodium sulfate, filtered, and concentrated under reduced pressure to give a brown solid compound 11-((R)-3-((tert-butyldimethylsilyl)oxy)-3-methylpiperidin-1-yl)-3,15-difluoro-13-(((2R,7aS)-2-fluorotetrahydro-1H-pyrazin-7a(5H)-yl)methoxy)-17-(methoxymethoxy)-9-(trideuteriomethyl)-4,5,8,9-tetrahydro-7H-naphtho[1′,8′:7,8,9][1]oxa[4]azacycloundeca[5,6-g]quinazoline (100 mg, 0.12 mmol, yield: 93%). LCMS (ESI): [M+H]⁺=839.4.

Step III: To a solution of 11-((R)-3-((tert-butyldimethylsilyl)oxy)-3-methylpiperidin-1-yl)-3,15-difluoro-13-(((2R,7aS)-2-fluorotetrahydro-1H-pyrazin-7a(5H)-yl)methoxy)-17-(methoxymethoxy)-9-(trideuteriomethyl)-4,5,8,9-tetrahydro-7H-naphtho[1′,8′:7,8,9][1]oxa[4]azacycloundeca[5,6-g]quinazoline (100 mg, 0.12 mmol) in acetonitrile (1.00 mL) was added hydrogen chloride (4 M solution in dioxane, 1.79 mL, 7.15 mmol). The mixture was stirred at 25° C. for 16 hours. The reaction mixture was concentrated under reduced pressure. The residue was dissolved in acetonitrile (1 mL) and neutralized with triethylamine (until pH>7). The solvent was removed under reduced pressure. The residue was purified by preparative HPLC to give a yellow solid compound 3,15-Difluoro-13-(((2R,7aS)-2-fluorotetrahydro-1H-pyrazin-7a(5H)-yl)methoxy)-11-((R)-3-hydroxy-3-methylpiperidin-1-yl)-9-(trideuteriomethyl)-4,5,8,9-tetrahydro-7H-naphtho[1′,8′:7,8,9][1]oxa[4]azacycloundeca[5,6-g]quinazoline-17-ol (42.1 mg, 0.06 mmol, yield: 52%). LCMS (ESI): [M+H]⁺=681.4. ¹H NMR (400 MHz, CD₃OD) δ ppm 7.87 (d, J=9.5 Hz, 1H), 7.70 (dd, J=6.0, 9.0 Hz, 1H), 7.32-7.20 (m, 2H), 6.89 (d, J=2.6 Hz, 1H), 5.36 (br s, 1H), 4.33-4.23 (m, 2H), 4.20 (d, J=10.5 Hz, 1H), 4.10 (br t, J=14.6 Hz, 1H), 3.66-3.52 (m, 2H), 3.50-3.32 (m, 4H), 3.29-3.13 (m, 3H), 3.12-2.95 (m, 3H), 2.84-2.66 (m, 2H), 2.39-2.09 (m, 4H), 2.03-1.82 (m, 4H), 1.81-1.70 (m, 2H), 1.29 (d, J=6.3 Hz, 3H).

### Example 47: 3,15-difluoro-13-(((2R,7aS)-2-fluorotetrahydro-1H-pyrazin-7a(5H)-yl)methoxy)-11-((R)-3-hydroxy-3-methylpiperidin-1-yl)-9-methyl-4,5,8,9-tetrahydro-7H-naphtho[1′,8′:7,8,9][1]oxa[4]azacycloundeca[5,6-g]quinazoline-17-ol

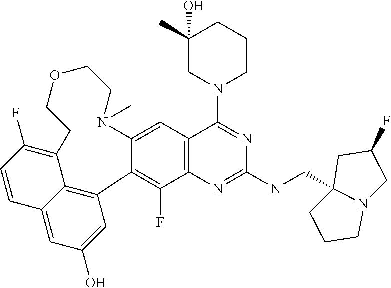 [SMILES: CN1CCOCCc2c(F)ccc3cc(O)cc(c23)-c2c1cc1c(N3CCC[C@@](C)(O)C3)nc(NC[C@@]34CCCN3C[C@H](F)C4)nc1c2F]

Example 47: Prepared using a similar synthetic route as described in Example 46: LCMS (ESI): [M+H]⁺=678.2. ¹H NMR (400 MHz, CD₃OD) δ ppm 7.90 (s, 1H), 7.70 (dd, J=6.1, 8.9 Hz, 1H), 7.30-7.21 (m, 2H), 6.89 (d, J=2.6 Hz, 1H), 5.39-5.20 (m, 1H), 4.35-4.07 (m, 4H), 3.64-3.54 (m, 2H), 3.47-3.33 (m, 4H), 3.29-3.13 (m, 3H), 3.12-2.94 (m, 3H), 2.86-2.67 (m, 5H), 2.39-2.10 (m, 4H), 2.03-1.94 (m, 2H), 1.92-1.69 (m, 4H), 1.30 (s, 3H).

### Example 48: 3,15-Difluoro-11-((R)-3-hydroxy-3-methylpiperidin-1-yl)-9-methyl-13-(((S)-(2-methylenetetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-17-ol

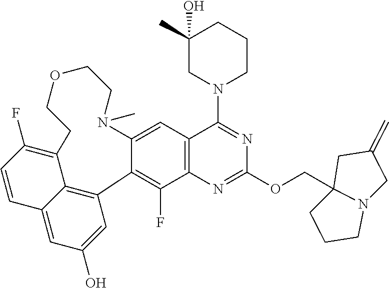 [SMILES: C=C1CN2CCCC2(COc2nc(N3CCC[C@@](C)(O)C3)c3cc4c(c(F)c3n2)-c2cc(O)cc3ccc(F)c(c23)CCOCCN4C)C1]

Example 48: Prepared using a similar synthetic route as described in Example 46: LCMS (ESI): [M+H]⁺=672.3. ¹H NMR (400 MHz, CD₃OD) δ ppm 8.56 (br s, 1H), 7.92 (d, J=13.33 Hz, 1H), 7.73 (dd, J=9.05, 6.11 Hz, 1H), 7.36-7.21 (m, 2H), 6.91 (s, 1H), 5.17 (br s, 2H), 4.66-4.34 (m, 3H), 4.27-4.05 (m, 2H), 3.70-3.57 (m, 2H), 3.55-3.36 (m, 6H), 3.18-2.91 (m, 4H), 2.77 (br d, J=10.03 Hz, 5H), 2.68 (br d, J=15.41 Hz, 1H), 2.33-2.01 (m, 5H), 1.94-1.73 (m, 3H), 1.32 (d, J=6.85 Hz, 3H).

### Example 49: 3,15-Difluoro-13-(((2R,7aS)-2-fluoro-6-methylenetetrahydro-1H-pyrazin-7a(5H)-yl)methoxy)-11-((R)-3-hydroxy-3-methylpiperidin-1-yl)-9-methyl-4,5,8,9-tetrahydro-7H-naphtho[1′,8′:7,8,9][1]oxa[4]azacycloundeca[5,6-g]quinazoline-17-ol

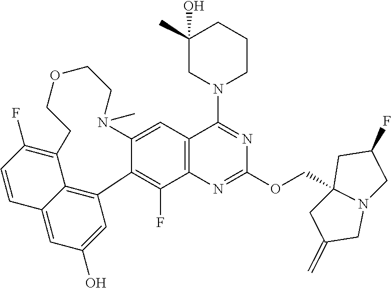 [SMILES: C=C1CN2C[C@H](F)C[C@]2(COc2nc(N3CCC[C@@](C)(O)C3)c3cc4c(c(F)c3n2)-c2cc(O)cc3ccc(F)c(c23)CCOCCN4C)C1]

Example 49: Prepared using a similar synthetic route as described in Example 46: LCMS (ESI): [M+H]⁺=690.3. ¹H NMR (400 MHz, CD₃OD) δ ppm 8.47 (br s, 1H), 7.91 (br d, J=12.1 Hz, 1H), 7.73 (dd, J=9.1, 6.2 Hz, 1H), 7.34-7.22 (m, 2H), 6.90 (br s, 1H), 5.54-5.32 (m, 1H), 5.08 (br s, 2H), 4.49-4.30 (m, 3H), 4.23-4.08 (m, 2H), 4.02 (br d, J=13.8 Hz, 1H), 3.78 (br d, J=13.7 Hz, 1H), 3.68-3.58 (m, 3H), 3.50-3.43 (m, 2H), 3.18-2.95 (m, 3H), 2.86 (br s, 1H), 2.76 (br d, J=11.7 Hz, 5H), 2.62-2.11 (m, 5H), 1.89-1.76 (m, 3H), 1.31 (d, J=6.7 Hz, 3H).

### Example 50: 3,15-Difluoro-13-(((2R,7aS)-2-fluorotetrahydro-1H-pyrazin-7a(5H)-yl)methoxy)-11-((R)-3-hydroxy-3-methylpiperidin-1-yl)-4,5,8,9-tetrahydro-7H-naphtho[1′,8′:7,8,9][1]oxa[4]azacycloundeca[5,6-g]quinazoline-17-yl methylcarbamate

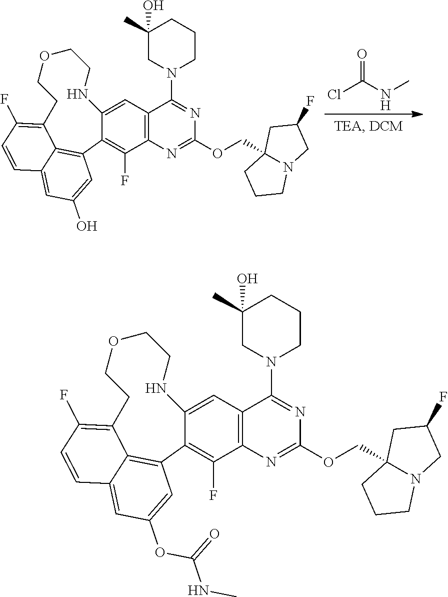

Step I: At 25° C., to a solution of 3,15-difluoro-13-(((2R,7aS)-2-fluoro-tetrahydro-1H-pyrazin-7a(5H)-yl)methoxy)-11-((R)-3-hydroxy-3-methylpiperidin-1-yl)-4,5,8,9-tetrahydro-7H-naphtho[1′,8′:7,8,9][1]oxa[4]azacycloundeca[5,6-g]quinazoline-17-ol (13 mg, 0.02 mmol) in dichloromethane (1300 μL) were added triethylamine (14 μL, 0.10 mmol) and methyl isocyanate (2.20 mg, 0.02 mmol). The mixture was stirred at 25° C. for 2 hours. The reaction mixture was diluted with water (1 mL) and extracted with dichloromethane (1 mL×3). The combined organic phases were dried over anhydrous sodium sulfate, filtered, and concentrated under reduced pressure. The resulting crude product was purified by preparative HPLC to give a yellow solid compound 3,15-difluoro-13-(((2R,7aS)-2-fluoro-tetrahydro-1H-pyrazin-7a(5H)-yl)methoxy)-11-((R)-3-hydroxy-3-methylpiperidin-1-yl)-4,5,8,9-tetrahydro-7H-naphtho[1′,8′:7,8,9][1]oxa[4]azacycloundeca[5,6-g]quinazoline-17-yl methylcarbamate (3.43 mg, 4.76 μmol, yield: 24%). LCMS (ESI): [M+H]⁺=721.3. ¹H NMR (400 MHz, CD₃OD) δ ppm 7.94 (dd, J=6.1, 8.9 Hz, 1H), 7.85-7.77 (m, 1H), 7.52 (s, 1H), 7.41 (br t, J=9.6 Hz, 1H), 7.22-7.13 (m, 1H), 5.41-5.20 (m, 1H), 4.37-4.28 (m, 1H), 4.26-4.17 (m, 2H), 4.09 (br d, J=13.3 Hz, 1H), 3.87-3.73 (m, 1H), 3.64-3.51 (m, 3H), 3.47-3.35 (m, 4H), 3.29-3.21 (m, 2H), 3.14-3.01 (m, 2H), 2.80 (s, 3H), 2.67 (br s, 2H), 2.43-1.71 (m, 10H), 1.29 (s, 3H).

### Example 51: 3,15-difluoro-11-((R)-3-hydroxy-3-methylpiperidin-1-yl)-9-methyl-13-(((S)-2-methylenetetrahydro-1H-pyrazin-7a(5H)-yl)methoxy)-4,5,8,9-tetrahydro-7H-naphtho[1′,8′:7,8,9111]oxa[4]azacycloundeca[5,6-g]quinazoline-17-yl decanoate

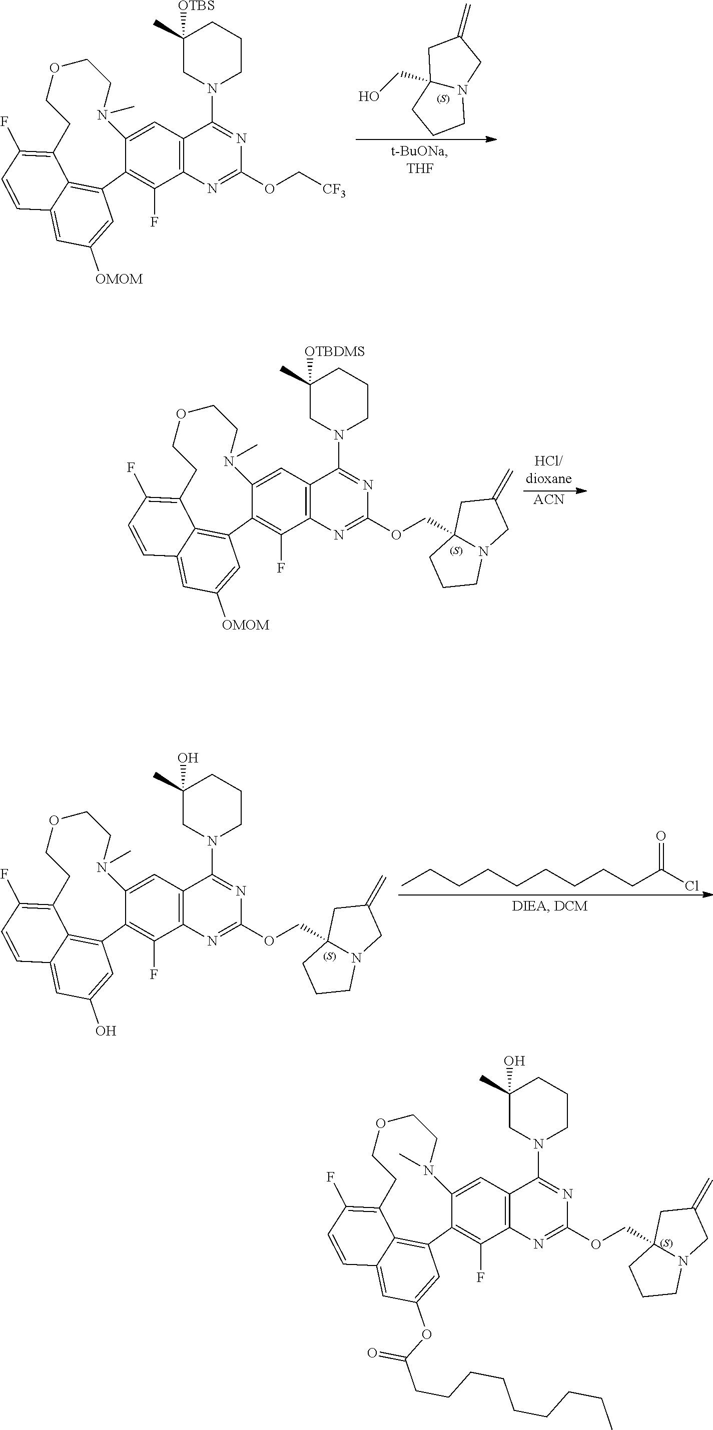

Step I: At room temperature, to a solution of (R)-11-(3-((tert-butyldimethylsilyl)oxy)-3-methylpiperidin-1-yl)-3,15-difluoro-17-(methoxymethoxy)-9-methyl-13-(2,2,2-trifluoroethoxy)-4,5,8,9-tetrahydro-7H-naphtho[1′,8′:7,8,9][1]oxa[4]azacycloundeca[5,6-g]quinazoline (500.0 mg, 0.68 mmol) and (S)-(2-methylene-tetrahydro-1H-pyrazin-7a(5H)-yl)methanol (209.0 mg, 1.36 mmol) in tetrahydrofuran (6.00 mL) were added 4A molecular sieves (600.0 mg) and sodium tert-butoxide (401.0 mg, 4.09 mmol). The solution was stirred at 60° C. for 16 hours. The reaction mixture was diluted with water (5 mL) and extracted with ethyl acetate (4 mL×3). The combined organic layers were dried over anhydrous sodium sulfate, filtered, and concentrated under reduced pressure. The residue was purified using flash column chromatography (silica gel, 0-20% gradient of methanol/dichloromethane) to give a yellow solid compound 11-((R)-3-((tert-butyldimethylsilyl)oxy)-3-methylpiperidin-1-yl)-3,15-difluoro-17-(methoxymethoxy)-9-methyl-13-(((S)-2-methylene-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline (400.0 mg, 0.48 mmol, yield: 70%). LCMS (ESI): [M+H]⁺=830.5.

Step II: To a solution of 11-((R)-3-((tert-butyldimethylsilyl)oxy)-3-methylpiperidin-1-yl)-3,15-difluoro-17-(methoxymethoxy)-9-methyl-13-(((S)-2-methylene-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline (150 mg, 0.18 mmol) in acetonitrile (2.00 mL) was added hydrogen chloride (4 M solution in dioxane, 4.06 mL, 16.2 mmol) was added. The reaction mixture was stirred at 20° C. for 16 hours. The solution was concentrated to give a compound 3,15-difluoro-11-((R)-3-hydroxy-3-methylpiperidin-1-yl)-9-methyl-13-(((S)-2-methylene-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-17-ol (150.0 mg, 0.22 mmol, yield: 123%) LCMS (ESI): [M+H]⁺=672.3.

Step III: At −40° C., to a solution of 3,15-difluoro-11-((R)-3-hydroxy-3-methylpiperidin-1-yl)-9-methyl-13-(((S)-2-methylene-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-17-ol (150 mg, 0.22 mmol) in dichloromethane (2.00 mL) were added diisopropylethylamine (116.0 μL, 0.66 mmol) and n-decanoyl chloride (57.0 μL, 0.29 mmol). The mixture was stirred at 20° C. for 0.5 hour. Water (3 mL) was added, and the reaction mixture was extracted with dichloromethane (1.5 mL×3). The combined organic phases were dried over anhydrous sodium sulfate, filtered, and concentrated under reduced pressure. The residue was purified by flash column chromatography (silica gel, 0-80% gradient of ethyl acetate/petroleum ether) to give a yellow solid compound (30.0 mg, 85% purity). The obtained compound was purified by preparative HPLC to give a yellow solid compound 3,15-difluoro-11-((R)-3-hydroxy-3-methylpiperidin-1-yl)-9-methyl-13-(((S)-2-methylene-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-17-yl decanoate (17.19 mg, 20.8 μmol, yield: 9%) LCMS (ESI): [M+H]⁺=826.5. ¹H NMR (400 MHz, CDCl3) δ ppm 7.78 (dd, J=8.6, 6.3 Hz, 1H), 7.65 (t, J=2.6 Hz, 1H), 7.59 (br d, J=16.5 Hz, 1H), 7.34-7.29 (m, 1H), 7.06 (dd, J=6.0, 2.5 Hz, 1H), 4.95 (br s, 2H), 4.35-4.14 (m, 4H), 3.84-3.55 (m, 3H), 3.50-3.21 (m, 6H), 3.20-3.04 (m, 3H), 2.94-2.73 (m, 6H), 2.70-2.54 (m, 5H), 2.41 (br d, J=15.9 Hz, 1H), 2.18 (td, J=12.0, 5.5 Hz, 1H), 2.07-1.87 (m, 7H), 1.80-1.71 (m, 5H), 1.69-1.57 (m, 2H), 1.44-1.33 (m, 7H), 0.88 (br t, J=6.7 Hz, 3H).

### Example 52: (R)-1-(3,15-Difluoro-13-(((2R,7aS)-2-fluoro-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-11-yl)-3-methylpiperidin-3-ol

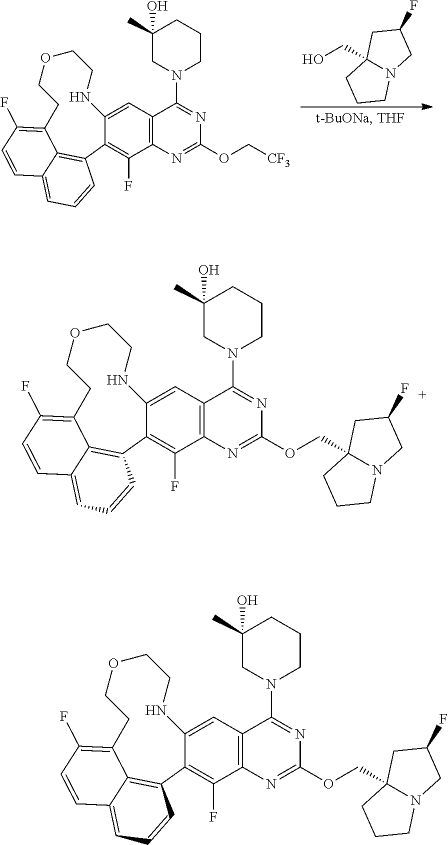

Step I: To a solution of (R)-1-(3,15-difluoro-13-(2,2,2-trifluoroethoxy)-4,5,8,9-tetrahydro-7H-naphtho[1′,8′:7,8,9][1]oxa[4]azacycloundeca[5,6-g]quinazoline-11-yl)-3-methylpiperidin-3-ol (20.0 mg, 0.03 mmol) and ((2R,7aS)-2-fluoro-tetrahydro-1H-pyrazin-7a(5H)-yl)methanol (10.8 mg, 0.07 mmol) in tetrahydrofuran (0.30 mL) was added sodium tert-butoxide (39.2 mg, 0.41 mmol). The mixture was stirred at 60° C. for 16 hours. After cooling to room temperature, water (0.5 mL) was added to dilute the solution. The mixture was extracted with ethyl acetate (0.5 mL×3), and the organic layers were combined, dried over anhydrous sodium sulfate, filtered, and concentrated under reduced pressure. The residue was purified by preparative HPLC to give two stereoisomers of the compound (R)-1-(3,15-difluoro-13-(((2R,7aS)-2-fluoro-tetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-1-yl)-3-methylpiperidin-3-ol. Isomer 1, Example 05-546A-100: The shorter retention time peak on HPLC, isolated as a yellow solid (1.99 mg, 3.07 μmol, yield: 9%). LCMS (ESI): [M+H]⁺=648.2.

Example 52A: LCMS (ESI): [M+H]⁺=648.3. ¹H NMR (400 MHz, CD₃OD) δ ppm 8.09 (d, J=7.2 Hz, 1H), 8.01 (dd, J=9.0, 6.1 Hz, 1H), 7.63-7.51 (m, 2H), 7.46-7.31 (m, 2H), 5.48-5.28 (m, 1H), 4.45-4.21 (m, 3H), 4.17-4.07 (m, 1H), 3.91-3.79 (m, 1H), 3.68-3.34 (m, 8H), 3.29-3.32 (m, 1H), 3.20-3.03 (m, 2H), 2.71 (br s, 2H), 2.50-2.05 (m, 6H), 2.02-1.71 (m, 4H), 1.32-1.25 (m, 3H).

Example 52B: LCMS (ESI): [M+H]⁺=648.3. ¹H NMR (400 MHz, CD₃OD) δ ppm 8.09 (d, J=7.3 Hz, 1H), 8.01 (dd, J=9.0, 6.2 Hz, 1H), 7.64-7.52 (m, 2H), 7.47-7.33 (m, 2H), 5.44-5.24 (m, 1H), 4.38-4.18 (m, 3H), 4.12 (br d, J=13.1 Hz, 1H), 3.85 (dt, J=12.6, 6.3 Hz, 1H), 3.66-3.34 (m, 8H), 3.29-3.22 (m, 1H), 3.15-3.01 (m, 2H), 2.71 (br d, J=3.9 Hz, 2H), 2.44-1.69 (m, 10H), 1.40-1.25 (m, 3H).

### Example 53: (R)-1-(3,15-difluoro-13-(((S)-2-methylenetetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-11-yl)-3-methylpiperidin-3-ol

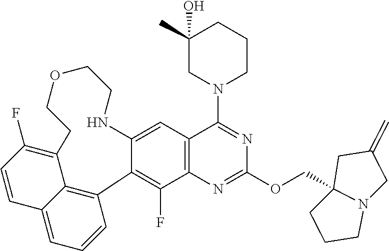 [SMILES: C=C1CN2CCC[C@@]2(COc2nc(N3CCC[C@@](C)(O)C3)c3cc4c(c(F)c3n2)-c2cccc3ccc(F)c(c23)CCOCCN4)C1]

Example 53: Prepared using a similar synthetic route as described in Example 52: LCMS (ESI): [M+H]⁺=642.3. ¹H NMR (400 MHz, CD₃OD) δ ppm 8.02 (m, 2H), 7.84-7.69 (m, 1H), 7.61-7.55 (m, 1H), 7.44-7.34 (m, 2H), 5.10 (br s, 2H), 4.48-4.06 (m, 3H), 4.03-3.71 (m, 2H), 3.69-3.37 (m, 6H), 3.32-3.03 (m, 3H), 2.98-2.85 (m, 1H), 2.81-2.47 (m, 3H), 2.35-1.63 (m, 10H), 1.30 (br d, J=8.7 Hz, 3H).

### Example 54: (R)-1-(3,15-difluoro-9-methyl-13-(((S)-2-methylenetetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-11-yl)-3-methylpiperidin-3-ol

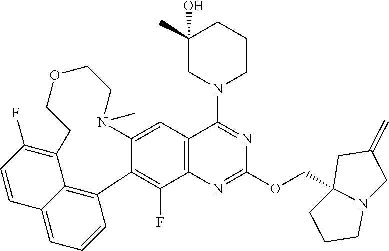 [SMILES: C=C1CN2CCC[C@@]2(COc2nc(N3CCC[C@@](C)(O)C3)c3cc4c(c(F)c3n2)-c2cccc3ccc(F)c(c23)CCOCCN4C)C1]

Example 54: Prepared using a similar synthetic route as described in Example 52: LCMS (ESI): [M+H]⁺=656.3. ¹H NMR (400 MHz, CD₃OD) δ ppm 8.08-7.83 (m, 3H), 7.50 (br t, J=7.7 Hz, 1H), 7.37 (br t, J=9.6 Hz, 1H), 7.29 (br d, J=6.9 Hz, 1H), 5.06 (br s, 2H), 4.48-4.24 (m, 3H), 4.20-4.10 (m, 1H), 3.91 (br d, J=12.8 Hz, 1H), 3.72-3.35 (m, 7H), 3.09-2.98 (m, 2H), 2.92-2.77 (m, 3H), 2.74-2.66 (m, 3H), 2.65-2.51 (m, 1H), 2.33-1.54 (m, 10H), 1.30 (br d, J=6.5 Hz, 3H).

### Example 55: 3,15-difluoro-13-(((2R,7aS)-2-fluorotetrahydro-1H-pyrazin-7a(5H)-yl)methoxy)-11-((R)-3-hydroxy-3-methylpiperidin-1-yl)-7-methyl-4,5,8,9-tetrahydro-7H-naphtho[1′,8′:7,8,9][1]oxa[4]azacycloundeca[5,6-g]quinazoline-17-ol

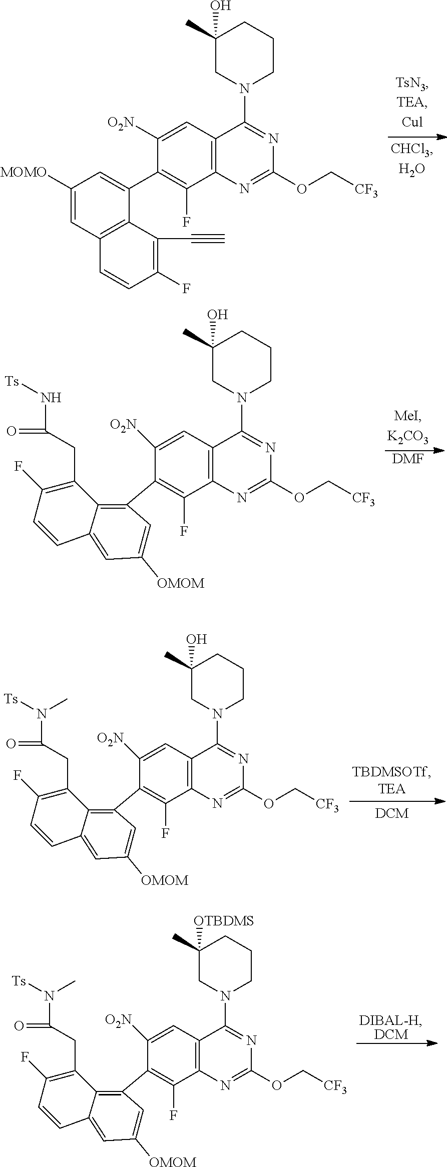

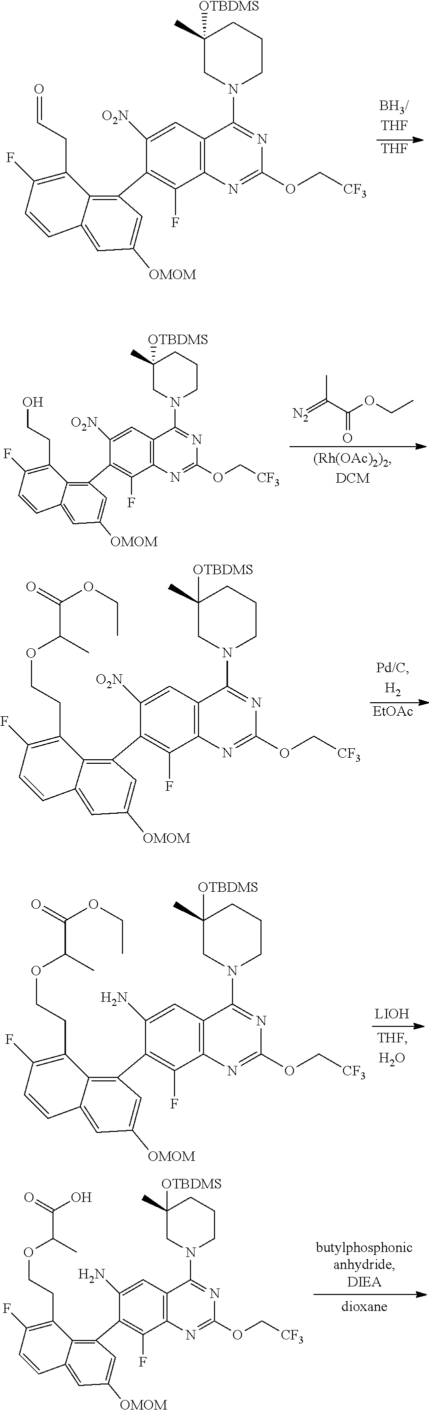

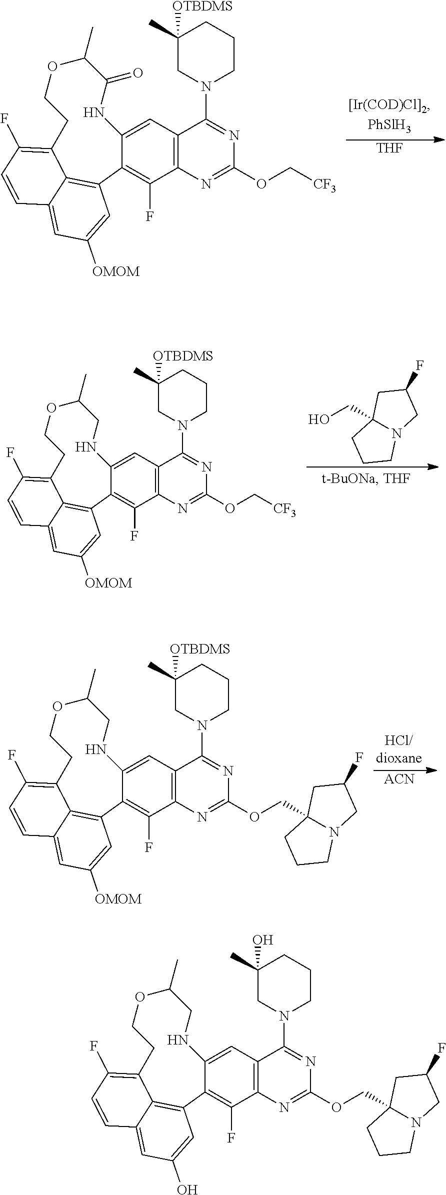

Step I: At 20° C. under a nitrogen atmosphere, a solution of (3R)-1-(7-(8-ethynyl-7-fluoro-3-(methoxymethoxy)naphthalen-1-yl)-8-fluoro-6-nitro-2-(2,2,2-trifluoroethoxy)quinazolin-4-yl)-3-methylpiperidin-3-ol (3.9 g, 6.17 mmol), 4-methylbenzenesulfonyl azide (1.95 g, 7.40 mmol), and copper(I) iodide (0.12 g, 0.62 mmol) in chloroform (50 mL) and water (0.28 mL, 15.41 mmol) was treated with triethylamine (1.03 mL, 7.40 mmol). The reaction mixture was stirred at 20° C. for 12 hours, then diluted with water (50 mL) and extracted with ethyl acetate (20 mL×3). The organic layers were combined, dried over anhydrous sodium sulfate, filtered, and concentrated under reduced pressure to give a crude compound 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-(p-toluenesulfonyl)acetamide (5.0 g) as a brown solid. LCMS (ESI): [M+H]⁺=820.0.

Step II: At 20° C., a solution of 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-(p-toluenesulfonyl)acetamide (5.4 g, 6.59 mmol) in N,N-dimethylformamide (50 mL) was treated with iodomethane (1.23 mL, 19.76 mmol) and potassium carbonate (2.73 g, 19.76 mmol). The reaction mixture was stirred at 20° C. for 12 hours, then diluted with water (100 mL) and extracted with ethyl acetate (50 mL×3). The organic layers were combined, dried over anhydrous sodium sulfate, filtered, and concentrated under reduced pressure. The residue was purified by flash column chromatography (silica gel, 0-30% gradient of tetrahydrofuran/petroleum ether) to give a brown solid compound 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-methyl-N-(p-toluenesulfonyl)acetamide (2.38 g, 2.85 mmol, yield: 43%). LCMS (ESI): [M+H]⁺=834.2.

Step III: At 0° C., a solution of 2-(2-fluoro-8-(8-fluoro-4-((R)-3-hydroxy-3-methylpiperidin-1-yl)-6-nitro-2-(2,2,2-trifluoroethoxy)quinazolin-7-yl)-6-(methoxymethoxy)naphthalen-1-yl)-N-methyl-N-(p-toluenesulfonyl)acetamide (11.5 g, 13.79 mmol) and triethylamine (9.59 mL, 68.96 mmol) in dichloromethane (140 mL) was treated dropwise with tert-butyldimethylsilyl trifluoromethanesulfonate (5.20 mL, 41.38 mmol). The reaction mixture was stirred at 20° C. for 16 hours. and the reaction mixture was diluted with water (40 mL), extracted with dichloromethane (40 mL×3). The combined organic phases were dried over anhydrous sodium sulfate, filtered, and concentrated under reduced pressure. The residue was purified using flash column chromatography (silica gel, 0-5% gradient of tetrahydrofuran in a mixture of petroleum ether and dichloromethane at a 3:1 ratio), to give a brown solid compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)-N-methyl-N-(p-toluenesulfonyl)acetamide (12.7 g, 13.41 mmol, yield: 97%). LCMS (ESI): [M+H]⁺=948.4.

Step IV: At −70° C., a solution of 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)-N-methyl-N-(p-toluenesulfonyl)acetamide (12.50 g, 13.19 mmol) in dichloromethane (155 mL) was treated with 1 M diisobutylaluminum hydride (42.19 mL, 42.19 mmol). The reaction mixture was stirred at −70° C. for 1 hour, then quenched with acetic acid (7.25 mL, 126.58 mmol). Water (100 mL) was added to dilute the mixture, followed by stirring and standing for 16 hours. The reaction mixture was extracted with dichloromethane (100 mL×3). The combined organic phases were dried over anhydrous sodium sulfate, filtered, and concentrated under reduced pressure. The residue was purified by flash column chromatography (silica gel, 0-20% gradient of tetrahydrofuran/(petroleum ether:dichloromethane=3:1)) to give a compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)acetaldehyde (7.70 g, 10.08 mmol, yield: 76%) as a brown solid. LCMS (ESI): [M+H]⁺=765.3.

Step V: At 0° C., borane/tetrahydrofuran (1 M, 41 mL, 40.71 mmol) was added to a solution of 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)acetaldehyde (18.50 g, 24.19 mmol) in tetrahydrofuran (190 mL). The reaction mixture was stirred at 10° C. for 16 hours. The reaction was quenched with methanol at 0° C. The mixture was concentrated under reduced pressure to give a brown solid compound 2-(8-(4-((R)-3-((tert-butyldimethylsilyloxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethanol (18.50 g, 21.71 mmol, yield: 90%). LCMS (ESI): [M+H]⁺=767.8.

Step VI: To the solution of 2-(8-(4-((R)-3-((tert-butyldimethylsilyl)oxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethan-1-ol (1.00 g, 1.30 mmol) and rhodium acetate dimer (28.8 mg, 0.07 mmol) in dichloromethane (10.0 mL) was added ethyl 2-diazopropionate (752 mg, 5.87 mmol) dropwise under a nitrogen atmosphere. The mixture was stirred at 25° C. for 1 hour under a nitrogen atmosphere. Methanol (5 mL) was added to quench the reaction, and the solvent was removed under reduced pressure. The residue was purified by flash column chromatography (silica gel, 0-10% gradient of tetrahydrofuran/petroleum ether) to give a yellow oily crude compound ethyl 2-(2-(8-(4-((R)-3-((tert-butyldimethylsilyl)oxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethoxy)propanoate (800 mg, 0.92 mmol, yield: 71%). LCMS (ESI): [M+H]⁺=867.3.

Step VII: To the solution of ethyl 2-(2-(8-(4-((R)-3-((tert-butyldimethylsilyl)oxy)-3-methylpiperidin-1-yl)-8-fluoro-6-nitro-2-(2,2,2-trifluoroethoxy)quinazolin-7-yl)-2-fluoro-6-(methoxymethoxy)naphthalen-1-yl)ethoxy)propionate (1.34 g, 1.55 mmol) in ethyl acetate (60 mL) was added palladium on carbon (wet) (300 mg). The mixture was stirred under hydrogen atmosphere (45 psi) at 20° C. for 16 hours. The reaction mixture was then filtered, and the filtrate was concentrated under reduced pressure to give a yellow oily compound ethyl 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)propanoate (1.28 g, 1.53 mmol, yield: 99%). LCMS (ESI): [M+H]⁺=837.3.

Step VIII: To a solution of ethyl 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)propionate (1.28 g, 1.53 mmol) in tetrahydrofuran (25.0 mL) and water (2.5 mL) was added lithium hydroxide (385 mg, 9.18 mmol). The mixture was stirred at 60° C. for 5 hours, and the pH was adjusted to 5-6 using 1 M hydrochloric acid solution. The mixture was extracted with ethyl acetate (10 mL×3), and the combined organic layers were dried over anhydrous sodium sulfate, filtered, and concentrated under reduced pressure to give a yellow solid compound 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)propanoic acid (1.23 g, 1.52 mmol, yield: 99%). LCMS (ESI): [M+H]⁺=809.3.

Step IX: To the solution of 2-(2-(8-(6-amino-4-((R)-3-((tert-butyldimethylsilyl)oxy)-3-methylpiperidin-1-yl)-8-fluoro-2-(2,2,2-trifluoroethoxy)quinazolin-7-yl)-2-fluoro-6-(methoxymethoxy)naphthalen-1-yl)ethoxy)propanoic acid (1.23 g, 1.52 mmol) and diisopropylethylamine (10.6 mL, 60.82 mmol) in dioxane (49.0 mL) was added butylphosphonic anhydride (50% ethyl acetate solution, 3.29 g, 4.56 mmol). The reaction mixture was stirred at 60° C. for 16 hours. The reaction mixture was cooled to room temperature, and water (20 mL) was added to the reaction solution. The mixture was extracted with ethyl acetate (20 mL×3), and the organic layers were combined, dried over anhydrous sodium sulfate, and filtered. The filtrate was concentrated to give a brown solid compound 11-((R)-3-((tert-butyldimethylsilyl)oxy)-3-methylpiperidin-1-yl)-3,15-difluoro-17-(methoxymethoxy)-7-methyl-13-(2,2,2-trifluoroethoxy)-4,5-dihydro-7H-naphtho[1′,8′:7,8,9][1]oxa[4]azacycloundeca[5,6-g]quinazoline-8(9H)-one (1.2 g, 1.52 mmol, yield: 100%). LCMS (ESI): [M+H]⁺=791.3.

Step X: To a solution of 11-((R)-3-((tert-butyldimethylsilyl)oxy)-3-methylpiperidin-1-yl)-3,15-difluoro-17-(methoxymethoxy)-7-methyl-13-(2,2,2-trifluoroethoxy)-4,5-dihydro-7H-naphtho[1′,8′:7,8,9][1]oxa[4]azacycloundeca[5,6-g]quinazoline-8(9H)-one (1.2 g, 1.52 mmol) and bis(1,5-cyclooctadiene)chloroiridium(I) dimer (102 mg, 0.15 mmol) in tetrahydrofuran (84 mL) was added phenylsilane (3.74 mL, 30.35 mmol) under a nitrogen atmosphere. The mixture was stirred at 25° C. for 16 hours. then concentrated under reduced pressure. The residue was purified by flash chromatography on silica gel (0-30% gradient of tetrahydrofuran/(3:1 petroleum ether/dichloromethane)) to afford a yellow solid, 11-(R)-(3-((tert-butyldimethylsilyl)oxy)-3-methylpiperidin-1-yl)-3,15-difluoro-17-(methoxymethoxy)-7-methyl-13-(2,2,2-trifluoroethoxy)-4,5,8,9-tetrahydro-7H-naphtho[1′,8′:7,8,9][1]oxa[4]azacycloundeca[5,6-g]quinazoline (520 mg, 0.67 mmol, yield: 44%). LCMS (ESI): [M+H]⁺=777.3.

Step XI: To a solution of 11-((R)-3-((tert-butyldimethylsilyl)oxy)-3-methylpiperidin-1-yl)-3,15-difluoro-17-(methoxymethoxy)-7-methyl-13-(2,2,2-trifluoroethoxy)-4,5,8,9-tetrahydro-7H-naphtho[1′,8′:7,8,9][1]oxa[4]azacycloundeca[5,6-g]quinazoline (460 mg, 0.59 mmol), 4A molecular sieves, and ((2R,7aS)-2-fluorotetrahydro-1H-pyrazin-7a(5H)-yl)methanol (160 mg, 1.01 mmol) in tetrahydrofuran (5.00 mL) was added sodium tert-butoxide (569 mg, 5.92 mmol). The mixture was stirred at 60° C. for 16 hours. After cooling to room temperature, water (3 mL) was added to dilute the solution. The mixture was extracted with ethyl acetate (3 mL×3), and the organic layers were combined, dried over anhydrous sodium sulfate, filtered, and concentrated under reduced pressure. The residue was purified by flash column chromatography (silica gel, 0-10% methanol/dichloromethane gradient) to give a brown solid compound 11-((R)-3-((tert-butyldimethylsilyl)oxy)-3-methylpiperidin-1-yl)-3,15-difluoro-13-(((2R,7aS)-2-fluorotetrahydro-1H-pyrazin-7a(5H)-yl)methoxy)-17-(methoxymethoxy)-7-methyl-4,5,8,9-tetrahydro-7H-naphtho[1′,8′:7,8,9][1]oxa[4]azacycloundeca[5,6-g]quinazoline (380 mg, 0.59 mmol, yield: 77%). LCMS (ESI): [M+H]⁺=836.7.

Step XII: To the solution of 11-((R)-3-((tert-butyldimethylsilyl)oxy)-3-methylpiperidin-1-yl)-3,15-difluoro-13-(((2R,7aS)-2-fluorotetrahydro-1H-pyrazin-7a(5H)-yl)methoxy)-17-(methoxymethoxy)-7-methyl-4,5,8,9-tetrahydro-7H-naphtho[1′,8′:7,8,9][1]oxa[4]azacycloundeca[5,6-g]quinazoline (380 mg, 0.45 mmol) in acetonitrile (3.00 mL) was added hydrogen chloride (2 M solution in dioxane, 27.3 mL, 54.54 mmol). The mixture was stirred at 25° C. for 16 hours. The reaction mixture was concentrated under reduced pressure. The residue was dissolved in acetonitrile (1 mL) and neutralized with triethylamine (until pH>7). The solvent was removed under reduced pressure. The residue was purified by preparative HPLC to give a yellow solid compound 3,15-Difluoro-13-(((2R,7aS)-2-fluorotetrahydro-1H-pyrazin-7a(5H)-yl)methoxy)-11-((R)-3-hydroxy-3-methylpiperidin-1-yl)-7-methyl-4,5,8,9-tetrahydro-7H-naphtho[1′,8′:7,8,9][1]oxa[4]azacycloundeca[5,6-g]quinazoline-17-ol (60.0 mg, 0.09 mmol, yield: 19%). LCMS (ESI): [M+H]⁺=678.2. ¹H NMR (400 MHz, CD₃OD) δ ppm 7.76 (dd, J=6.2, 8.7 Hz, 1H), 7.59-7.39 (m, 1H), 7.36-7.22 (m, 2H), 7.05-6.90 (m, 1H), 5.42-5.24 (m, 1H), 4.43-3.99 (m, 4H), 3.92-3.80 (m, 1H), 3.73 (br s, 1H), 3.64-3.34 (m, 3H), 3.31-3.23 (m, 3H), 3.09-3.03 (m, 1H), 2.93-2.52 (m, 4H), 2.43-2.10 (m, 4H), 2.07-1.84 (m, 4H), 1.83-1.70 (m, 2H), 1.31 (s, 3H), 1.10-0.91 (m, 3H).

### Example 56: (R)-1-(17-amino-3,15-difluoro-9-methyl-13-(((S)-2-methylenetetrahydro-1H-pyrazin-7a(5H)-yl)methoxy)-4,5,8,9-tetrahydro-7H-naphtho[1′,8′:7,8,9][1]oxa[4]azacycloundeca[5,6-g]quinazoline-11-yl)-3-methylpiperidin-3-ol

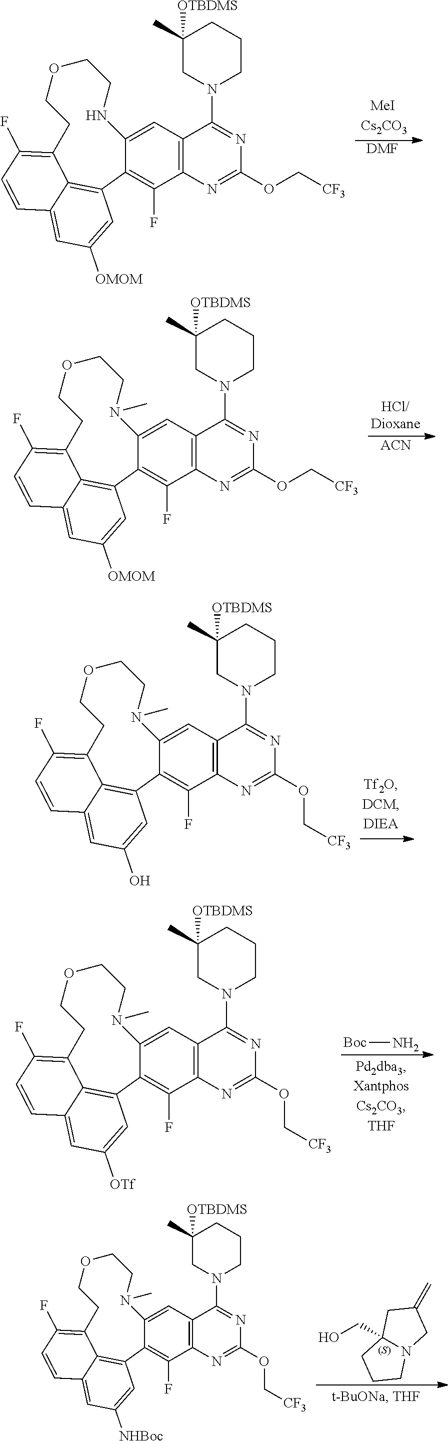

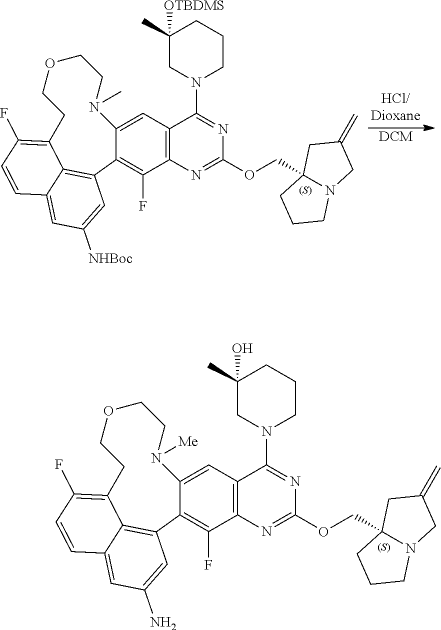

Step I: To a solution of (R)-11-(3-((tert-butyldimethylsilyl)oxy)-3-methylpiperidin-1-yl)-3,15-difluoro-17-(methoxymethoxy)-13-(2,2,2-trifluoroethoxy)-4,5,8,9-tetrahydro-7H-naphtho[1′,8′:7,8,9][1]oxa[4]azacycloundeca[5,6-g]quinazoline (500 mg, 0.66 mmol) in N,N-dimethylformamide (10 mL) were added iodomethane (81.0 μL, 1.32 mmol) and cesium carbonate (235 mg, 0.72 mmol). The reaction mixture was stirred at 25° C. for 16 hours. then diluted with water (3 mL) and extracted with ethyl acetate (5 mL×3). The organic layers were combined, dried over anhydrous sodium sulfate, filtered, and concentrated under reduced pressure. The residue was purified by flash column chromatography on silica gel (gradient elution: 0-15% tetrahydrofuran in a mixture of petroleum ether and dichloromethane in a 3:1 ratio) to give a yellow solid compound (R)-11-(3-((tert-butyldimethylsilyloxy)-3-methylpiperidin-1-yl)-3,15-difluoro-17-(methoxymethoxy)-9-methyl-13-(2,2,2-trifluoroethoxy)-4,5,8,9-tetrahydro-7H-naphtho[1′,8′:7,8,9][1]oxa[4]azacycloundeca[5,6-g]quinazoline (460 mg, 0.59 mmol, yield: 90%). LCMS (ESI): [M+H]⁺=777.3.

Step II: To the solution of (R)-11-(3-((tert-butyldimethylsilyl)oxy)-3-methylpiperidin-1-yl)-3,15-difluoro-17-(methoxymethoxy)-9-methyl-13-(2,2,2-trifluoroethoxy)-4,5,8,9-tetrahydro-7H-naphtho[1′,8′:7,8,9][1]oxa[4]azacycloundeca[5,6-g]quinazoline (460 mg, 0.59 mmol) in acetonitrile (8.00 mL) was added 4 M hydrochloric acid in dioxane (1.04 mL, 4.14 mmol). The reaction mixture was stirred at 20° C. for 16 hours. The reaction mixture was concentrated under reduced pressure to give a brown solid compound (R)-11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-ol (430 mg, 0.59 mmol, yield: 99%). LCMS (ESI): [M+H]⁺=733.2.

Step III: To a solution of the compound (R)-11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-ol (135 mg, 0.18 mmol) in dichloromethane (1.35 mL) was added diisopropylethylamine (98 μL, 0.55 mmol). The mixture was cooled to −78° C., and trifluoromethanesulfonic anhydride (46.5 μL, 0.28 mmol) was slowly added. The reaction was stirred at −70° C. for 4 hours. Upon completion of the reaction, the mixture was quenched with water (1 mL) and extracted with dichloromethane (1 mL×3). The combined organic layers were dried over anhydrous sodium sulfate, filtered, and concentrated to give a crude yellow solid compound (R)-11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-yl trifluoromethanesulfonate (195 mg). LCMS (ESI): [M+H]⁺=865.2.

Step IV: Under a nitrogen atmosphere, a solution of (R)-11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-yl trifluoromethanesulfonate (180 mg, 0.21 mmol) in tetrahydrofuran (1.80 mL) was combined with tert-butyl carbamate (34.1 mg, 0.29 mmol), cesium carbonate (203 mg, 0.62 mmol), 4,5-bis(diphenylphosphino)-9,9-dimethylxanthene (12.0 mg, 0.02 mmol), and tris(dibenzylideneacetone)dipalladium (19.1 mg, 0.02 mmol). The mixture was stirred at 70° C. for 2 h. After naturally cooling to room temperature, the reaction mixture was concentrated under reduced pressure. The residue was purified by flash column chromatography (silica gel, gradient elution with 0-15% tetrahydrofuran/petroleum ether-dichloromethane (3:1, v/v)) to give a green solid compound tert-butyl (R)-(11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-yl)carbamate (144 mg, 0.17 mmol, yield: 83%). LCMS (ESI): [M+H]⁺=832.4.

Step V: To a solution of tert-butyl (R)-(11-(3-((tert-butyldimethylsilyl)oxy)-3-methylpiperidin-1-yl)-3,15-difluoro-9-methyl-13-(2,2,2-trifluoroethoxy)-4,5,8,9-tetrahydro-7H-benzo[1′,8′:7,8,9][1]oxa[4]azacycloundeca[5,6-g]quinazoline-17-yl)carbamate (70.0 mg, 0.08 mmol) and (S)-(2-methylenetetrahydro-1H-pyrazin-7a(5H)-yl)methanol (21.9 mg, 0.14 mmol) in tetrahydrofuran (1.00 mL) were added 4A molecular sieves (50 mg) and sodium tert-butoxide (66.1 mg, 0.67 mmol). The mixture was stirred at 60° C. for 16 h. After naturally cooling to room temperature, the reaction mixture was diluted with water (1 mL) and extracted with ethyl acetate (1 mL×3). The combined organic layers were dried over anhydrous sodium sulfate, filtered, and concentrated under reduced pressure. The residue was purified by flash column chromatography (silica gel, gradient elution with 0-10% methanol/dichloromethane) to give a brown solid compound tert-butyl (11-((R)-3-((tert-butyldimethylsilyl)oxy)-3-methylpiperidin-1-yl)-3,15-difluoro-9-methyl-13-((S)-2-methylenetetrahydro-1H-pyrazin-7a(5H)-yl)methoxy)-4,5,8,9-tetrahydro-7H-benzo[1′,8′:7,8,9][1]oxa[4]azacycloundeca[5,6-g]quinazoline-17-yl)carbamate (20.0 mg, 0.02 mmol, yield: 27%). LCMS (ESI): [M+H]⁺=885.5.

Step VI: To a solution of tert-butyl (11-((R)-3-((tert-butyldimethylsilyl)oxy)-3-methylpiperidin-1-yl)-3,15-difluoro-9-methyl-13-((S)-2-methylenetetrahydro-1H-pyrazin-7a(5H)-yl)methoxy)-4,5,8,9-tetrahydro-7H-benzo[1′,8′:7,8,9][1]oxa[4]azacycloundeca[5,6-g]quinazoline-17-yl)carbamate (20.0 mg, 0.02 mmol) in dichloromethane (200 μL) was added hydrochloric acid (4 M solution in dioxane, 339 μL, 1.36 mmol). The mixture was stirred at 25° C. for 16 hours. The reaction mixture was then concentrated under vacuum, and the residue was dissolved in dichloromethane (1 mL), neutralized with triethylamine (until pH>7), and concentrated under reduced pressure to remove the solvent. The residue was purified by preparative HPLC to give a yellow solid compound (R)-1-(17-amino-3,15-difluoro-9-methyl-13-(((S)-2-methylenetetrahydro-1H-pyrazin-7a(5H)-yl)methoxy)-4,5,8,9-tetrahydro-7H-benzo[1′,8′:7,8,9][1]oxa[4]azacycloundeca[5,6-g]quinazoline-11-yl)-3-methylpiperidin-3-ol (formate salt, 7.58 mg, 11.30 μmol, yield: 50%). LCMS (ESI): [M+H]⁺=671.2. 1H NMR (400 MHz, CD₃OD) δ ppm 8.54 (s, 1H), 7.85 (d, J=9.2 Hz, 1H), 7.62 (dd, J=6.1, 9.0 Hz, 1H), 7.35-7.08 (m, 2H), 6.84 (d, J=2.3 Hz, 1H), 5.11 (br s, 2H), 4.61-4.26 (m, 3H), 4.15 (br t, J=14.1 Hz, 1H), 4.00 (br d, J=13.6 Hz, 1H), 3.72-3.47 (m, 4H), 3.46-3.35 (m, 4H), 3.18-3.07 (m, 1H), 3.05-2.85 (m, 3H), 2.84-2.66 (m, 5H), 2.61 (br d, J=16.3 Hz, 1H), 2.28-1.72 (m, 8H), 1.30 (d, J=5.1 Hz, 3H).

### Example 57: 13-((2,2-difluoro-6-methylenetetrahydro-1H-pyrazin-7a(5H)-yl)methoxy)-3,15-difluoro-11-((R)-3-hydroxy-3-methylpiperidin-1-yl)-4,5,6,7,8,9-hexahydronaphtho[1′,8′:4,5,6][1]azacycloundecano[2,3-g]quinazolin-17-ol

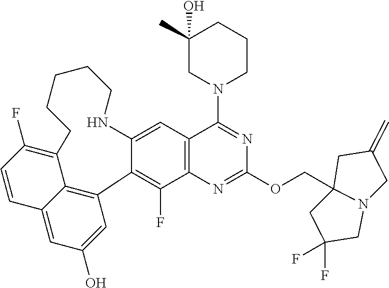 [SMILES: C=C1CN2CC(F)(F)CC2(COc2nc(N3CCC[C@@](C)(O)C3)c3cc4c(c(F)c3n2)-c2cc(O)cc3ccc(F)c(c23)CCCCCN4)C1]

Example 57: Prepared using a similar synthetic route as described in Example 16: LCMS (ESI): [M+H]⁺=692.7. ¹H NMR (400 MHz, CD₃OD) δ ppm 7.61-7.53 (m, 1H), 7.28-7.07 (m, 3H), 6.83 (d, J=2.6 Hz, 1H), 4.92 (br d, J=3.8 Hz, 2H), 4.23-4.06 (m, 3H), 3.97 (br d, J=13.1 Hz, 1H), 3.65 (br t, J=14.6 Hz, 2H), 3.41-3.32 (m, 2H), 3.30-3.24 (m, 1H), 3.10-2.98 (m, 1H), 2.93-2.81 (m, 1H), 2.75-2.67 (m, 1H), 2.60-2.46 (m, 3H), 2.31 (q, J=14.4 Hz, 2H), 2.14-2.00 (m, 1H), 1.80-1.60 (m, 4H), 1.55-1.38 (m, 2H), 1.35-1.16 (m, 6H), 0.95-0.84 (m, 1H).

### Effect Example 1: AlphaLISA Compound Screening Assay

### Experimental Method

### 5. Preparation of Assay Buffers:

Buffer 1: Composition and final concentrations: 25 mM HEPES, pH 7.5; 10 mM MgCl₂; 0.01% Triton X-100.
Buffer 2: Composition and final concentrations: 25 mM HEPES, pH 7.5; 10 mM MgCl₂; 0.01% Triton X-100; 1 mM DTT.

### 6. Experimental Design and Compound Preparation:

4> Use the ECHO555 automated liquid handler to prepare compounds. Test concentrations: The maximum compound concentration is 10 μM or 1 μM, serially diluted 3-fold, with 10 concentrations tested. Each concentration is tested in duplicate, with 0.5% DMSO as the solvent.
5> High Signal Control Group (High Control): 30 replicates with 0.5% DMSO. KRAS protein is included in the reaction, representing 0% inhibition
6> Low Signal Control Group (Low Control): 30 replicates with 0.5% DMSO. KRAS protein is not involved in the reaction, representing 100% inhibition

### 7. Experimental Procedure:

d. KRAS-GDP Protein Assay Procedure
6> Add 5 μL of KRAS solution prepared with Buffer 1 to each well of the high-signal control group and compound test wells. Add 5 μL of Buffer 1 to each well of the low-signal control group. The final concentration of KRAS in the reaction is 5 nM (initially 40 nM, optimized to 5 nM). Incubate at room temperature for 1 hour.
7> Add 5 μL of a solution containing SOS1 and GTP prepared with Buffer 2 to each well. Final concentrations in the reaction: 50 nM for SOS1 (initially 250 nM) and 125 μM for GTP (initially 625 μM). Incubate at room temperature for 1 hour.
8> Add 5 μL of a solution containing cRAF and AlphaLISA Nickel Acceptor beads prepared with Buffer 2 to each well. Final concentrations in the reaction: 50 nM for cRAF and 20 μg/mL for AlphaLISA Nickel Acceptor beads. Incubate at room temperature for 1 hour.
9> Add 5 μL of an AlphaScreen GSH Donor beads solution prepared with Buffer 2 to each well. Final concentration in the reaction: 20 μg/mL. Incubate at room temperature for 1 hour.
10> Measure the signal values using an EnVision plate reader. Excitation wavelength: 680 nm, Emission wavelength: 615 nm.
   e. KRAS-GTP Protein Assay Procedure
6> Mix the prepared protein solution with the SOS1-GTP solution (at the same concentration as used for the GDP protein assay) in a 1:1 volume ratio. Pre-incubate the mixture at room temperature for 1 hour.
7> Add 10 μL of the mixture from Step I to each well of the high-signal control group and compound test wells. For the low-signal control group, add 5 μL of Buffer 1 and 5 μL of the SOS1-GTP mixture to each well. Incubate at room temperature for 1 hour.
8> Add 5 μL of a solution containing cRAF and AlphaLISA Nickel Acceptor beads prepared with Buffer 2 to each well. Final concentrations in the reaction: 50 nM for cRAF and 20 μg/mL for AlphaLISA Nickel Acceptor beads. Incubate at room temperature for 1 hour.
9> Add 5 μL of an AlphaScreen GSH Donor beads solution prepared with Buffer 2 to each well. Final concentration in the reaction: 20 μg/mL. Incubate at room temperature for 1 hour.
10> Measure the signal values using an EnVision plate reader. Excitation wavelength: 680 nm, Emission wavelength: 615 nm.
   f. KRAS-GCP Protein Assay Procedure
6> Add 5 μL of KRAS solution prepared with Buffer 1 to each well of the high-signal control group and compound test wells; add 5 μL of KRAS solution prepared with Buffer 1 to each well of the low-signal control group and compound test wells. The final concentration of KRAS in the reaction is 5 nM. Incubate at room temperature for 1 hour.
7> Add 5 μL of Buffer 2 to each well. Incubate at room temperature for 1 hour.
8> Add 5 μL of a solution containing cRAF and AlphaLISA Nickel Acceptor beads prepared with Buffer 2 to each well. Final concentrations in the reaction: 50 nM for cRAF and 20 μg/mL for AlphaLISA Nickel Acceptor beads. Incubate at room temperature for 1 hour.
9> Add 5 μL of an AlphaScreen GSH Donor beads solution prepared with Buffer 2 to each well. Final concentration in the reaction: 20 μg/mL. Incubate at room temperature for 1 hour.
10> Measure the signal values using an EnVision plate reader. Excitation wavelength: 680 nm, Emission wavelength: 615 nm.

### 8. Experimental DataAnalysis

4> Compute the average signal values for the high-signal control group (High control average) and the low-signal control group (Low control average) based on the raw data.
5> The inhibition rate (% inhibition) for each compound sample well is calculated using the following formula:

(High control average−Sample signal/High control average−Low control average)*100%
6> Use the four-parameter logistic regression model (log(inhibitor) vs. response—Variable slope (four parameters)) in Graphad Prism software to fit the dose-response curve based on the inhibition rates of the compound at different concentrations. Determine the IC₅₀ value for the compound.

### Experimental Materials

| Experimental Reagents | Company | Cat No. |
|---|---|---|
| 1M HEPES pH 7.5 | TEKNOVA | H1035 |
| 2M MgCl₂ | Sigma | 7786-30-3 |
| Triton X-100 | Aladdin | 9002-93-1 |
| DTT 1M | Invitrogen | P2325 |
| GTP | Thermo | R0461 |
| AlphaScreen GSH Donor beads | Perkin Elmer | 6765301 |
| AlphaLISA Nickel Acceptor beads | Perkin Elmer | AL108M |
| DMSO | Sigma | D2650 |
| 384-well opaque white plate | Perkin Elmer | 6007290 |
| His-tag-KRAS G12D | WuXi AppTec | In house |
| SOS1 | WuXi AppTec | In house |
| GST- tag-cRAF | WuXi AppTec | In house |
| sealing film | Axygen | PCR-AS-200 |

### Experimental Instruments

| Instruments | Company |
|---|---|
| Lab Water Purification Systems | MILLIPORE |
| Envision Multible Reader | Perkin Elmer |
| ECHO555 | Labcyte |
| Centrifuge 5810R | Eppendorf |

### Experimental Results

The inhibitory effects of the compounds described in the present invention on KRAS G12D GDP, GTP, and GCP proteins are summarized in Table 1.

| | GDP-format | GTP-format | GCP-format |
|---|---|---|---|
| | IC₅₀ (nM)/ | IC₅₀ (nM)/ | IC₅₀ (nM)/ |
| Example | Binding assay | Binding assay | Binding assay |
| 2 | 2.87 | 3.23 | >1000 |
| 3 | 2.31 | 3.24 | >1000 |
| 4 | 2.2 | 3.5 | 94.43 |
| 5 | 2.06 | 2.63 | 232.5 |
| 9B | 1.6 | 1.95 | 58.51 |
| 11 | 500.3 | 725.7 | 909.2 |

### Effect Example 2: ERK Phosphorylation Cell Assay

### AGS_ERK Protein Phosphorylation Detection

Day 1: AGS cells were seeded into a 384-well cell culture plate and incubated overnight at 37° C. with 5% CO₂ in a cell culture incubator.
Day 2: Compounds were added to the plate using an Echo550 liquid handler, followed by incubation at 37° C. with 5% CO₂ in a cell culture incubator for 3 hours. Subsequently, the cells were fixed with paraformaldehyde, permeabilized with methanol, and blocked with blocking buffer. A primary antibody mixture (rabbit anti-pERK, mouse anti-GAPDH) was added, and the plate was incubated overnight at 4° C.
Day 3: The primary antibodies were removed, and a secondary antibody mixture (goat anti-rabbit 800CW, goat anti-mouse 680RD) was added. The plate was incubated at room temperature in the dark. After three washes with PBST, the plate was inverted and centrifuged for 1 minute. Fluorescence signal values were read using an Odyssey CLx imaging system.

### Calculation of ERK Phosphorylation:

$$Relative Expression Level : ( Compound Fluorescence Signal Ratio - Positive Control Mean ) / Negative Control Mean - Positive Control Mean )$$

Negative control: DMSO
Positive control: 10 μM Reference
Fitted using XLFit 5.0 with the following parameters:

$$Y = Bottom + ( Top - Bottom ) / ( 1 + {10}^{⋀} ( ( Log {IC}_{50} - X ) \times HillSlope ) )$$

X: Log of compound concentration
Y: Mean relative expression levels of p-ERK across replicates

The inhibitory effects of the compounds described in the present invention on pERK in AGS and AsPC-1 cells are summarized in Table 2.

| | pERK | pERK |
|---|---|---|
| | AGS_IC₅₀ | AsPC-1_IC₅₀ |
| Example | (nM) | (nM) |
| 1 | 2152 | 2454 |
| 2 | 154 | 710 |
| 3 | 47 | 104 |
| 4 | 330.56 | |
| 5 | 12 | 41 |
| 6A | 30 | |
| 6B | 450 | |
| 7 | 3468 | |
| 8 | 73 | 278 |
| 9 | 18 | 118 |
| 9A | 687 | 1960 |
| 9B | 17 | 70 |
| 10 | >10000 | >10000 |
| 12 | 5433 | >10000 |
| 13 | 3954 | 8044 |
| 14 | 2769 | 3843 |
| 15 | >10000 | |
| 16 | 31 | 102 |
| 17A | 5268 | >10000 |
| 17B | >10000 | >10000 |
| 18A | >10000 | >10000 |
| 18B | >10000 | >10000 |
| 19 | >10000 | >10000 |
| 20A | >10000 | >10000 |
| 20B | >10000 | >10000 |
| 21A | 93/99 | 938/721 |
| 21B | 517 | 4618 |
| 22A | 26 | 85 |
| 22B | 430 | 898 |
| 23 | 135/157 | 728/754 |
| 24B | 1915 | 7184 |
| 24A | 91 | 352 |
| 25 | 29/62 | 101/228 |
| 26 | 51/74 | 218/234 |
| 27 | 99 | 338 |
| 28A | 194 | 776 |
| 28B | >10000 | >10000 |
| 29 | 5421 | 4526 |
| 30 | 55 | 171 |
| 31 | 136 | 225 |
| 32 | 652 | 1135 |
| 33A | >10000 | >10000 |
| 33B | >10000 | >10000 |
| 34 | 8962 | >10000 |
| 35 | 4582 | >10000 |
| 36 | 1390 | 3907 |
| 37 | 546 | 3510 |
| 38 | >10000 | >10000 |
| 39 | >10000 | >10000 |
| 40 | 9110 | >10000 |
| 41 | 307 | 2794 |
| 42A | 26 | 134 |
| 42B | 12 | 74 |
| 42C | 162/27 | 101 |
| 42D | 2.2 | 12 |
| 43A | 4.6 | 15 |
| 43B | 61 | 108 |
| 44 | 490 | |
| 45 | 3394 | |
| 46 | 5.8 | 11 |
| 47 | 2.7 | 7.9 |
| 48 | 3.3 | 17 |
| 49 | 20 | 17 |
| 51 | 2.4 | 6.1 |
| 52A | 2002 | 3104 |
| 52B | >10000 | >10000 |
| 53 | 2183 | 3303 |
| 54 | 659 | 892 |
| 56 | 206 | 707 |
| 57A | 1138 | >10000 |
| 57B | >10000 | >10000 |

NT: Not tested

### Effect Example 3: 3D Cell Proliferation Assay

### 3D Proliferation Assay of AGS and AsPC-1 Cells

Using a nanoliter liquid handling system (LABCYTE, P-0200), the diluted test compounds were added to a 384-well low-adsorption cell culture plate. Cells were seeded into the wells, and the plate was placed in an incubator at 37° C. with 5% CO₂. After co-incubating the compounds and cells for 5 days, CellTiter-Glo® 3D reagent was added. Luminescence values were measured using an EnVision Multilabel Plate Reader (luminescence is proportional to the ATP content in the system, and ATP content directly reflects the number of viable cells). Finally, the IC₅₀ values (half-maximal inhibitory concentrations) of the compounds were determined using non-linear fitting equations in XLFit software with the following parameters:

$$Y = Bottom + ( Top - Bottom ) / ( 1 + {10}^{⋀} ( ( Log {IC}_{50} - X ) \times HillSlope ) )$$

X: Logarithm of compound concentration
Y: Inhibition rate (%)

$$Inhibition Rate ( \% ) = 100 \times ( Negative Control Mean - Compound Reading ) / ( Negative Control Mean - Positive Control Mean )$$

Negative control: DMSO
Positive control: Medium only

The 3D proliferation inhibition effect of the compounds described in the present invention in AGS and AsPC-1 cells are summarized in Table 3.

| | 3D Proliferation | 3D Proliferation |
|---|---|---|
| | Inhibition | Inhibition |
| Example | AGS_IC₅₀ (nM) | AsPC-1_IC₅₀ (nM) |
| 3 | 329 | 393 |
| 4 | 848 | 4867 |
| 5 | 128 | 196 |
| 6A | 127 | 202 |
| 6B | 1043 | 1373 |
| 7 | 2291 | 2865 |
| 8 | 254.99 | 682.33 |
| 9 | 51 | 613 |
| 9B | 93 | 222 |
| 16 | 83 | 288 |
| 21A | 226.42 | 2627.58 |
| 22A | 90 | 166 |
| 22B | 553 | 1787 |
| 23 | 346.13 | 2096.3 |
| 24A | 508.44 | 847.04 |
| 25 | 112 | 336 |
| 26 | 393 | 607 |
| 27 | 314 | 1001 |
| 28A | 430 | 1760 |
| 28B | 3364 | 3554 |
| 30 | 177 | 534 |
| 33A | 2311 | 1487 |
| 33B | 3081 | 4669 |
| 34 | 3655 | 3347 |
| 35 | 2694 | 2832 |
| 36 | 1883 | 2481 |
| 39 | 6386 | >10000 |
| 40 | 2054 | 2174 |
| 42A | 125 | 456 |
| 42B | 61 | 341 |
| 42D | 5.9 | 39 |
| 43A | 22/24 | 60/55 |
| 43B | 148 | 434 |
| 44 | 1859 | 3897 |
| 45 | 3212 | 4090 |
| 46 | 12 | 30 |
| 47 | 8.8 | 28 |
| 48 | 7.4 | 42 |
| 49 | 83 | 112 |
| 52B | >10000 | >10000 |
| 54 | 885 | 1262 |
| 56 | 668 | 1461 |

What is claimed is:

1. A heterocyclic compound represented by Formula I-1 or Formula I-2, a pharmaceutically acceptable salt thereof, a stereoisomer thereof, a deuterate thereof, or a solvate thereof: 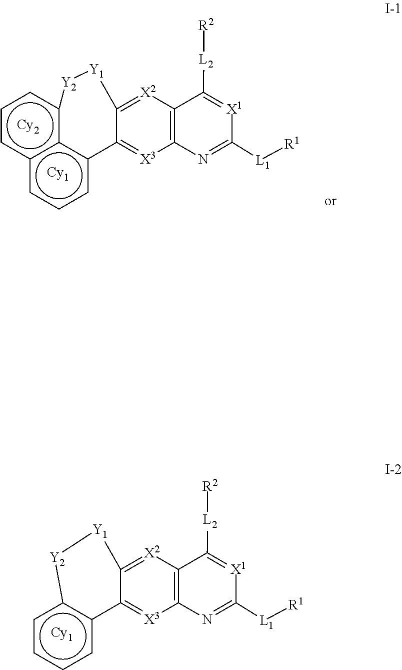
wherein, in Formula I-1 and Formula I-2, Cy₁ represents a 6-membered aryl group or a 6-membered heteroaryl group; further, Cy₁ is unsubstituted or substituted with 0-3 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein, in Formula I-1, Cy₂ represents a 6-membered aryl group or a 6-membered heteroaryl group; further, Cy₂ is unsubstituted or substituted with 0-3 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein X¹ is N or CR^{X1}; X² is N or CR^{X2}; X³ is N or CR^{X3};
wherein R^{X1}, R^{X2}, and R^{X3} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein L₁ and L₂ each independently represent absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀-C₆ alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR^{a}—(C₁-C₆ alkylene)-, —(C₁-C₆ alkylene)-NR^{a}—, —(C₁-C₆ alkylene)-C(O)—, —NR^{a}C(O)(C₁-C₆ alkylene)-, —(C₁-C₆ alkylene)-C(O)NR^{a}—, —O—(C₀-C₆ alkylene)-(CR^{T}R^{T′})—(C₀-C₆ alkylene)-, wherein R^{T} and R^{T′} together with a carbon atom attaching to the R^{T} and the R^{T′} form a 3-6 membered saturated or unsaturated ring containing 0, 1, or 2 heteroatoms selected from O, N, and S or not-containing any heteroatoms;
wherein R¹ and R² each independently represent a 5-16 membered saturated or unsaturated cycloalkyl or a 5-16 membered saturated or unsaturated heterocycloalkyl, and substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein Y¹ and Y² together form a C₁-C₁₀ alkylene or C₂-C₁₀ alkenylene, wherein CR^{a}R^{b} is unsubstituted or substituted with O, NH, NR^{a}, —C(O)—, —OC(O)—, —C(O)O—, —CONR^{a}—, —NR^{a}CO—, —N(S(O)₂CH₃), —N(C(O)CH₃)—, —S(O)—, —S(O)₂—, or —P(O)R^{a}—;
or, Y¹ and Y² together form a 5-10 membered saturated or unsaturated ring, a 6-10 membered aromatic ring, or a 5-10 membered heteroaromatic ring;
wherein R^{a} and R^{b} each independently represent hydrogen, C₁-C₆ alkyl, halogenated (C₁-C₆ alkyl), or C₃-C₆-cycloalkyl; or R^{a} and R^{b} together with an atom attaching to the R^{a} and the R^{b} form a 3-6 membered ring containing 0, 1, or 2 heteroatoms selected from O, N, and S or not-containing any heteroatoms.

2. A heterocyclic compound represented by Formula I-1′ or Formula I-2′, a pharmaceutically acceptable salt thereof, a stereoisomer thereof, a deuterate thereof, or a solvate thereof: 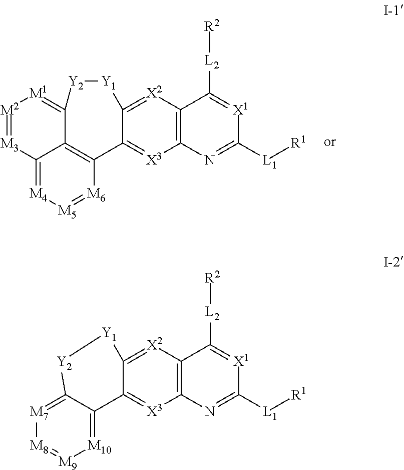
wherein, in Formula I-1′ and Formula I-2′,
M¹ represents CR^{M1} or N; M² represents CR^{M2} or N; M³ represents CR^{M3} or N; M⁴ represents CR^{M4} or N; M⁵ represents CR^{M5} or N; M⁶ represents CR^{M6} or N; M⁷ represents CR^{M7} or N; M⁸ represents CR^{M8} or N; M⁹ represents CR^{M9} or N; M¹⁰ represents CR^{M10} or N;
wherein each of R^{M1}, R^{M2}, R^{M3}, R^{M4}, R^{M5}, R^{M6}, R^{M7}, R^{M8}, R^{M9}, and R^{M10} independently represents hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}, which is unsubstituted or substituted with substituents;
wherein X¹ is N or CR^{X1}; X² is N or CR^{X2}; X³ is N or CR^{X3};
wherein R^{X1}, R^{X2}, and R^{X3} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein L₁ and L₂ each independently represent absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀-C₆ alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR—(C₁-C₆ alkylene)-, —(C₁-C₆ alkylene)-NR^{a}—, —(C₁-C₆ alkylene)-C(O)—, —NR^{a}C(O)(C₁-C₆ alkylene)-, —(C₁-C₆ alkylene)-C(O)NR^{a}—, —O—(C₀-C₆ alkylene)-(CR^{T}R^{T′})—(C₀-C₆ alkylene)-, wherein R^{T} and R^{T′} together with a carbon atom attaching to the R^{T} and the R^{T′} form a 3-6 membered saturated or unsaturated ring containing 0, 1, or 2 heteroatoms selected from O, N, and S or not-containing any heteroatoms;
wherein R¹ and R² each independently represent a 5-16 membered saturated or unsaturated cycloalkyl or a 5-16 membered saturated or unsaturated heterocycloalkyl, and substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
wherein Y¹ and Y² together form a C₁-C₁₀ alkylene or C₂-C₁₀ alkenylene, wherein CR^{a}R^{b} is unsubstituted or substituted with O, NH, NR^{a}, —C(O)—, —OC(O)—, —C(O)O—, —CONR^{a}—, —NR^{a}CO—, —N(S(O)₂CH₃)—, —N(C(O)CH₃)—, —S(O)—, —S(O)₂—, or —P(O)R^{a}—;
or, Y¹ and Y² together form a 5-10 membered saturated or unsaturated ring, a 6-10 membered aromatic ring, or a 5-10 membered heteroaromatic ring;
wherein R^{a} and R^{b} each independently represent hydrogen, C₁-C₆ alkyl, halogenated (C₁-C₆ alkyl), or C₃-C₆-cycloalkyl; or R^{a} and R^{b} together with an atom attaching to the R^{a} and the R^{b} form a 3-6 membered ring containing 0, 1, or 2 heteroatoms selected from O, N, and S or not-containing any heteroatoms.

3. The heterocyclic compound represented by Formula I-1′ or Formula I-2′ according to claim 2, the pharmaceutically acceptable salt thereof, the stereoisomer thereof, the deuterate thereof, or the solvate thereof, wherein X² represents CH or N.

4. The heterocyclic compound represented by Formula I-1′ or Formula I-2′ according to claim 2, the pharmaceutically acceptable salt thereof, the stereoisomer thereof, the deuterate thereof, or the solvate thereof, wherein X³ represents CR^{X3} or N, and R^{X3} represents hydrogen, halogen, —OR^{a}, cyano, —NO₂, —SF₅, —POMe₂, —NH₂, C₁-C₆ alkyl, halogenated (C₁-C₆ alkyl), halogenated (C₁-C₆ alkoxy), or hydroxy (C₁-C₆ alkyl).

5. The heterocyclic compound represented by Formula I-1′ or Formula I-2′ according to claim 2, the pharmaceutically acceptable salt thereof, the stereoisomer thereof, the deuterate thereof, or the solvate thereof, wherein L₁ represents absent, —O—(C₁-C₆ alkylene)-, —O—(C₀-C₆ alkylene)-(C₃-C₆ cycloalkyl)-(C₀-C₆ alkylene)-, —O—(C₀-C₆ alkylene)-(3-6 membered heterocycloalkyl)-(C₀-C₆ alkylene)-, —NR^{a}—(C₀-C₆ alkylene)-(C₃-C₆ cycloalkyl)-(C₀-C₆ alkylene)-, —NR^{a}—(C₀-C₆ alkylene)-(3-6 membered heterocycloalkyl)-(C₀-C₆ alkylene)-, —(C₀-C₆ alkylene)-(C₃-C₆ cycloalkyl)-(C₀—C alkylene)-NR^{a}—, —(C₀-C₆ alkylene)-(3-6 membered heterocycloalkyl)-(C₀-C₆ alkylene)-NR^{a}—, —(C₁-C₆ alkylene)-, —(C₁-C₆ alkylene)-O—, —NR^{a}—(C₁-C₆ alkylene)-, —(C₁-C₆ alkylene)-NR^{a}—, or —O—(C₀-C₆ alkylene)-(CR^{T}R^{T′})—(C₀-C₆ alkylene)-, wherein R^{T} and R^{T′} together with the carbon atom attaching to the R^{T} and the R^{T′} form the 3-6 membered saturated or unsaturated ring containing 0, 1, or 2 heteroatoms selected from O, N, and S or not-containing any heteroatoms.

6. The heterocyclic compound represented by Formula I-1′ or Formula I-2′ according to claim 2, the pharmaceutically acceptable salt thereof, the stereoisomer thereof, the deuterate thereof, or the solvate thereof, wherein L₁ represents —O—(C₁-C₆ alkylene)-.

7. The heterocyclic compound represented by Formula I-1′ or Formula I-2′ according to claim 2, the pharmaceutically acceptable salt thereof, the stereoisomer thereof, the deuterate thereof, or the solvate thereof, wherein L₁ represents —O—(C₀-C₆ alkylene)-(CR^{T}R^{T′})—(C₀-C₆ alkylene)-, wherein R^{T} and R^{T′} together with the carbon atom attaching to the R^{T} and the R^{T′} form a 3-6 membered saturated or unsaturated ring.

8. The heterocyclic compound represented by Formula I-1′ or Formula I-2′ according to claim 2, the pharmaceutically acceptable salt thereof, the stereoisomer thereof, the deuterate thereof, or the solvate thereof, wherein R¹ represents a 5-16 membered saturated or unsaturated heterocycloalkyl, which is unsubstituted or substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

9. The heterocyclic compound represented by Formula I-1′ or Formula I-2′ according to claim 2, the pharmaceutically acceptable salt thereof, the stereoisomer thereof, the deuterate thereof, or the solvate thereof, wherein R¹ has one of the following structures: 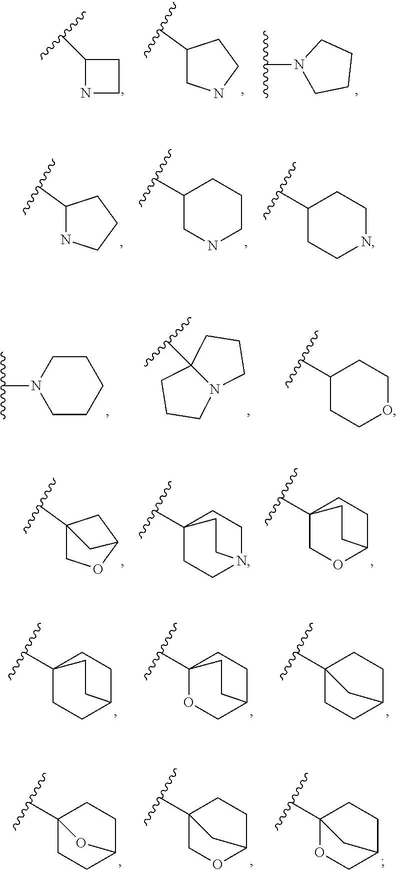
furthermore, R¹ is unsubstituted or substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), hydroxy (C₂-C₆ alkenyl group), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

10. The heterocyclic compound represented by Formula I-1′ or Formula I-2 according to claim 2, the pharmaceutically acceptable salt thereof, the stereoisomer thereof, the deuterate thereof, or the solvate thereof, wherein R¹ represents: 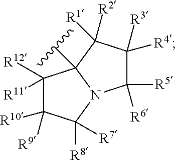
wherein R^{1′}, R^{2′}, R^{3′}, R⁴′, R^{5′}, R^{6′}, R^{7′}, R^{8′}, R^{9′}, R^{10′}, R^{11′}, and R^{12′} each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
or R^{1′} and R^{2′} together with a carbon atom attaching to the R^{1′} and the R^{2′} form a double bond (C═) or a C═O group, or R^{1′} and R^{2′} together with atoms attaching to the R^{1′} and the R^{2′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or R^{3′} and R^{4′} together with a carbon atom attaching to the R^{3′} and the R^{4′} form a double bond (C═) or a C═O group, or R^{3′} and R^{4′} together with atoms attaching to the R^{3′} and the R^{4′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or R^{5′} and R^{6′} together with a carbon atom attaching to the R^{5′} and the R^{6′} form a double bond (C═) or a C═O group, or R^{5′} and R^{6′} together with atoms attaching to the R^{5′} and the R^{6′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or R^{7′} and R^{8′} together with a carbon atom attaching to the R^{7′} and the R^{8′} form a double bond (C═) or a C═O group, or R^{7′} and R^{8′} together with atoms attaching to the R^{7′} and the R^{8′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or R^{9′} and R^{10′} together with a carbon atom attaching to the R^{9′} and the R^{10′} form a double bond (C═) or a C═O group, or R^{9′} and R^{10′} together with atoms attaching to the R^{9′} and the R^{10′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or R^{11′} and R^{12′} together with a carbon atom attaching to the R^{11′} and the R^{12′} form a double bond (C═) or a C═O group, or R^{11′} and R^{12′} together with atoms attaching to the R^{11′} and the R^{12′} form a substituted or unsubstituted 3-10 membered saturated or unsaturated ring containing 0, 1, or 2 heteroatoms selected from O, N, and S; the substituted or unsubstituted 3-10 membered saturated or unsaturated ring is further unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{2′} and R^{3′} together with atoms respectively attaching to the R^{2′} and the R^{3′}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{4′} and R^{5′} together with atoms respectively attaching to the R^{4′} and the R^{5′}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{8′} and R^{9′} together with atoms respectively attaching to the R^{8′} and the R^{9′}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{10′} and R^{11′} together with atoms respectively attaching to the R^{10′} and the R^{11′}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group.

11. The heterocyclic compound represented by Formula I-1′ or Formula I-2′ according to claim 10, the pharmaceutically acceptable salt thereof, the stereoisomer thereof, the deuterate thereof, or the solvate thereof, wherein R^{1′}, R^{2′}, R^{3′}, R^{4′}, R^{5′}, R^{6′}, R^{7′}, R^{8′}, R^{9′}, R^{10′}, R^{11′}, and R^{12′} each independently represent hydrogen, halogen, hydroxy, carbonyl, C₁-C₆ alkyl, halogenated (C₁-C₆ alkyl), halogenated (C₁-C₆ alkoxy), hydroxy (C₁-C₆ alkyl), C₂-C₆ alkenyl, or C₂-C₆ alkynyl.

12. The heterocyclic compound represented by Formula I-1′ or Formula I-2′ according to claim 10, the pharmaceutically acceptable salt thereof, the stereoisomer thereof, the deuterate thereof, or the solvate thereof, wherein R¹ has the following structure: 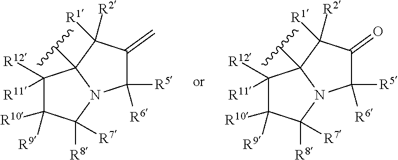
wherein R^{1′}, R^{2′}, R^{5′}, R^{6′}, R^{7′}, R^{8′}, R^{9′}, R^{10′}, R^{11′}, and R^{12′} are defined as in claim 10.

13. The heterocyclic compound represented by Formula I-1′ or Formula I-2′ according to claim 10, the pharmaceutically acceptable salt thereof, the stereoisomer thereof, the deuterate thereof, or the solvate thereof, wherein R¹ has the following structure: 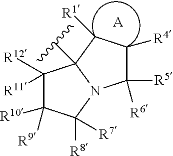
wherein ring A is a 3-6 membered ring, substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}
wherein R^{1′}, R^{4′}, R^{5′}, R^{6′}, R^{7′}, R^{8′}, R^{9′}, R^{10′}, R^{11′}, and R^{12′} are defined as in claim 10.

14. The heterocyclic compound represented by Formula I-1′ or Formula I-2′ according to claim 10, the pharmaceutically acceptable salt thereof, the stereoisomer thereof, the deuterate thereof, or the solvate thereof, wherein R¹ has the following structure: 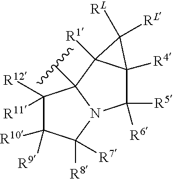
wherein R^{1′}, R^{2′}, R^{5′}, R^{6′}, R^{7′}, R^{8′}, R^{9′}, R^{10′}, R^{11′}, and R^{12′} are defined as in claim 10, and R^{L} and R^{L′} each independently represent hydrogen, C₁-C₆ alkyl, or halogen.

15. The heterocyclic compound represented by Formula I-1′ or Formula I-2′ according to claim 2, the pharmaceutically acceptable salt thereof, the stereoisomer thereof, the deuterate thereof, or the solvate thereof, wherein R¹ has the following structure:
wherein R^{1′}, R^{2′}, R^{5′}, R^{6′}, R^{7′}, R^{8′}, R^{9′}, R^{10′}, R^{11′}, and R^{12′} each independently represent hydrogen, halogen, hydroxy, C₁-C₆ alkyl, halogenated (C₁-C₆ alkyl), halogenated (C₁-C₆ alkoxy), or hydroxy (C₁-C₆ alkyl); R^{3′} and R^{4′} together with atoms respectively attaching to the R^{3′} and the R^{4′} form a 3-6 membered ring, containing 0, 1, or 2 heteroatoms selected from O, N, and S; further, the 3-6 membered ring is unsubstituted or substituted with 0-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}:

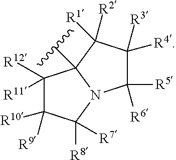

16. The heterocyclic compound represented by Formula I-1′ or Formula I-2′ according to claim 2, the pharmaceutically acceptable salt thereof, the stereoisomer thereof, the deuterate thereof, or the solvate thereof, wherein L₂ represents absent, —O—(C₁-C₆ alkylene)-, —(C₁-C₆ alkylene)-, —(C₁-C₆ alkylene)-O—, —NR^{a}—(C₁-C₆ alkylene)-, or —(C₁-C₆ alkylene)-NR^{a}—.

17. The heterocyclic compound represented by Formula I-1′ or Formula I-2′ according to claim 2, the pharmaceutically acceptable salt thereof, the stereoisomer thereof, the deuterate thereof, or the solvate thereof, wherein L₂ represents absence.

18. The heterocyclic compound represented by Formula I-1′ or Formula I-2′ according to claim 2, the pharmaceutically acceptable salt thereof, the stereoisomer thereof, the deuterate thereof, or the solvate thereof, wherein R² is acyclic structure of Formula (i), Formula (ii), or Formula (iii): 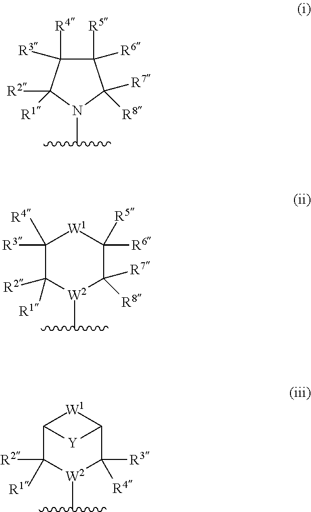
wherein W¹ represents —(CR^{W1}R^{W2})ₚ—, —(CR^{W1}R^{W2}), —O—, —O—(CR^{W1}R^{W2})ₚ—, —NR^{a}—(CR^{W1}R^{W2})ₚ—, —(CR^{W1}R^{W2})ₚ—NR^{a}—, —CH═CH—, or —NR^{W1};
wherein R^{W1} and R^{W2}, each independently, represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}; or R^{W1} and R^{W2} together with a carbon atom attaching to the R^{W1} and the R^{W2}, form a 3-8 membered saturated or unsaturated ring containing 0, 1, or 2 heteroatoms selected from O, N, and S; furthermore, the 3-8 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the 3-8 membered saturated or unsaturated ring is unsubstituted or substituted with a C═O group;
wherein R^{1″}, R^{2″}, R^{3″}, R^{4″}, R^{5″}, R^{6″}, R^{7″} and Rr each independently represent hydrogen, C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, and —NR^{a}R^{b};
or, R^{1″} and R^{2″} together with atoms respectively attaching to the R^{1″} and the R^{2″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{3″} and R^{4″} together with atoms respectively attaching to the R^{3″} and the R^{4″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{5″} and R^{6″} together with atoms respectively attaching to the R^{5″} and the R^{6″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{7″} and R^{8″} together with atoms respectively attaching to the R^{7″} and the R^{8″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{2″} and R^{3″} together with atoms respectively attaching to the R^{2″} and the R^{3″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{4″} and R^{5″} together with atoms respectively attaching to the R^{4″} and the R^{5″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{6″} and R^{7″} together with atoms respectively attaching to the R^{6″} and the R^{7″}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆ cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;
or, R^{5″} and R^{W1} together with atoms respectively attaching to the R^{5″} and the R^{W1}, form a substituted or unsubstituted 3-10 membered saturated or unsaturated ring, containing 0, 1, or 2 heteroatoms selected from O, N, and S; furthermore, the substituted or unsubstituted 3-10 membered saturated or unsaturated ring is unsubstituted or substituted with 1-2 substituents selected from halogen, cyano, C₁-C₆ alkyl, C₂-C₆ alkenyl, C₂-C₆ alkynyl, halogenated (C₁-C₆ alkyl), halogenated (C₁-C₆ alkoxy), hydroxy (C₁-C₆ alkyl), C₃-C₆ cycloalkyl, C₃-C₆cycloalkenyl, 3-6 membered heterocycloalkyl, 3-6 membered heterocycloalkenyl, —OR^{a}, —SO₃R^{a}, —NR^{a}R^{b}, —C(O)R^{a}, —C(O)OR^{a}, —C(O)NR^{a}R^{b}, or —NR^{a}C(O)R^{a}; or CH₂ in the substituted or unsubstituted 3-10 membered saturated or unsaturated ring is unsubstituted or substituted with a C═O group;

19. The heterocyclic compound represented by Formula I-1′ or Formula I-2′ according to claim 2, the pharmaceutically acceptable salt thereof, the stereoisomer, the deuterate thereof, or the solvate thereof, wherein R² is one of the following cyclic structures: 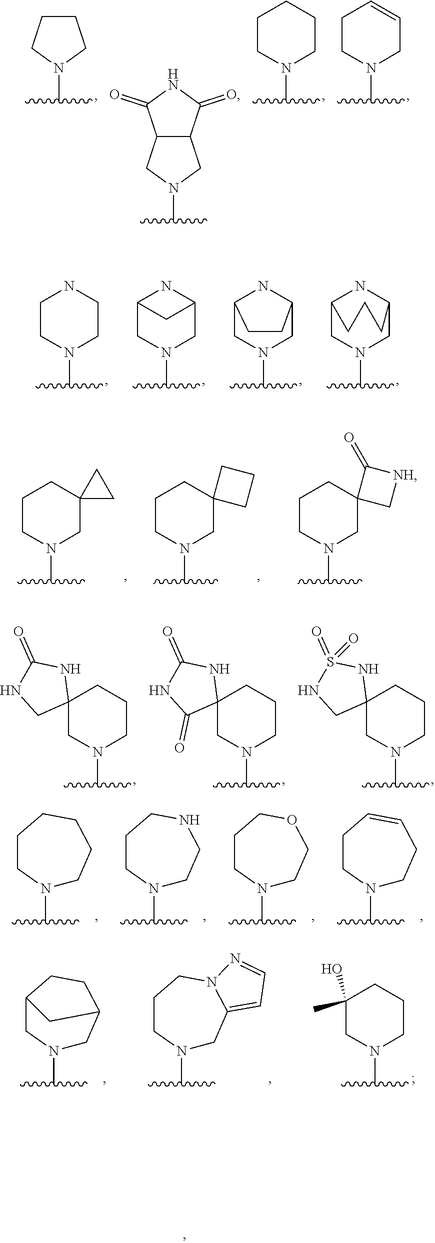
furthermore, R² is unsubstituted or substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

20. The heterocyclic compound represented by Formula I-1′ or Formula I-2′ according to claim 2, the pharmaceutically acceptable salt thereof, the stereoisomer, the deuterate thereof, or the solvate thereof, wherein R² is one of the following cyclic structures: 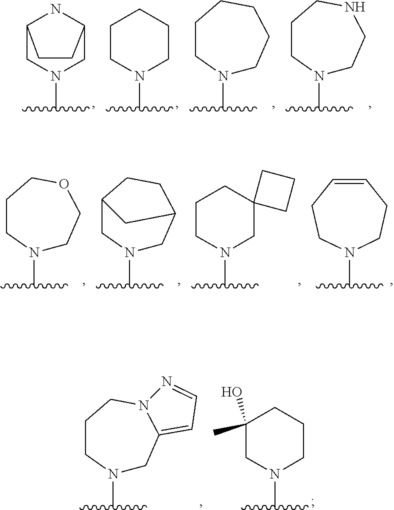
furthermore, R² is unsubstituted or substituted with 0-4 substituents selected from C₁-C₆ alkyl, C₂-C₆ alkenyl, C₂-C₆ alkynyl, C₃-C₆ cycloalkyl, C₃-C₆ cycloalkenyl, 3-6 membered heterocycloalkyl, 3-6 membered heterocycloalkenyl, halogen, halogenated (C₁-C₆ alkyl), halogenated (C₁-C₆ alkoxy), hydroxy (C₁-C₆ alkyl), —OR^{a}, —C(O)R^{a}, —OC(O)R^{a}, —C(O)OR^{a}, —NO₂, —SF₅, —SO₃R^{a}, —S(O)₂R^{a}, cyano, —C(O)NR^{a}R^{b}, —NR^{a}C(O)R^{a}, or —NR^{a}R^{b}.

21. The heterocyclic compound represented by Formula I-1′ or Formula I-2′ according to claim 2, the pharmaceutically acceptable salt thereof, the stereoisomer thereof, the deuterate thereof, or the solvate thereof, wherein R² is one of the following cyclic structures:

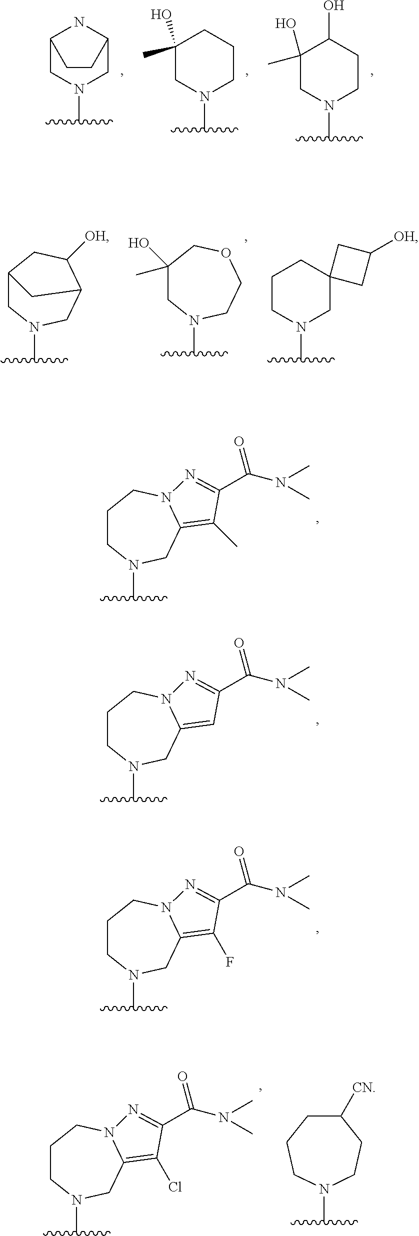

22. The heterocyclic compound represented by Formula I-1′ or Formula I-2′ according to claim 2, the pharmaceutically acceptable salt thereof, the stereoisomer thereof, the deuterate thereof, or the solvate thereof, wherein M¹ to M¹⁰ each represent CH or N.

23. The heterocyclic compound represented by Formula I-1′ or Formula I-2′ according to claim 2, the pharmaceutically acceptable salt thereof, the stereoisomer thereof, the deuterate thereof, or the solvate thereof, wherein M¹ to M¹⁰ each represent CH.

24. The heterocyclic compound represented by Formula I-1′ or Formula I-2′ according to claim 2, the pharmaceutically acceptable salt thereof, the stereoisomer thereof, the deuterate thereof, or the solvate thereof, wherein:
Y¹ and Y² together form a C₁-C₁₀ alkylene or a C₂-C₁₀ alkenylene, and CR^{a}R^{b} is substituted with O, NH, —C(O)—, —OC(O)—, —C(O)O—, —S(O)—, —S(O)₂—, or —P(O)R^{a}—.

25. The heterocyclic compound represented by Formula I-1′ or Formula I-2′ according to claim 24, the pharmaceutically acceptable salt thereof, the stereoisomer thereof, the deuterate thereof, or the solvate thereof, wherein Y¹ and Y² together form: —(C₁-C₁₀ alkylene)-NR^{a}—, —O—(C₁-C₁₀ alkylene)-NR^{a}—, —NR^{a}(C₁-C₁₀ alkylene)-O—, —(C₁-C₁₀ alkylene)-O—, —(C₁-C₁₀ alkylene)-C(O)NR^{a}—, —(C₁-C₁₀ alkylene)-NR^{a}C(O)—, —(C₁-C₁₀ alkylene)-O—(C₁-C₁₀ alkylene)-O—, —(C₁-C₁₀ alkylene)-O—(C₁-C₁₀ alkylene)-, —(C₁-C₁₀ alkylene)-NR^{a}—(C₁-C₁₀ alkylene)-, —(C₁-C₁₀ alkylene)-NR^{a}—(C₁-C₁₀ alkylene)-O—, —(C₁-C₁₀ alkylene)-O—(C₁-C₁₀ alkylene)-NR^{a}—, —NR^{a}—(C₁-C₁₀ alkylene)-, —O—(C₁-C₁₀ alkylene)-, —C(O)NR^{a}—(C₁-C₁₀ alkylene)-, —NR^{a}C(O)—(C₁-C₁₀ alkylene)-, —(C₁-C₁₀ alkylene)-O—(C₁-C₁₀ alkylene)-, —O—(C₁-C₁₀ alkylene)-O—(C₁-C₁₀ alkylene)-, —(C₁-C₁₀ alkylene)-O—(C₁-C₁₀ alkylene)-C(O)—, —(C₁-C₁₀ alkylene)-C(O)—, —O—(C₁-C₁₀ alkylene)-C(O)—, —C(O)—(C₁-C₁₀ alkylene)-, —C(O)—(C₁-C₁₀ alkylene)-O—, —O—(C₁-C₁₀ alkylene)-C(O)NR^{a}—, —C(O)NR^{a}—(C₁-C₁₀ alkylene)-O—, —(C₁-C₁₀ alkylene)-S—, or —S—(C₁-C₁₀ alkylene)-.

26. The heterocyclic compound represented by Formula I-1′ or Formula I-2′ according to claim 2, the pharmaceutically acceptable salt thereof, the stereoisomer thereof, the deuterate thereof, or the solvate thereof, wherein Y¹ and Y² together form a 5-10 membered saturated or unsaturated ring, a 6-10 membered aromatic ring, or a 5-10 membered heteroaromatic ring.

27. The heterocyclic compound represented by Formula I-1′ or Formula I-2′ according to claim 2, the pharmaceutically acceptable salt thereof, the stereoisomer thereof, the deuterate thereof, or the solvate thereof, wherein Y¹ and Y² together form one of the following structures:

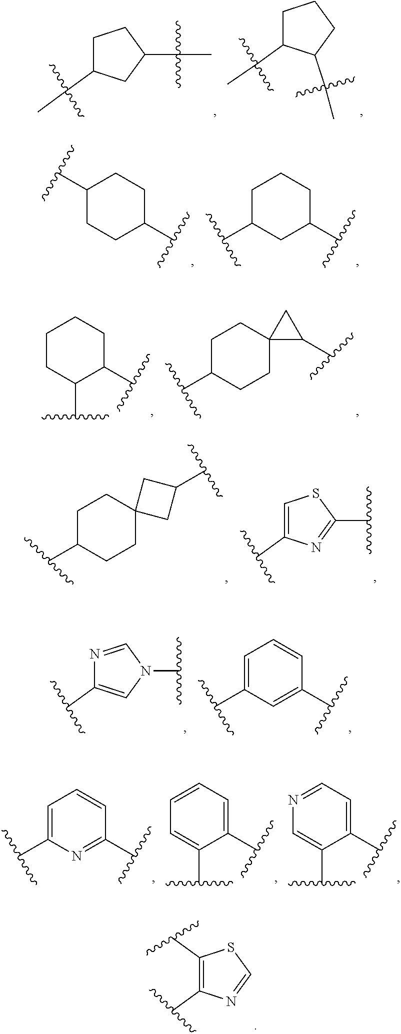

28. A compound, a pharmaceutically acceptable salt thereof, a stereoisomer thereof, a deuterate thereof, or a solvate thereof, having the following structure: 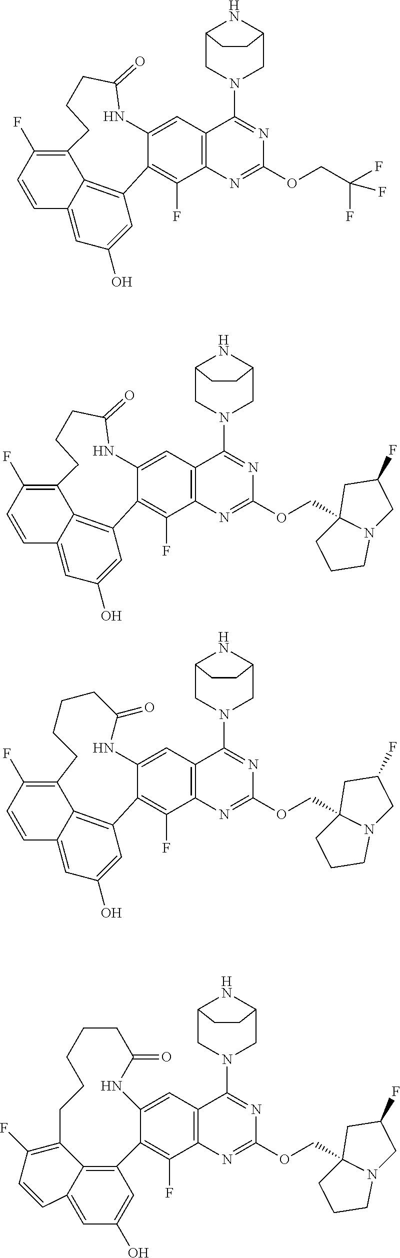 [SMILES: O=C1CCCc2c(F)ccc3cc(O)cc(c23)-c2c(cc3c(N4CC5CCC(C4)N5)nc(OCC(F)(F)F)nc3c2F)N1 ; O=C1CCCc2c(F)ccc3cc(O)cc(c23)-c2c(cc3c(N4CC5CCC(C4)N5)nc(OC[C@@]45CCCN4C[C@H](F)C5)nc3c2F)N1 ; O=C1CCCCc2c(F)ccc3cc(O)cc(c23)-c2c(cc3c(N4CC5CCC(C4)N5)nc(OC[C@@]45CCCN4C[C@@H](F)C5)nc3c2F)N1 ; O=C1CCCCCc2c(F)ccc3cc(O)cc(c23)-c2c(cc3c(N4CC5CCC(C4)N5)nc(OC[C@@]45CCCN4C[C@H](F)C5)nc3c2F)N1] 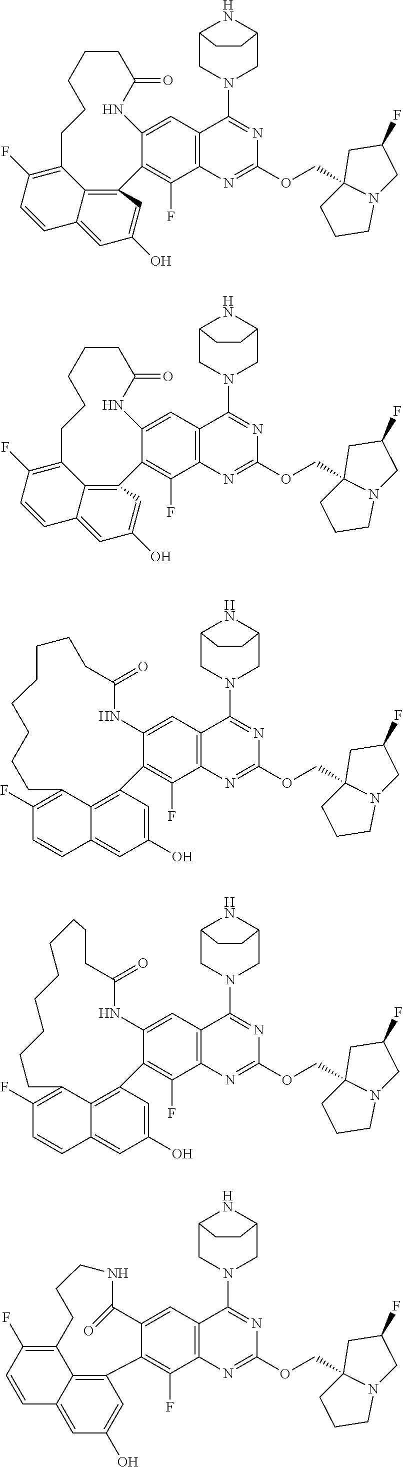 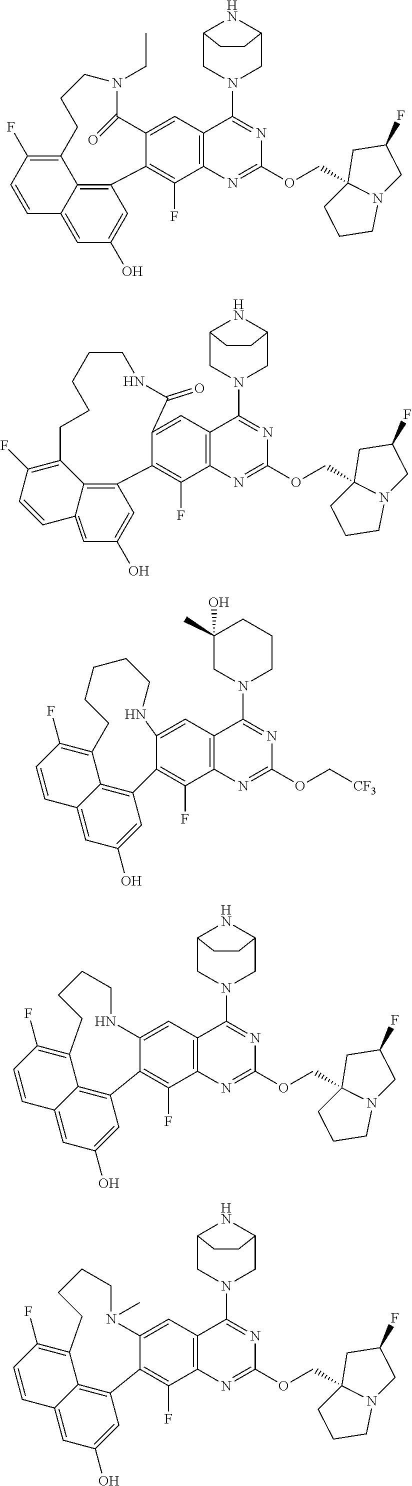 [SMILES: CCN1CCCc2c(F)ccc3cc(O)cc(c23)-c2c(cc3c(N4CC5CCC(C4)N5)nc(OC[C@@]45CCCN4C[C@H](F)C5)nc3c2F)C1=O ; O=C1NCCCCCc2c(F)ccc3cc(O)cc(c23)-c2c1cc1c(N3CC4CCC(C3)N4)nc(OC[C@@]34CCCN3C[C@H](F)C4)nc1c2F ; C[C@@]1(O)CCCN(c2nc(OCC(F)(F)F)nc3c(F)c4c(cc23)NCCCCCc2c(F)ccc3cc(O)cc-4c23)C1 ; Oc1cc2c3c(c(F)ccc3c1)CCCCNc1cc3c(N4CC5CCC(C4)N5)nc(OC[C@@]45CCCN4C[C@H](F)C5)nc3c(F)c1-2 ; CN1CCCCc2c(F)ccc3cc(O)cc(c23)-c2c1cc1c(N3CC4CCC(C3)N4)nc(OC[C@@]34CCCN3C[C@H](F)C4)nc1c2F] 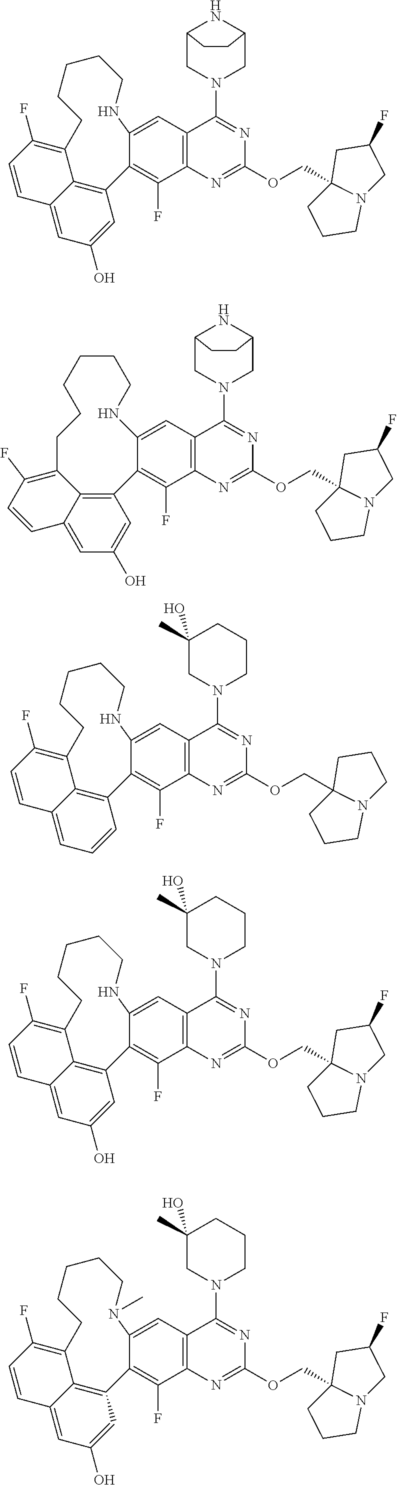 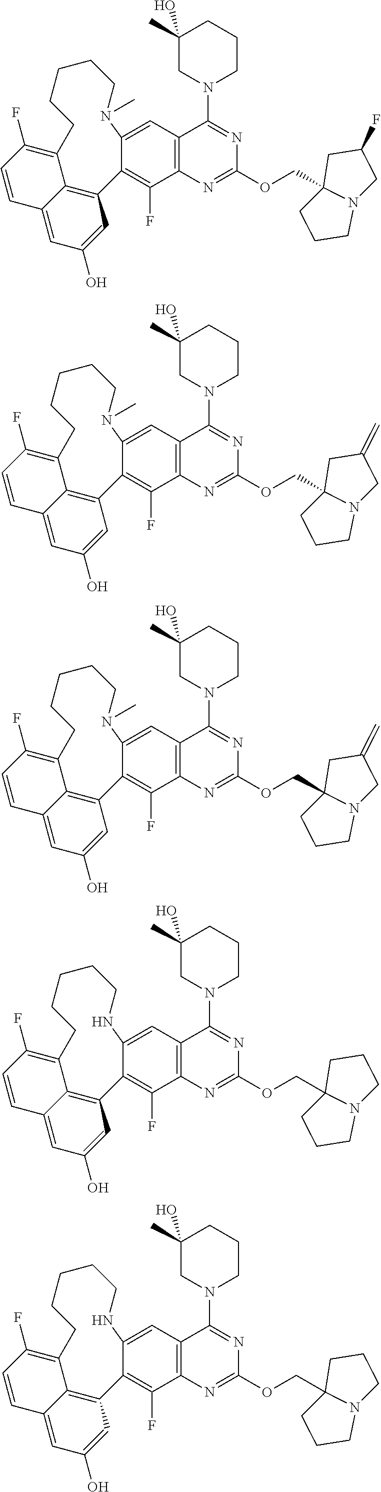 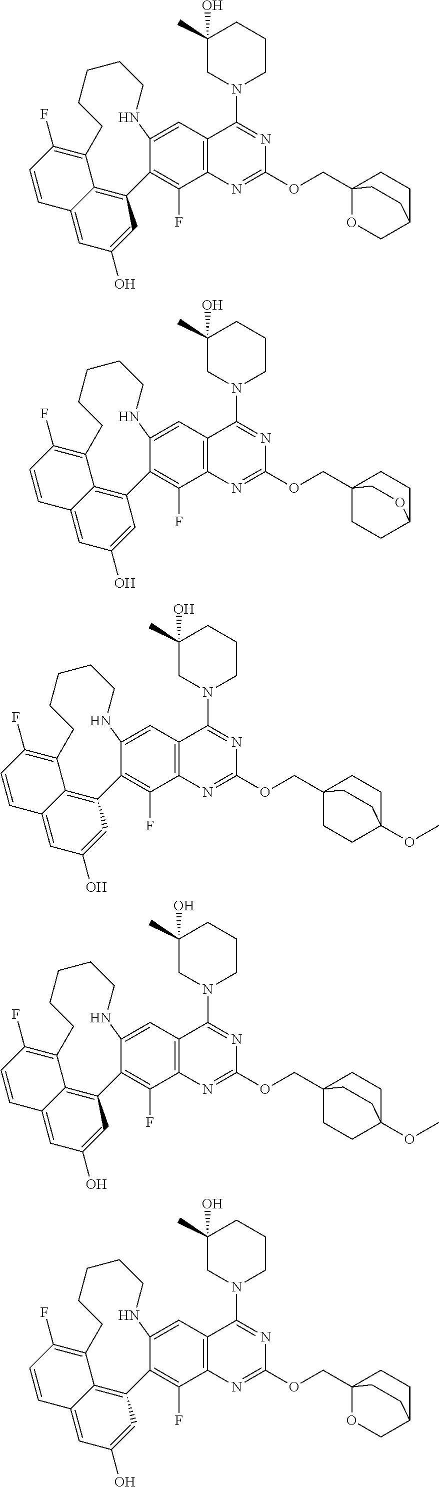 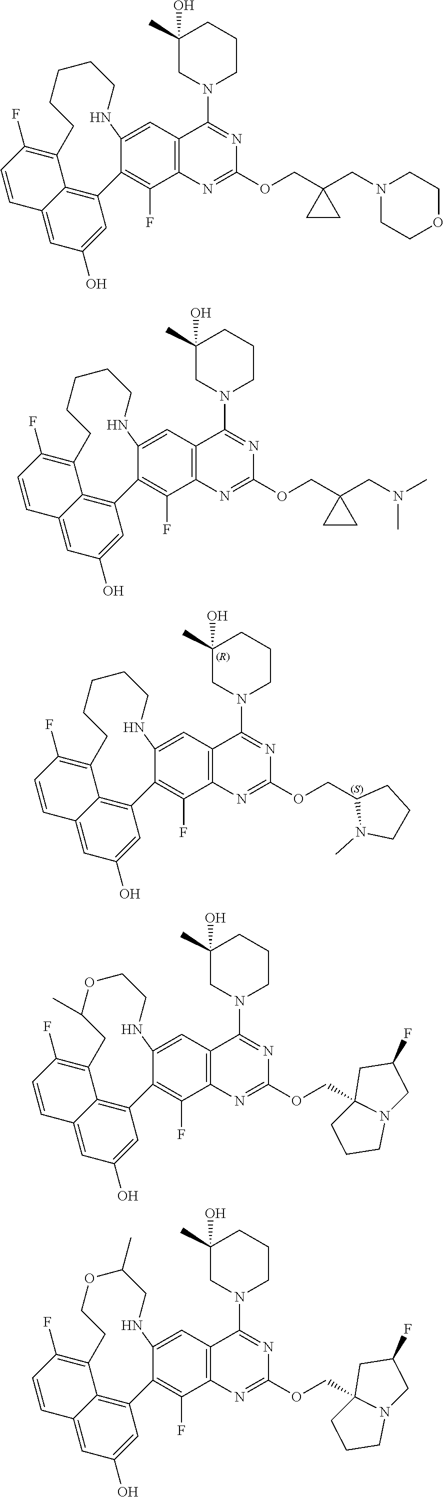 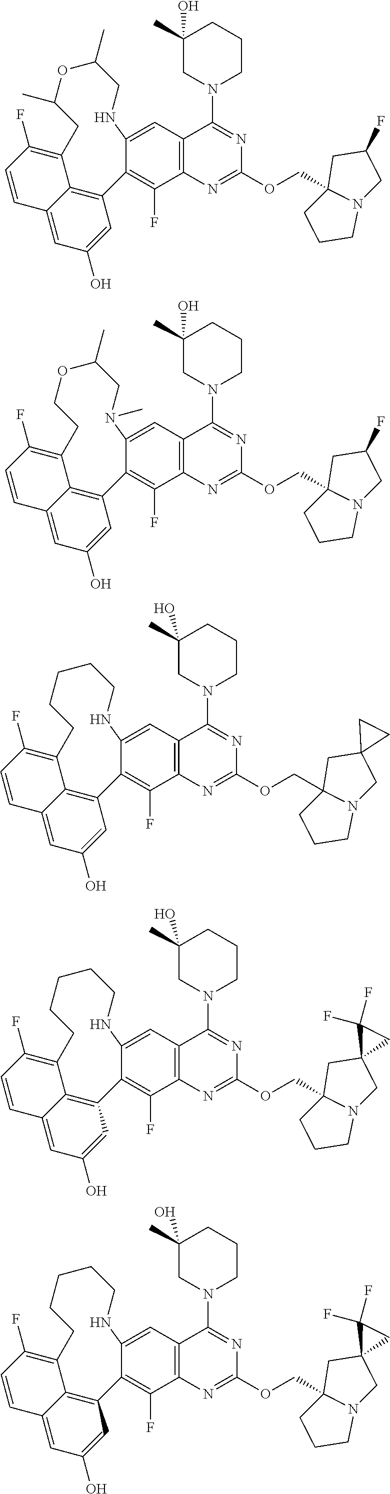 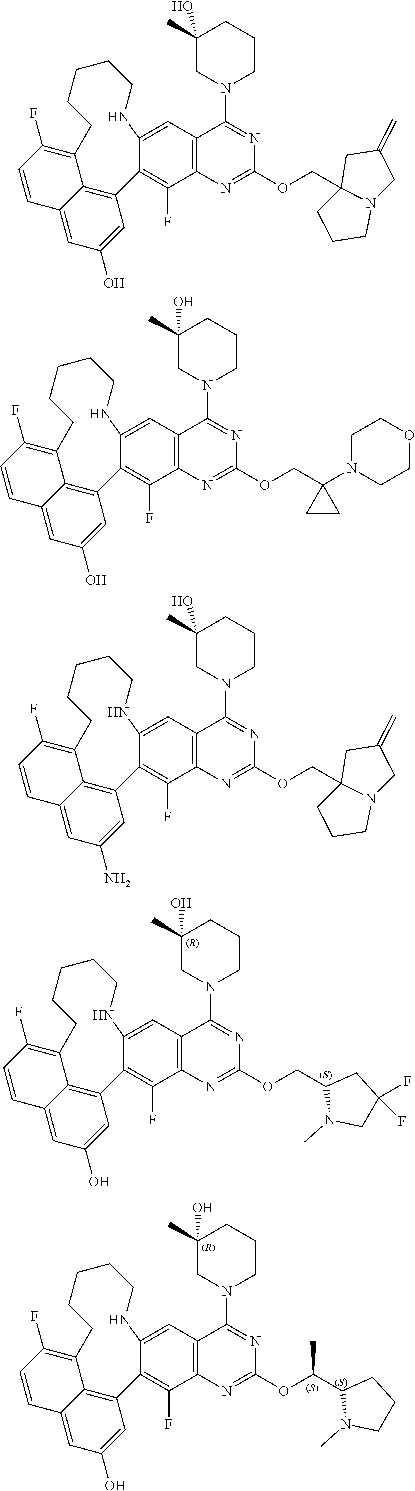 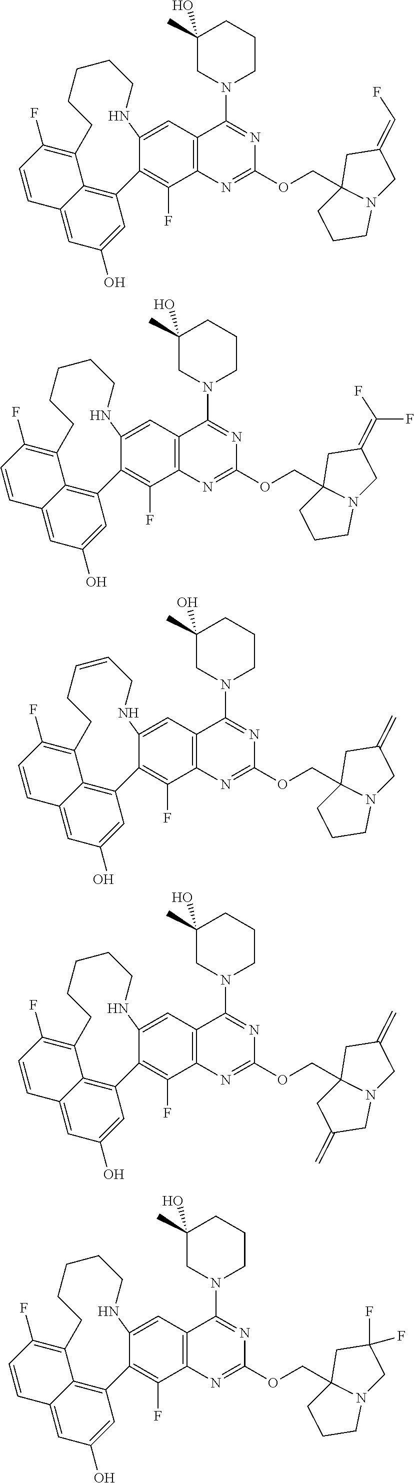 [SMILES: C[C@@]1(O)CCCN(c2nc(OCC34CCCN3C/C(=C/F)C4)nc3c(F)c4c(cc23)NCCCCCc2c(F)ccc3cc(O)cc-4c23)C1 ; C[C@@]1(O)CCCN(c2nc(OCC34CCCN3CC(=C(F)F)C4)nc3c(F)c4c(cc23)NCCCCCc2c(F)ccc3cc(O)cc-4c23)C1 ; C=C1CN2CCCC2(COc2nc(N3CCC[C@@](C)(O)C3)c3cc4c(c(F)c3n2)-c2cc(O)cc3ccc(F)c(c23)CC/C=C\CN4)C1 ; C=C1CN2CC(=C)CC2(COc2nc(N3CCC[C@@](C)(O)C3)c3cc4c(c(F)c3n2)-c2cc(O)cc3ccc(F)c(c23)CCCCCN4)C1 ; C[C@@]1(O)CCCN(c2nc(OCC34CCCN3CC(F)(F)C4)nc3c(F)c4c(cc23)NCCCCCc2c(F)ccc3cc(O)cc-4c23)C1] 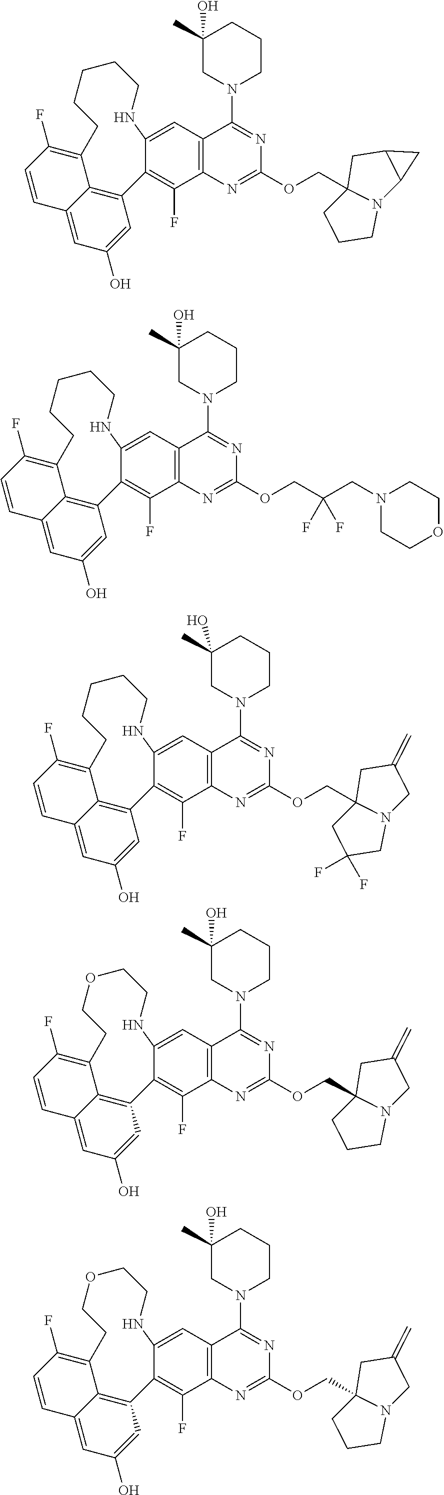 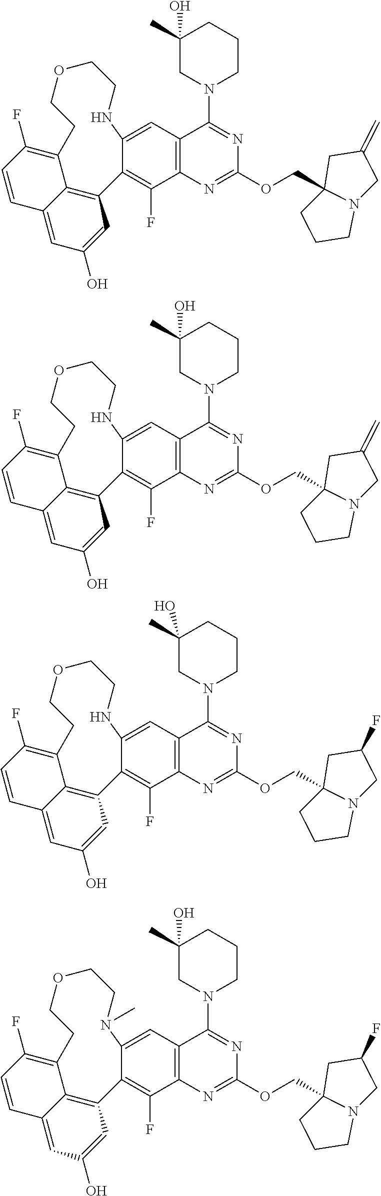 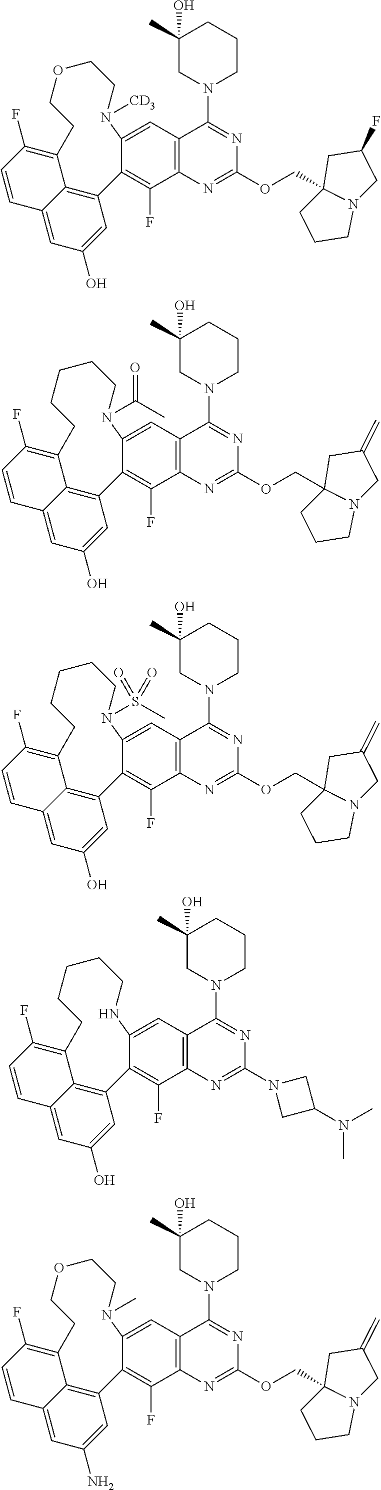 [SMILES: [2H]C([2H])([2H])N1CCOCCc2c(F)ccc3cc(O)cc(c23)-c2c1cc1c(N3CCC[C@@](C)(O)C3)nc(OC[C@@]34CCCN3C[C@H](F)C4)nc1c2F ; C=C1CN2CCCC2(COc2nc(N3CCC[C@@](C)(O)C3)c3cc4c(c(F)c3n2)-c2cc(O)cc3ccc(F)c(c23)CCCCCN4C(C)=O)C1 ; C=C1CN2CCCC2(COc2nc(N3CCC[C@@](C)(O)C3)c3cc4c(c(F)c3n2)-c2cc(O)cc3ccc(F)c(c23)CCCCCN4S(C)(=O)=O)C1 ; CN(C)C1CN(c2nc(N3CCC[C@@](C)(O)C3)c3cc4c(c(F)c3n2)-c2cc(O)cc3ccc(F)c(c23)CCCCCN4)C1 ; C=C1CN2CCC[C@@]2(COc2nc(N3CCC[C@@](C)(O)C3)c3cc4c(c(F)c3n2)-c2cc(N)cc3ccc(F)c(c23)CCOCCN4C)C1] 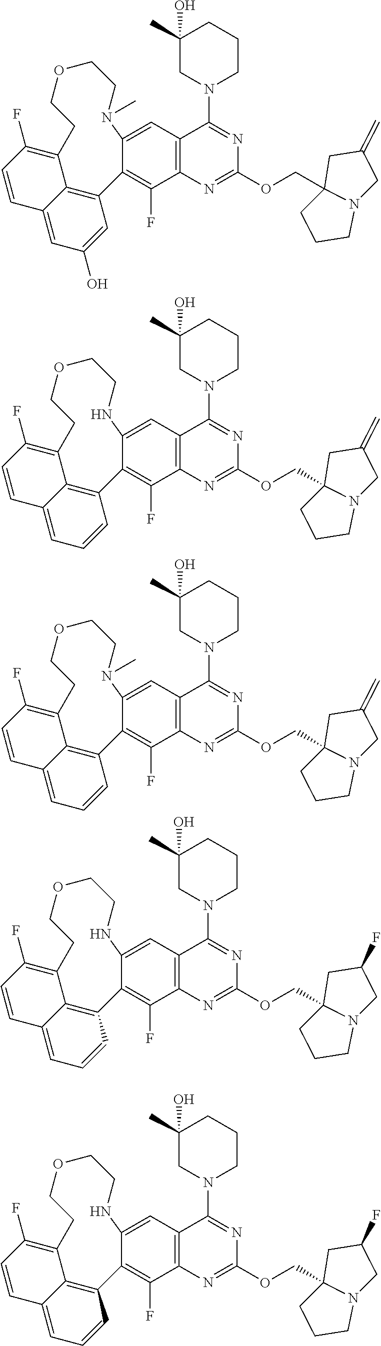 [chemical structure]
